(19) United States
(12) Patent Application Publication
(10) Pub. No.: US 2023/0021448 A1
(43) Pub. Date: Jan. 26, 2023

(54) COMPOUNDS AND COMPOSITIONS FOR TREATING CONDITIONS ASSOCIATED WITH STING ACTIVITY

(71) Applicant: IFM Due, Inc., Boston, MA (US)

(72) Inventors: Hans Martin Seidel, Concord, MA (US); William R. Roush, Boston, MA (US); Jason Katz, Newton, MA (US); Shankar Venkatraman, Lansdale, PA (US)

(21) Appl. No.: 17/766,149

(22) PCT Filed: Oct. 2, 2020
(86) PCT No.: PCT/US2020/054069

§ 371 Date: Apr. 1, 2022

Related U.S. Application Data
(60) Provisional application No. 62/910,160, filed on Oct. 3, 2019.
(60) Provisional application No. 62/955,867, filed on Dec. 31, 2019.

(51) Int. Cl. C07D 209/40; C07D 401/12; C07D 401/14; C07D 403/04; C07D 498/04; C07D 403/12; C07D 471/04; C07D 405/12; C07D 495/04; C07D 413/12; C07D 491/107

(52) U.S. Cl. CPC C07D 209/40; C07D 401/12; C07D 401/14; C07D 403/04; C07D 498/04; C07D 403/12; C07D 471/04; C07D 405/12; C07D 495/04; C07D 413/12; C07D 491/107

(57) ABSTRACT

This disclosure features chemical entities (e.g., a compound or a pharmaceutically acceptable salt, and/or hydrate, and/or cocrystal, and/or drug combination of the compound) that inhibit (e.g., antagonize) Stimulator of Interferon Genes (STING). Said chemical entities are useful, e.g., for treating a condition, disease or disorder in which increased (e.g., excessive) STING activation (e.g., STING signaling) contributes to the pathology and/or symptoms and/or progression of the condition, disease or disorder (e.g., cancer) in a subject (e.g., a human). This disclosure also features compositions containing the same as well as methods of using and making the same.

### CROSS REFERENCE TO RELATED APPLICATIONS

This application claims the benefit of U.S. Provisional Application Ser. No. 62/910,160, filed on Oct. 3, 2019; and U.S. Provisional Application Ser. No. 62/955,867, filed on Dec. 31, 2019; each of which is incorporated herein by reference in its entirety.

### TECHNICAL FIELD

This disclosure features chemical entities (e.g., a compound or a pharmaceutically acceptable salt, and/or hydrate, and/or cocrystal, and/or prodrug, and/or tautomer, and/or drug combination of the compound) that inhibit (e.g., antagonize) Stimulator of Interferon Genes (STING). Said chemical entities are useful, e.g., for treating a condition, disease or disorder in which increased (e.g., excessive) STING activation (e.g., STING signaling) contributes to the pathology and/or symptoms and/or progression of the condition, disease or disorder (e.g., cancer) in a subject (e.g., a human). This disclosure also features compositions containing the same as well as methods of using and making the same.

### BACKGROUND

STING, also known as transmembrane protein 173 (TMEM173) and MPYS/MITA/ERIS, is a protein that in humans is encoded by the TMEM173 gene. STING has been shown to play a role in innate immunity. STING induces type I interferon production when cells are infected with intracellular pathogens, such as viruses, mycobacteria and intracellular parasites. Type I interferon, mediated by STING, protects infected cells and nearby cells from local infection in an autocrine and paracrine manner.

The STING pathway is pivotal in mediating the recognition of cytosolic DNA. In this context, STING, a transmembrane protein localized to the endoplasmic reticulum (ER), acts as a second messenger receptor for 2′, 3′ cyclic GMP-AMP (hereafter cGAMP), which is produced by cGAS after dsDNA binding. In addition, STING can also function as a primary pattern recognition receptor for bacterial cyclic dinucleotides (CDNs) and small molecule agonists. The recognition of endogenous or prokaryotic CDNs proceeds through the carboxy-terminal domain of STING, which faces into the cytosol and creates a V-shaped binding pocket formed by a STING homodimer. Ligand-induced activation of STING triggers its re-localization to the Golgi, a process essential to promote the interaction of STING with TBK1. This protein complex, in turn, signals through the transcription factors IRF-3 to induce type I interferons (IFNs) and other co-regulated antiviral factors. In addition, STING was shown to trigger NF-κB and MAP kinase activation. Following the initiation of signal transduction, STING is rapidly degraded, a step considered important in terminating the inflammatory response.

Excessive activation of STING is associated with a subset of monogenic autoinflammatory conditions, the so-called type I interferonopathies. Examples of these diseases include a clinical syndrome referred to as STING-associated vasculopathy with onset in infancy (SAVI), which is caused by gain-of-function mutations in TMEM173 (the gene name of STING). Moreover, STING is implicated in the pathogenesis of Aicardi-Goutieres Syndrome (AGS) and genetic forms of lupus. As opposed to SAVI, it is the dysregulation of nucleic acid metabolism that underlies continuous innate immune activation in AGS. Apart from these genetic disorders, emerging evidence points to a more general pathogenic role for STING in a range of inflammation-associated disorders such as systemic lupus erythematosus, rheumatoid arthritis and cancer. Thus, small molecule-based pharmacological interventions into the STING signaling pathway hold significant potential for the treatment of a wide spectrum of diseases

### SUMMARY

This disclosure features chemical entities (e.g., a compound or a pharmaceutically acceptable salt, and/or hydrate, and/or cocrystal, and/or prodrug, and/or tautomer, and/or drug combination of the compound) that inhibit (e.g., antagonize) Stimulator of Interferon Genes (STING). Said chemical entities are useful, e.g., for treating a condition, disease or disorder in which increased (e.g., excessive) STING activation (e.g., STING signaling) contributes to the pathology and/or symptoms and/or progression of the condition, disease or disorder (e.g., cancer) in a subject (e.g., a human). This disclosure also features compositions containing the same as well as methods of using and making the same.

An “antagonist” of STING includes compounds that, at the protein level, directly bind or modify STING such that an activity of STING is decreased, e.g., by inhibition, blocking or dampening agonist-mediated responses, altered distribution, or otherwise. STING antagonists include chemical entities, which interfere or inhibit STING signaling.

In one aspect, compounds of Formula (I), or a pharmaceutically acceptable salt thereof, are featured:

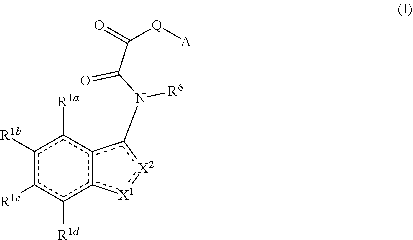

in which R^{1a}, R^{1b}, R^{1c}, R^{1d}, X¹, X², Q, A, and R⁶ can be as defined anywhere herein; and

each [symbol] is independently a single bond or a double bond, provided that the five-membered ring comprising X¹ and X² is heteroaryl (i.e., one or more of X¹ and X² is an independently selected heteroatom; and the 5-membered ring comprising X¹, and X² is aromatic (as a non-limiting example, the ring comprising X¹ and X² can be pyrrole)).

In one aspect, compounds of Formula (I), or a pharmaceutically acceptable salt thereof, or a prodrug thereof, or a tautomer thereof, or any combination of the foregoing, are featured “Prodrug” is meant to indicate a compound that may be converted under physiological conditions or by solvolysis to a biologically active compound described herein (e.g., compound of Formula (I)). Thus, the term “prodrug” refers to a precursor of a biologically active compound that is pharmaceutically acceptable. In some aspects, a prodrug is inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein.

In one aspect, pharmaceutical compositions are featured that include a chemical entity described herein (e.g., a compound described generically or specifically herein or a pharmaceutically acceptable salt thereof or compositions containing the same) and one or more pharmaceutically acceptable excipients.

In one aspect, methods for inhibiting (e.g., antagonizing) STING activity are featured that include contacting STING with a chemical entity described herein (e.g., a compound described generically or specifically herein or a pharmaceutically acceptable salt thereof or compositions containing the same). Methods include in vitro methods, e.g., contacting a sample that includes one or more cells comprising STING (e.g., innate immune cells, e.g., mast cells, macrophages, dendritic cells (DCs), and natural killer cells) with the chemical entity. Methods can also include in vivo methods; e.g., administering the chemical entity to a subject (e.g., a human) having a disease in which increased (e.g., excessive) STING signaling contributes to the pathology and/or symptoms and/or progression of the disease.

In one aspect, methods of treating a condition, disease or disorder ameliorated by antagonizing STING are featured, e.g., treating a condition, disease or disorder in which increased (e.g., excessive) STING activation (e.g., STING signaling) contributes to the pathology and/or symptoms and/or progression of the condition, disease or disorder (e.g., cancer) in a subject (e.g., a human). The methods include administering to a subject in need of such treatment an effective amount of a chemical entity described herein (e.g., a compound described generically or specifically herein or a pharmaceutically acceptable salt thereof or compositions containing the same).

In another aspect, methods of treating cancer are featured that include administering to a subject in need of such treatment an effective amount of a chemical entity described herein (e.g., a compound described generically or specifically herein or a pharmaceutically acceptable salt thereof or compositions containing the same).

In a further aspect, methods of treating other STING-associated conditions are featured, e.g., type I interferonopathies (e.g., STING-associated vasculopathy with onset in infancy (SAVI)), Aicardi-Goutieres Syndrome (AGS), genetic forms of lupus, and inflammation-associated disorders such as systemic lupus erythematosus, and rheumatoid arthritis. The methods include administering to a subject in need of such treatment an effective amount of a chemical entity described herein (e.g., a compound described generically or specifically herein or a pharmaceutically acceptable salt thereof or compositions containing the same).

In another aspect, methods of suppressing STING-dependent type I interferon production in a subject in need thereof are featured that include administering to the subject an effective amount of a chemical entity described herein (e.g., a compound described generically or specifically herein or a pharmaceutically acceptable salt thereof or compositions containing the same).

In a further aspect, methods of treating a disease in which increased (e.g., excessive) STING activation (e.g., STING signaling) contributes to the pathology and/or symptoms and/or progression of the disease are featured. The methods include administering to a subject in need of such treatment an effective amount of a chemical entity described herein (e.g., a compound described generically or specifically herein or a pharmaceutically acceptable salt thereof or compositions containing the same).

In another aspect, methods of treatment are featured that include administering an effective amount of a chemical entity described herein (e.g., a compound described generically or specifically herein or a pharmaceutically acceptable salt thereof or compositions containing the same) to a subject; wherein the subject has (or is predisposed to have) a disease in which increased (e.g., excessive) STING activation (e.g., STING signaling) contributes to the pathology and/or symptoms and/or progression of the disease.

In a further aspect, methods of treatment that include administering to a subject a chemical entity described herein (e.g., a compound described generically or specifically herein or a pharmaceutically acceptable salt thereof or compositions containing the same), wherein the chemical entity is administered in an amount effective to treat a disease in which increased (e.g., excessive) STING activation (e.g., STING signaling) contributes to the pathology and/or symptoms and/or progression of the disease, thereby treating the disease.

Embodiments can include one or more of the following features.

The chemical entity can be administered in combination with one or more additional therapeutic agents and/or regimens. For examples, methods can further include administering one or more (e.g., two, three, four, five, six, or more) additional agents.

The chemical entity can be administered in combination with one or more additional therapeutic agents and/or regimens that are useful for treating other STING-associated conditions, e.g., type I interferonopathies (e.g., STING-associated vasculopathy with onset in infancy (SAVI)), Aicardi-Goutieres Syndrome (AGS), genetic forms of lupus, and inflammation-associated disorders such as systemic lupus erythematosus, and rheumatoid arthritis.

The chemical entity can be administered in combination with one or more additional cancer therapies (e.g., surgery, radiotherapy, chemotherapy, toxin therapy, immunotherapy, cryotherapy or gene therapy, or a combination thereof, e.g., chemotherapy that includes administering one or more (e.g., two, three, four, five, six, or more) additional chemotherapeutic agents. Non-limiting examples of additional chemotherapeutic agents is selected from an alkylating agent (e.g., cisplatin, carboplatin, mechlorethamine, cyclophosphamide, chlorambucil, ifosfamide and/or oxaliplatin); an anti-metabolite (e.g., azathioprine and/or mercaptopurine); a terpenoid (e.g., a vinca alkaloid and/or a taxane; e.g., Vincristine, Vinblastine, Vinorelbine and/or Vindesine Taxol, Pacllitaxel and/or Docetaxel); a topoisomerase (e.g., a type I topoisomerase and/or a type 2 topoisomerase; e.g., camptothecins, such as irinotecan and/or topotecan; amsacrine, etoposide, etoposide phosphate and/or teniposide); a cytotoxic antibiotic (e.g., actinomycin, anthracyclines, doxorubicin, daunorubicin, valrubicin, idarubicin, epirubicin, bleomycin, plicamycin and/or mitomycin); a hormone (e.g., a lutenizing hormone releasing hormone agonist; e.g., leuprolidine, goserelin, triptorelin, histrelin, bicalutamide, flutamide and/or nilutamide); an antibody (e.g., Abciximab, Adalimumab, Alemtuzumab, Atlizumab, Basiliximab, Belimumab, Bevacizumab, Bretuximab vedotin, Canakinumab, Cetuximab, Ceertolizumab pegol, Daclizumab, Denosumab, Eculizumab, Efalizumab, Gemtuzumab, Golimumab, Golimumab, Ibritumomab tiuxetan, Infliximab, Ipilimumab, Muromonab-CD3, Natalizumab, Ofatumumab, Omalizumab, Palivizumab, Panitumuab, Ranibizumab, Rituximab, Tocilizumab, Tositumomab and/or Trastuzumab); an anti-angiogenic agent; a cytokine; a thrombotic agent; a growth inhibitory agent; an anti-helminthic agent; and an immune checkpoint inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155 (e.g., CTLA-4 or PD1 or PD-L1).

The subject can have cancer; e.g., the subject has undergone and/or is undergoing and/or will undergo one or more cancer therapies.

Non-limiting examples of cancer include melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma. In certain embodiments, the cancer can be a refractory cancer.

The chemical entity can be administered intratumorally.

The methods can further include identifying the subject.

Other embodiments include those described in the Detailed Description and/or in the claims.

### Additional Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

As used herein, the term “STING” is meant to include, without limitation, nucleic acids, polynucleotides, oligonucleotides, sense and antisense polynucleotide strands, complementary sequences, peptides, polypeptides, proteins, homologous and/or orthologous STING molecules, isoforms, precursors, mutants, variants, derivatives, splice variants, alleles, different species, and active fragments thereof.

The term “acceptable” with respect to a formulation, composition or ingredient, as used herein, means having no persistent detrimental effect on the general health of the subject being treated.

“API” refers to an active pharmaceutical ingredient.

The terms “effective amount” or “therapeutically effective amount,” as used herein, refer to a sufficient amount of a chemical entity being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. The “treatment of cancer”, refers to one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

The term “halo” refers to fluoro (F), chloro (Cl), bromo (Br), or iodo (I).

The term “alkyl” refers to a saturated acyclic hydrocarbon radical that may be a straight chain or branched chain, containing the indicated number of carbon atoms. For example, C₁₋₁₀ indicates that the group may have from 1 to 10 (inclusive) carbon atoms in it. Alkyl groups can either be unsubstituted or substituted with one or more substituents. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein.

The term “haloalkyl” refers to an alkyl, in which one or more hydrogen atoms is/are replaced with an independently selected halo.

The term “alkoxy” refers to an —O-alkyl radical (e.g., —OCH₃).

The term “alkylene” refers to a divalent alkyl (e.g., —CH₂—).

The term “alkenyl” refers to an acyclic hydrocarbon chain that may be a straight chain or branched chain having one or more carbon-carbon double bonds. The alkenyl moiety contains the indicated number of carbon atoms. For example, C₂₋₆ indicates that the group may have from 2 to 6 (inclusive) carbon atoms in it. Alkenyl groups can either be unsubstituted or substituted with one or more substituents.

The term “alkynyl” refers to an acyclic hydrocarbon chain that may be a straight chain or branched chain having one or more carbon-carbon triple bonds. The alkynyl moiety contains the indicated number of carbon atoms. For example, C₂₋₆ indicates that the group may have from 2 to 6 (inclusive) carbon atoms in it. Alkynyl groups can either be unsubstituted or substituted with one or more substituents.

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system); and wherein 0, 1, 2, 3, or 4 atoms of each ring may be substituted by a substituent. Examples of aryl groups include phenyl, naphthyl, tetrahydronaphthyl, and the like.

The term “cycloalkyl” as used herein refers to cyclic saturated hydrocarbon groups having, e.g., 3 to 20 ring carbons, preferably 3 to 16 ring carbons, and more preferably 3 to 12 ring carbons or 3-10 ring carbons or 3-6 ring carbons, wherein the cycloalkyl group may be optionally substituted. Examples of cycloalkyl groups include, without limitation, cyclopropyl, cyclobutyl, cyclopentyl, cyclohexyl, cycloheptyl, and cyclooctyl. Cycloalkyl may include multiple fused and/or bridged rings. Non-limiting examples of fused/bridged cycloalkyl includes: bicyclo[1.1.0]butane, bicyclo[2.1.0]pentane, bicyclo[1.1.1]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like. The term “saturated” as used in this context means only single bonds present between constituent carbon atoms.

The term “cycloalkenyl” as used herein means partially unsaturated cyclic hydrocarbon groups having 3 to 20 ring carbons, preferably 3 to 16 ring carbons, and more preferably 3 to 12 ring carbons or 3-10 ring carbons or 3-6 ring carbons, wherein the cycloalkenyl group may be optionally substituted. Examples of cycloalkenyl groups include, without limitation, cyclopentenyl, cyclohexenyl, cycloheptenyl, and cyclooctenyl. As partially unsaturated cyclic hydrocarbon groups, cycloalkenyl groups may have any degree of unsaturation provided that one or more double bonds is present in the ring, none of the rings in the ring system are aromatic, and the cycloalkenyl group is not fully saturated overall. Cycloalkenyl may include multiple fused and/or bridged and/or spirocyclic rings.

The term “heteroaryl”, as used herein, means a mono-, bi-, tri- or polycyclic group having 5 to 20 ring atoms, alternatively 5, 6, 9, 10, or 14 ring atoms; and having 6, 10, or 14 pi electrons shared in a cyclic array; wherein at least one ring in the system is aromatic, and at least one ring in the system contains one or more heteroatoms independently selected from the group consisting of N, O, and S (but does not have to be a ring which contains a heteroatom, e.g. tetrahydroisoquinolinyl, e.g., tetrahydroquinolinyl). Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl.

The term “heterocyclyl” refers to a mon-, bi-, tri-, or polycyclic saturated ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like. Heterocyclyl may include multiple fused and bridged rings. Non-limiting examples of fused/bridged heteorocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like. The term “saturated” as used in this context means only single bonds present between constituent ring atoms and other available valences occupied by hydrogen and/or other substituents as defined herein.

The term “heterocycloalkenyl” as used herein means partially unsaturated cyclic ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocycloalkenyl groups include, without limitation, tetrahydropyridyl, dihydropyrazinyl, dihydropyridyl, dihydropyrrolyl, dihydrofuranyl, dihydrothiophenyl. As partially unsaturated cyclic groups, heterocycloalkenyl groups may have any degree of unsaturation provided that one or more double bonds is present in the ring, none of the rings in the ring system are aromatic, and the heterocycloalkenyl group is not fully saturated overall. Heterocycloalkenyl may include multiple fused and/or bridged and/or spirocyclic rings.

As used herein, when a ring is described as being “aromatic”, it means said ring has a continuous, delocalized π-electron system. Typically, the number of out of plane π-electrons corresponds to the Hückel rule (4n+2). Examples of such rings include: benzene, pyridine, pyrimidine, pyrazine, pyridazine, pyridone, pyrrole, pyrazole, oxazole, thioazole, isoxazole, isothiazole, and the like.

As used herein, when a ring is described as being “partially unsaturated”, it means said ring has one or more additional degrees of unsaturation (in addition to the degree of unsaturation attributed to the ring itself, e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, heterocycloalkenyl, cycloalkenyl, cycloalkyl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge (e.g.,

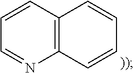

(ii) a single ring atom (spiro-fused ring systems) (e.g.,

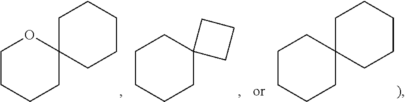

or (iii) a contiguous array of ring atoms (bridged ring
systems having all bridge lengths >0) (e.g.,

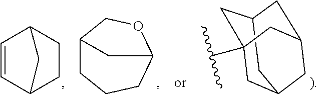

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

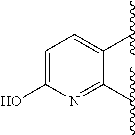 [SMILES: *c1ccc(O)nc1*]

encompasses the tautomeric form containing the moiety:

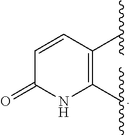 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

Some non-limiting exemplified compounds of the formulae described herein include one or more stereogenic carbon atoms. This disclosure provides examples of stereoisomer mixtures (e.g., racemic and non-racemic mixture of enantiomers; mixture of diastereomers, meso compounds). This disclosure also describes and exemplifies methods for separating individual components of said stereoisomer mixtures (e.g., resolving the enantiomers of a racemic mixture). In some instances, stereoisomers are graphically depicted using hashed and solid wedge three-dimensional representations. Unless otherwise indicated with “(R)” or “(S)” labels, the hashed and solid wedge three-dimensional representation are intended to convey relative stereochemistry only. Likewise, and unless otherwise indicated, reaction schemes showing resolution of a racemic mixture, the above-mentioned representations are intended only to convey that the constituent enantiomers were resolved in enantiopure pure form (about 98% ee or greater) and are not intended to disclose or imply any correlation between absolute configuration and order of elution.

The definitions of certain variables herein include -L¹-L²-R^{h} and -L³-L⁴-Rⁱ. For avoidance of doubt, when a variable is -L¹-L²-R^{h}; -L¹ is a bond; and -L² is a bond, then said variable is —R^{h} that is connected to the rest of the compound via a single bond. As a non-limiting example, when one occurrence of R^{b} is -L¹-L²-R^{h}; -L¹ is a bond; and -L² is a bond, then said occurrence of R^{b} is —R^{h} that is connected to the rest of the compound via a single bond. Similarly, when a variable is -L³-L⁴-Rⁱ; -L³ is a bond; and -L⁴ is a bond, then said variable is —R that is connected to the rest of the compound via a single bond.

The details of one or more embodiments of the invention are set forth in the accompanying drawings and the description below. Other features and advantages of the invention will be apparent from the description and drawings, and from the claims.

### DETAILED DESCRIPTION

This disclosure features chemical entities (e.g., a compound or a pharmaceutically acceptable salt, and/or hydrate, and/or cocrystal, and/or prodrug, and/or tautomer, and/or drug combination of the compound) that inhibit (e.g., antagonize) Stimulator of Interferon Genes (STING). Said chemical entities are useful, e.g., for treating a condition, disease or disorder in which increased (e.g., excessive) STING activation (e.g., STING signaling) contributes to the pathology and/or symptoms and/or progression of the condition, disease or disorder (e.g., cancer) in a subject (e.g., a human). This disclosure also features compositions containing the same as well as methods of using and making the same.

Formula I Compounds

In one aspect, compounds of Formula (I), or a pharmaceutically acceptable salt thereof, are featured:

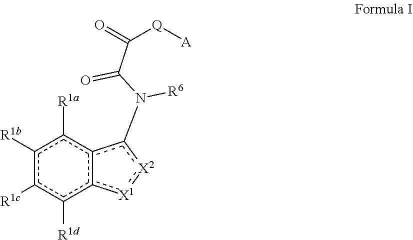

wherein:

X¹ is selected from the group consisting of O, S, N, NR², and CR⁵;

X² is selected from the group consisting of O, S, N, NR⁴, and CR⁵;

each [symbol] is independently a single bond or a double bond, provided that the five-membered ring comprising X¹ and X² is heteroaryl; and

the 6-membered ring

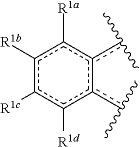

is aromatic;

Q-A is defined according to (A) or (B) below:
(A)

Q is selected from the group consisting of: NH and N(C₁₋₆ alkyl) wherein the C₁₋₆ alkyl is optionally substituted with 1-2 independently selected R^{a}; and

A is:

(i) —(Y^{A1})ₙY^{A2}, wherein:
n is 0 or 1;
Y^{A1} is C₁₋₆ alkylene, which is optionally substituted with 1-6 substituents each independently selected from the group consisting of:
   oxo;
   R^{a};
   C₆₋₁₀ aryl optionally substituted with 1-4 independently selected C₁₋₄ alkyl; and
   heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl ring is optionally substituted with 1-4 independently selected C₁₋₄ alkyl; or
Y^{A1} is —Y^{A3}—Y^{A4}—Y^{A5} which is connected to Q via Y^{A3} wherein:
   Y^{A3} is a C₁₋₃ alkylene optionally substituted with 1-2 substituents each independently selected from the group consisting of oxo and R^{a};
   Y^{A4} is —O—, —NH—, —N(C₁₋₆ alkyl)-, or —S—; and
   Y^{A5} is a bond or C₁₋₃ alkylene which is optionally substituted with 1-2 independently selected R^{a}; and
Y^{A2} is:
   (a) C₃₋₂₀ cycloalkyl or C₃₋₂₀ cycloalkenyl, each of which is optionally substituted with 1-4 R^{b},
   (b) C₆₋₂₀ aryl which is optionally substituted with 1-4 R^{c};
   (c) heteroaryl of 5-20 ring atoms, wherein 1-3 ring atoms are heteroatoms, each independently selected from the group consisting of N, N(H), N(R^{d}), O, and S(O)₀₋₂, and wherein the heteroaryl ring is optionally substituted with 1-4 independently selected R^{c}; or
   (d) heterocyclyl or heterocycloalkenyl of 3-16 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl ring is optionally substituted with 1-4 independently selected R^{b},

or

(ii) —Z¹—Z²—Z³, wherein:
Z¹ is C₁₋₃ alkylene, which is optionally substituted with 1-4 R^{a};
Z² is —N(H)—, —N(R^{d})—, —O—, or —S—; and
Z³ is C₂₋₇ alkyl, which is optionally substituted with 1-4 R^{a};

or

(iii) C₁₋₂₀ alkyl, which is optionally substituted with 1-6 independently selected R^{a}, or

Q and A, taken together, form:

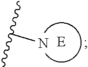

and

E is a ring of 3-16 ring atoms, wherein 0-3 ring atoms are heteroatoms (in addition to the nitrogen atom this is already present), each independently selected from the group consisting of N, N(H), N(R^{d}), O, and S(O)₀₋₂, and wherein the ring is optionally substituted with 1-4 independently selected R^{b},

each of R^{1a}, R^{1b}, R^{1c}, and R^{1d} is independently selected from the group consisting of: H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; -L³-L⁴-Rⁱ; —S(O)₁₋₂ (C₁₋₄ alkyl); —S(O)(═NH)(C₁₋₄ alkyl); SF₅; —NR^{e}R^{f}; —OH; oxo; —S(O)₁₋₂ (NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″); or

R^{1a} and R^{1b}, R^{1b} and R^{1c}, or R^{1c} and R^{1d}, taken together with the atoms connecting them, form a ring of 3-10 ring atoms, wherein 0-2 ring atoms are heteroatoms each independently selected from the group consisting of N, N(H), N(R^{d}), O, and S(O)₀₋₂; and wherein the ring is optionally substituted with 1-4 substituents each independently selected from the group consisting of C₁₋₆ alkyl, halo, C₁₋₆ haloalkyl, —OH, NR^{e}R^{f}, C₁₋₆ alkoxy, and C₁₋₆ haloalkoxy,

each occurrence of R² is independently selected from the group consisting of:

(i) C₁₋₆ alkyl, which is optionally substituted with 1-2 independently selected R^{a};

(ii) C₃₋₆ cycloalkyl or C₃₋₆ cycloalkenyl;

(iii) heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂;

(iv) C₆₋₁₀ aryl;

(v) heteroaryl of 5-10 ring atoms, wherein 1-3 ring atoms are heteroatoms, each independently selected from the group consisting of N, N(H), N(R^{d}), O, and S(O)₀₋₂;

(vi) —C(O)(C₁₋₄ alkyl);

(vii) —C(O)O(C₁₋₄ alkyl);

(viii) —CON(R′)(R″);

(ix) —S(O)₁₋₂(NR′R″);

(x) —S(O)₁₋₂(C₁₋₄ alkyl);

(xi) —OH;

(xii) C₁₋₄ alkoxy; and

(xiii) H;

R⁴ is selected from the group consisting of H and C₁₋₆ alkyl optionally substituted with 1-3 independently selected R^{a};

R⁵ is selected from the group consisting of H; halo; —OH; —C₁₋₄ alkyl; —C₁₋₄ haloalkyl; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and C₃₋₆ cycloalkyl or C₃₋₆ cycloalkenyl, each optionally substituted with 1-4 independently selected C₁₋₄ alkyl;

R⁶ is selected from the group consisting of H; C₁₋₆ alkyl optionally substituted with 1-3 independently selected R^{a}; —OH; C₁₋₄ alkoxy; C(═O)H; C(═O)(C₁₋₄ alkyl); C₆₋₁₀ aryl optionally substituted with 1-4 independently selected C₁₋₄ alkyl; and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 independently selected C₁₋₄ alkyl;

each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano, and C₃₋₆ cycloalkyl or C₃₋₆ cycloalkenyl, each optionally substituted with 1-4 independently selected C₁₋₄ alkyl;

each occurrence of R^{b} is independently selected from the group consisting of: C₁₋₁₀ alkyl optionally substituted with 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₁₀ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};

each occurrence of R^{c} is independently selected from the group consisting of: halo; cyano; C₁₋₁₀ alkyl which is optionally substituted with 1-6 independently selected R^{a}; C₂₋₆ alkenyl; C₂₋₆ alkynyl; oxo; C₁₋₄ alkoxy optionally substituted with 1-2 independently selected R^{a}; C₁₋₄ haloalkoxy; —S(O)₁₋₂(C₁₋₄ alkyl) or —S(O)₁₋₂(C₁₋₄ haloalkyl); —NR^{e}R^{f}; —OH; —S(O)₁₋₂(NR′R″); —C₁₋₄ thioalkoxy or —C₁₋₄ thiohaloalkoxy; —NO₂; —SF₅; —C(═O)(C₁₋₁₀ alkyl); —C(═O)O(C₁₋₄ alkyl); —C(═O)OH; —C(═O)N(R′)(R″); and -L¹-L²-R^{h};

R^{d} is selected from the group consisting of: C₁₋₆ alkyl optionally substituted with 1-3 substituents each independently selected from the group consisting of halo and OH; C₃₋₆ cycloalkyl or C₃₋₆ cycloalkenyl, each optionally substituted with 1-3 substituents each independently selected from the group consisting of halo and OH; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;
each occurrence of R^{e} and R^{f} is independently selected from the group consisting of: H; C₁₋₆ alkyl; C₁₋₆ haloalkyl; C₃-6 cycloalkyl or C₃₋₆ cycloalkenyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or R^{e} and R^{f} together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R^{e} and R^{f}), which are each independently selected from the group consisting of N(R^{d}), NH, 0, and S;

-L¹ is a bond or C₁₋₃ alkylene;

-L² is —O—, —N(H)—, —N(C₁₋₃ alkyl)-, —S(O)₀₋₂-, or a bond;

R^{h} is selected from the group consisting of:
C₃₋₈ cycloalkyl or C₃₋₈ cycloalkenyl, each optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy; and
C₆₋₁₀ aryl, which is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy;

-L³ is a bond or C₁₋₃ alkylene;

-L⁴ is —O—, —N(H)—, —N(C₁₋₃ alkyl)-, —S(O)₀₋₂—, or a bond;

Rⁱ is selected from the group consisting of:
C₃₋₈ cycloalkyl or C₃₋₈ cycloalkenyl, each optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy; and

each occurrence of R′ and R″ is independently selected from the group consisting of: H, C₁₋₄ alkyl, C₆₋₁₀ aryl optionally substituted with 1-2 substituents selected from the group consisting of halo, C₁₋₄ alkyl, and C₁₋₄ haloalkyl, and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo, —OH, NH₂, NH(C₁₋₄ alkyl), N(C₁₋₄ alkyl)₂, C₁₋₄ alkyl, and C₁₋₄ haloalkyl; or R′ and R″ together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R′ and R″), which are each independently selected from the group consisting of N(H), N(C₁₋₆ alkyl), O, and S.

In one aspect, compounds of Formula (I), a pharmaceutically acceptable salt thereof, or a tautomer thereof are featured:

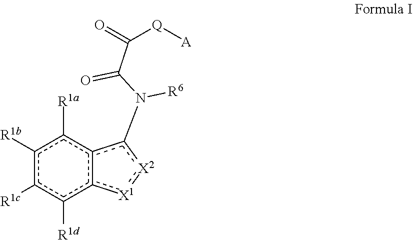

wherein:

X¹ is selected from the group consisting of O, S, N, NR², and CR⁵;

X² is selected from the group consisting of O, S, N, NR⁴, and CR⁵;

each [symbol] is independently a single bond or a double bond, provided that the five-membered ring comprising X¹ and X² is heteroaryl; and the 6-membered ring is aromatic:

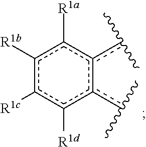

Q-A is defined according to (A) or (B) below:
(A)

Q is selected from the group consisting of: NH and N(C₁₋₆ alkyl) wherein the C₁₋₆ alkyl is optionally substituted with 1-2 independently selected R^{a}; and

A is:

(i) —(Y^{A1})ₙ—Y^{A2}, wherein:
n is 0 or 1;
Y^{A1} is C₁₋₆ alkylene, which is optionally substituted with 1-6 substituents each independently selected from the group consisting of:
   R^{a};
   C₆₋₁₀ aryl optionally substituted with 1-4 independently selected C₁₋₄ alkyl; and
   heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl ring is optionally substituted with 1-4 independently selected C₁₋₄ alkyl; and
Y^{A1} is —Y^{A3}—Y^{A4}—Y^{A5} which is connected to Q via Y^{A3} wherein:
   Y^{A3} is a C₁₋₃ alkylene optionally substituted with 1-2 independently selected R^{a};
   Y^{A4} is —O—, —NH—, or —S—; and
   Y^{A5} is a bond or C₁₋₃ alkylene which is optionally substituted with 1-2 independently selected R^{a}; and
Y^{A2} is:
   (a) C₃₋₂₀ cycloalkyl or C₃₋₂₀ cycloalkenyl, each of which is optionally substituted with 1-4 R^{b},
   (b) C₆₋₂₀ aryl, which is optionally substituted with 1-4 R^{c};
   (c) heteroaryl of 5-20 ring atoms, wherein 1-3 ring atoms are heteroatoms, each independently selected from the group consisting of N, N(H), N(R^{d}), O, and S(O)₀₋₂, and wherein the heteroaryl ring is optionally substituted with 1-4 independently selected R^{c}; or
   (d) heterocyclyl or heterocycloalkenyl of 3-16 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl ring is optionally substituted with 1-4 independently selected R^{b},

or

(ii) —Z¹—Z²—Z³, wherein:
Z¹ is C₁₋₃ alkylene, which is optionally substituted with 1-4 R^{a};
Z² is —N(H)—, —N(R^{d})—, —O—, or —S—; and
Z³ is C₂₋₇ alkyl, which is optionally substituted with 1-4 R^{a};

or

(iii) C₁₋₂₀ alkyl, which is optionally substituted with 1-6 independently selected R^{a}, or

Q and A, taken together, form:

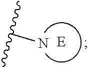

and

E is a ring of 3-16 ring atoms, wherein 0-3 ring atoms are heteroatoms (in addition to the nitrogen atom this is already present), each independently selected from the group consisting of N, N(H), N(R^{d}), O, and S(O)₀₋₂, and wherein the ring is optionally substituted with 1-4 independently selected R^{b},

each of R^{1a}, R^{1b}, R^{1c}, and R^{1d} is independently selected from the group consisting of H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; -L³-L⁴-Rⁱ; —S(O)₁₋₂(C₁₋₄ alkyl); —S(O)(═NH)(C₁₋₄ alkyl); SF₅; —NR^{e}R^{f}; —OH; oxo; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″); or

R^{1a} and R^{1b}, R^{1b} and R^{1c}, or R^{1c} and R^{1d}, taken together with the atoms connecting them, form a ring of 3-10 ring atoms, wherein 0-2 ring atoms are heteroatoms each independently selected from the group consisting of N, N(H), N(R^{d}), O, and S(O)₀₋₂; and wherein the ring is optionally substituted with 1-4 substituents each independently selected from the group consisting of C₁₋₆ alkyl, halo, C₁₋₆ haloalkyl, —OH, NR^{e}R^{f}, C₁₋₆ alkoxy, and C₁₋₆ haloalkoxy,

each occurrence of R² is independently selected from the group consisting of:

(i) C₁₋₆ alkyl, which is optionally substituted with 1-2 independently selected R^{a};

(ii) C₃₋₆ cycloalkyl or C₃₋₆ cycloalkenyl;

(iii) heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂;

(iv) C₆₋₁₀ aryl;

(v) heteroaryl of 5-10 ring atoms, wherein 1-3 ring atoms are heteroatoms, each independently selected from the group consisting of N, N(H), N(R^{d}), O, and S(O)₀₋₂; (vi) —C(O)(C₁₋₄ alkyl);

(vii) —C(O)O(C₁₋₄ alkyl);

(viii) —CON(R′)(R″);

(ix) —S(O)₁₋₂(NR′R″);

(x) —S(O)₁₋₂(C₁₋₄ alkyl);

(xi) —OH;

(xii) C₁₋₄ alkoxy; and

(xiii) H;

R⁴ is selected from the group consisting of H and C₁₋₆ alkyl optionally substituted with 1-3 independently selected R^{a};

R⁵ is selected from the group consisting of H; halo; —OH; —C₁₋₄ alkyl; —C₁₋₄ haloalkyl; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano, and C₃₋₆ cycloalkyl or C₃₋₆ cycloalkenyl, each optionally substituted with 1-4 independently selected C₁₋₄ alkyl;

R⁶ is selected from the group consisting of H; C₁₋₆ alkyl optionally substituted with 1-3 independently selected R^{a}; —OH; C₁₋₄ alkoxy; C(═O)H; C(═O)(C₁₋₄ alkyl); C₆₋₁₀ aryl optionally substituted with 1-4 independently selected C₁₋₄ alkyl; and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 independently selected C₁₋₄ alkyl;

each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano, and C₃₋₆ cycloalkyl or C₃₋₆ cycloalkenyl, each optionally substituted with 1-4 independently selected C₁₋₄ alkyl;

each occurrence of R^{b} is independently selected from the group consisting of: C₁₋₁₀ alkyl optionally substituted with 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₁₀ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};

each occurrence of R^{e} is independently selected from the group consisting of:

(a) halo; (b) cyano; (c) C₁₋₁₀ alkyl which is optionally substituted with 1-6 independently selected R^{a}; (d) C₂₋₆ alkenyl; (e) C₂₋₆ alkynyl; (g) C₁₋₄ alkoxy optionally substituted with 1-2 independently selected R^{a}; (h) C₁₋₄ haloalkoxy; (i) —S(O)₁₋₂(C₁₋₄ alkyl) or —S(O)₁₋₂(C₁₋₄ haloalkyl); (j) —NR^{e}R^{f}; (k) —OH; (1) —S(O)₁₋₂(NR′R″); (m) —C₁₋₄ thioalkoxy or —C₁₋₄ thiohaloalkoxy; (n) —NO₂; (o) —SF₅; (p) —C(═O)(C₁₋₁₀ alkyl); (q) —C(═O)O(C₁₋₄ alkyl); (r) —C(═O)OH; (s) —C(═O)N(R′)(R″); and (t) -L¹-L²-R^{h};

R^{d} is selected from the group consisting of: C₁₋₆ alkyl optionally substituted with 1-3 substituents each independently selected from the group consisting of halo and OH; C₃₋₆ cycloalkyl or C₃₋₆ cycloalkenyl, each optionally substituted with 1-3 substituents each independently selected from the group consisting of halo and OH; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;

each occurrence of R^{e} and R^{f} is independently selected from the group consisting of: H; C₁₋₆ alkyl; C₁₋₆ haloalkyl; C₃₋₆ cycloalkyl or C₃₋₆ cycloalkenyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or R^{e} and R^{f} together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R^{e} and R^{f}), which are each independently selected from the group consisting of N(R^{d}), NH, 0, and S;

-L¹ is a bond or C₁₋₃ alkylene;

-L² is —O—, —N(H)—, —S(O)₀₋₂—, or a bond;

R^{h} is selected from the group consisting of:
C₃₋₈ cycloalkyl or C₃₋₈ cycloalkenyl, each optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy (in certain embodiments, it is provided that when R^{h} is C₃-6 cycloalkyl or C₃₋₆ cycloalkenyl, each optionally substituted with 1-4 substituents independently selected C₁₋₄ alkyl, -L¹ is a bond, or -L² is —O—, —N(H)—, or —S—);
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;

-L³ is a bond or C₁₋₃ alkylene;

-L⁴ is —O—, —N(H)—, —S(O)₀₋₂—, or a bond;

Rⁱ is selected from the group consisting of:
C₃₋₈ cycloalkyl or C₃₋₈ cycloalkenyl, each optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy (in certain embodiments, it is provided that when Rⁱ is C₃₋₆ cycloalkyl or C₃₋₆ cycloalkenyl, each optionally substituted with 1-4 substituents independently selected C₁₋₄ alkyl, -L¹ is a bond, or -L² is —O—, —N(H)—, or —S—);
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy; and

each occurrence of R′ and R″ is independently selected from the group consisting of: H, C₁₋₄ alkyl, C₆₋₁₀ aryl optionally substituted with 1-2 substituents selected from the group consisting of halo, C₁₋₄ alkyl, and C₁₋₄ haloalkyl, and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo, —OH, NH₂, NH(C₁₋₄ alkyl), N(C₁₋₄ alkyl)₂, C₁₋₄ alkyl, and C₁₋₄ haloalkyl; or R′ and R″ together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R′ and R″), which are each independently selected from the group consisting of N(H), N(C₁₋₆ alkyl), O, and S.

In one aspect, compounds of Formula (I), or a pharmaceutically acceptable salt thereof, are featured:

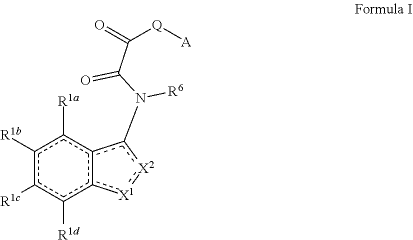

or a pharmaceutically acceptable salt thereof or a tautomer thereof, wherein:

X¹ is selected from the group consisting of O, S, N, NR², and CR⁵;

X² is selected from the group consisting of O, S, N, NR⁴, and CR⁵;

each [symbol] is independently a single bond or a double bond, provided that the five-membered ring comprising X¹ and X² is heteroaryl; and

the 6-membered ring is aromatic:

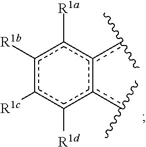

Q-A is defined according to (A) or (B) below:
(A)

Q is selected from the group consisting of: NH and N(C₁₋₆ alkyl) wherein the C₁₋₆ alkyl is optionally substituted with 1-2 independently selected R^{a}; and

A is:

(i) —(Y^{A1})ₙ—Y^{A2}, wherein:
n is 0 or 1;
Y^{A1} is C₁₋₆ alkylene, which is optionally substituted with 1-6 substituents each independently selected from the group consisting of:
   R^{a};
   C₆₋₁₀ aryl optionally substituted with 1-4 independently selected C₁₋₄ alkyl; and
   heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl ring is optionally substituted with 1-4 independently selected C₁₋₄ alkyl; and
Y^{A2} is:

(a) C₃₋₂₀ cycloalkyl or C₃-20 cycloalkenyl, each of which is optionally substituted with 1-4 R^{b},

(b) C₆₋₂₀ aryl, which is optionally substituted with 1-4 R^{c};

(c) heteroaryl of 5-20 ring atoms, wherein 1-3 ring atoms are heteroatoms, each independently selected from the group consisting of N, N(H), N(R^{d}), O, and S(O)₀₋₂, and wherein the heteroaryl ring is optionally substituted with 1-4 independently selected R^{c}; or

(d) heterocyclyl or heterocycloalkenyl of 3-16 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl ring is optionally substituted with 1-4 independently selected R^{b},

or

(ii) —Z¹—Z²—Z³, wherein:
Z¹ is C₁₋₃ alkylene, which is optionally substituted with 1-4 R^{a};
Z² is —N(H)—, —N(R^{d})—, —O—, or —S—; and
Z³ is C₂₋₇ alkyl, which is optionally substituted with 1-4 R^{a};

or

(iii) C₁₋₁₀ alkyl, which is optionally substituted with 1-6 independently selected R^{a}, or
(B)

Q and A, taken together, form:

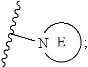

and

E is a ring of 3-16 ring atoms, wherein 0-3 ring atoms are heteroatoms (in addition to the nitrogen atom that is present), each independently selected from the group consisting of N, N(H), N(R^{d}), O, and S(O)₀₋₂, and wherein the ring is optionally substituted with 1-4 independently selected R^{b},

each of R^{1a}, R^{1b}, R^{1c}, and R^{1d} is independently selected from the group consisting of H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; -L³-L⁴-Rⁱ; —S(O)₁₋₂(C₁₋₄ alkyl); —S(O)(═NH)(C₁₋₄ alkyl); SF₅; —NR^{e}R^{f}; —OH; oxo; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″); or

R^{1a} and R^{1b}, R^{1b} and R^{1c}, or R^{1c} and R^{1d}, taken together with the atoms connecting them, form a ring of 3-10 ring atoms, wherein 0-2 ring atoms are heteroatoms each independently selected from the group consisting of N, N(H), N(R^{d}), 0, and S(O)₀₋₂; and wherein the ring is optionally substituted with 1-4 substituents each independently selected from the group consisting of C₁₋₆ alkyl, halo, C₁₋₆ haloalkyl, —OH, NR^{e}R^{f}, C₁₋₆ alkoxy, and C₁₋₆ haloalkoxy,

each occurrence of R² is independently selected from the group consisting of:

(i) C₁₋₆ alkyl, which is optionally substituted with 1-2 independently selected R^{a};

(ii) C₃₋₆ cycloalkyl or C₃₋₆ cycloalkenyl;

(iii) heterocyclyl or heterocycloalkenyl of 3-10 ring atoms, wherein 1-3 ring atoms are heteroatoms, each independently selected from the group consisting of N, N(H), N(R^{d}), 0, and S(O)₀₋₂;

(iv) C₆₋₁₀ aryl;

(v) heteroaryl of 5-10 ring atoms, wherein 1-3 ring atoms are heteroatoms, each independently selected from the group consisting of N, N(H), N(R^{d}), O, and S(O)₀₋₂; (vi) —C(O)(C₁₋₄ alkyl);

(vii) —C(O)O(C₁₋₄ alkyl);

(viii) —CON(R′)(R″);

(ix) —S(O)₁₋₂(NR′R″);

(x) —S(O)₁₋₂(C₁₋₄ alkyl);

(xi) —OH;

(xii) C₁₋₄ alkoxy; and

(xiii) H;

R⁴ is selected from the group consisting of H and C₁₋₆ alkyl optionally substituted with 1-3 independently selected R^{a};

R⁵ is selected from the group consisting of H; halo; —OH; —C₁₋₄ alkyl; —C₁₋₄ haloalkyl; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano, and C₃₋₆ cycloalkyl or C₃₋₆ cycloalkenyl, each optionally substituted with 1-4 independently selected C₁₋₄ alkyl;

R⁶ is selected from the group consisting of H; C₁₋₆ alkyl optionally substituted with 1-3 independently selected R^{a}; —OH; C₁₋₄ alkoxy; C(═O)H; C(═O)(C₁₋₄ alkyl); C₆₋₁₀ aryl optionally substituted with 1-4 independently selected C₁₋₄ alkyl; and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 independently selected C₁₋₄ alkyl;

each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano, and C₃₋₆ cycloalkyl or C₃₋₆ cycloalkenyl, each optionally substituted with 1-4 independently selected C₁₋₄ alkyl;

each occurrence of R^{b} is independently selected from the group consisting of: C₁₋₁₀ alkyl optionally substituted with 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₁₀ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};

each occurrence of R^{e} is independently selected from the group consisting of:

(a) halo; (b) cyano; (c) C₁₋₁₀ alkyl which is optionally substituted with 1-6 independently selected R^{a}; (d) C₂₋₆ alkenyl; (e) C₂₋₆ alkynyl; (g) C₁₋₄ alkoxy; (h) C₁₋₄ haloalkoxy; (i) —S(O)₁₋₂(C₁₋₄ alkyl); (j) —NR^{e}R^{f}; (k) —OH; (1) —S(O)₁₋₂(NR′R″); (m) —C₁₋₄ thioalkoxy; (n) —NO₂; (o) —C(═O)(C₁₋₁₀ alkyl); (p) —C(═O)O(C₁₋₄ alkyl); (q) —C(═O)OH; (r) —C(═O)N(R′)(R″); and (s) -L¹-L²-R^{h};

R^{d} is selected from the group consisting of: C₁₋₆ alkyl optionally substituted with 1-3 substituents each independently selected from the group consisting of halo and OH; C₃₋₆ cycloalkyl or C₃₋₆ cycloalkenyl, each optionally substituted with 1-3 substituents each independently selected from the group consisting of halo and OH; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;

each occurrence of R^{e} and R^{f} is independently selected from the group consisting of: H; C₁₋₆ alkyl; C₁₋₆ haloalkyl; C₃₋₆ cycloalkyl or C₃₋₆ cycloalkenyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or R^{e} and R^{f} together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R^{e} and R^{f}), which are each independently selected from the group consisting of N(R^{d}), NH, O, and S;

-L¹ is a bond or C₁₋₃ alkylene;

-L² is —O—, —N(H)—, —S(O)₀₋₂-, or a bond;

R^{h} is selected from the group consisting of:
C₃₋₈ cycloalkyl or C₃₋₈ cycloalkenyl, each optionally substituted with 1-4 substituents independently selected from the group consisting of halo, C₁₋₄ alkyl, and C₁₋₄ haloalkyl (in certain embodiments, it is provided that when R^{h} is C₃₋₆ cycloalkyl or C₃₋₆ cycloalkenyl, each optionally substituted with 1-4 substituents independently selected C₁₋₄ alkyl, -L¹ is a bond, or -L² is —O—, —N(H)—, or —S—);
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo, C₁₋₄ alkyl, and C₁₋₄ haloalkyl;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo, C₁₋₄ alkyl, and C₁₋₄ haloalkyl; and
C₆₋₁₀ aryl, which is optionally substituted with 1-4 substituents independently selected from the group consisting of halo, C₁₋₄ alkyl, and C₁₋₄ haloalkyl;

-L³ is a bond or C₁₋₃ alkylene;

-L⁴ is —O—, —N(H)—, —S(O)₀₋₂—, or a bond;

Rⁱ is selected from the group consisting of:
C₃₋₈ cycloalkyl or C₃₋₈ cycloalkenyl, each optionally substituted with 1-4 substituents independently selected from the group consisting of halo, C₁₋₄ alkyl, and C₁₋₄ haloalkyl (in certain embodiments, it is provided that when R is C₃₋₆ cycloalkyl or C₃₋₆ cycloalkenyl, each optionally substituted with 1-4 substituents independently selected C₁₋₄ alkyl, -L¹ is a bond, or -L² is —O—, —N(H)—, or —S—);
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo, C₁₋₄ alkyl, and C₁₋₄ haloalkyl;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo, C₁₋₄ alkyl, and C₁₋₄ haloalkyl; and
C₆₋₁₀ aryl, which is optionally substituted with 1-4 substituents independently selected from the group consisting of halo, C₁₋₄ alkyl, or C₁₋₄ haloalkyl; and

each occurrence of R′ and R″ is independently selected from the group consisting of: H, —OH; C₁₋₄ alkyl, C₆₋₁₀ aryl optionally substituted with 1-2 substituents selected from the group consisting of halo, C₁₋₄ alkyl, and C₁₋₄ haloalkyl, and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo, —OH, NH₂, NH(C₁₋₄ alkyl), N(C₁₋₄ alkyl)₂, C₁₋₄ alkyl, and C₁₋₄ haloalkyl; or R′ and R″ together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R′ and R″), which are each independently selected from the group consisting of N(H), N(C₁₋₆ alkyl), O, and S.

Embodiments can include any one or more of the features delineated below and/or in the claims.

The Variables X¹ and X²

In some embodiments, X¹ is NR². In certain of these embodiments, X¹ is NH.

In some embodiments, X² is CR⁵. In certain of these embodiments, X² is CH. In other embodiments, R⁵ is other than H.

In certain embodiments, X¹ is NR²; and X² is CR⁵.

In certain embodiments, X¹ is NH; and X² is CH.

The

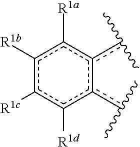

### Moiety

In some embodiments, the

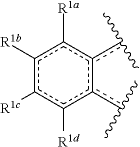

moiety

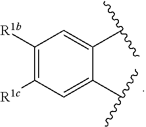

In certain embodiments, the

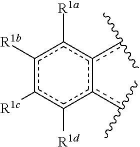

moiety is

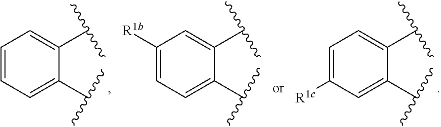

Non-Limiting Combinations of X¹, X², and the

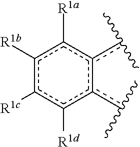

### Moiety

In certain embodiments, the compound is a compound of Formula (I-a):

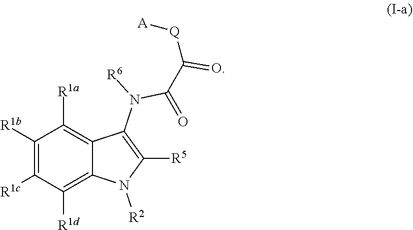

In certain of these embodiments, compound has formula (I-a1):

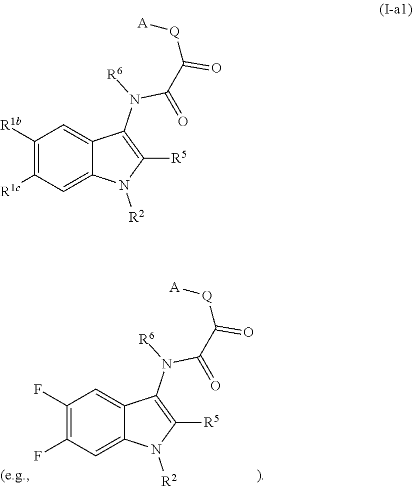

In certain embodiments of Formula (I-a), the compound has formula (I-a2):

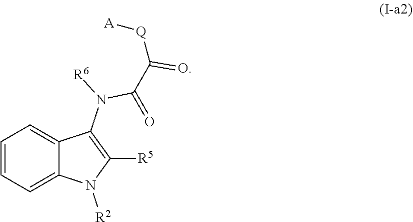

In certain embodiments of Formula (I-a), the compound has formula (I-a3) or (I-a4):

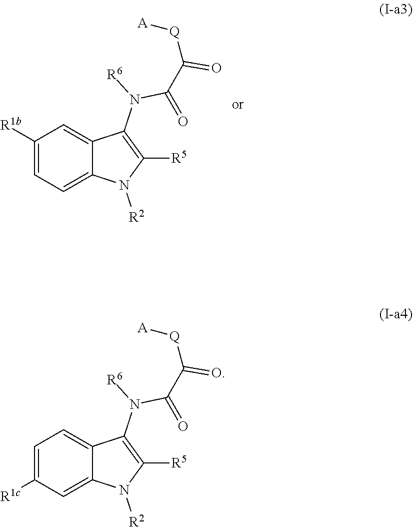

In certain embodiments, the compound is a compound of Formula (I-a5):

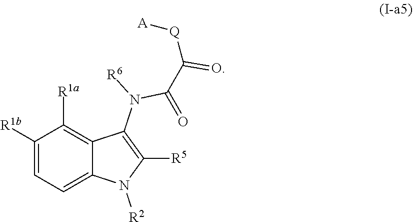

In certain embodiments of Formula (I-a) (e.g., when the compound has Formula (I-a1), (I-a2), (I-a3), (I-a4), or (I-a5)), R² is H; and R^{I} is H.

The Variables R^{1a}, R^{1b}, R^{1c}, and R^{1d}

In some embodiments, each of R^{1a}, R^{1b}, R^{1c}, and R^{1d} is independently selected from the group consisting of: H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; -L³-L⁴-Rⁱ; —S(O)₁₋₂(C₁₋₄ alkyl); —S(O)(═NH)(C₁₋₄ alkyl); SF₅; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″).

In certain embodiments, 0-3 (e.g., 0, 1, 2, or 3) of R^{1a}, R^{1b}, R^{1c}, and R^{1d} is other than H; and each of the remaining of R^{1a}, R^{1b}, R^{1c}, and R^{1d} is H.

In certain embodiments, each of R^{1a}, R^{1b}, R^{1c}, and R^{1d} is H.

In certain other embodiments, 1-2 occurrences of R^{1a}, R^{1b}, R^{1c}, and R^{1d} is other than H (e.g., R^{1b} and/or R^{1c} is other than H). As a non-limiting example of the foregoing embodiments, two of R^{1a}, R^{1b}, R^{1c}, and R^{1d} are other than H (e.g., R^{1b} and R^{1c} are other than H).

In certain embodiments, 1-2 of R^{1a}, R^{1b}, R^{1c}, and R^{1d} is selected from the group consisting of: halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; —S(O)₁₋₂(C₁₋₄ alkyl); —S(O)₁₋₂(NR′R″); —NO₂; —C(═O)(C₁₋₄ alkyl); —C(═O)O(C₁₋₄ alkyl); —C(═O)OH; and —C(═O)N(R′)(R″).

In certain embodiments, 1-2 occurrence of R^{1a}, R^{1b}, R^{1c}, and R^{1d} is halo (e.g., F or Cl (e.g., F)). In certain embodiments, 2 occurrences of R^{1a}, R^{1b}, R^{c}, and R^{1d} are halo (e.g., -F).

In certain embodiments, one occurrence of R^{1a}, R^{1b}, R^{1c}, and R^{1d} is halo (e.g., —F, —Cl, or —Br).

In certain embodiments, one of R^{1a}, R^{1b}, R^{1c}, and R^{1d} is -L³-L⁴-Rⁱ. In certain of these embodiments, L³ is a bond; and/or L⁴ is a bond. As a non-limiting example, one of R^{1a}, R^{1b}, R^{1c}, and R^{1d} is R (e.g., R is heteroaryl of 5-10 ring atoms, wherein 1-4 ring atoms are heteroatoms, each independently selected from the group consisting of N, N(H), N(R^{d}), 0, and S(O)₀₋₂ and wherein the heteroaryl ring is optionally substituted with 1-4 substituents independently selected from the group consisting of halo, C₁₋₄ alkyl, and C₁₋₄ haloalkyl; or Rⁱ is C₆₋₁₀ aryl, which is optionally substituted with 1-4 substituents independently selected from the group consisting of halo, C₁₋₄ alkyl, or C₁₋₄ haloalkyl).

In certain embodiments, one occurrence of R^{1a}, R^{1b}, R^{1c}, and R^{1d} is -L³-L⁴-Rⁱ, such as R^{1b} is -L³-L⁴-Rⁱ; and each remaining occurrences of R^{1a}, R^{1b}, R^{1c}, and R^{1d} is H. In certain of these embodiments, -L³ is a bond; and/or -L⁴ is a bond.

In certain of the foregoing embodiments, —Rⁱ is selected from the group consisting of:
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy.

In certain of these embodiments, —Rⁱ is selected from the group consisting of:
heteroaryl of 5-6 ring atoms (e.g., pyrazolyl), wherein 1-4 ring atoms are heteroatoms, each independently selected from the group consisting of N, N(H), N(R^{d}), O, and S(O)₀₋₂ and wherein the heteroaryl ring is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy; and
phenyl, which is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy.

In certain embodiments, one of R^{1a}, R^{1b}, R^{1c}, and R^{1d} (such as R^{1b}) is selected from the group consisting of:
heteroaryl of 5-6 ring atoms (such as pyrazolyl), wherein 1-4 ring atoms are heteroatoms, each independently selected from the group consisting of N, N(H), N(R^{d}), O, and S(O)₀₋₂ and wherein the heteroaryl ring is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy
   (e.g.,

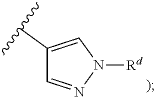

and
phenyl, which is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy (e.g.,

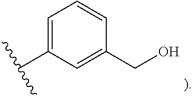 [SMILES: *c1cccc(CO)c1]

In certain of these embodiments, each remaining R^{1a}, R^{1b}, R^{1c}, and R^{1d} is H.
The Variables R², R⁵, and R⁶

In some embodiments, R² is H.

In some embodiments, R² is selected from the group consisting of:

heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂; and

heteroaryl of 5-10 ring atoms, wherein 1-3 ring atoms are heteroatoms, each independently selected from the group consisting of N, N(H), N(R^{d}), O, and S(O)₀₋₂.

In certain embodiments, R² is heterocyclyl or heterocycloalkenyl of 3-10 (e.g., 3, 4, 5, 6, 7, 8, 9, or 10) ring atoms, wherein 1-3 ring atoms are heteroatoms, each independently selected from the group consisting of N, N(H), N(R^{d}), O, and S(O)₀₋₂. In certain of these embodiments, R² is heterocyclyl or heterocycloalkenyl of 4-6 (e.g., 4, 5, or 6) ring atoms, wherein 1-3 (e.g., 1-2) ring atoms are heteroatoms, each independently selected from the group consisting of N, N(H), N(R^{d}), O, and S(O)₀₋₂. As a non-limiting example, R² can be azetidinyl, pyrolindyl, piperazinyl, morpholinyl, or piperidinyl (e.g., R² can be piperidinyl such as piperidin-4-yl).

In certain embodiments, R² is heteroaryl of 5-10 (e.g., 5, 6, 7, 8, 9, or 10) ring atoms, wherein 1-3 ring atoms are heteroatoms, each independently selected from the group consisting of N, N(H), N(R^{d}), O, and S(O)₀₋₂. In certain of these embodiments, R² is heteroaryl of 5-10 (e.g., 5 or 6) ring atoms, wherein 1-3 (e.g., 1-2) ring atoms are heteroatoms, each independently selected from the group consisting of N, N(H), N(R^{d}), O, and S(O)₀₋₂. As a non-limiting example, R² can be pyridyl, pyrimidyl, or pyrazolyl (e.g., R² can be pyrazolyl such as pyrazol-4-yl).

In some embodiments, R⁵ is H or halo. In certain embodiments, R⁵ is H.

In some embodiments, R⁶ is H.

### The Variables Q-A

In some embodiments, Q-A is defined according to (A).

In some embodiments, Q is NH. In some other embodiments, Q is N(C₁₋₃ alkyl) (e.g., NMe or NEt).

In some embodiments, A is —(Y^{A1})ₙ—Y^{A2}. In certain of these embodiments, n is 0.

In certain other embodiments (when A is —(Y^{A1})—Y^{A2}), n is 1. In certain of these embodiments, Y^{A1} is C₁₋₆ alkylene, which is optionally substituted with 1-4 R^{a}.

In certain of these embodiments, Y^{A1} is —CH₂—, —CH₂CH₂—, —CH₂CH₂CH₂—, —CH(CF₃)—, —CH₂CH(OH)—,

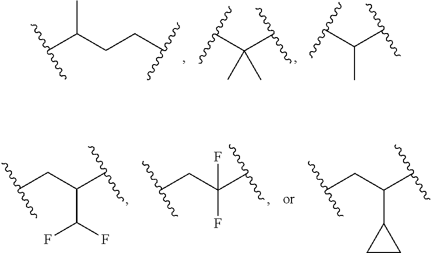

(e.g., Y^{A1} is CH₂).

In certain of these embodiments, Y^{A1} is —CH₂—, —CH₂CH₂—, —CH₂CH₂CH₂—, —CH(CF₃)—, —CH₂CHOH—,

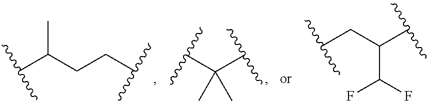

(e.g., Y^{A1} is CH₂). As a non-limiting example of the foregoing embodiments, Y^{A1} can be —CH₂— or —CH₂CH₂—.

As a non-limiting example, Y^{A1} can be —CH₂—. As another non-limiting example, Y^{A1} can be

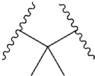 [SMILES: *C(*)(C)C]

or —CH₂CH₂—. As another non-limiting example, Y^{A1} can be

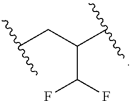 [SMILES: *CC(*)C(F)F]

In certain embodiments, Y^{A1} is Y^{A3}—Y^{A4}—Y^{A5}. In certain of these embodiments, Y^{A3} is C₂₋₃ alkylene; and/or Y^{A4} is —O—; and/or Y^{A5} is a bond. As a non-limiting example, Y^{A1} can be

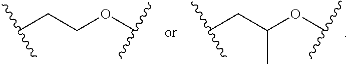

In certain embodiments, Y^{A2} is C₆₋₁₀ aryl, which is optionally substituted with 1-3 R^{c}.

In certain of these embodiments, Y^{A2} is C₆ aryl, which is optionally substituted with 1-3 R^{c}.

In certain embodiments, Y^{A2} is C₆ aryl, which is substituted with 1-3 R^{c}.

In certain embodiments, Y^{A2} is phenyl substituted with 1-3 R^{c}, wherein one R^{c} is at the ring carbon para to the point of attachment to Y^{A1}.

In certain embodiments, Y^{A2} is phenyl substituted with 1-3 R^{c}, wherein 1-2 R^{c} is at the ring carbons meta to the point of attachment to Y^{A1}.

In certain embodiments, Y^{A2} is phenyl substituted with 1-3 R^{c}, wherein 1-2 R^{c} is at the ring carbons ortho to the point of attachment to Y^{A1}.

In certain other embodiments, Y^{A2} is unsubstituted phenyl.

In certain embodiments, Y^{A2} is C₇₋₁₀ bicyclic aryl, which is optionally substituted with 1-3 R^{c} (e.g., Y^{A2} is naphthyl (e.g.,

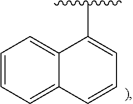 [SMILES: *c1cccc2ccccc12]

indanyl (e.g.,

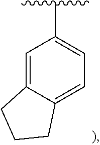 [SMILES: *c1ccc2c(c1)CCC2]

or tetrahydronapthyl, each of which is optionally substituted with 1-3 R^{c}).

In certain embodiments, Y^{A2} is heteroaryl of 5-14 ring atoms, wherein 1-3 ring atoms are heteroatoms, each independently selected from the group consisting of N, N(H), N(R^{d}), O, and S(O)₀₋₂, and wherein the heteroaryl ring is optionally substituted with 1-4 independently selected R^{c}.

In certain of the foregoing embodiments, Y^{A2} is heteroaryl of 6 ring atoms (e.g., pyridyl or pyrimidinyl (e.g., pyridyl)), wherein 1-2 ring atoms are ring nitrogen atoms, and wherein the heteroaryl ring is optionally substituted with 1-3 independently selected R^{c}.

In certain of the foregoing embodiments, Y^{A2} is heteroaryl of 6 ring atoms (e.g., pyridyl or pyrimidinyl (e.g., pyridyl)), Y^{A2} is substituted with 1-3 independently selected R^{c}; and one occurrence of R^{c} is at the ring carbon atom para to the point of attachment to Y^{A1} (e.g.,

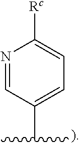

In certain of the foregoing embodiments, Y^{A2} is heteroaryl of 6 ring atoms (e.g., pyridyl or pyrimidinyl (e.g., pyridyl)), Y^{A2} is substituted with 1-3 independently selected R^{c}; and one occurrence of R^{c} is at the ring carbon atom meta to the point of attachment to Y^{A1}.

In certain embodiments, Y^{A2} is bicyclic or tricyclic heteroaryl of 7-14 (e.g., 9-12 (e.g., 9, 10, 11, or 12)) ring atoms, wherein 1-3 ring atoms are heteroatoms, each independently selected from the group consisting of N, N(H), N(R^{d}), O, and S(O)₀₋₂, and wherein the heteroaryl ring is optionally substituted with 1-4 independently selected R^{c}.

In certain of these embodiments, Y^{A2} is bicyclic heteroaryl of 9-10 ring atoms, wherein 1-3 ring atoms are heteroatoms, each independently selected from the group consisting of N, N(H), N(R^{d}), O, and S(O)₀₋₂, and wherein the heteroaryl ring is optionally substituted with 1-4 independently selected R^{c}.

As a non-limiting example of the foregoing embodiments, Y^{A2} can be bicyclic heteroaryl of 10 ring atoms, wherein 1-3 ring atoms are heteroatoms, each independently selected from the group consisting of N, N(H), N(R^{d}), O, and S(O)₀₋₂ (e.g.,

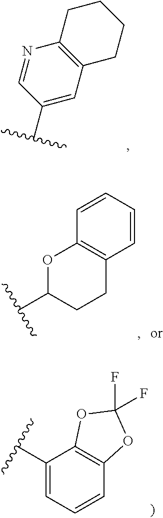 [SMILES: *c1cnc2c(c1)CCCC2 ; *C1CCc2ccccc2O1 ; *c1cccc2c1OC(F)(F)O2]

and wherein the heteroaryl ring is optionally substituted with 1-4 independently selected R^{c}.

In certain of the foregoing embodiments (wherein Y^{A2} is aryl or heteroaryl as described supra), each occurrence of R^{c} is independently selected from the group consisting of: halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —NR^{e}R^{f}; —C₁₋₄ thioalkoxy; —C(═O)(C₁₋₁₀ alkyl); —C(═O)(OH); —C(═O)O(C₁₋₄ alkyl); and -L¹-L²-R^{h}.

In certain embodiments, one occurrence of R^{e} is halo (e.g., F or Cl (e.g., Cl)).

In certain embodiments, one occurrence of R^{c} is C₂₋₆ alkynyl (e.g.,

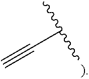 [SMILES: *C#C]

In certain embodiments, one occurrence of R^{c} is C₁₋₄ alkoxy or C₁₋₄ haloalkoxy (e.g., OCF₃). In certain embodiments, one occurrence of R^{c} is SF₅. In certain embodiments, one occurrence of R^{c} is S(O)₂(C₁₋₄ haloalkyl) (e.g., S(O)₂CF₃). In certain embodiments, one occurrence of R^{c} is C₁₋₄ thiohaloalkoxy (e.g., SCF₃).

In certain embodiments, one occurrence of R^{e} is C₁₋₁₀ alkyl which is optionally substituted with 1-6 independently selected R^{a}.

In certain embodiments, one occurrence of R^{e} is unsubstituted C₁₋₁₀ alkyl (e.g., C₂, C₃, C₄, C₅, C₆, or C₇₋₁₀). As a non-limiting example, one occurrence of R^{c} can be ethyl, propyl (e.g., n-propyl), butyl (e.g., n-butyl, iso-butyl, sec-butyl, tert-butyl), or octyl (e.g., n-octyl).

In certain embodiments (when one occurrence of R^{c} is C₁₋₁₀ alkyl which is optionally substituted with 1-6 independently selected R^{a}), the occurrence of R^{e} is C₁₋₁₀ alkyl which is substituted with 1-6 independently selected R^{a}. In certain of these embodiments, each occurrence of R^{a} is independently selected from halo, OH, C₁₋₄ alkoxy, and C₁₋₄ haloalkoxy. As a non-limiting example, each occurrence of R^{a} is halo (e.g., F). In certain embodiments (e.g., when one occurrence of R^{e} is C₁₋₁₀ alkyl which is substituted with 1-6 independently selected R^{a}), the occurrence of R^{e} is CF₃.

In certain embodiments, one occurrence of R^{e} is -L¹-L²-R^{h}. In certain of these embodiments, L¹ is a bond and/or L² is a bond.

In certain embodiments (when one occurrence of R^{c} is -L¹-L²-R^{h}), R^{h} is C₆₋₁₀ aryl, which is optionally substituted with 1-4 substituents independently selected from the group consisting of halo, C₁₋₄ alkyl, or C₁₋₄ haloalkyl.

In certain embodiments (when one occurrence of R^{c} is -L¹-L²-R^{h}), R^{h} is C₆ aryl, which is optionally substituted with 1-2 substituents independently selected from the group consisting of halo, C₁₋₄ alkyl, or C₁₋₄ haloalkyl (e.g.

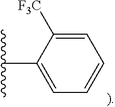 [SMILES: *c1ccccc1C(F)(F)F]

In certain embodiments (when one occurrence of R^{c} is -L¹-L²-R^{h}), R^{h} is heterocyclyl or heterocycloalkenyl, wherein the heterocyclyl or heterocycloalkenyl has 3-10 (e.g., 5-6) ring atoms, wherein 1-3 (e.g., 1-2) ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo, C₁₋₄ alkyl, and C₁₋₄ haloalkyl. As a non-limiting example of the foregoing embodiments, R^{h} can be

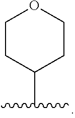 [SMILES: *C1CCOCC1]

As another non-limiting example, R^{h} can be

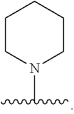 [SMILES: *N1CCCCC1]

In certain embodiments (when one occurrence of R^{c} is -L¹-L²-R^{h}), R^{h} is C₃₋₈ cycloalkyl or C₃₋₈ cycloalkenyl, each optionally substituted with 1-4 substituents independently selected from the group consisting of halo, C₁₋₄ alkyl, and C₁₋₄ haloalkyl. In certain of these embodiments, R^{h} is C₃₋₆ cycloalkyl or C₃₋₆ cycloalkenyl, each optionally substituted with 1-4 substituents independently selected from the group consisting of halo, C₁₋₄ alkyl, and C₁₋₄ haloalkyl. As non-limiting examples, R^{h} can be selected from the group consisting of:

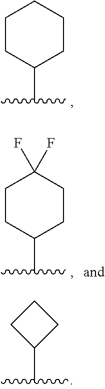

In any one of more of the foregoing embodiments of R^{c}, each of the remaining occurrences of R^{c} is C₁₋₆ alkyl or halo.

In certain embodiments, Y^{A2} is monocyclic C₃₋₁₀ cycloalkyl or C₃₃10 cycloalkenyl, each of which is optionally substituted with 1-4 R^{b}.

In certain embodiments, Y^{A2} is C₃₋₆(e.g., C₃, C₅, or C₆) cycloalkyl or C₃₋₆(e.g., C₃, C₅, or C₆) cycloalkenyl, each of which is substituted with 1-4 (e.g., 1-2) R^{b}. In certain of these embodiments, Y^{A2} is C₃₋₆(e.g., C₃, C₅, or C₆) cycloalkyl which is substituted with 1-4 (e.g., 1-2) R^{b} (e.g., Y^{A2} is cyclopropyl, cyclopentyl, or cyclohexyl, each of which is optionally substituted with 1-2 R^{b}).

In certain embodiments, Y^{A2} is cyclohexyl which is optionally substituted with 1-2 R^{b}.

In certain embodiments (when Y^{A2} is cyclohexyl which is optionally substituted with 1-2 R^{b}), one occurrence of R^{b} is at the ring carbon atom para to the point of attachment to Y^{A1}; or one occurrence of R^{b} is at the ring carbon atom meta to the point of attachment to Y^{A1}. For example, Y^{A2} can be

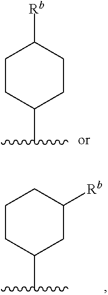

such as

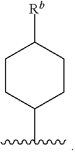

In certain embodiments (when Y^{A2} is cyclohexyl which is optionally substituted with 1-2 R^{b}), two occurrences of R^{b} are at the ring carbon atom para to the point of attachment to Y^{A1}; or two occurrences of R^{b} are at the ring carbon atom meta to the point of attachment to Y^{A1}.

In certain other embodiments, Y^{A2} is unsubstituted cyclohexyl.

In certain embodiments, Y^{A2} is cyclobutyl which is substituted with 1-2 R^{b}, such as wherein Y^{A2} is

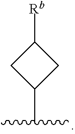

In certain embodiments, Y^{A2} is cyclopropyl which is substituted with 1-2 R^{b}. As a non-limiting example, Y^{A2} can be cyclopropyl substituted with -L¹-L²-R^{h} (e.g.,

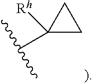

As another example, Y^{A2} can be

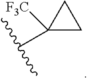 [SMILES: *C1(C(F)(F)F)CC1]

In certain embodiments, Y^{A2} is bicyclic, tricyclic, or polycyclic C₇₋₂₀ cycloalkyl or C₇₋₂₀ cycloalkenyl, each optionally substituted with 1-2 R^{b}. In certain embodiments, Y^{A2} is bicyclic, tricyclic, or polycyclic C₇₋₁₂ cycloalkyl or C₇₋₁₂ cycloalkenyl, each optionally substituted with 1-2 R^{b}. In certain embodiments, Y^{A2} is bicyclic C₇₋₈ cycloalkyl, optionally substituted with 1-2 R^{b}. In certain embodiments, Y^{A2} is bicyclic, tricyclic, or polycyclic C₉₋₁₂ cycloalkyl, optionally substituted with 1-2 R^{b}.

In certain embodiments, Y^{A2} is spirobicyclic C₇₋₁₂ cycloalkyl, optionally substituted with 1-2 R^{b} (e.g., spiro[5.5]undecanyl (e.g.,

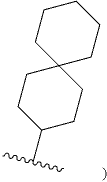 [SMILES: *C1CCC2(CCCCC2)CC1]

or spiro[2.5]octanyl (e.g.,

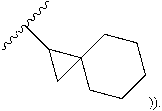 [SMILES: *C1CC12CCCCC2]

In certain of these embodiments, Y^{A2} is selected from the group consisting of: spiro[5.5]undecanyl (e.g.,

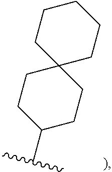 [SMILES: *C1CCC2(CCCCC2)CC1]

bicyclo[2.2.1]hept-2-enyl (e.g.,

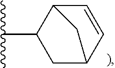 [SMILES: *C1CC2C=CC1C2]

bicyclo[2.2.1]heptanyl (e.g.,

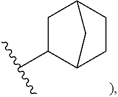 [SMILES: *C1CC2CCC1C2]

spiro[2.5]octanyl (e.g.,

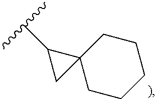 [SMILES: *C1CC12CCCCC2]

and adamantly (e.g.,

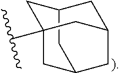 [SMILES: *C12CC3CC(CC(C3)C1)C2]

For example, Y^{A2} can be

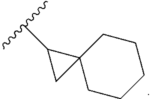 [SMILES: *C1CC12CCCCC2]

In certain embodiments, Y^{A2} is heterocyclyl or heterocycloalkenyl of 3-16 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl ring is optionally substituted with 1-3 independently selected R^{b}.

In certain embodiments, Y^{A2} is heterocyclyl or heterocycloalkenyl of 4-10 ring atoms, wherein 1-2 ring atoms are heteroatoms, each independently selected from the group consisting of N, N(H), N(R^{d}), O, and S(O)₀₋₂, and wherein the heterocyclyl or heterocycloalkenyl ring is optionally substituted with 1-3 independently selected R^{b}.

As non-limiting examples, Y^{A2} can be

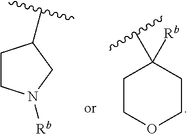

In certain embodiments, each occurrence of R^{b} substituent of Y^{A2} is independently selected from the group consisting of: C₁₋₁₀ alkyl optionally substituted with 1-6 independently selected R^{a}; C₁₋₄ haloalkyl; —F; —Cl; —Br; cyano; C₁₋₄ alkoxy; C₁₋₄ haloalkoxy; —C(═O)(C₁₋₁₀ alkyl); —C(═O)O(C₁₋₄ alkyl); —S(O)₁₋₂(C₁₋₄ alkyl); oxo; cyano; and -L¹-L²-R^{h}.

In certain of these embodiments, one occurrence of R^{b} substituent of Y^{A2} is C₁₋₁₀ alkyl which is optionally substituted with 1-6 independently selected R^{a}.

In certain of these embodiments, one occurrence of R^{b} substituent of Y^{A2} is unsubstituted C₁₋₁₀ alkyl (e.g., C₂, C₃, C₄, C₅, C₆, or C₇₋₁₀). As a non-limiting example of the foregoing embodiments, one occurrence of R^{b} substituent of Y^{A2} can be ethyl, propyl (e.g., n-propyl), butyl (e.g., n-butyl; or sec-butyl; or tert-butyl; or iso-butyl), or octyl (e.g., n-octyl).

In certain embodiments, one occurrence of R^{b} substituent of Y^{A2} is C₁₋₁₀ alkyl which is substituted with 1-6 independently selected R^{a}. In certain of these embodiments, each occurrence of R^{a} is independently selected from halo, OH, C₁₋₄ alkoxy, and C₁₋₄ haloalkoxy. For example, one or more occurrences of R^{a} can be an independently selected halo (which can be the same or different halo); e.g., fluro, and R^{b} can be CF₃ or —CF₂CH₃.

In certain embodiments, one occurrence of R^{b} substituent of Y^{A2} is -L¹-L²-R^{h} (e.g., —R^{h} or —CH₂—R^{h} such as benzyl).

In certain embodiments, one occurrence of R^{b} substituent of Y^{A2} is C₁₋₄ alkoxy or C₁₋₄ haloalkoxy (e.g.,

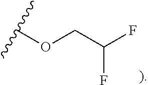 [SMILES: *OCC(F)F]

In certain embodiments, one occurrence of R^{b} is —F or —Cl (e.g., —F).

In certain embodiments, Y^{A2} is

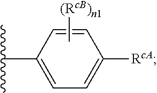

n1 is 0, 1, or 2; and each of R^{cA} and R^{cB} is an independently selected R^{c}.

In certain embodiments, Y^{A2} is

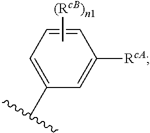

n1 is 0, 1, or 2; and each of R^{cA} and R^{cB} is an independently selected R^{c}.

In certain embodiments, Y^{A2} is

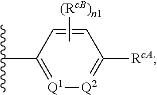

one of Q¹ and Q² is N; the other one of Q¹ and Q² is CH; n1 is 0, 1, or 2; and each of R^{cA} and R^{cB} is an independently selected R^{c}.

In certain embodiments, Y^{A2} is

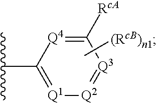

one of Q¹, Q², Q³, and Q⁴ is N; each of the remaining of Q¹, Q², Q³, and Q⁴ is CH; n1 is 0, 1, or 2; and each of R^{cA} and R^{cB} is an independently selected R^{c}.

In certain embodiments (when Y^{A2} is

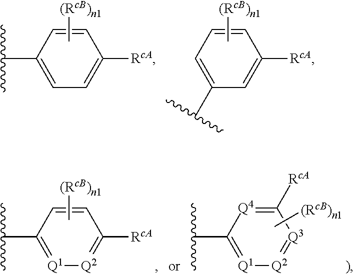

R^{cA} is selected from the group consisting of: halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —NR^{e}R^{f}; —C₁₋₄ thioalkoxy; —C(═O)(C₁₋₁₀ alkyl); —C(═O)(OH); —C(═O)O(C₁₋₄ alkyl); and -L¹-L²-R^{h}.

In certain embodiments when Y^{A2} is

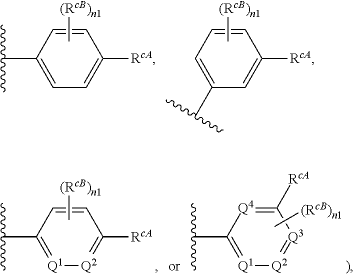

R^{cA} is unsubstituted C₁₋₁₀ alkyl (e.g., C₂, C₃, C₄, C₅, C₆, or C₇₋₁₀), such as ethyl, propyl (e.g., n-propyl), butyl (e.g., n-butyl, iso-butyl, sec-butyl, tert-butyl), or octyl (e.g., n-octyl).

In certain embodiments (when Y^{A2} is

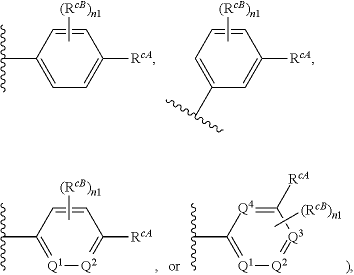

R^{cA} is C₁₋₁₀ alkyl which is substituted with 1-6 independently selected R^{a} (e.g., each occurrence of R^{a} is independently selected from halo, OH, C₁₋₄ alkoxy, and C₁₋₄ haloalkoxy). In certain of these embodiments, R^{cA} is C₁₋₁₀ alkyl which is substituted with 1-6 independently selected halo (e.g., R^{cA} is CF₃).

In certain embodiments (when Y^{A2} is

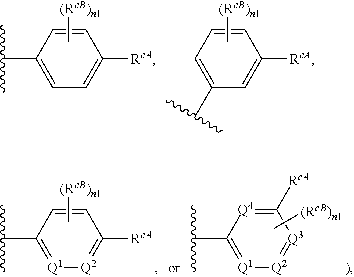

R^{cA} is C₂₋₆ alkynyl (e.g.,

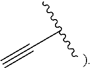 [SMILES: *C#C]

In certain embodiments (when Y^{A2} is

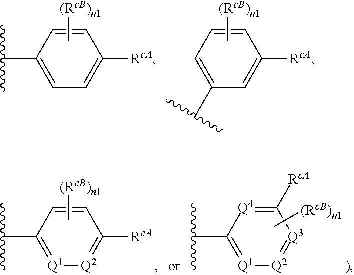

R^{cA} is C₁₋₄ haloalkoxy (e.g., —OCF₃ or

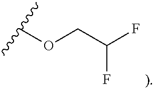 [SMILES: *OCC(F)F]

In certain embodiments (when Y^{A2} is

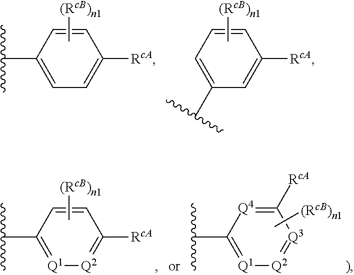

R^{cA} is -L¹-L²-R^{h}.

In certain of these embodiments, -L¹ is a bond. In certain embodiments (when R^{cA} is -L¹-L²-R^{h}), -L² is a bond.

In certain embodiments (when R^{cA} is -L¹-L²-R^{h}), R^{h} is C₆₋₁₀ aryl, which is optionally substituted with 1-4 substituents independently selected from the group consisting of halo, C₁. 4 alkyl, or C₁₋₄ haloalkyl, such as C₆ aryl, which is optionally substituted with 1-2 substituents independently selected from the group consisting of halo, C₁₋₄ alkyl, or C₁₋₄ haloalkyl (e.g.,

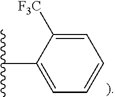 [SMILES: *c1ccccc1C(F)(F)F]

In certain embodiments (when R^{cA} is -L¹-L²-R^{h}), R^{h} is heterocyclyl or heterocycloalkenyl, wherein the heterocyclyl or heterocycloalkenyl has 3-10 (e.g., 5-6) ring atoms, wherein 1-3 (e.g., 1-2) ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo, C₁₋₄ alkyl, and C₁₋₄ haloalkyl, such as

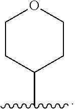 [SMILES: *C1CCOCC1]

In certain embodiments (when R^{cA} is -L¹-L²-R^{h}), R^{h} is C₃₋₈(e.g., C₃₋₆) cycloalkyl or C₃₋₈ (e.g., C₃₋₆) cycloalkenyl, each optionally substituted with 1-4 substituents independently selected from the group consisting of halo, C₁₋₄ alkyl, and C₁₋₄ haloalkyl (e.g., R^{h} is cyclohexyl).

In certain embodiments (when Y^{A2} is

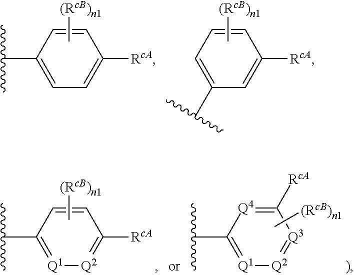

In certain embodiments (when Y^{A2} is

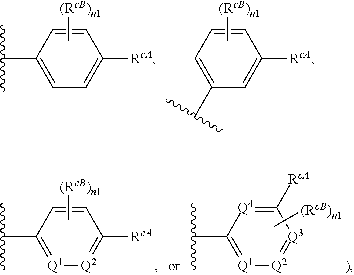

n1 is 1 or 2 (e.g., 1). In certain of these embodiments, each occurrence of R^{cB} is independently halo or C₁₋₃ alkyl (e.g., halo).

In certain embodiments, Y^{A2} is

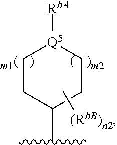

wherein Q⁵ is N or CH; m1 and m2 are independently 0, 1, or 2; n2 is 0, 1, or 2; and each of R^{bA} and R^{bB} is an independently selected R^{b}.

In certain of these embodiments, Q⁵ is CH.

In certain embodiments, Y^{A2} is

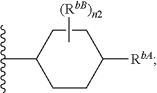

n2 is 0, 1, or 2; and each of R^{bA} and R^{bB} is an independently selected R^{b}.

In certain embodiments, Y^{A2} is

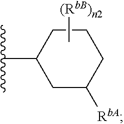

n2 is 0, 1, or 2; and each of R^{bA} and R^{bB} is an independently selected R^{b}.

In certain embodiments, Y^{A2} is

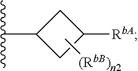

n2 is 0, 1, or 2; and each of R^{bA} and R^{bB} is an independently selected R^{b}.

In certain embodiments, Y^{A2} is

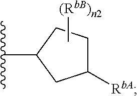

n2 is 0, 1, or 2; and each of R^{bA} and R^{bB} is an independently selected R^{b}.

In certain embodiments, Y^{A2} is

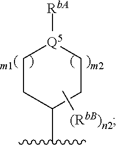

and Q⁵ is N. In certain of these embodiments, Y^{A2} is

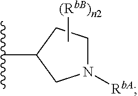

n2 is 0, 1, or 2; and each of R^{bA} and R^{bB} is an independently selected R^{b}.

In certain embodiments (when Y^{A2} is

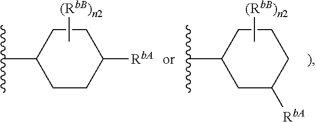

R^{bA} is C₁₋₁₀ alkyl which is optionally substituted with 1-6 independently selected R^{a}.

In certain embodiments (when Y^{A2} is

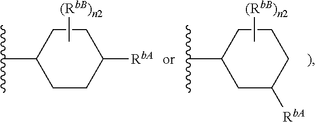

R^{bA} is unsubstituted C₁₋₁₀ alkyl (e.g., C₂, C₃, C₄, C₅, C₆, or C₇₋₁₀), such as ethyl, propyl (e.g., n-propyl), butyl (e.g., n-butyl; or sec-butyl; or tert-butyl; or iso-butyl), or octyl (e.g., n-octyl).

In certain embodiments (when Y^{A2} is

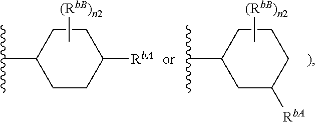

R^{bA} is C₁₋₁₀ alkyl which is substituted with 1-6 independently selected R^{a} (e.g., each R^{a} is selected from the group consisting of halo, OH, C₁₋₄ alkoxy, and C₁₋₄ haloalkoxy) (e.g., R^{bA} is CF₃ or —CF₂CH₃).

In certain embodiments (when Y^{A2} is

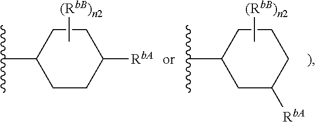

### R^{bA} is —F or —Cl.

In certain embodiments (when Y^{A2} is

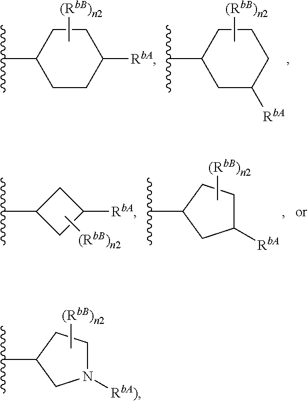

R^{bA} is -L¹-L²-R^{h} (e.g., —R^{h} or —CH₂—R^{h} such as benzyl). In certain of these embodiments, R^{bA} is —R^{h}, —O—R^{h}, or —CH₂—R^{h}. In certain of the foregoing embodiments, R^{h} is selected from the group consisting of:

R^{h} is heteroaryl of 6 ring atoms, wherein 1-2 ring atoms are ring nitrogen atoms and wherein the heteroaryl ring is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy; and

C₆ aryl, which is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy.

In certain embodiments (when Y^{A2} is

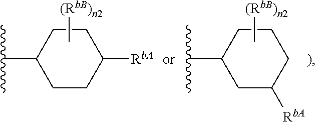

R^{bA} is -L¹-L²-R^{h} (e.g., —R^{h} or —CH₂—R^{h} such as benzyl).

In certain embodiments (when Y^{A2} is

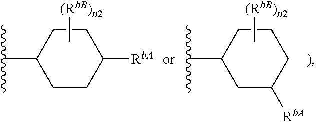

R^{bA} is C₁₋₄ alkoxy or C₁₋₄ haloalkoxy (e.g.,

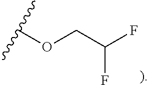 [SMILES: *OCC(F)F]

In certain embodiments (when Y^{A2} is

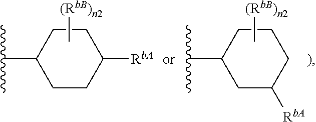

n2 is 0.

In certain other embodiments, n2 is 1 or 2. In certain of these embodiments, each occurrence R^{bB} is selected from the group consisting of —F, —Cl, and C₁₋₃ alkyl.

Non-limiting examples of A include:

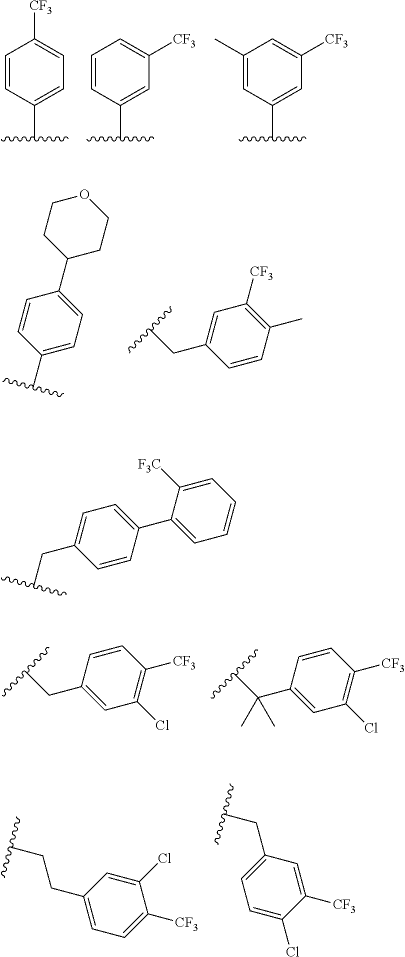

Further non-limiting examples of A include:

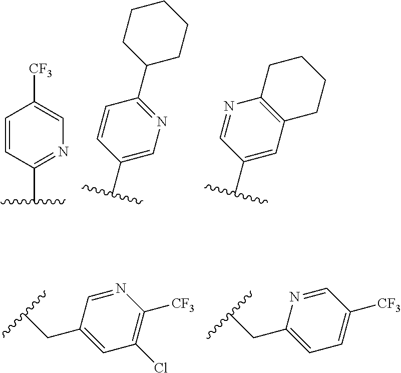

Further non-limiting examples of A include

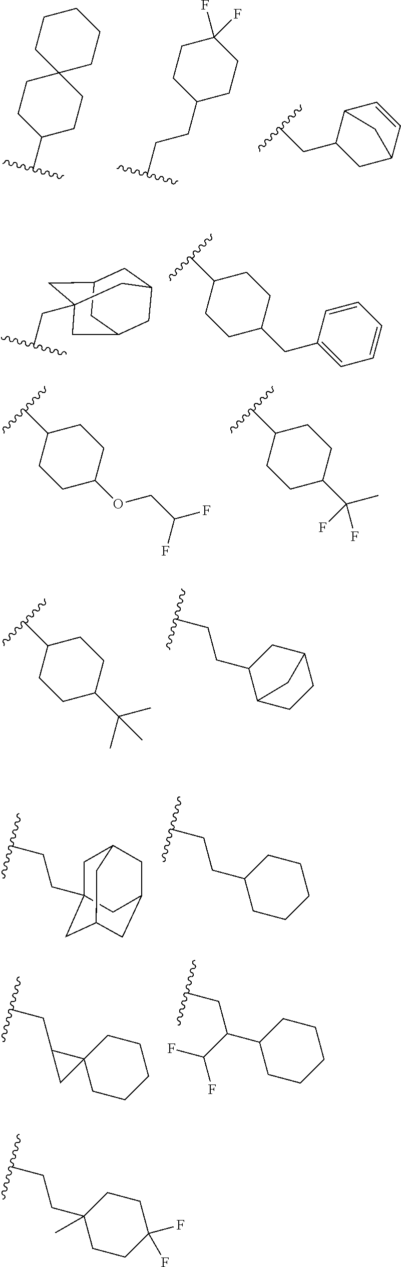

In some embodiments, Q-A is as defined according to (B).

In certain embodiments, E is a saturated or partially unsaturated ring of 3-16 ring atoms, wherein 0-3 ring atoms are heteroatoms (in addition to the nitrogen atom that is present), each independently selected from the group consisting of N, N(H), N(R^{d}), O, and S(O)₀₋₂, and wherein the carbon portion of the ring is optionally substituted with 1-4 independently selected R^{b}.

In certain embodiments, E a ring of 5-8 ring atoms, wherein aside from the nitrogen atom present, 0-3 additional ring atoms are heteroatoms, each independently selected from the group consisting of N, N(H), N(R^{d}), O, and S(O)₀₋₂, and wherein the ring is optionally substituted with 1-4 independently selected R^{b} (e.g., E is piperidinyl which is optionally substituted with 1-2 independently selected R^{b} (e.g., E is

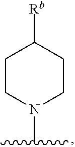

wherein R^{b} is C₁₋₆ alkyl)).

As a non-limiting example, E can be

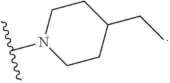 [SMILES: *N1CCC(CC)CC1]

### Non-Limiting Combinations

In certain embodiments, the compound has the following formula:

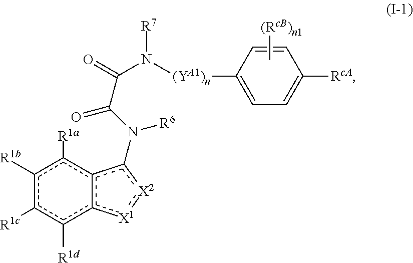

wherein n1 is 0, 1, or 2; each of R^{cA} and R^{cB} is an independently selected R^{c}; and R⁷ is H or C₁₋₄ alkyl.

In certain embodiments, the compound has the following formula:

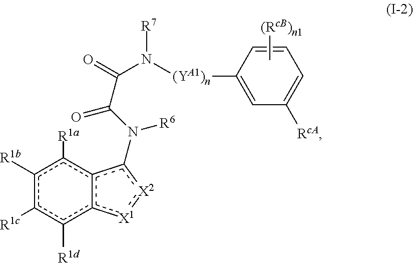

wherein n1 is 0, 1, or 2; each of R^{cA} and R^{cB} is an independently selected R^{c}; and R⁷ is H or C₁₋₄ alkyl.

In certain embodiments, the compound has the following formula:

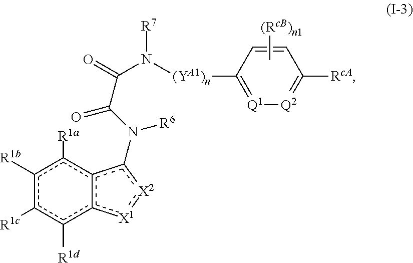

wherein one of Q¹ and Q² is N; the other one of Q¹ and Q² is CH; n1 is 0, 1, or 2; each of R^{cA} and R^{cB} is an independently selected R^{c}; and R⁷ is H or C₁₋₄ alkyl.

In certain embodiments, the compound has the following formula:

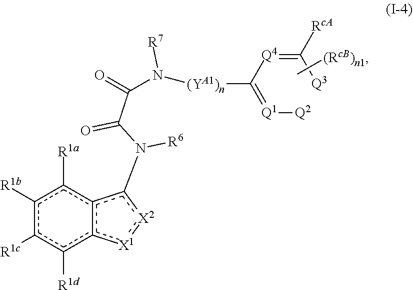

wherein one of Q¹, Q², Q³, and Q⁴ is N; each of the remaining of Qi, Q², Q³, Q⁴ is CH; n1 is 0, 1, or 2; and each of R^{cA} and R^{cB} is an independently selected R^{c}; and R⁷ is H or C₁₋₄ alkyl.

In certain embodiments, the compound has the following formula:

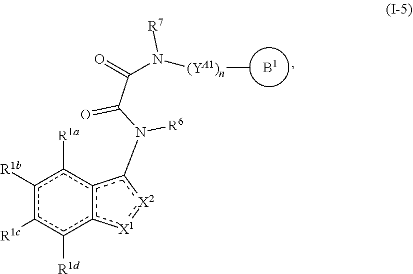

wherein B1 is selected from the group consisting of:

(a) bicyclic or tricyclic heteroaryl of 7-14 (e.g., 9-12 (e.g., 9, 10, 11, or 12)) ring atoms, wherein 1-3 ring atoms are heteroatoms, each independently selected from the group consisting of N, N(H), N(R^{d}), O, and S(O)₀₋₂, and wherein the heteroaryl ring is optionally substituted with 1-4 independently selected R^{c}; and

(b) C₇₋₁₀ bicyclic aryl, which is optionally substituted with 1-3 R^{c}; and R⁷ is H or C₁₋₄ alkyl.

In certain embodiments of Formula (I-5), B1 is bicyclic or tricyclic heteroaryl of 9-10 (e.g., 10) ring atoms, wherein 1-3 ring atoms are heteroatoms, each independently selected from the group consisting of N, N(H), N(R^{d}), O, and S(O)₀₋₂, and wherein the heteroaryl ring is optionally substituted with 1-4 independently selected R^{c}.

As a non-limiting example of the foregoing embodiments, B1 can be

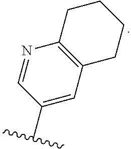 [SMILES: *c1cnc2c(c1)CCCC2]

In certain embodiments of any one or more of Formulae (I-1), (I-2), (I-3), and (I-4), R^{cA} is selected from the group consisting of: halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —NR^{e}R^{f}; —C₁₋₄ thioalkoxy; —C(═O)(C₁₋₁₀ alkyl); —C(═O)(OH); —C(═O)O(C₁₋₄ alkyl); and -L¹-L²-R^{h}.

In certain embodiments of any one or more of Formulae (I-1), (I-2), (I-3), and (I-4), R^{cA} is unsubstituted C₁₋₁₀ alkyl (e.g., C₂, C₃, C₄, C₅, C₆, or C₇₋₁₀), such as ethyl, propyl (e.g., n-propyl), butyl (e.g., n-butyl, iso-butyl, sec-butyl, tert-butyl), or octyl (e.g., n-octyl).

In certain embodiments of any one or more of Formulae (I-1), (I-2), (I-3), and (I-4), R^{cA} is C₁₋₁₀ alkyl which is substituted with 1-6 independently selected R^{a} (e.g., each occurrence of R^{a} is independently selected from halo, OH, C₁₋₄ alkoxy, and C₁₋₄ haloalkoxy).

In certain of these embodiments, R^{cA} is C₁₋₁₀ alkyl which is substituted with 1-6 independently selected halo (e.g., R^{cA} is CF₃).

In certain embodiments of any one or more of Formulae (I-1), (I-2), (I-3), and (I-4), R^{cA} is C₂₋₆ alkynyl (e.g.,

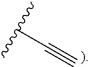 [SMILES: *C#C]

In certain embodiments of any one or more of Formulae (I-1), (I-2), (I-3), and (I-4), RcA is C₁₋₄ alkoxy or C₁₋₄ haloalkoxy.

In certain embodiments of any one or more of Formulae (I-1), (I-2), (I-3), and (I-4), R^{cA} is -L¹-L²-R^{h}. In certain of these embodiments, -L¹ is a bond. In certain embodiments (when R^{cA} is -L¹-L²-R^{h}), -L² is a bond.

In certain embodiments of any one or more of Formulae (I-1), (I-2), (I-3), and (I-4) (when R^{cA} is -L¹-L²-R^{h}), R^{h} is C₆₋₁₀ aryl, which is optionally substituted with 1-4 substituents independently selected from the group consisting of halo, C₁₋₄ alkyl, or C₁₋₄ haloalkyl, such as C₆ aryl, which is optionally substituted with 1-2 substituents independently selected from the group consisting of halo, C₁₋₄ alkyl, or C₁₋₄ haloalkyl (e.g.,

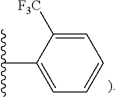 [SMILES: *c1ccccc1C(F)(F)F]

In certain embodiments of any one or more of Formulae (I-1), (I-2), (I-3), and (I-4) (when R^{cA} is -L¹-L²-R^{h}), R^{h} is heterocyclyl or heterocycloalkenyl, wherein the heterocyclyl or heterocycloalkenyl has 3-10 (e.g., 5-6) ring atoms, wherein 1-3 (e.g., 1-2) ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo, C₁₋₄ alkyl, and C₁₋₄ haloalkyl, such as

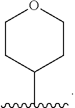 [SMILES: *C1CCOCC1]

In certain embodiments of any one or more of Formulae (I-1), (I-2), (I-3), and (I-4) (when R^{cA} is -L¹-L²-R^{h}), R^{h} is C₃₋₈(e.g., C₃₋₆) cycloalkyl or C₃₋₈ (e.g., C₃₋₆) cycloalkenyl, each optionally substituted with 1-4 substituents independently selected from the group consisting of halo, C₁₋₄ alkyl, and C₁₋₄ haloalkyl (e.g., R^{h} is cyclohexyl).

In certain embodiments of any one or more of Formulae (I-1), (I-2), (I-3), and (I-4), n1 is 0.

In certain other embodiments, n1 is 1 or 2 (e.g., 1). In certain of these embodiments, each occurrence of R^{cB} is independently halo or C₁₋₃ alkyl (e.g., halo).

In certain embodiments, the compound has the following formula:

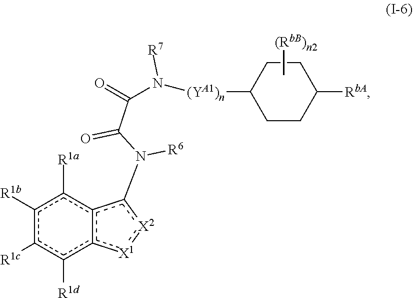

wherein n2 is 0, 1, or 2; each of R^{bA} and R^{bB} is an independently selected R^{h}; and R⁷ is H or C₁₋₄ alkyl.

In certain embodiments, the compound has the following formula:

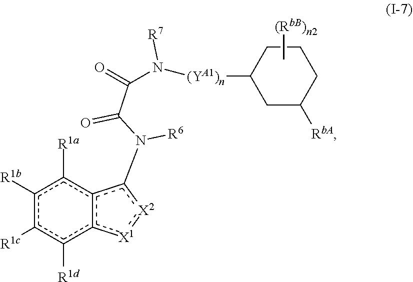

wherein n2 is 0, 1, or 2; each of R^{bA} and R^{bB} is an independently selected R^{b}; and R⁷ is H or C₁₋₄ alkyl.

In certain embodiments, the compound has the following formula:

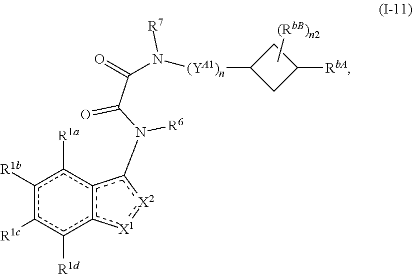

wherein n2 is 0, 1, or 2; each of R^{bA} and R^{bB} is an independently selected R^{b}; and R⁷ is H or C₁₋₄ alkyl.

In certain embodiments, the compound has the following formula:

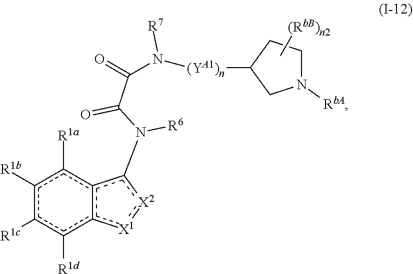

wherein n2 is 0, 1, or 2; each of R^{bA} and R^{bB} is an independently selected R^{b}; and R⁷ is H or C₁₋₄ alkyl.

In certain embodiments, the compound has the following formula:

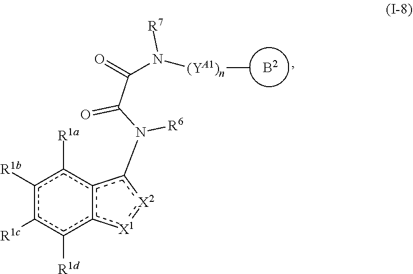

wherein B² is selected from the group consisting of:

bicyclic, tricyclic, or polycyclic C₇₋₂₀ (e.g., C₇₋₁₂) cycloalkyl or C₇₋₂₀ (e.g., C₇₋₁₂) cycloalkenyl, each optionally substituted with 1-2 R^{b}; and

bicyclic, tricyclic, or polycyclic heterocyclyl of 8-16 ring atoms, wherein 1-3 ring atoms are heteroatoms, each independently selected from the group consisting of N, N(H), N(R^{d}), O, and S(O)₀₋₂, and wherein the heterocyclyl ring is optionally substituted with 1-4 independently selected R^{b};

R⁷ is H or C₁₋₄ alkyl.

In certain embodiments, the compound has the following formula:

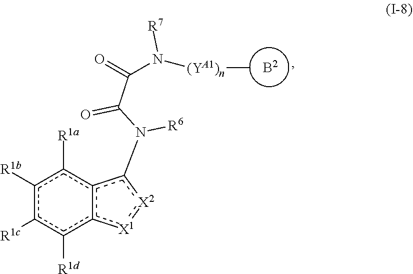

wherein B² is: bicyclic, tricyclic, or polycyclic C₇₋₂₀ cycloalkyl or C₇₋₂₀ cycloalkenyl, each optionally substituted with 1-2 R^{b}; and

R⁷ is H or C₁₋₄ alkyl.

In certain of these embodiments, B² is: bicyclic, tricyclic, or polycyclic C₇₋₁₂ cycloalkyl or C₇₋₁₂ cycloalkenyl, each optionally substituted with 1-2 R^{b}.

In certain embodiments, B² is bicyclic C₇₋₈ cycloalkyl optionally substituted with 1-2 R^{b}.

In certain embodiments, B² is bicyclic C₉₋₁₂ cycloalkyl optionally substituted with 1-2 R^{b}.

In certain embodiments, B² is spirobicyclic C₇₋₁₂ cycloalkyl optionally substituted with 1-2 R^{b}. For example, B² can be spiro[5.5]undecanyl (e.g.,

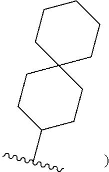 [SMILES: *C1CCC2(CCCCC2)CC1]

or spiro[2.5]octanyl (e.g.,

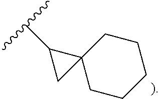 [SMILES: *C1CC12CCCCC2]

In certain embodiments of Formula (I-8), B² is selected from the group consisting of: spiro[5.5]undecanyl (e.g.,

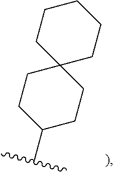 [SMILES: *C1CCC2(CCCCC2)CC1]

bicyclo[2.2.1]hept-2-enyl (e.g.,

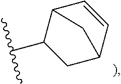 [SMILES: *C1CC2C=CC1C2]

bicyclo[2.2.1]heptanyl (e.g.,

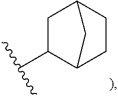 [SMILES: *C1CC2CCC1C2]

spiro[2.5]octanyl (e.g.,

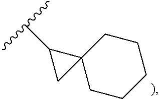 [SMILES: *C1CC12CCCCC2]

and adamantly (e.g.,

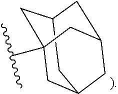 [SMILES: *C12CC3CC(CC(C3)C1)C2]

In certain embodiments of any one or more of Formulae (I-6) and (I-7), R^{bA} is C₁₋₁₀ alkyl which is optionally substituted with 1-6 independently selected R^{a}.

In certain of these embodiments, R^{bA} is unsubstituted C₁₋₁₀ alkyl (e.g., C₂, C₃, C₄, C₅, C₆, or C₇₋₁₀), such as ethyl, propyl (e.g., n-propyl), butyl (e.g., n-butyl; or sec-butyl; or tert-butyl; or iso-butyl), or octyl (e.g., n-octyl).

In certain other embodiments, R^{bA} is C₁₋₁₀ alkyl which is substituted with 1-6 independently selected R^{a} (e.g., each R^{a} is selected from the group consisting of halo, OH, C₁₋₄ alkoxy, and C₁₋₄ haloalkoxy) (e.g., R^{bA} is CF₃).

In certain embodiments of any one or more of Formulae (I-6) and (I-7), R^{bA} is —F or —Cl.

In certain embodiments of any one or more of Formulae (I-6) and (I-7), R^{bA} is -L¹-L²-R^{h} (e.g., —R^{h} or —CH₂—R^{h} such as benzyl).

In certain embodiments of any one or more of Formulae (I-6) and (I-7), R^{bA} is C₁₋₄ alkoxy or C₁₋₄ haloalkoxy (e.g.,

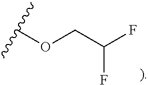 [SMILES: *OCC(F)F]

In certain embodiments of any one or more of Formulae (I-6) and (I-7), n2 is 0.

In certain other embodiments, n2 is 1 or 2. In certain of these embodiments, each occurrence R^{bB} is selected from the group consisting of —F, —Cl, and C₁₋₃ alkyl.

In some embodiments, the compound has the following formula:

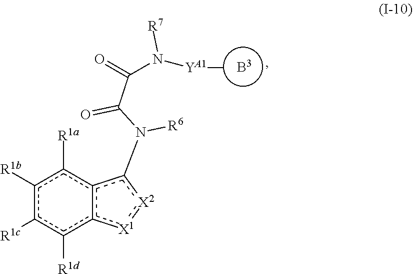

wherein B² is:

(a) C₃₋₁₀ cycloalkyl or C₃₋₁₀ cycloalkenyl, each of which is optionally substituted with 1-2 R^{b},

(b) phenyl, which is optionally substituted with 1-2 R^{c};

(c) heteroaryl of 5-6 ring atoms, wherein 1-3 ring atoms are heteroatoms, each independently selected from the group consisting of N, N(H), N(R^{d}), O, and S(O)₀₋₂, and wherein the heteroaryl ring is optionally substituted with 1-2 independently selected R^{c}; and

R⁷ is H or C₁₋₄ alkyl.

In certain embodiments of Formula (I-10), B² is C₃₋₁₀ cycloalkyl or C₃₋₁₀ cycloalkenyl, each of which is optionally substituted with 1-2 R^{b}. As a non-limiting example, B² can be C₅. 7 cycloalkyl which is unsubstituted, such as unsubstituted cyclohexyl.

In certain embodiments of Formula (I-10), B² is phenyl, which is optionally substituted with 1-2 R^{e}. As a non-limiting example, B² can be unsubstituted phenyl.

In certain embodiments of any one or more of Formulae (I-1), (I-2), (I-3), (I-4), (I-5), (I-6), (I-7), (I-8), (I-11), and (I-12), n is 0.

In certain embodiments of any one or more of Formulae (I-1), (I-2), (I-3), (I-4), (I-5), (I-6), (I-7), (I-8), (I-11), and (I-12),n is 1. In certain of these embodiments, Y^{A1} is C₁₋₆ alkylene, which is optionally substituted with 1-2 R^{a}.

In certain of these embodiments, Y^{A1} is —CH₂—, —CH₂CH₂—, —CH₂CH₂CH₂—, —CH(CF₃)—, —CH₂CH(OH)—,

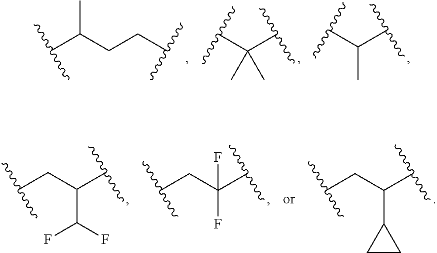

In certain of these embodiments, Y^{A1} is —CH₂—, —CH₂CH₂—, —CH₂CH₂CH₂—, —CH(CF₃)—, —CH₂CH(OH)—,

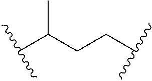 [SMILES: *CCC(*)C]

### (e.g., CH₂),

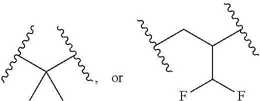

(e.g., Y^{A1} is —CH₂— or —CH₂CH₂—).

As a non-limiting example, Y^{A1} can be —CH₂-. As another non-limiting example, Y^{A1} can be —CH₂CH₂—. As another non-limiting example, Y^{A1} can be

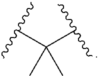 [SMILES: *C(*)(C)C]

As another non-limiting example, Y^{A1} can be

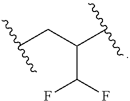 [SMILES: *CC(*)C(F)F]

In certain embodiments of any one or more of Formulae (I-1), (I-2), (I-3), (I-4), (I-5), (I-6), (I-7), (I-8), (I-10), (I-11), and (I-12) Y^{A1} is Y^{A3}—Y^{A4}—Y^{A5}. In certain of these embodiments, Y^{A3} is C₂₋₃ alkylene; and/or Y^{A4} can be —O—; and/or Y^{A5} is a bond. As a non-limiting example, Y^{A1} can be

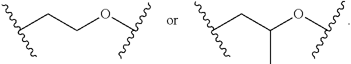

In certain embodiments, the compound has the following formula:

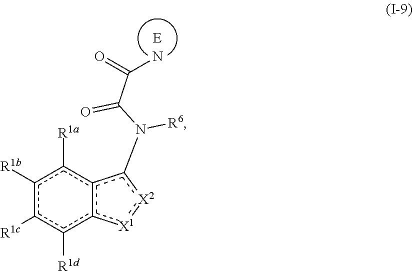

wherein:

E is a ring of 3-16 ring atoms, wherein aside from the nitrogen atom present, 0-3 additional ring atoms are heteroatoms, each independently selected from the group consisting of N, N(H), N(R^{d}), O, and S(O)₀₋₂, and wherein the ring is optionally substituted with 1-4 independently selected R^{b}.

In certain of these embodiments, E is a saturated or partially unsaturated ring of 3-16 ring atoms, wherein 0-3 ring atoms are heteroatoms (in addition to the nitrogen atom that is present), each independently selected from the group consisting of N, N(H), N(R^{d}), O, and S(O)₀₋₂, and wherein the carbon portion of the ring is optionally substituted with 1-4 independently selected R^{b}.

In certain embodiments of Formula (I-9), E is a ring of 5-8 ring atoms, wherein aside from the nitrogen atom present, 0-3 additional ring atoms are heteroatoms, each independently selected from the group consisting of N, N(H), N(R^{d}), O, and S(O)₀₋₂, and wherein the ring is optionally substituted with 1-4 independently selected R^{b} (e.g., E is piperidinyl which is optionally substituted with 1-2 independently selected R^{b} (e.g., E is

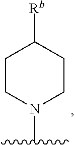

wherein R^{b} is C₁₋₆ alkyl)).

In certain embodiments of any one or more of Formulae (I-1), (I-2), (I-3), (I-4), (I-5), (I-6), (I-7), (I-8), (I-9), (I-10), (I-11), and (I-12), the

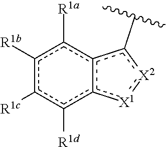

moiety is

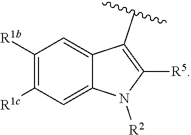

In certain embodiments of any one or more of Formulae (I-1), (I-2), (I-3), (I-4), (I-5), (I-6), (I-7), (I-8), (I-9), (I-10), (I-11), and (I-12), the

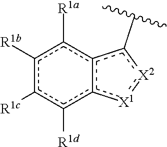

moiety is

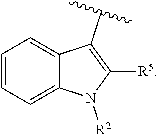

In certain embodiments of any one or more of Formulae (I-1), (I-2), (I-3), (I-4), (I-5), I-6), (I-7), (1-8), (I-9 (I-10), (I-11), and (I-12), the

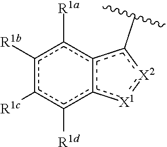

moiety is

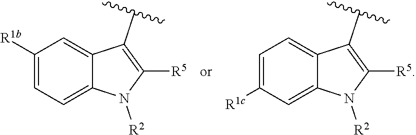

In certain embodiments of any one or more of Formulae (I-1), (I-2), (I-3), (I-4), (I-5), (I-6), (I-7), (I-8), (I-9), (I-10), (I-11), and (I-12), R² is H.

In certain embodiments of any one or more of Formulae (I-1), (I-2), (I-3), (I-4), (I-5), (I-6), (I-7), (I-8), (I-9), (I-10), (I-11), and (I-12), R⁵ is H.

In certain embodiments of any one or more of Formulae (I-1), (I-2), (I-3), (I-4), (I-5), (I-6), (I-7), (I-8), (I-10), (I-11), and (I-12), R⁷ is H.

In certain embodiments of any one or more of Formulae (I-1), (I-2), (I-3), (I-4), (I-5), (I-6), (I-7), (I-8), (I-9), (I-10), (I-11), and (I-12), each of R^{1a}, R^{1b}, R^{1c}, and R^{1d} is independently selected from the group consisting of: H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; -L³-L⁴-Rⁱ; —S(O)₁₋₂(C₁₋₄ alkyl); —S(O)(═NH)(C₁₋₄ alkyl); SF₅; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″).

In certain embodiments of any one or more of Formulae (I-1), (I-2), (I-3), (I-4), (I-5), (I-6), (I-7), (I-8), (I-9), (I-10), (I-11), and (I-12), each of R^{1a}, R^{1b}, R^{1c}, and R^{1d} is H.

In certain embodiments of any one or more of Formulae (I-1), (I-2), (I-3), (I-4), (I-5), (I-6), (I-7), (I-8), (I-9), (I-10), (I-11), and (I-12), 1-2 of R^{1a}, R^{1b}, R^{1c}, and R^{1d} is other than H.

In certain embodiments, 1-2 of R^{1a}, R^{1b}, R^{1c}, and R^{1d} is halo (e.g., F).

In certain embodiments of any one or more of Formulae (I-1), (I-2), (I-3), (I-4), (I-5), (I-6), (I-7), (I-8), (I-9), (I-10), (I-11), and (I-12), one of R^{1a}, R^{1b}, R^{1c}, and R^{1d} is -L³-L⁴-Rⁱ.

In certain of these embodiments, L³ is a bond; and/or L⁴ is a bond. As a non-limiting example, one of R^{1a}, R^{1b}, R^{1c}, and R^{1d} is R (e.g., R is heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo, C₁₋₄ alkyl, and C₁₋₄ haloalkyl; or R is C₆₋₁₀ aryl, which is optionally substituted with 1-4 substituents independently selected from the group consisting of halo, C₁₋₄ alkyl, or C₁₋₄ haloalkyl).

In certain embodiments of any one or more of Formulae (I-1), (I-2), (I-3), (I-4), (I-5), (I-6), (I-7), (I-8), (I-9), (I-10), (I-11), and (I-12), one of R^{1a}, R^{1b}, R^{1c}, and R^{1d} is -L³-L⁴-Rⁱ (e.g., R^{1b} is -L³-L⁴-Rⁱ); and each remaining R^{1a}, R^{1b}, R^{1c}, and R^{1d} is H.

In certain embodiments of any one or more of Formulae (I-1), (I-2), (I-3), (I-4), (I-5), (I-6), (I-7), (I-8), (I-9), (I-10), (I-11), and (I-12), one of R^{1a}, R^{1b}, R^{1c}, and R^{1d} (such as R^{1b}) is selected from the group consisting of:
heteroaryl of 5-6 ring atoms (such as pyrazolyl), wherein 1-4 ring atoms are heteroatoms, each independently selected from the group consisting of N, N(H), N(R^{d}), O, and S(O)₀₋₂ and wherein the heteroaryl ring is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy (e.g.,

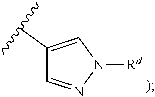

and
phenyl, which is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy (e.g.,).

In certain embodiments of any one or more of Formulae (I-1), (I-2), (I-3), (I-4), (I-5), (I-6), (I-7), (I-8), (I-9), (I-10), (I-11), and (I-12), R⁶ is H.

In certain embodiments, the compound is a compound of Formula (I-13):

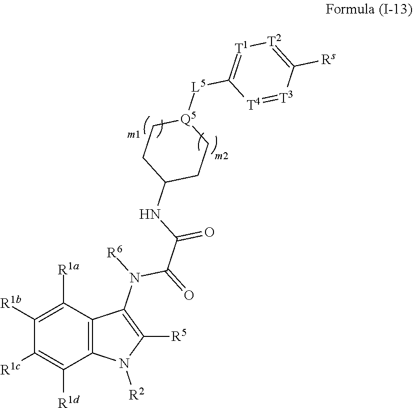

or a pharmaceutically acceptable salt thereof,

wherein:

m1 and m2 are independently 0, 1, or 2;

Q⁵ is N or CH;

L⁵ is a bond, CH₂, —O—, —N(H)—, or —N(C₁₋₃ alkyl), provided that when Q⁵ is N, then L⁵ is a bond or CH₂;

T¹, T², T³, and T⁴ are each independently N, CH, or CR^{t}, provided that 1-4, such as 2, 3, or 4, of T¹-T⁴ is CH; and

each of R^{t} and R^{s} is independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy.

In certain embodiments of Formula (I-13), R² is H; and R⁵ is H. In certain embodiments of Formula (I-13), R⁶ is H.

In certain embodiments of Formula (I-13), Q⁵ is CH. In certain of these embodiments, L⁵ is —O—. In certain embodiments of Formula (I-13), L⁵ is —N(H)— or —N(C₁₋₃ alkyl), such as —N(H)—. In certain embodiments of Formula (I-13), L⁵ is CH₂ or a bond.

In certain embodiments of Formula (I-13), Q⁵ is N. In certain of these embodiments, L⁵ is CH₂. In certain embodiments, L⁵ is a bond.

In certain embodiments of Formula (I-13), ml is 1; and m2 is 1. In certain embodiments of Formula (I-13), ml is 1; and m2 is 0. In certain embodiments of Formula (I-13), m1 is 2; and m2 is 1. In certain embodiments of Formula (I-13), ml is 0; and m2 is 0.

In certain embodiments of Formula (I-13), ml is 1; m2 is 1; Q⁵ is CH; and L⁵ is —O—.

In certain embodiments of Formula (I-13), ml is 0; m2 is 0; Q⁵ is CH; and L⁵ is —O—.

In certain embodiments of Formula (I-13), ml is 1; m2 is 0; Q⁵ is N; and L⁵ is a bond or CH₂.

In certain embodiments of Formula (I-13), each of T¹, T², T³, and T⁴ is independently CH or CR^{t}, such as each of T1, T², T³, and T⁴ is CH. In certain embodiments of Formula (I-13), T¹ is N; and T², T³, and T⁴ are independently CH or CR^{t}, such as wherein T¹ is N; and T², T³, and T⁴ are CH. In certain embodiments of Formula (I-13), T² is N; and T¹, T³, and T⁴ are independently CH or CR^{t}, such as wherein T² is N; and T¹, T³, and T⁴ are CH.

In certain embodiments of Formula (I-13), R^{s} is C₁₋₄ alkyl, such as methyl. In certain embodiments of Formula (I-13), R^{s} is C₁₋₄ haloalkyl, such as CF₃.

In certain embodiments of Formula (I-13), R^{a} is H; and R^{1d} is H or halo, such as: wherein R^{1a} is H, and R^{1d} is H; or wherein R^{1a} is H, and R^{1d} is halo such as —F or —Cl.

In certain embodiments of Formula (I-13), each of R^{1b} and R^{1c} is an independently selected halo (e.g., —F or —Cl), such as wherein R^{1b} is —Cl; and R^{1c} is —F; or wherein R^{1b} is —F; and R^{1c} is —F. In certain embodiments of Formula (I-13), R^{1b} is halo; and R^{1c} is H, such as: wherein R^{1b} is —Cl, and R^{1c} is H; or wherein R^{1b} is —F, and R^{1c} is H. In certain embodiments of Formula (I-13), R^{1b} is H; and R^{1c} is halo, such as: wherein R^{1b} is H, and R^{1c} is —F; or wherein R^{1b} is H, and R^{1c} is —Cl.

In certain embodiments of Formula (I-13), R^{1b} is Rⁱ; and R^{1c} is H or halo, such as H; such as: wherein R^{1b} is selected from the group consisting of:
heteroaryl of 5-6 ring atoms (such as pyrazolyl), wherein 1-4 ring atoms are heteroatoms, each independently selected from the group consisting of N, N(H), N(R^{d}), O, and S(O)₀₋₂ and wherein the heteroaryl ring is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy (e.g.,

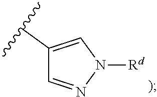

and
phenyl, which is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy (e.g.,

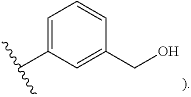 [SMILES: *c1cccc(CO)c1]

In certain embodiments of Formula (I-13), one of R^{1b} and R^{1c} is selected from the group consisting of: cyano, C₁₋₃ alkyl optionally substituted with R^{a}, and C₁₋₃ haloalkyl; and the other of R^{1b} and R^{1c} is H or halo, such as —H, —F, or —Cl.

In certain embodiments of Formula (I-13), R^{1a}, R^{1b}, R^{1c}, and R^{1d} are independently H or halo; R², R⁵, and R⁶ are H; and R^{s} is C₁₋₄ alkyl such as methyl or C₁₋₄ haloalkyl, such as CF₃. In certain of these embodiments, each of T¹, T², T³, and T⁴ is N or CH; and L⁵ is a bond or —O—.

Compound Provisions

In some embodiments, the compound is other than the compounds disclosed in Diao, Peng-Cheng; Jian, Xie-Er; Chen, Peng; Huang, Chuan; Yin, Jie; Huang, Jie Chun; Li, Jun-Sheng; Zhao, Pei-Liang, “Design, synthesis and biological evaluation of novel indole-based oxalamide and aminoacetamide derivatives as tubulin polymerization inhibitors”, Bioorganic & Medicinal Chemistry Letters Volume 30, Issue 2, 15 Jan. 2020, 126816 (DOI: 10.1016/j.bmcl.2019.126816), which is incorporated by reference herein in its entirety.

In some embodiments, the compound is other than

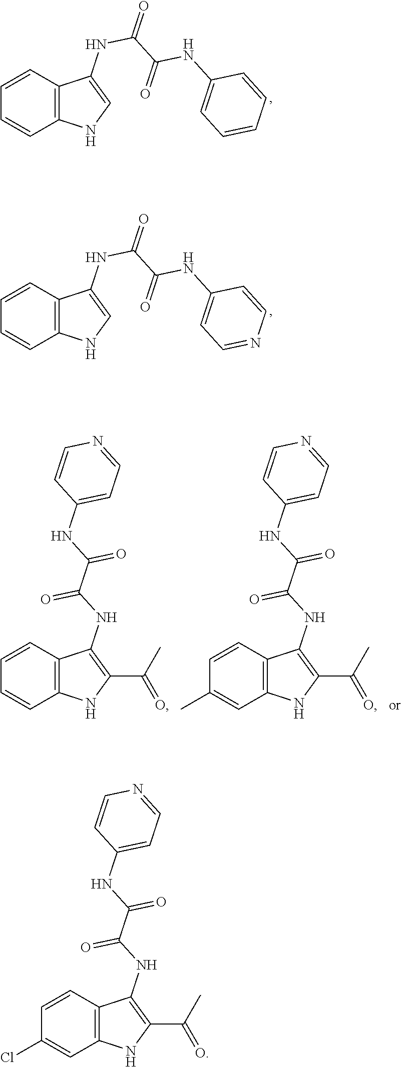

In some embodiments, when R^{1a}, R^{1b}, and R^{1d} are each H; R^{1c} is H, Me, or Cl; X¹ is NH; X² is CH or C—C(═O)Me; Q-A is as defined according to (A); A is —(Y^{A1})ₙ—Y^{A2}; and n is 0, then Y^{A2} is other than unsubstituted phenyl or unsubstituted 4-pyridyl.

In some embodiments, when X¹ is NH; X² is CH or C—C(═O)Me; Q-A is as defined according to (A); A is —(Y^{A1})ₙ—Y^{A2}; and n is 0, then Y^{A2} is other than unsubstituted phenyl or unsubstituted 4-pyridyl.

In some embodiments, when Q-A is as defined according to (A); A is —(Y^{A1})ₙ—Y^{A2}; and n is 0, then Y^{A2} is other than unsubstituted phenyl or unsubstituted 4-pyridyl.

Non-Limiting Exemplary Compounds

In certain embodiments, the compound is selected from the group consisting of the compounds delineated in Table C1, or a pharmaceutically acceptable salt thereof.

TABLE C1

| Compound No. | Structure |
|---|---|
| 101 | 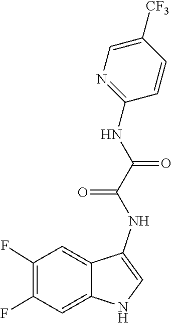 [SMILES: O=C(Nc1ccc(C(F)(F)F)cn1)C(=O)Nc1c[nH]c2cc(F)c(F)cc12] |
| 102 | 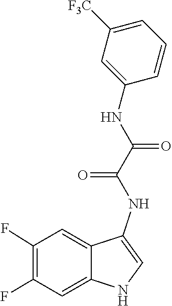 [SMILES: O=C(Nc1cccc(C(F)(F)F)c1)C(=O)Nc1c[nH]c2cc(F)c(F)cc12] |
| 103 | 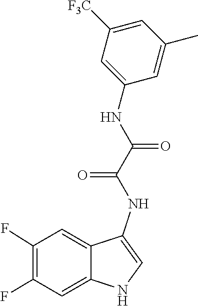 [SMILES: Cc1cc(NC(=O)C(=O)Nc2c[nH]c3cc(F)c(F)cc23)cc(C(F)(F)F)c1] |
| 104 | 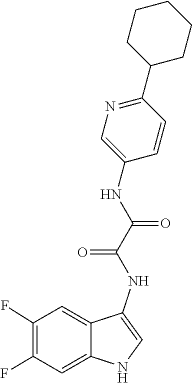 [SMILES: O=C(Nc1ccc(C2CCCCC2)nc1)C(=O)Nc1c[nH]c2cc(F)c(F)cc12] |
| 105 | 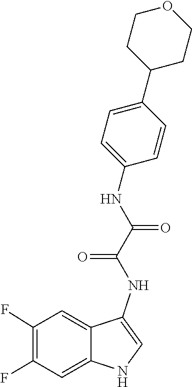 [SMILES: O=C(Nc1ccc(C2CCOCC2)cc1)C(=O)Nc1c[nH]c2cc(F)c(F)cc12] |
| 106 | 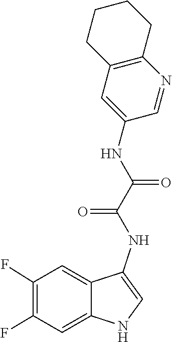 [SMILES: O=C(Nc1cnc2c(c1)CCCC2)C(=O)Nc1c[nH]c2cc(F)c(F)cc12] |
| 107 | 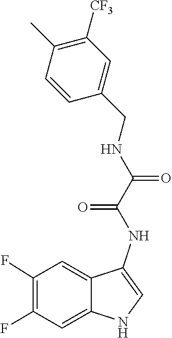 [SMILES: Cc1ccc(CNC(=O)C(=O)Nc2c[nH]c3cc(F)c(F)cc23)cc1C(F)(F)F] |
| 108 | 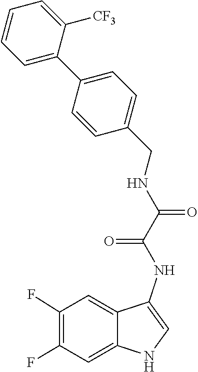 [SMILES: O=C(NCc1ccc(-c2ccccc2C(F)(F)F)cc1)C(=O)Nc1c[nH]c2cc(F)c(F)cc12] |
| 109 | 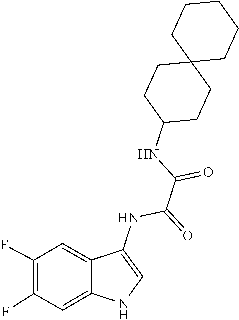 [SMILES: O=C(Nc1c[nH]c2cc(F)c(F)cc12)C(=O)NC1CCC2(CCCCC2)CC1] |
| 110 | 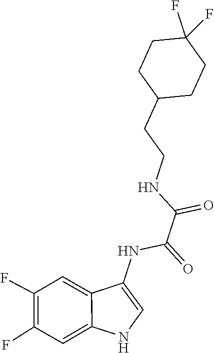 [SMILES: O=C(NCCC1CCC(F)(F)CC1)C(=O)Nc1c[nH]c2cc(F)c(F)cc12] |
| 111 | 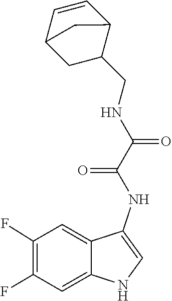 [SMILES: O=C(NCC1CC2C=CC1C2)C(=O)Nc1c[nH]c2cc(F)c(F)cc12] |
| 112 | 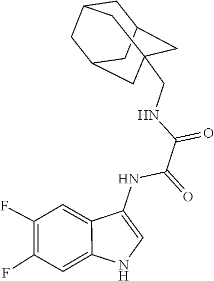 [SMILES: O=C(NCC12CC3CC(CC(C3)C1)C2)C(=O)Nc1c[nH]c2cc(F)c(F)cc12] |
| 113 | 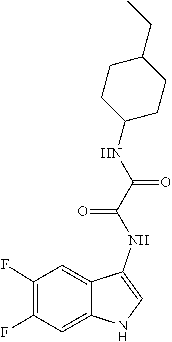 [SMILES: CCC1CCC(NC(=O)C(=O)Nc2c[nH]c3cc(F)c(F)cc23)CC1] |
| 114 | 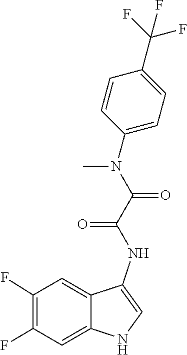 [SMILES: CN(C(=O)C(=O)Nc1c[nH]c2cc(F)c(F)cc12)c1ccc(C(F)(F)F)cc1] |
| 115 | 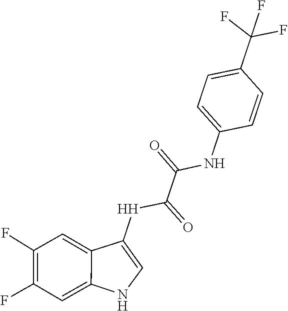 |
| 116 | 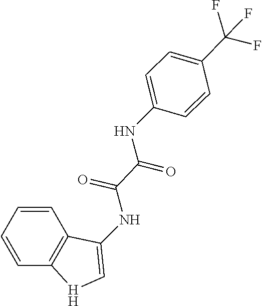 |
| 117 | 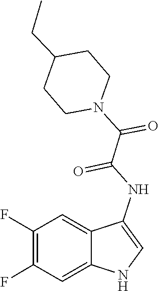 [SMILES: CCC1CCN(C(=O)C(=O)Nc2c[nH]c3cc(F)c(F)cc23)CC1] |
| 118 | 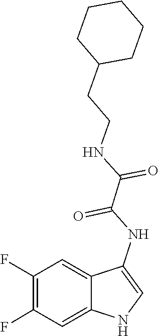 [SMILES: O=C(NCCC1CCCCC1)C(=O)Nc1c[nH]c2cc(F)c(F)cc12] |
| 119 | 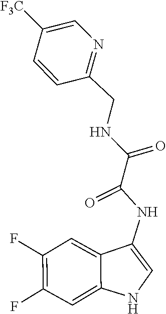 [SMILES: O=C(NCc1ccc(C(F)(F)F)cn1)C(=O)Nc1c[nH]c2cc(F)c(F)cc12] |
| 120 | 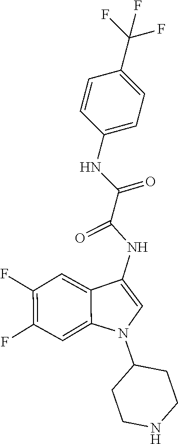 [SMILES: O=C(Nc1ccc(C(F)(F)F)cc1)C(=O)Nc1cn(C2CCNCC2)c2cc(F)c(F)cc12] |
| 121 | 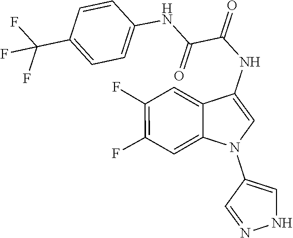 [SMILES: O=C(Nc1ccc(C(F)(F)F)cc1)C(=O)Nc1cn(-c2cn[nH]c2)c2cc(F)c(F)cc12] |
| 122 | 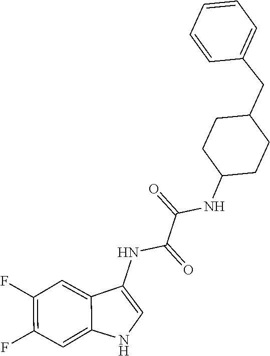 [SMILES: O=C(Nc1c[nH]c2cc(F)c(F)cc12)C(=O)NC1CCC(Cc2ccccc2)CC1] |
| 123 | 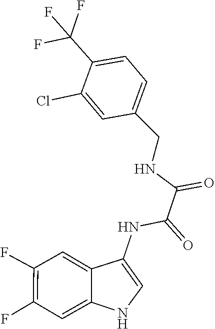 [SMILES: O=C(NCc1ccc(C(F)(F)F)c(Cl)c1)C(=O)Nc1c[nH]c2cc(F)c(F)cc12] |
| 124 | 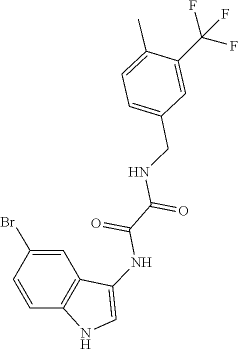 [SMILES: Cc1ccc(CNC(=O)C(=O)Nc2c[nH]c3ccc(Br)cc23)cc1C(F)(F)F] |
| 125 | 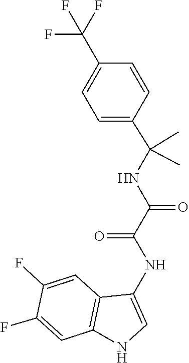 [SMILES: CC(C)(NC(=O)C(=O)Nc1c[nH]c2cc(F)c(F)cc12)c1ccc(C(F)(F)F)cc1] |
| 126 | 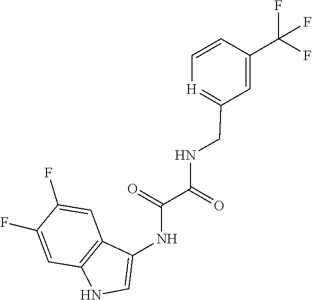 |
| 127 | 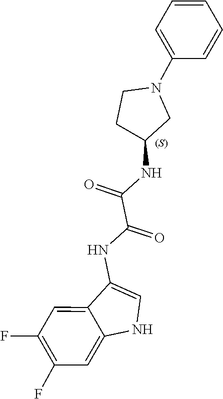 |
| 128 | 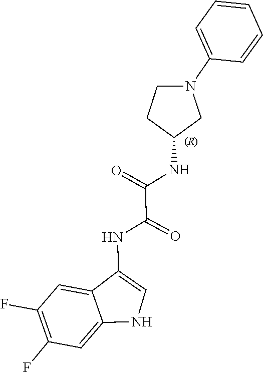 |
| 129 | 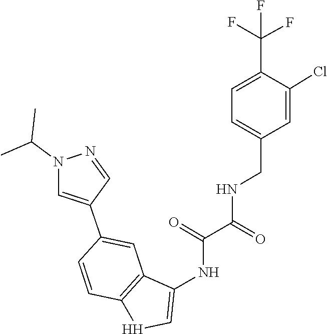 |
| 130 | 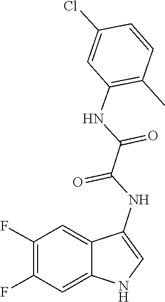 [SMILES: Cc1ccc(Cl)cc1NC(=O)C(=O)Nc1c[nH]c2cc(F)c(F)cc12] |
| 131 | 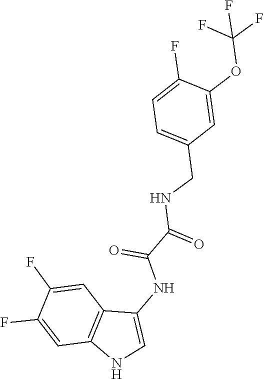 [SMILES: O=C(NCc1ccc(F)c(OC(F)(F)F)c1)C(=O)Nc1c[nH]c2cc(F)c(F)cc12] |
| 132 | 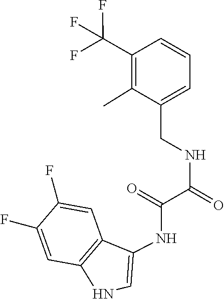 [SMILES: Cc1c(CNC(=O)C(=O)Nc2c[nH]c3cc(F)c(F)cc23)cccc1C(F)(F)F] |
| 133 | 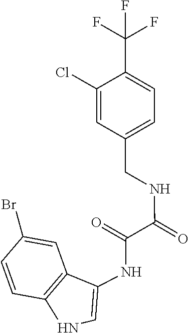 [SMILES: O=C(NCc1ccc(C(F)(F)F)c(Cl)c1)C(=O)Nc1c[nH]c2ccc(Br)cc12] |
| 134 | 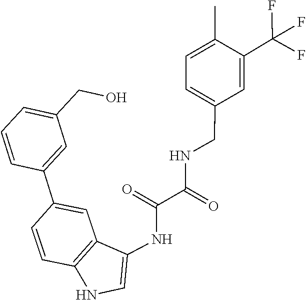 [SMILES: Cc1ccc(CNC(=O)C(=O)Nc2c[nH]c3ccc(-c4cccc(CO)c4)cc23)cc1C(F)(F)F] |
| 135 | 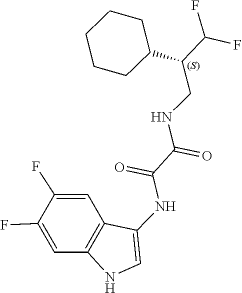 |
| 136 | 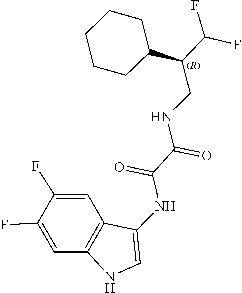 |
| 137 | 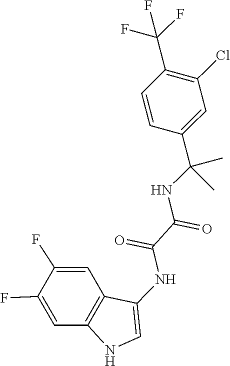 [SMILES: CC(C)(NC(=O)C(=O)Nc1c[nH]c2cc(F)c(F)cc12)c1ccc(C(F)(F)F)c(Cl)c1] |
| 138 | 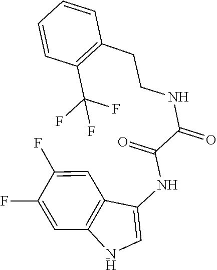 [SMILES: O=C(NCCc1ccccc1C(F)(F)F)C(=O)Nc1c[nH]c2cc(F)c(F)cc12] |
| 139 | 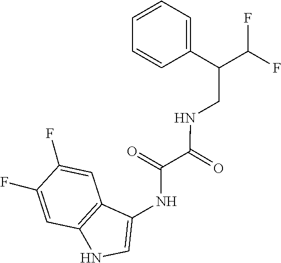 [SMILES: O=C(NCC(c1ccccc1)C(F)F)C(=O)Nc1c[nH]c2cc(F)c(F)cc12] |
| 140 | 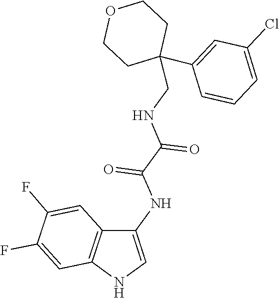 [SMILES: O=C(NCC1(c2cccc(Cl)c2)CCOCC1)C(=O)Nc1c[nH]c2cc(F)c(F)cc12] |
| 141 | 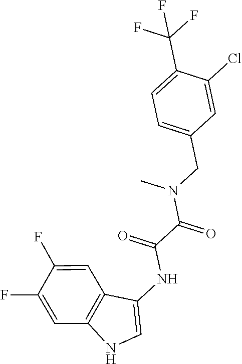 [SMILES: CN(Cc1ccc(C(F)(F)F)c(Cl)c1)C(=O)C(=O)Nc1c[nH]c2cc(F)c(F)cc12] |
| 142 | 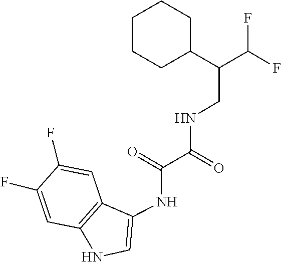 [SMILES: O=C(NCC(C(F)F)C1CCCCC1)C(=O)Nc1c[nH]c2cc(F)c(F)cc12] |
| 143 | 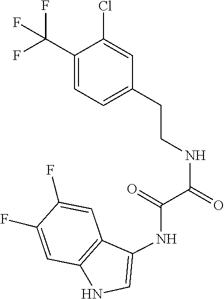 [SMILES: O=C(NCCc1ccc(C(F)(F)F)c(Cl)c1)C(=O)Nc1c[nH]c2cc(F)c(F)cc12] |
| 144 | 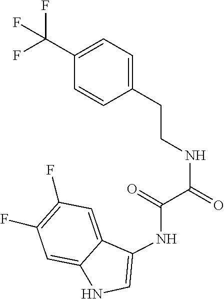 [SMILES: O=C(NCCc1ccc(C(F)(F)F)cc1)C(=O)Nc1c[nH]c2cc(F)c(F)cc12] |
| 145 | 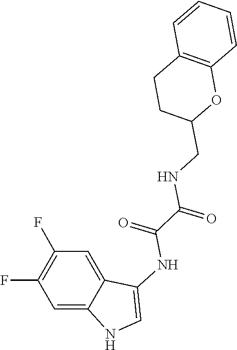 [SMILES: O=C(NCC1CCc2ccccc2O1)C(=O)Nc1c[nH]c2cc(F)c(F)cc12] |
| 146 | 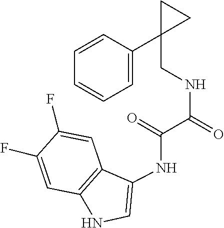 [SMILES: O=C(NCC1(c2ccccc2)CC1)C(=O)Nc1c[nH]c2cc(F)c(F)cc12] |
| 147 | 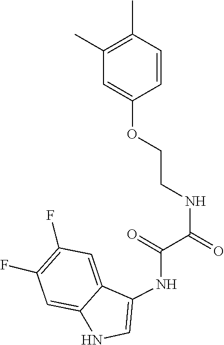 [SMILES: Cc1ccc(OCCNC(=O)C(=O)Nc2c[nH]c3cc(F)c(F)cc23)cc1C] |
| 148 | 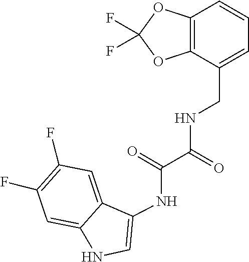 [SMILES: O=C(NCc1cccc2c1OC(F)(F)O2)C(=O)Nc1c[nH]c2cc(F)c(F)cc12] |
| 149 | 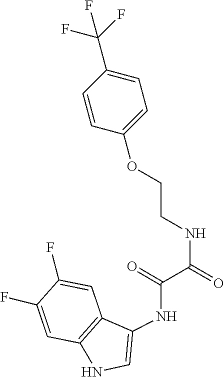 [SMILES: O=C(NCCOc1ccc(C(F)(F)F)cc1)C(=O)Nc1c[nH]c2cc(F)c(F)cc12] |
| 150 | 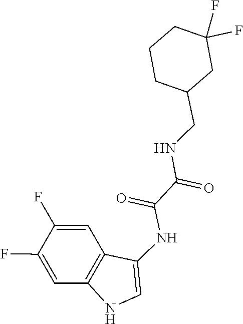 [SMILES: O=C(NCC1CCCC(F)(F)C1)C(=O)Nc1c[nH]c2cc(F)c(F)cc12] |
| 151 | 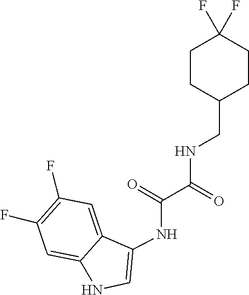 [SMILES: O=C(NCC1CCC(F)(F)CC1)C(=O)Nc1c[nH]c2cc(F)c(F)cc12] |
| 152 | 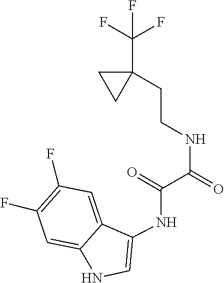 [SMILES: O=C(NCCC1(C(F)(F)F)CC1)C(=O)Nc1c[nH]c2cc(F)c(F)cc12] |
| 153 | 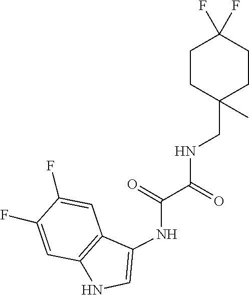 [SMILES: CC1(CNC(=O)C(=O)Nc2c[nH]c3cc(F)c(F)cc23)CCC(F)(F)CC1] |
| 154 | 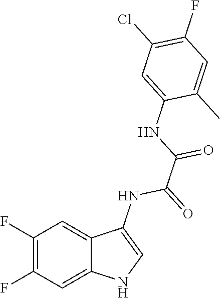 [SMILES: Cc1cc(F)c(Cl)cc1NC(=O)C(=O)Nc1c[nH]c2cc(F)c(F)cc12] |
| 155 | 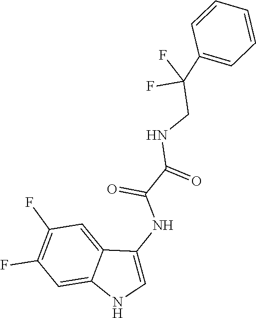 [SMILES: O=C(NCC(F)(F)c1ccccc1)C(=O)Nc1c[nH]c2cc(F)c(F)cc12] |
| 156 | 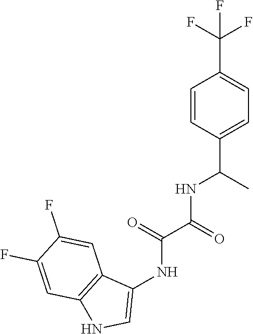 [SMILES: CC(NC(=O)C(=O)Nc1c[nH]c2cc(F)c(F)cc12)c1ccc(C(F)(F)F)cc1] |
| 157 | 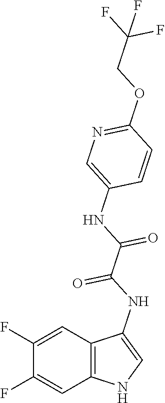 [SMILES: O=C(Nc1ccc(OCC(F)(F)F)nc1)C(=O)Nc1c[nH]c2cc(F)c(F)cc12] |
| 158 | 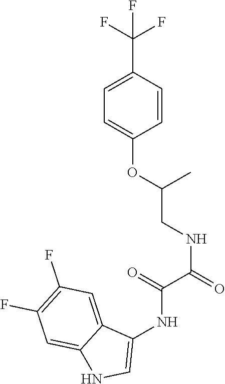 [SMILES: CC(CNC(=O)C(=O)Nc1c[nH]c2cc(F)c(F)cc12)Oc1ccc(C(F)(F)F)cc1] |
| 159 | 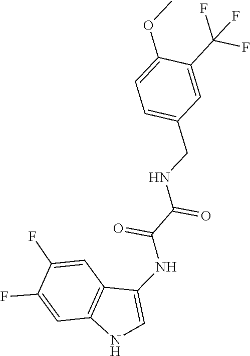 [SMILES: COc1ccc(CNC(=O)C(=O)Nc2c[nH]c3cc(F)c(F)cc23)cc1C(F)(F)F] |
| 160 | 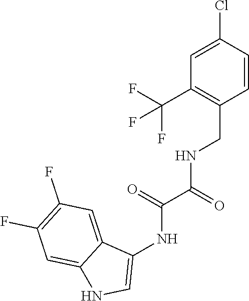 [SMILES: O=C(NCc1ccc(Cl)cc1C(F)(F)F)C(=O)Nc1c[nH]c2cc(F)c(F)cc12] |
| 161 | 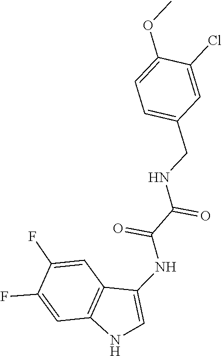 [SMILES: COc1ccc(CNC(=O)C(=O)Nc2c[nH]c3cc(F)c(F)cc23)cc1Cl] |
| 162 | 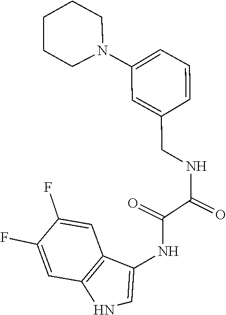 [SMILES: O=C(NCc1cccc(N2CCCCC2)c1)C(=O)Nc1c[nH]c2cc(F)c(F)cc12] |
| 163 | 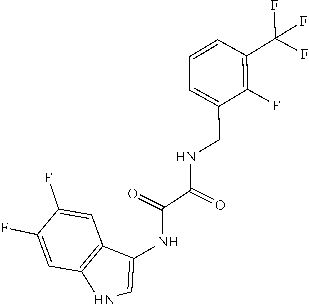 [SMILES: O=C(NCc1cccc(C(F)(F)F)c1F)C(=O)Nc1c[nH]c2cc(F)c(F)cc12] |
| 164 | 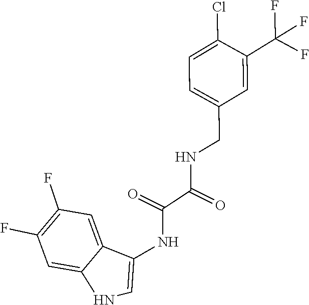 [SMILES: O=C(NCc1ccc(Cl)c(C(F)(F)F)c1)C(=O)Nc1c[nH]c2cc(F)c(F)cc12] |
| 165 | 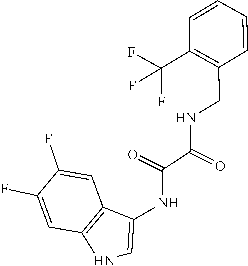 [SMILES: O=C(NCc1ccccc1C(F)(F)F)C(=O)Nc1c[nH]c2cc(F)c(F)cc12] |
| 166 | 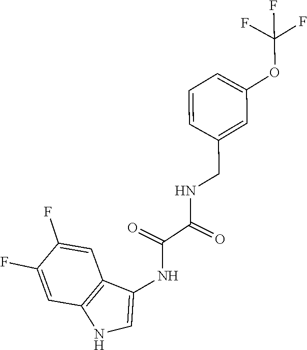 [SMILES: O=C(NCc1cccc(OC(F)(F)F)c1)C(=O)Nc1c[nH]c2cc(F)c(F)cc12] |
| 167 | 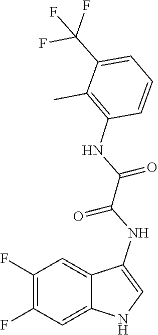 [SMILES: Cc1c(NC(=O)C(=O)Nc2c[nH]c3cc(F)c(F)cc23)cccc1C(F)(F)F] |
| 168 | 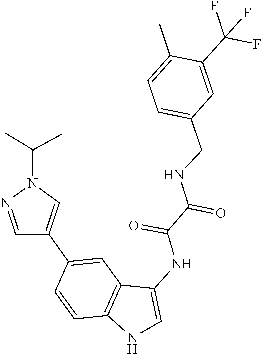 [SMILES: Cc1ccc(CNC(=O)C(=O)Nc2c[nH]c3ccc(-c4cnn(C(C)C)c4)cc23)cc1C(F)(F)F] |
| 169 | 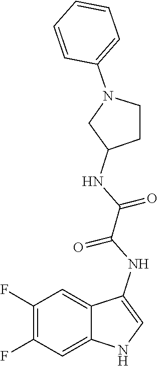 [SMILES: O=C(Nc1c[nH]c2cc(F)c(F)cc12)C(=O)NC1CCN(c2ccccc2)C1] |
| 170 | 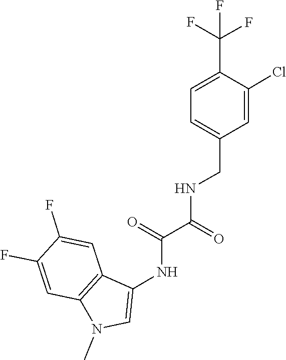 [SMILES: Cn1cc(NC(=O)C(=O)NCc2ccc(C(F)(F)F)c(Cl)c2)c2cc(F)c(F)cc21] |
| 171 | 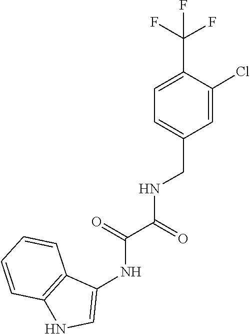 [SMILES: O=C(NCc1ccc(C(F)(F)F)c(Cl)c1)C(=O)Nc1c[nH]c2ccccc12] |
| 172 | 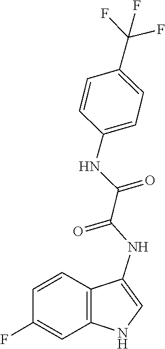 [SMILES: O=C(Nc1ccc(C(F)(F)F)cc1)C(=O)Nc1c[nH]c2cc(F)ccc12] |
| 173 | 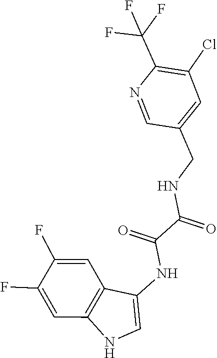 [SMILES: O=C(NCc1cnc(C(F)(F)F)c(Cl)c1)C(=O)Nc1c[nH]c2cc(F)c(F)cc12] |
| 174 | 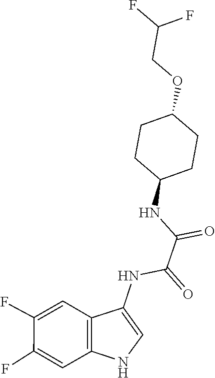 [SMILES: O=C(Nc1c[nH]c2cc(F)c(F)cc12)C(=O)N[C@H]1CC[C@H](OCC(F)F)CC1] |
| 175 | 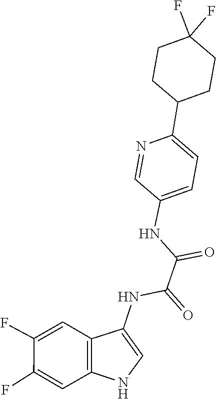 [SMILES: O=C(Nc1ccc(C2CCC(F)(F)CC2)nc1)C(=O)Nc1c[nH]c2cc(F)c(F)cc12] |
| 176 | 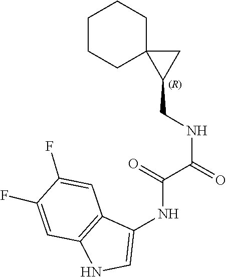 |
| 177 | 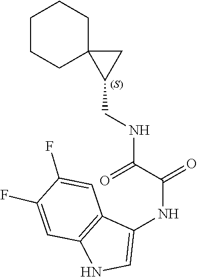 |
| 178 | 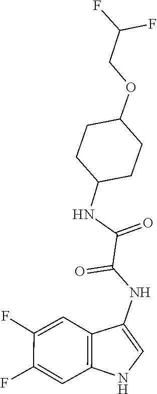 [SMILES: O=C(Nc1c[nH]c2cc(F)c(F)cc12)C(=O)NC1CCC(OCC(F)F)CC1] |
| 179 | 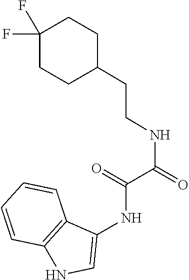 [SMILES: O=C(NCCC1CCC(F)(F)CC1)C(=O)Nc1c[nH]c2ccccc12] |
| 180 | 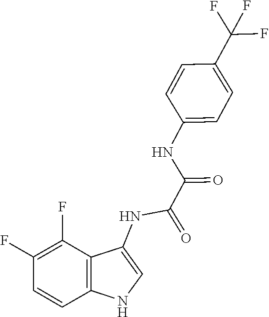 [SMILES: O=C(Nc1ccc(C(F)(F)F)cc1)C(=O)Nc1c[nH]c2ccc(F)c(F)c12] |
| 181 | 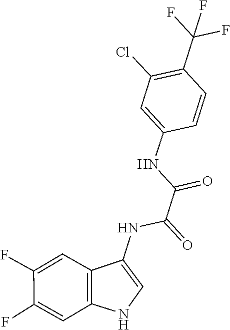 [SMILES: O=C(Nc1ccc(C(F)(F)F)c(Cl)c1)C(=O)Nc1c[nH]c2cc(F)c(F)cc12] |
| 182 | 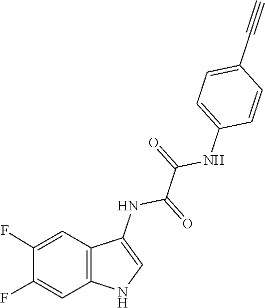 [SMILES: C#Cc1ccc(NC(=O)C(=O)Nc2c[nH]c3cc(F)c(F)cc23)cc1] |
| 183 | 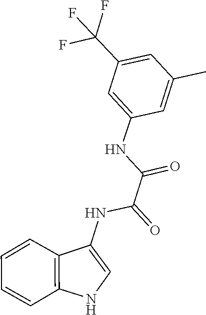 [SMILES: Cc1cc(NC(=O)C(=O)Nc2c[nH]c3ccccc23)cc(C(F)(F)F)c1] |
| 184 | 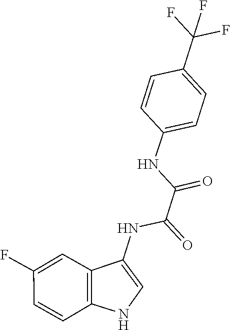 [SMILES: O=C(Nc1ccc(C(F)(F)F)cc1)C(=O)Nc1c[nH]c2ccc(F)cc12] |
| 185 | 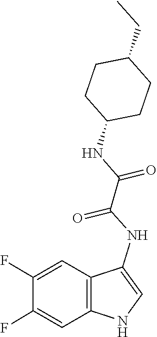 [SMILES: CC[C@H]1CC[C@@H](NC(=O)C(=O)Nc2c[nH]c3cc(F)c(F)cc23)CC1] |
| 186 | 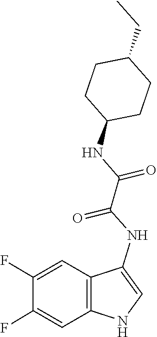 [SMILES: CC[C@H]1CC[C@H](NC(=O)C(=O)Nc2c[nH]c3cc(F)c(F)cc23)CC1] |
| 187 | 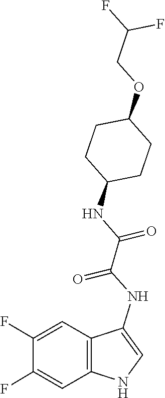 [SMILES: O=C(Nc1c[nH]c2cc(F)c(F)cc12)C(=O)N[C@H]1CC[C@@H](OCC(F)F)CC1] |
| 188 | 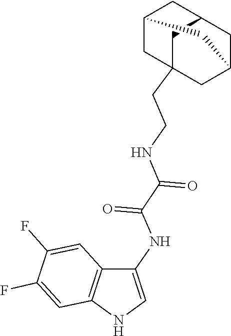 [SMILES: O=C(NCCC12C[C@H]3C[C@@H](C1)C[C@@H](C2)C3)C(=O)Nc1c[nH]c2cc(F)c(F)cc12] |
| 189 | 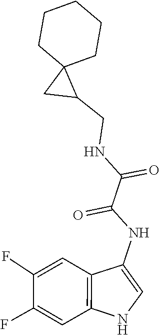 [SMILES: O=C(NCC1CC12CCCCC2)C(=O)Nc1c[nH]c2cc(F)c(F)cc12] |
| 190 | 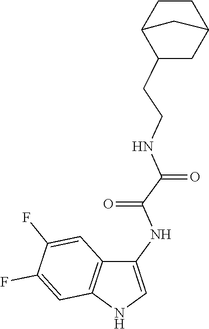 [SMILES: O=C(NCCC1CC2CCC1C2)C(=O)Nc1c[nH]c2cc(F)c(F)cc12] |
| 191 | 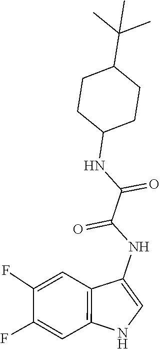 [SMILES: CC(C)(C)C1CCC(NC(=O)C(=O)Nc2c[nH]c3cc(F)c(F)cc23)CC1] |
| 192 | 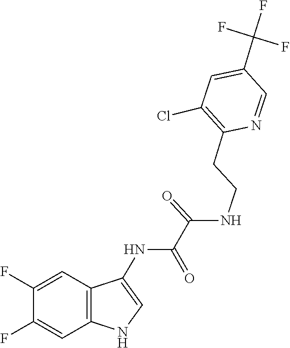 [SMILES: O=C(NCCc1ncc(C(F)(F)F)cc1Cl)C(=O)Nc1c[nH]c2cc(F)c(F)cc12] |
| 193 | 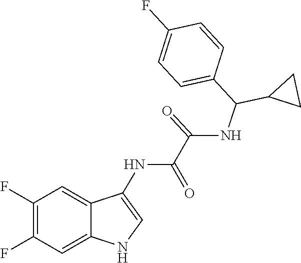 [SMILES: O=C(Nc1c[nH]c2cc(F)c(F)cc12)C(=O)NC(c1ccc(F)cc1)C1CC1] |
| 194 | 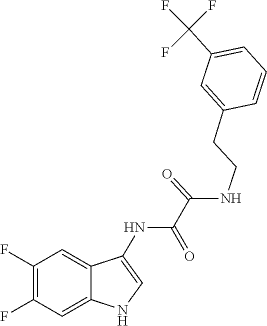 [SMILES: O=C(NCCc1cccc(C(F)(F)F)c1)C(=O)Nc1c[nH]c2cc(F)c(F)cc12] |
| 195 | 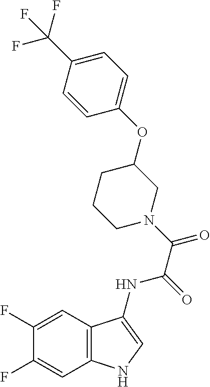 [SMILES: O=C(Nc1c[nH]c2cc(F)c(F)cc12)C(=O)N1CCCC(Oc2ccc(C(F)(F)F)cc2)C1] |
| 196 | 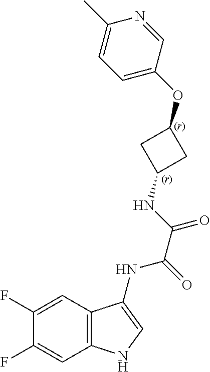 [SMILES: Cc1ccc(O[C@H]2C[C@H](NC(=O)C(=O)Nc3c[nH]c4cc(F)c(F)cc34)C2)cn1] |
| 197 | 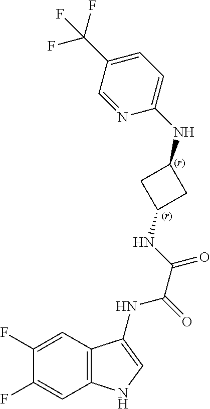 [SMILES: O=C(Nc1c[nH]c2cc(F)c(F)cc12)C(=O)N[C@H]1C[C@H](Nc2ccc(C(F)(F)F)cn2)C1] |
| 198 | 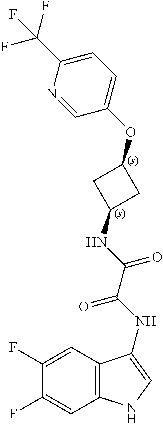 [SMILES: O=C(Nc1c[nH]c2cc(F)c(F)cc12)C(=O)N[C@H]1C[C@@H](Oc2ccc(C(F)(F)F)nc2)C1] |
| 199 | 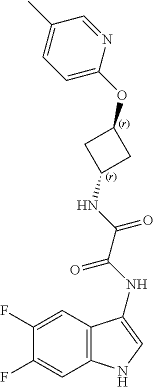 [SMILES: Cc1ccc(O[C@H]2C[C@H](NC(=O)C(=O)Nc3c[nH]c4cc(F)c(F)cc34)C2)nc1] |
| 200 | 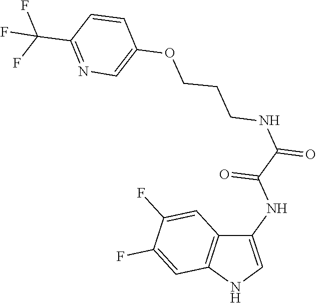 [SMILES: O=C(NCCCOc1ccc(C(F)(F)F)nc1)C(=O)Nc1c[nH]c2cc(F)c(F)cc12] |
| 201 | 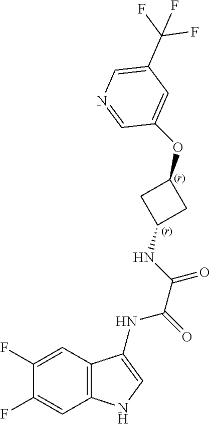 [SMILES: O=C(Nc1c[nH]c2cc(F)c(F)cc12)C(=O)N[C@H]1C[C@H](Oc2cncc(C(F)(F)F)c2)C1] |
| 202 | 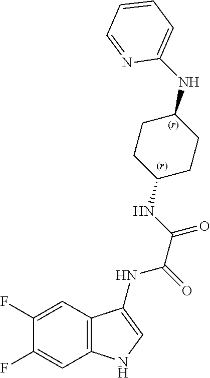 [SMILES: O=C(Nc1c[nH]c2cc(F)c(F)cc12)C(=O)N[C@H]1CC[C@H](Nc2ccccn2)CC1] |
| 203 | 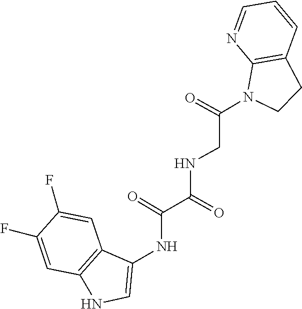 [SMILES: O=C(NCC(=O)N1CCc2cccnc21)C(=O)Nc1c[nH]c2cc(F)c(F)cc12] |
| 204 | 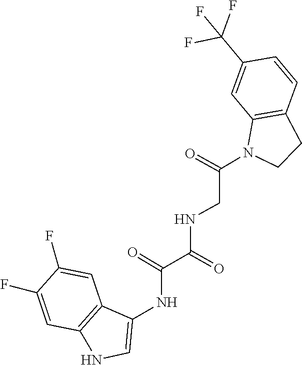 [SMILES: O=C(NCC(=O)N1CCc2ccc(C(F)(F)F)cc21)C(=O)Nc1c[nH]c2cc(F)c(F)cc12] |
| 205 | 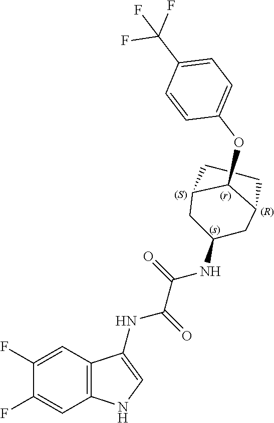 |
| 206 | 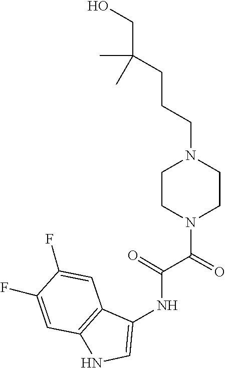 [SMILES: CC(C)(CO)CCCN1CCN(C(=O)C(=O)Nc2c[nH]c3cc(F)c(F)cc23)CC1] |
| 207 | 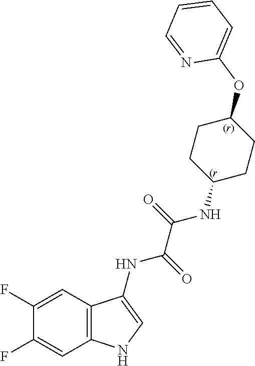 [SMILES: O=C(Nc1c[nH]c2cc(F)c(F)cc12)C(=O)N[C@H]1CC[C@H](Oc2ccccn2)CC1] |
| 208 | 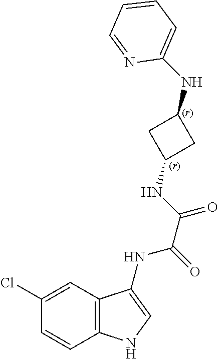 [SMILES: O=C(Nc1c[nH]c2ccc(Cl)cc12)C(=O)N[C@H]1C[C@H](Nc2ccccn2)C1] |
| 209 | 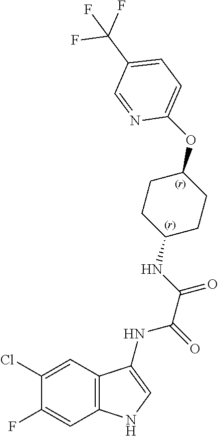 [SMILES: O=C(Nc1c[nH]c2cc(F)c(Cl)cc12)C(=O)N[C@H]1CC[C@H](Oc2ccc(C(F)(F)F)cn2)CC1] |
| 210 | 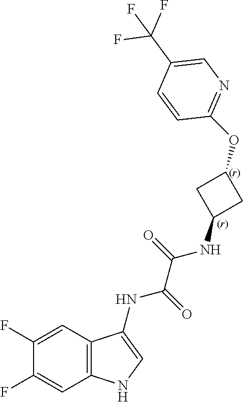 [SMILES: O=C(Nc1c[nH]c2cc(F)c(F)cc12)C(=O)N[C@H]1C[C@H](Oc2ccc(C(F)(F)F)cn2)C1] |
| 211 | 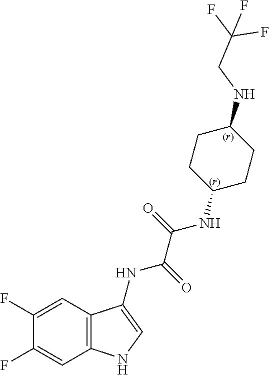 [SMILES: O=C(Nc1c[nH]c2cc(F)c(F)cc12)C(=O)N[C@H]1CC[C@H](NCC(F)(F)F)CC1] |
| 212 | 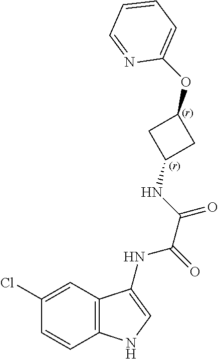 [SMILES: O=C(Nc1c[nH]c2ccc(Cl)cc12)C(=O)N[C@H]1C[C@H](Oc2ccccn2)C1] |
| 213 | 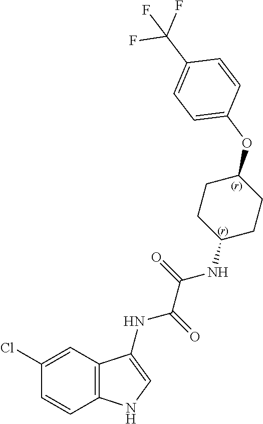 [SMILES: O=C(Nc1c[nH]c2ccc(Cl)cc12)C(=O)N[C@H]1CC[C@H](Oc2ccc(C(F)(F)F)cc2)CC1] |
| 215 | 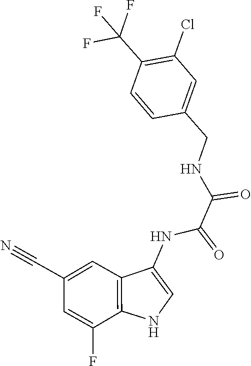 [SMILES: N#Cc1cc(F)c2[nH]cc(NC(=O)C(=O)NCc3ccc(C(F)(F)F)c(Cl)c3)c2c1] |
| 216 | 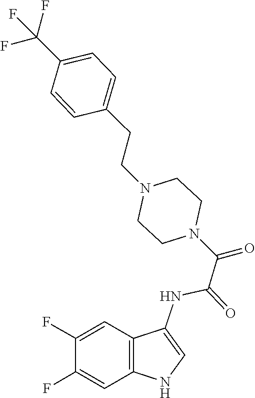 [SMILES: O=C(Nc1c[nH]c2cc(F)c(F)cc12)C(=O)N1CCN(CCc2ccc(C(F)(F)F)cc2)CC1] |
| 217 | 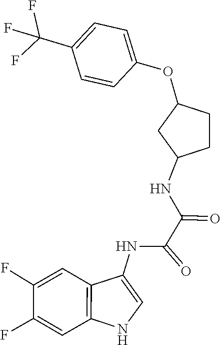 [SMILES: O=C(Nc1c[nH]c2cc(F)c(F)cc12)C(=O)NC1CCC(Oc2ccc(C(F)(F)F)cc2)C1] |
| 218 | 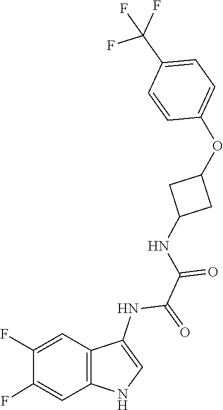 [SMILES: O=C(Nc1c[nH]c2cc(F)c(F)cc12)C(=O)NC1CC(Oc2ccc(C(F)(F)F)cc2)C1] |
| 219 | 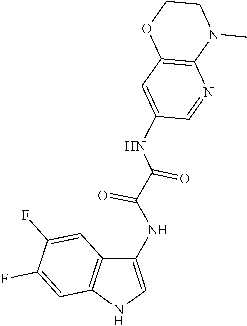 [SMILES: CN1CCOc2cc(NC(=O)C(=O)Nc3c[nH]c4cc(F)c(F)cc34)cnc21] |
| 220 | 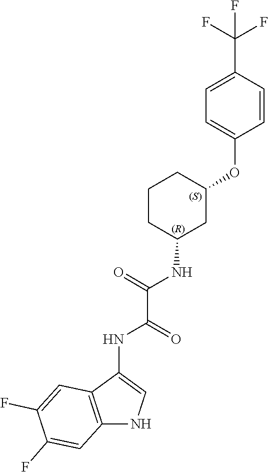 |
| 221 | 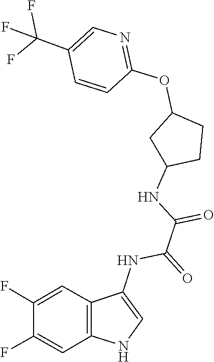 [SMILES: O=C(Nc1c[nH]c2cc(F)c(F)cc12)C(=O)NC1CCC(Oc2ccc(C(F)(F)F)cn2)C1] |
| 222 | 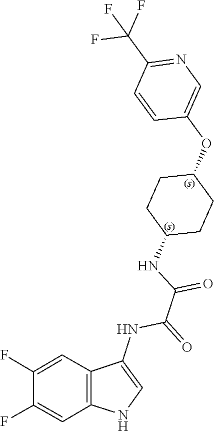 [SMILES: O=C(Nc1c[nH]c2cc(F)c(F)cc12)C(=O)N[C@H]1CC[C@@H](Oc2ccc(C(F)(F)F)nc2)CC1] |
| 223 | 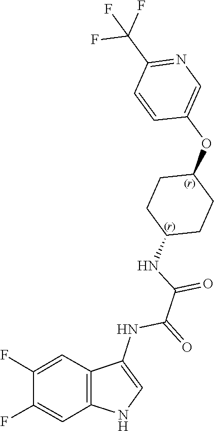 [SMILES: O=C(Nc1c[nH]c2cc(F)c(F)cc12)C(=O)N[C@H]1CC[C@H](Oc2ccc(C(F)(F)F)nc2)CC1] |
| 224 | 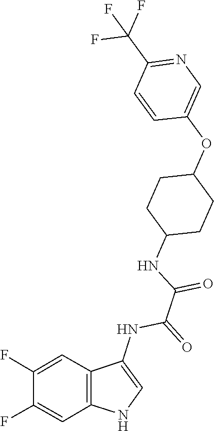 [SMILES: O=C(Nc1c[nH]c2cc(F)c(F)cc12)C(=O)NC1CCC(Oc2ccc(C(F)(F)F)nc2)CC1] |
| 225 | 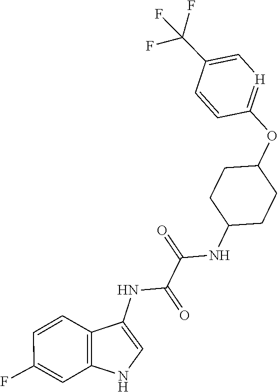 |
| 226 | 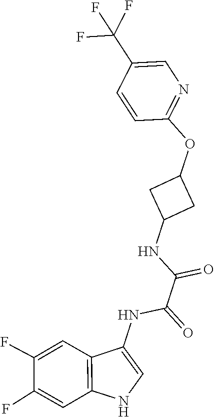 [SMILES: O=C(Nc1c[nH]c2cc(F)c(F)cc12)C(=O)NC1CC(Oc2ccc(C(F)(F)F)cn2)C1] |
| 227 | 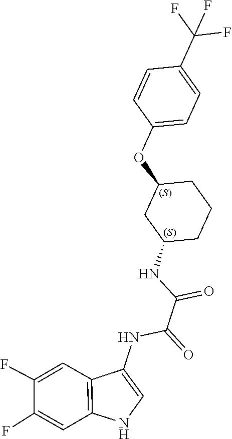 |
| 228 | 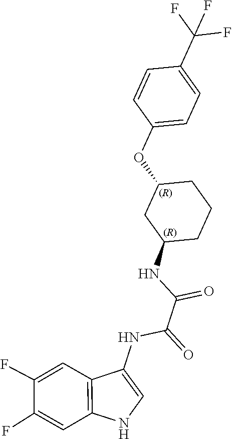 |
| 229 | 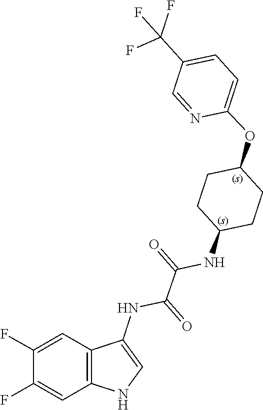 |
| 230 | 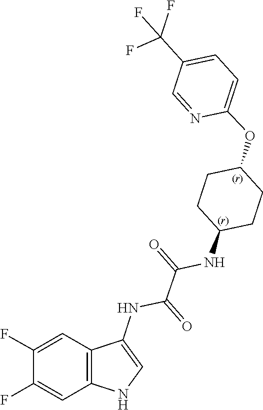 |
| 231 | 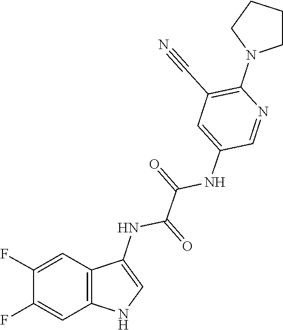 [SMILES: N#Cc1cc(NC(=O)C(=O)Nc2c[nH]c3cc(F)c(F)cc23)cnc1N1CCCC1] |
| 232 | 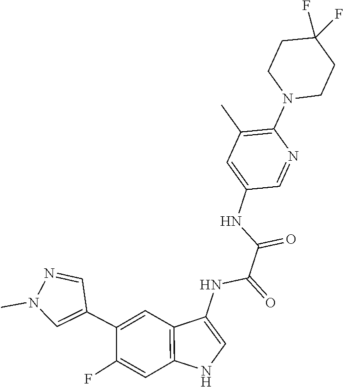 [SMILES: Cc1cc(NC(=O)C(=O)Nc2c[nH]c3cc(F)c(-c4cnn(C)c4)cc23)cnc1N1CCC(F)(F)CC1] |
| 233 | 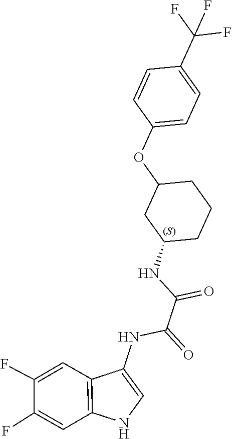 |
| 234 | 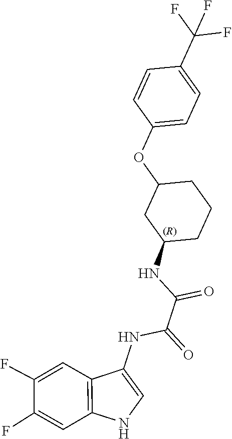 |
| 235 | 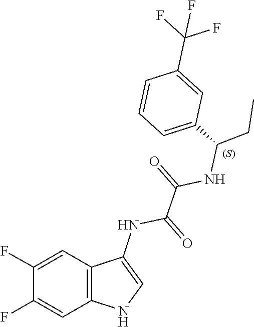 |
| 236 | 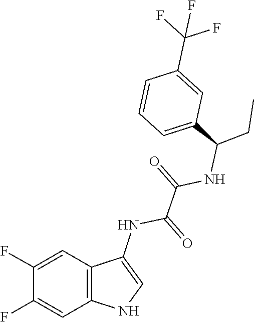 [SMILES: CC[C@@H](NC(=O)C(=O)Nc1c[nH]c2cc(F)c(F)cc12)c1cccc(C(F)(F)F)c1] |
| 238 | 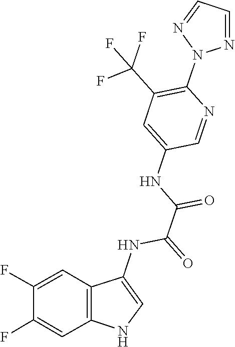 [SMILES: O=C(Nc1cnc(-n2nccn2)c(C(F)(F)F)c1)C(=O)Nc1c[nH]c2cc(F)c(F)cc12] |
| 239 | 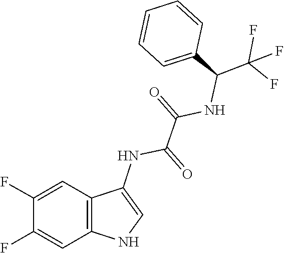 [SMILES: O=C(Nc1c[nH]c2cc(F)c(F)cc12)C(=O)N[C@@H](c1ccccc1)C(F)(F)F] |
| 240 | 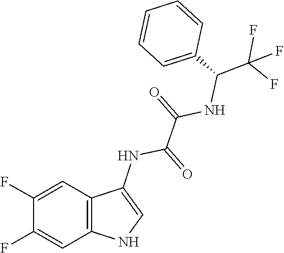 [SMILES: O=C(Nc1c[nH]c2cc(F)c(F)cc12)C(=O)N[C@H](c1ccccc1)C(F)(F)F] |
| 241 | 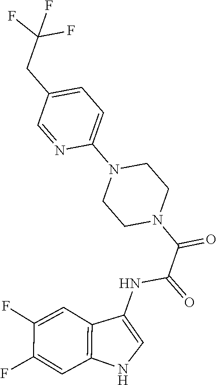 [SMILES: O=C(Nc1c[nH]c2cc(F)c(F)cc12)C(=O)N1CCN(c2ccc(CC(F)(F)F)cn2)CC1] |
| 242 | 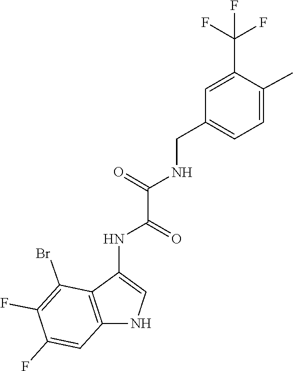 [SMILES: Cc1ccc(CNC(=O)C(=O)Nc2c[nH]c3cc(F)c(F)c(Br)c23)cc1C(F)(F)F] |
| 243 | 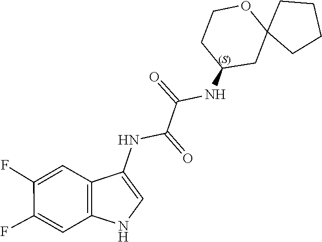 |
| 244 | 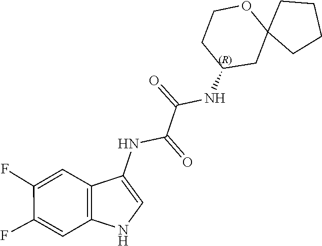 |
| 245 | 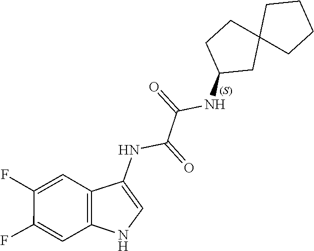 |
| 246 | 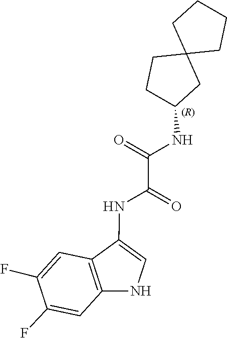 |
| 247 | 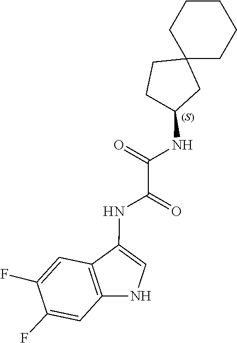 |
| 248 | 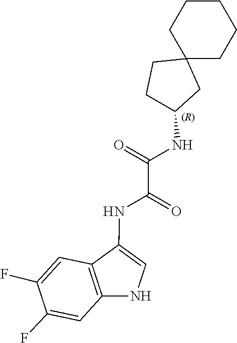 |
| 249 | 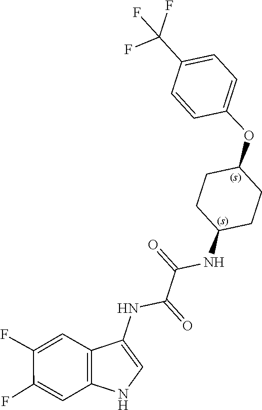 [SMILES: O=C(Nc1c[nH]c2cc(F)c(F)cc12)C(=O)N[C@H]1CC[C@@H](Oc2ccc(C(F)(F)F)cc2)CC1] |
| 250 | 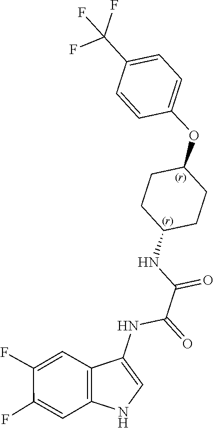 [SMILES: O=C(Nc1c[nH]c2cc(F)c(F)cc12)C(=O)N[C@H]1CC[C@H](Oc2ccc(C(F)(F)F)cc2)CC1] |
| 251 | 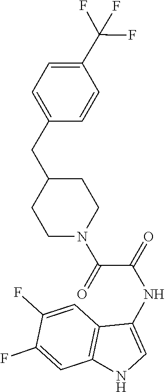 [SMILES: O=C(Nc1c[nH]c2cc(F)c(F)cc12)C(=O)N1CCC(Cc2ccc(C(F)(F)F)cc2)CC1] |
| 252 | 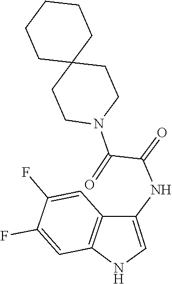 [SMILES: O=C(Nc1c[nH]c2cc(F)c(F)cc12)C(=O)N1CCC2(CCCCC2)CC1] |
| 253 | 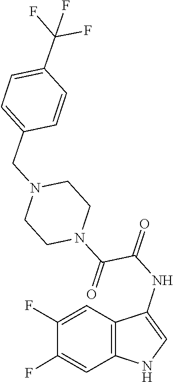 [SMILES: O=C(Nc1c[nH]c2cc(F)c(F)cc12)C(=O)N1CCN(Cc2ccc(C(F)(F)F)cc2)CC1] |
| 254 | 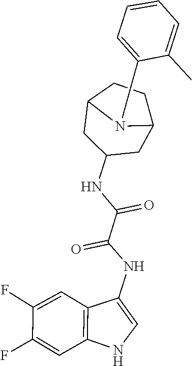 [SMILES: Cc1ccccc1N1C2CCC1CC(NC(=O)C(=O)Nc1c[nH]c3cc(F)c(F)cc13)C2] |
| 255 | 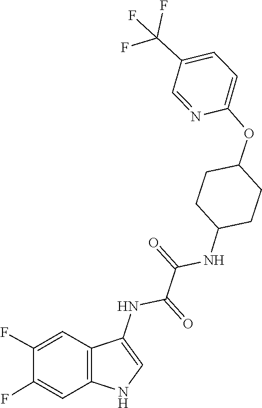 [SMILES: O=C(Nc1c[nH]c2cc(F)c(F)cc12)C(=O)NC1CCC(Oc2ccc(C(F)(F)F)cn2)CC1] |
| 256 | 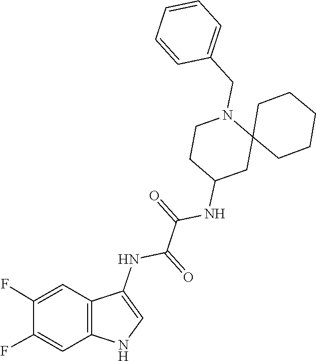 [SMILES: O=C(Nc1c[nH]c2cc(F)c(F)cc12)C(=O)NC1CCN(Cc2ccccc2)C2(CCCCC2)C1] |
| 257 | 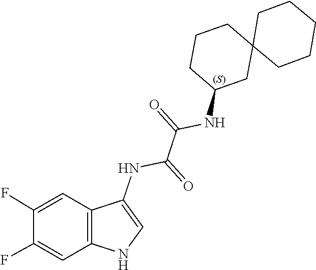 |
| 258 | 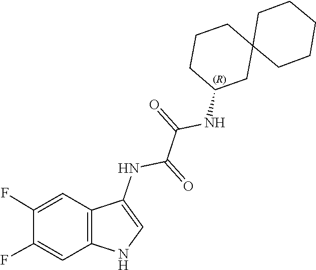 |
| 259 | 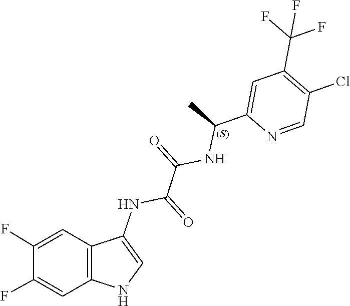 |
| 260 | 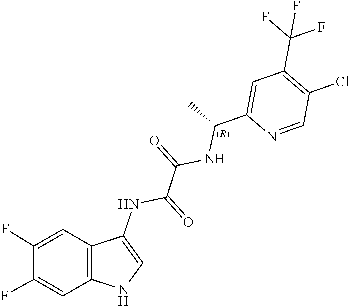 |
| 261 | 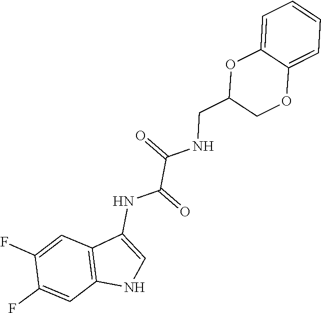 [SMILES: O=C(NCC1COc2ccccc2O1)C(=O)Nc1c[nH]c2cc(F)c(F)cc12] |
| 262 | 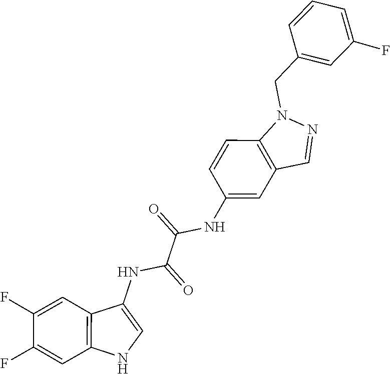 [SMILES: O=C(Nc1ccc2c(cnn2Cc2cccc(F)c2)c1)C(=O)Nc1c[nH]c2cc(F)c(F)cc12] |
| 263 | 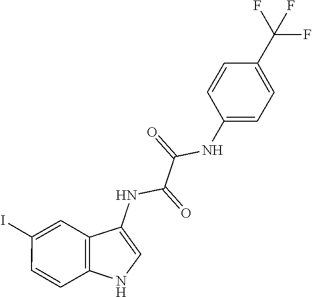 [SMILES: O=C(Nc1ccc(C(F)(F)F)cc1)C(=O)Nc1c[nH]c2ccc(I)cc12] |
| 264 | 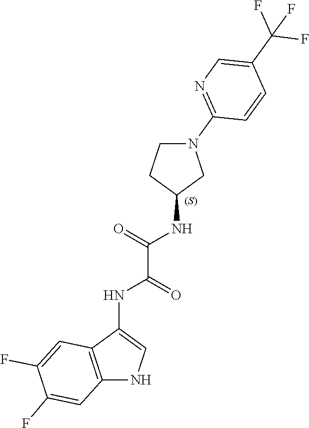 |
| 265 | 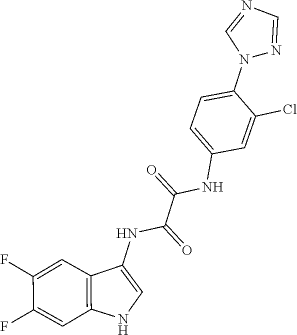 [SMILES: O=C(Nc1ccc(-n2cncn2)c(Cl)c1)C(=O)Nc1c[nH]c2cc(F)c(F)cc12] |
| 266 | 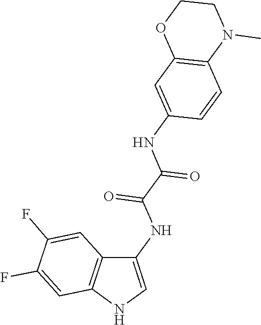 [SMILES: CN1CCOc2cc(NC(=O)C(=O)Nc3c[nH]c4cc(F)c(F)cc34)ccc21] |
| 267 | 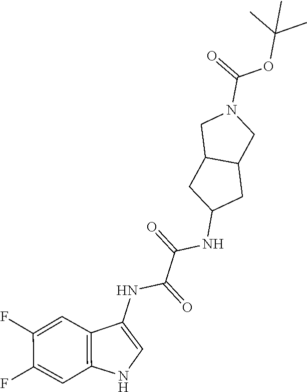 [SMILES: CC(C)(C)OC(=O)N1CC2CC(NC(=O)C(=O)Nc3c[nH]c4cc(F)c(F)cc34)CC2C1] |
| 268 | 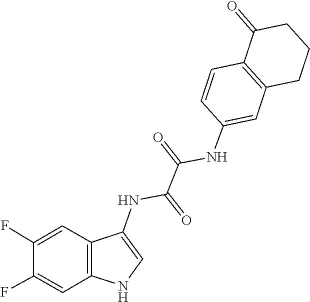 [SMILES: O=C(Nc1ccc2c(c1)CCCC2=O)C(=O)Nc1c[nH]c2cc(F)c(F)cc12] |
| 269 | 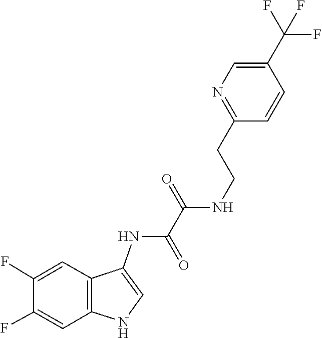 [SMILES: O=C(NCCc1ccc(C(F)(F)F)cn1)C(=O)Nc1c[nH]c2cc(F)c(F)cc12] |
| 270 | 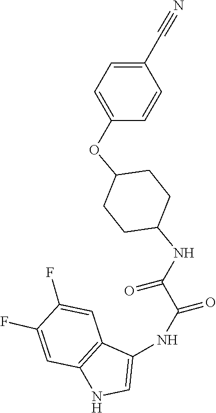 [SMILES: N#Cc1ccc(OC2CCC(NC(=O)C(=O)Nc3c[nH]c4cc(F)c(F)cc34)CC2)cc1] |
| 271 | 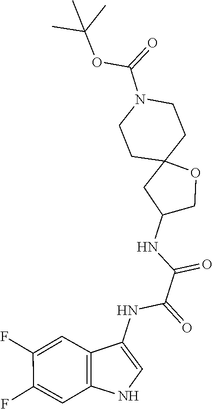 [SMILES: CC(C)(C)OC(=O)N1CCC2(CC1)CC(NC(=O)C(=O)Nc1c[nH]c3cc(F)c(F)cc13)CO2] |
| 272 | 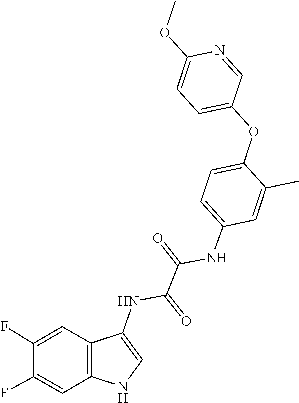 [SMILES: COc1ccc(Oc2ccc(NC(=O)C(=O)Nc3c[nH]c4cc(F)c(F)cc34)cc2C)cn1] |
| 273 | 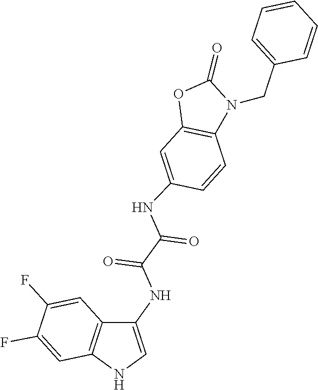 [SMILES: O=C(Nc1ccc2c(c1)oc(=O)n2Cc1ccccc1)C(=O)Nc1c[nH]c2cc(F)c(F)cc12] |
| 274 | 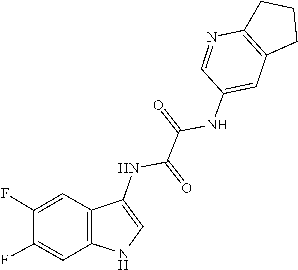 [SMILES: O=C(Nc1cnc2c(c1)CCC2)C(=O)Nc1c[nH]c2cc(F)c(F)cc12] |
| 275 | 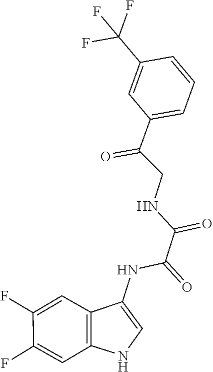 [SMILES: O=C(NCC(=O)c1cccc(C(F)(F)F)c1)C(=O)Nc1c[nH]c2cc(F)c(F)cc12] |
| 276 | 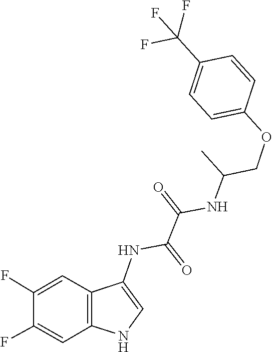 [SMILES: CC(COc1ccc(C(F)(F)F)cc1)NC(=O)C(=O)Nc1c[nH]c2cc(F)c(F)cc12] |
| 277 | 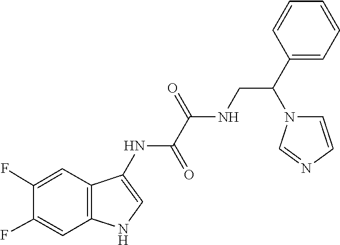 [SMILES: O=C(NCC(c1ccccc1)n1ccnc1)C(=O)Nc1c[nH]c2cc(F)c(F)cc12] |
| 278 | 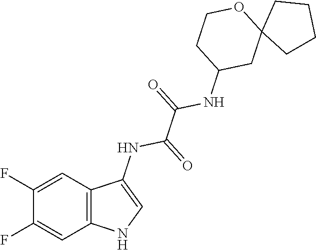 [SMILES: O=C(Nc1c[nH]c2cc(F)c(F)cc12)C(=O)NC1CCOC2(CCCC2)C1] |
| 279 | 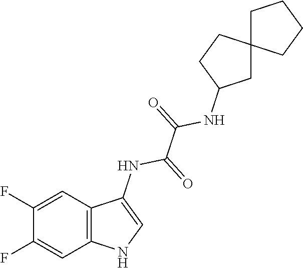 [SMILES: O=C(Nc1c[nH]c2cc(F)c(F)cc12)C(=O)NC1CCC2(CCCC2)C1] |
| 280 | 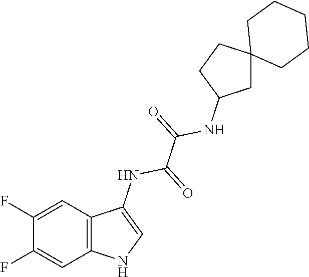 [SMILES: O=C(Nc1c[nH]c2cc(F)c(F)cc12)C(=O)NC1CCC2(CCCCC2)C1] |
| 281 | 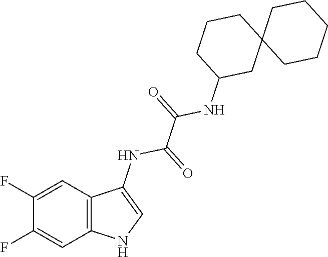 [SMILES: O=C(Nc1c[nH]c2cc(F)c(F)cc12)C(=O)NC1CCCC2(CCCCC2)C1] |
| 282 | 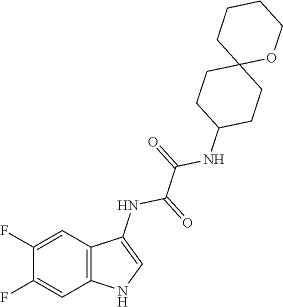 [SMILES: O=C(Nc1c[nH]c2cc(F)c(F)cc12)C(=O)NC1CCC2(CCCCO2)CC1] |
| 284 | 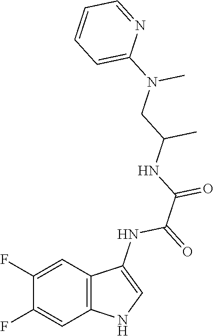 [SMILES: CC(CN(C)c1ccccn1)NC(=O)C(=O)Nc1c[nH]c2cc(F)c(F)cc12] |
| 285 | 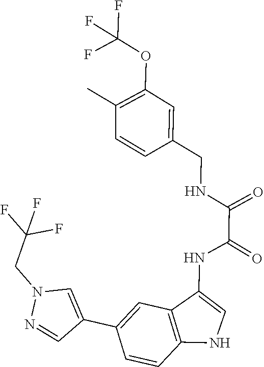 [SMILES: Cc1ccc(CNC(=O)C(=O)Nc2c[nH]c3ccc(-c4cnn(CC(F)(F)F)c4)cc23)cc1OC(F)(F)F] |
| 286 | 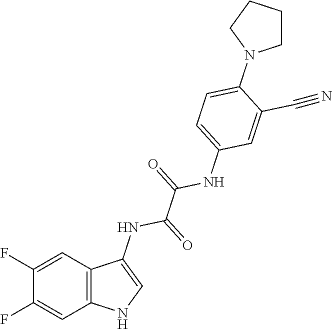 [SMILES: N#Cc1cc(NC(=O)C(=O)Nc2c[nH]c3cc(F)c(F)cc23)ccc1N1CCCC1] |
| 287 | 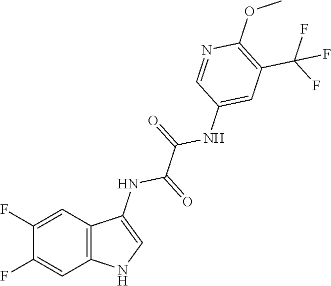 [SMILES: COc1ncc(NC(=O)C(=O)Nc2c[nH]c3cc(F)c(F)cc23)cc1C(F)(F)F] |
| 288 | 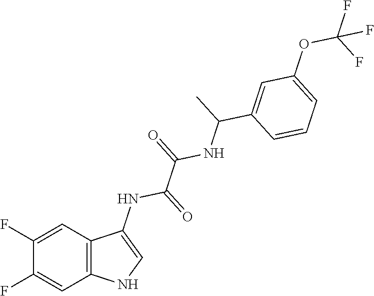 [SMILES: CC(NC(=O)C(=O)Nc1c[nH]c2cc(F)c(F)cc12)c1cccc(OC(F)(F)F)c1] |
| 289 | 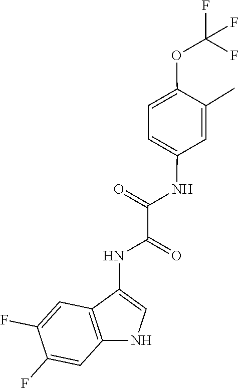 [SMILES: Cc1cc(NC(=O)C(=O)Nc2c[nH]c3cc(F)c(F)cc23)ccc1OC(F)(F)F] |
| 290 | 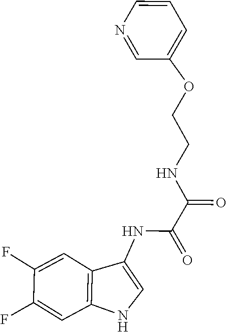 [SMILES: O=C(NCCOc1cccnc1)C(=O)Nc1c[nH]c2cc(F)c(F)cc12] |
| 291 | 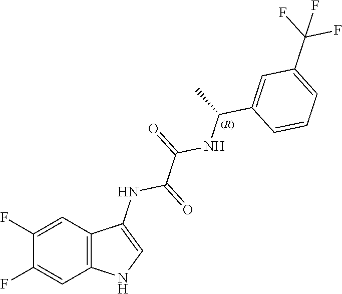 |
| 292 | 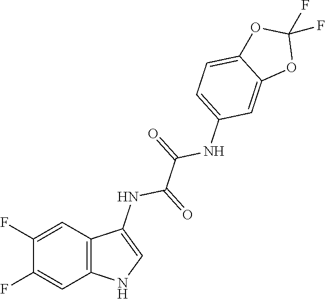 [SMILES: O=C(Nc1ccc2c(c1)OC(F)(F)O2)C(=O)Nc1c[nH]c2cc(F)c(F)cc12] |
| 293 | 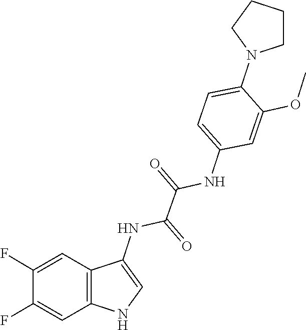 [SMILES: COc1cc(NC(=O)C(=O)Nc2c[nH]c3cc(F)c(F)cc23)ccc1N1CCCC1] |
| 294 | 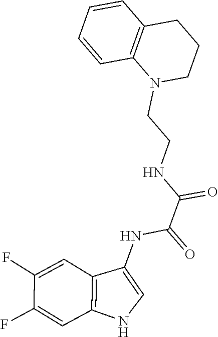 [SMILES: O=C(NCCN1CCCc2ccccc21)C(=O)Nc1c[nH]c2cc(F)c(F)cc12] |
| 295 | 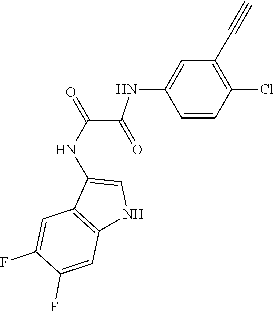 [SMILES: C#Cc1cc(NC(=O)C(=O)Nc2c[nH]c3cc(F)c(F)cc23)ccc1Cl] |
| 296 | 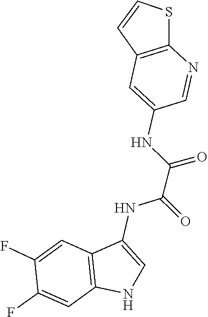 [SMILES: O=C(Nc1cnc2sccc2c1)C(=O)Nc1c[nH]c2cc(F)c(F)cc12] |
| 297 | 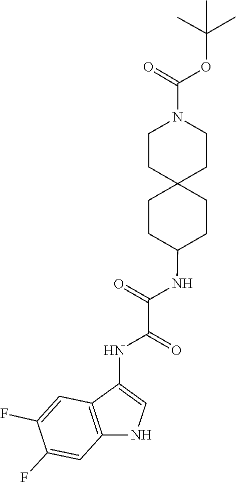 [SMILES: CC(C)(C)OC(=O)N1CCC2(CCC(NC(=O)C(=O)Nc3c[nH]c4cc(F)c(F)cc34)CC2)CC1] |
| 298 | 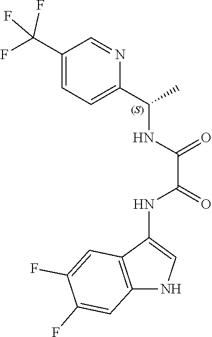 |
| 299 | 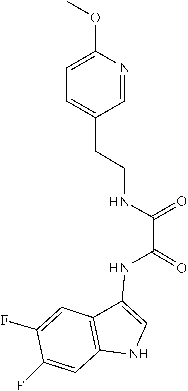 [SMILES: COc1ccc(CCNC(=O)C(=O)Nc2c[nH]c3cc(F)c(F)cc23)cn1] |
| 300 | 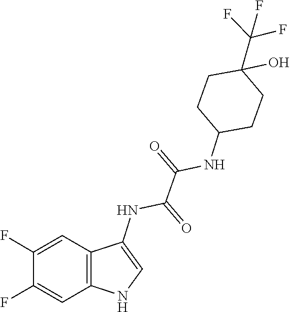 [SMILES: O=C(Nc1c[nH]c2cc(F)c(F)cc12)C(=O)NC1CCC(O)(C(F)(F)F)CC1] |
| 301 | 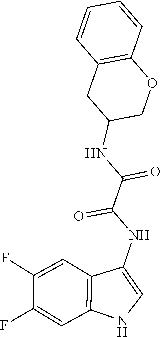 [SMILES: O=C(Nc1c[nH]c2cc(F)c(F)cc12)C(=O)NC1COc2ccccc2C1] |
| 302 | 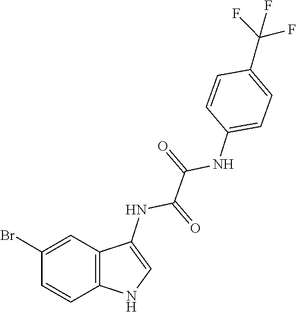 [SMILES: O=C(Nc1ccc(C(F)(F)F)cc1)C(=O)Nc1c[nH]c2ccc(Br)cc12] |
| 303 | 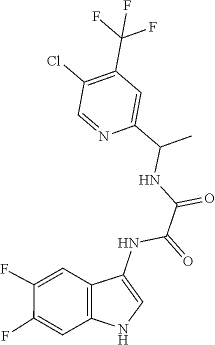 [SMILES: CC(NC(=O)C(=O)Nc1c[nH]c2cc(F)c(F)cc12)c1cc(C(F)(F)F)c(Cl)cn1] |
| 304 | 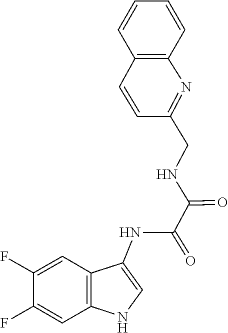 [SMILES: O=C(NCc1ccc2ccccc2n1)C(=O)Nc1c[nH]c2cc(F)c(F)cc12] |
| 305 | 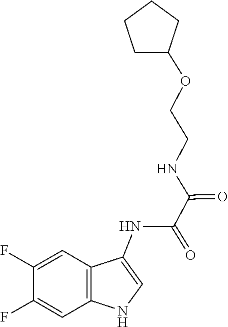 [SMILES: O=C(NCCOC1CCCC1)C(=O)Nc1c[nH]c2cc(F)c(F)cc12] |
| 306 | 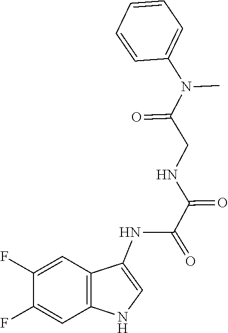 [SMILES: CN(C(=O)CNC(=O)C(=O)Nc1c[nH]c2cc(F)c(F)cc12)c1ccccc1] |
| 307 | 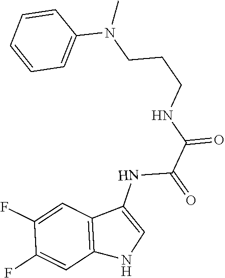 [SMILES: CN(CCCNC(=O)C(=O)Nc1c[nH]c2cc(F)c(F)cc12)c1ccccc1] |
| 308 | 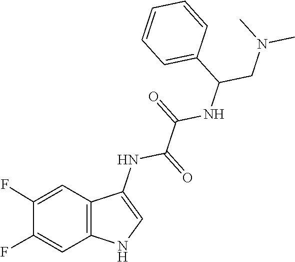 [SMILES: CN(C)CC(NC(=O)C(=O)Nc1c[nH]c2cc(F)c(F)cc12)c1ccccc1] |
| 309 | 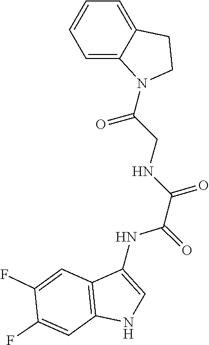 [SMILES: O=C(NCC(=O)N1CCc2ccccc21)C(=O)Nc1c[nH]c2cc(F)c(F)cc12] |
| 310 | 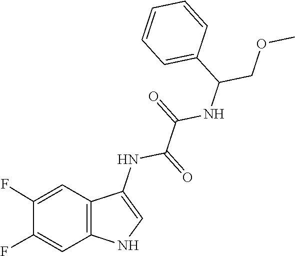 [SMILES: COCC(NC(=O)C(=O)Nc1c[nH]c2cc(F)c(F)cc12)c1ccccc1] |
| 311 | 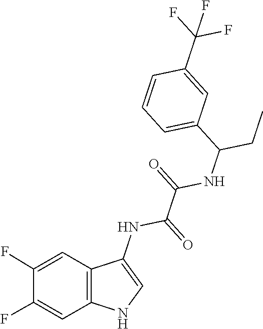 [SMILES: CCC(NC(=O)C(=O)Nc1c[nH]c2cc(F)c(F)cc12)c1cccc(C(F)(F)F)c1] |
| 312 | 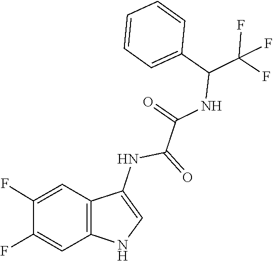 [SMILES: O=C(Nc1c[nH]c2cc(F)c(F)cc12)C(=O)NC(c1ccccc1)C(F)(F)F] |
| 313 | 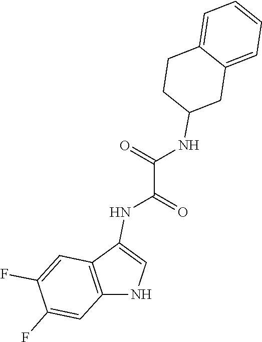 [SMILES: O=C(Nc1c[nH]c2cc(F)c(F)cc12)C(=O)NC1CCc2ccccc2C1] |
| 314 | 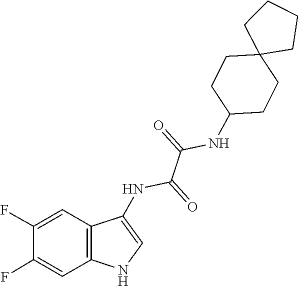 [SMILES: O=C(Nc1c[nH]c2cc(F)c(F)cc12)C(=O)NC1CCC2(CCCC2)CC1] |
| 315 | 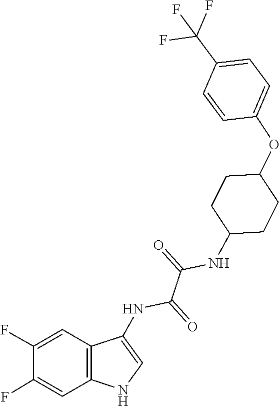 [SMILES: O=C(Nc1c[nH]c2cc(F)c(F)cc12)C(=O)NC1CCC(Oc2ccc(C(F)(F)F)cc2)CC1] |
| 316 | 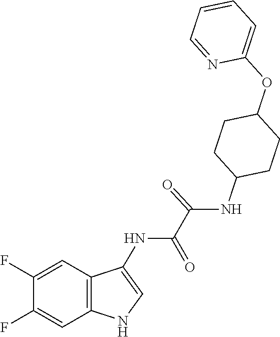 [SMILES: O=C(Nc1c[nH]c2cc(F)c(F)cc12)C(=O)NC1CCC(Oc2ccccn2)CC1] |
| 317 | 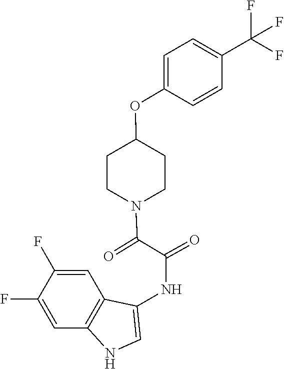 [SMILES: O=C(Nc1c[nH]c2cc(F)c(F)cc12)C(=O)N1CCC(Oc2ccc(C(F)(F)F)cc2)CC1] |
| 318 | 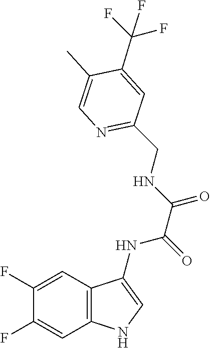 [SMILES: Cc1cnc(CNC(=O)C(=O)Nc2c[nH]c3cc(F)c(F)cc23)cc1C(F)(F)F] |
| 319 | 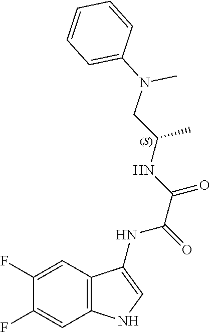 |
| 320 | 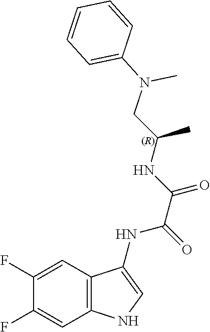 |
| 321 | 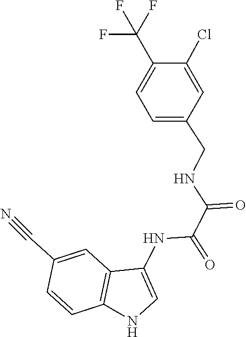 [SMILES: N#Cc1ccc2[nH]cc(NC(=O)C(=O)NCc3ccc(C(F)(F)F)c(Cl)c3)c2c1] |
| 322 | 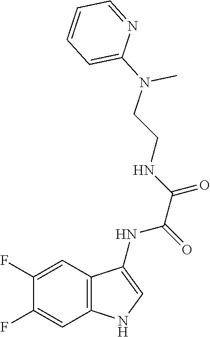 [SMILES: CN(CCNC(=O)C(=O)Nc1c[nH]c2cc(F)c(F)cc12)c1ccccn1] |
| 323 | 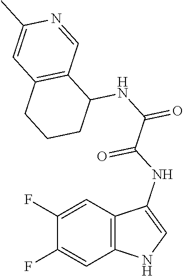 [SMILES: Cc1cc2c(cn1)C(NC(=O)C(=O)Nc1c[nH]c3cc(F)c(F)cc13)CCC2] |
| 324 | 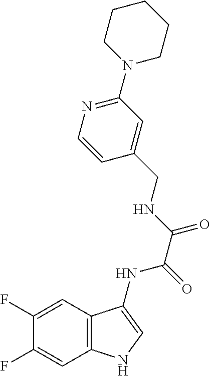 [SMILES: O=C(NCc1ccnc(N2CCCCC2)c1)C(=O)Nc1c[nH]c2cc(F)c(F)cc12] |
| 325 | 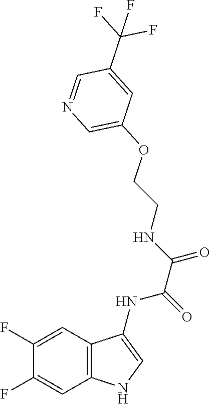 [SMILES: O=C(NCCOc1cncc(C(F)(F)F)c1)C(=O)Nc1c[nH]c2cc(F)c(F)cc12] |
| 326 | 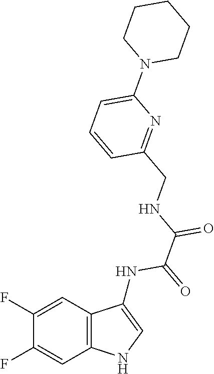 [SMILES: O=C(NCc1cccc(N2CCCCC2)n1)C(=O)Nc1c[nH]c2cc(F)c(F)cc12] |
| 327 | 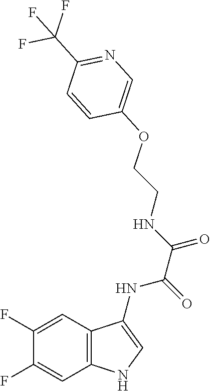 [SMILES: O=C(NCCOc1ccc(C(F)(F)F)nc1)C(=O)Nc1c[nH]c2cc(F)c(F)cc12] |
| 328 | 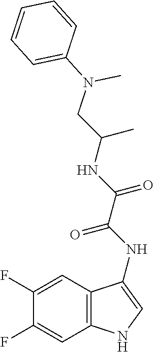 [SMILES: CC(CN(C)c1ccccc1)NC(=O)C(=O)Nc1c[nH]c2cc(F)c(F)cc12] |
| 329 | 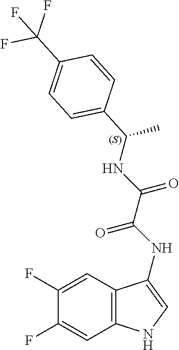 |
| 330 | 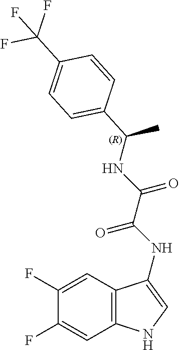 |
| 331 | 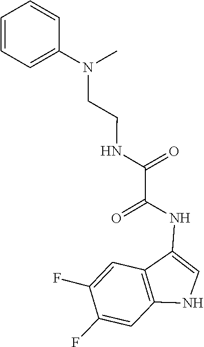 [SMILES: CN(CCNC(=O)C(=O)Nc1c[nH]c2cc(F)c(F)cc12)c1ccccc1] |
| 332 | 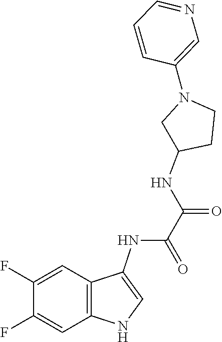 [SMILES: O=C(Nc1c[nH]c2cc(F)c(F)cc12)C(=O)NC1CCN(c2cccnc2)C1] |
| 333 | 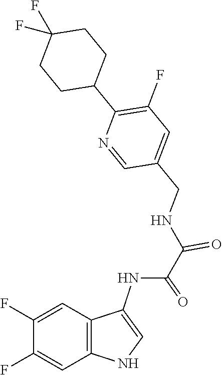 [SMILES: O=C(NCc1cnc(C2CCC(F)(F)CC2)c(F)c1)C(=O)Nc1c[nH]c2cc(F)c(F)cc12] |
| 334 | 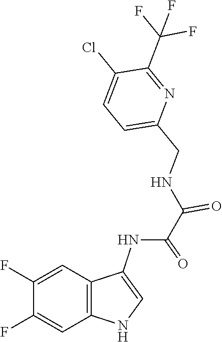 [SMILES: O=C(NCc1ccc(Cl)c(C(F)(F)F)n1)C(=O)Nc1c[nH]c2cc(F)c(F)cc12] |
| 335 | 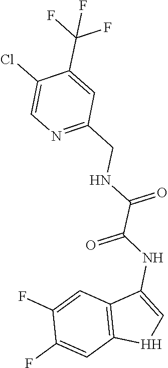 |
| 336 | 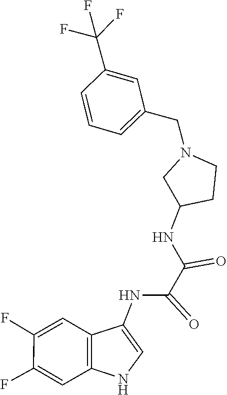 [SMILES: O=C(Nc1c[nH]c2cc(F)c(F)cc12)C(=O)NC1CCN(Cc2cccc(C(F)(F)F)c2)C1] |
| 337 | 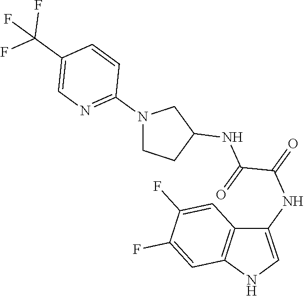 [SMILES: O=C(Nc1c[nH]c2cc(F)c(F)cc12)C(=O)NC1CCN(c2ccc(C(F)(F)F)cn2)C1] |
| 338 | 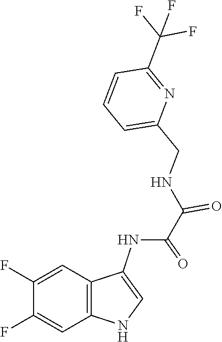 [SMILES: O=C(NCc1cccc(C(F)(F)F)n1)C(=O)Nc1c[nH]c2cc(F)c(F)cc12] |
| 339 | 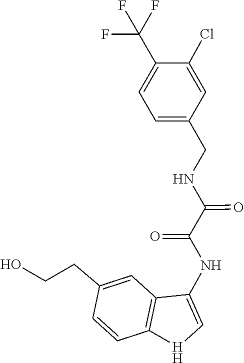 |
| 340 | 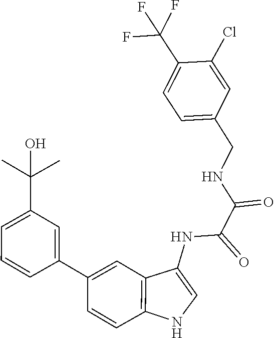 [SMILES: CC(C)(O)c1cccc(-c2ccc3[nH]cc(NC(=O)C(=O)NCc4ccc(C(F)(F)F)c(Cl)c4)c3c2)c1] |
| 341 | 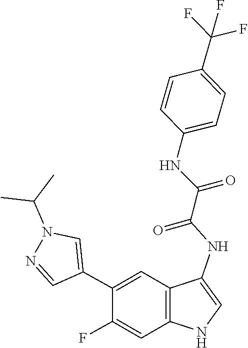 [SMILES: CC(C)n1cc(-c2cc3c(NC(=O)C(=O)Nc4ccc(C(F)(F)F)cc4)c[nH]c3cc2F)cn1] |
| 342 | 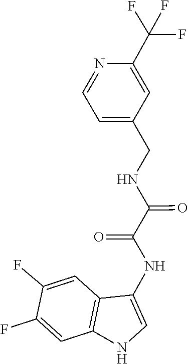 [SMILES: O=C(NCc1ccnc(C(F)(F)F)c1)C(=O)Nc1c[nH]c2cc(F)c(F)cc12] |
| 343 | 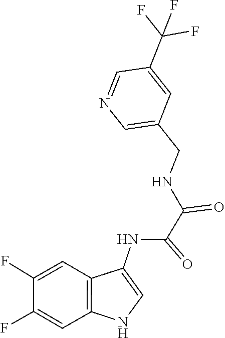 [SMILES: O=C(NCc1cncc(C(F)(F)F)c1)C(=O)Nc1c[nH]c2cc(F)c(F)cc12] |
| 344 | 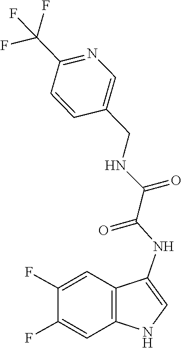 [SMILES: O=C(NCc1ccc(C(F)(F)F)nc1)C(=O)Nc1c[nH]c2cc(F)c(F)cc12] |
| 346 | 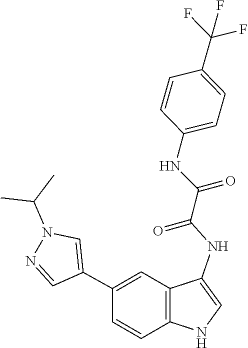 [SMILES: CC(C)n1cc(-c2ccc3[nH]cc(NC(=O)C(=O)Nc4ccc(C(F)(F)F)cc4)c3c2)cn1] |
| 347 | 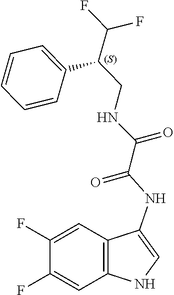 |
| 348 | 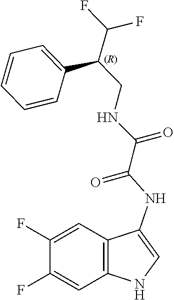 |
| 349 | 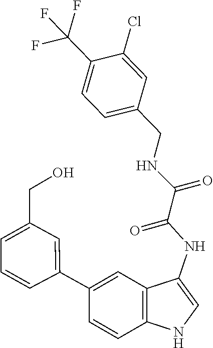 [SMILES: O=C(NCc1ccc(C(F)(F)F)c(Cl)c1)C(=O)Nc1c[nH]c2ccc(-c3cccc(CO)c3)cc12] |
| 350 | 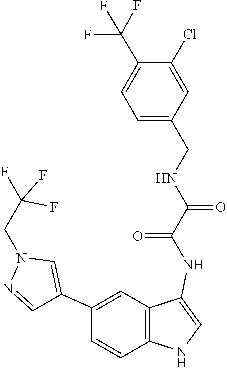 [SMILES: O=C(NCc1ccc(C(F)(F)F)c(Cl)c1)C(=O)Nc1c[nH]c2ccc(-c3cnn(CC(F)(F)F)c3)cc12] |
| 351 | 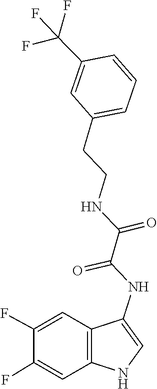 [SMILES: O=C(NCCc1cccc(C(F)(F)F)c1)C(=O)Nc1c[nH]c2cc(F)c(F)cc12] |
| 352 | 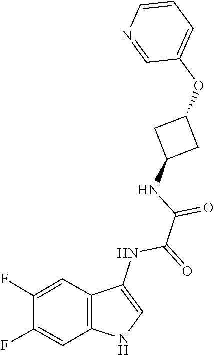 [SMILES: O=C(Nc1c[nH]c2cc(F)c(F)cc12)C(=O)N[C@H]1C[C@H](Oc2cccnc2)C1] |
| 353 | 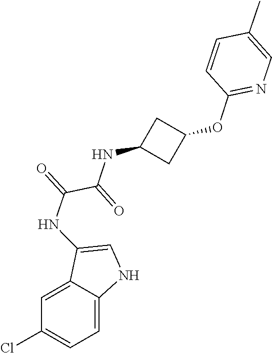 [SMILES: Cc1ccc(O[C@H]2C[C@H](NC(=O)C(=O)Nc3c[nH]c4ccc(Cl)cc34)C2)nc1] |
| 354 | 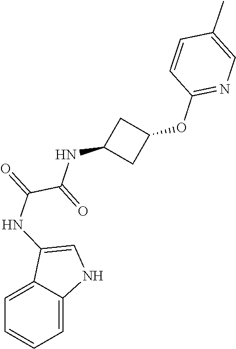 [SMILES: Cc1ccc(O[C@H]2C[C@H](NC(=O)C(=O)Nc3c[nH]c4ccccc34)C2)nc1] |
| 355 | 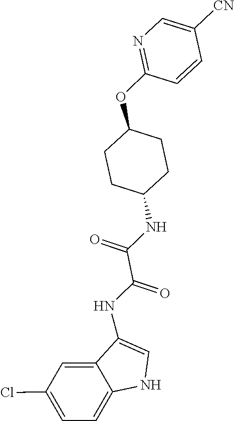 [SMILES: N#Cc1ccc(O[C@H]2CC[C@H](NC(=O)C(=O)Nc3c[nH]c4ccc(Cl)cc34)CC2)nc1] |
| 356 | 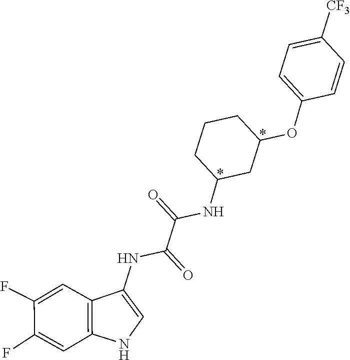 [SMILES: O=C(Nc1c[nH]c2cc(F)c(F)cc12)C(=O)NC1CCCC(Oc2ccc(C(F)(F)F)cc2)C1] |
| 357 | 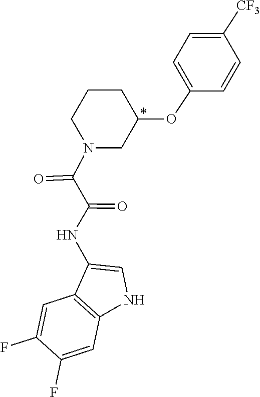 [SMILES: O=C(Nc1c[nH]c2cc(F)c(F)cc12)C(=O)N1CCCC(Oc2ccc(C(F)(F)F)cc2)C1] |
| 358 | 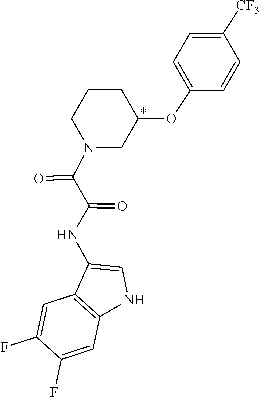 [SMILES: O=C(Nc1c[nH]c2cc(F)c(F)cc12)C(=O)N1CCCC(Oc2ccc(C(F)(F)F)cc2)C1] |

Pharmaceutical Compositions and Administration

General

In some embodiments, a chemical entity (e.g., a compound that inhibits (e.g., antagonizes) STING, or a pharmaceutically acceptable salt, and/or hydrate, and/or cocrystal, and/or drug combination thereof) is administered as a pharmaceutical composition that includes the chemical entity and one or more pharmaceutically acceptable excipients, and optionally one or more additional therapeutic agents as described herein.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-o-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

Routes of Administration and Composition Components

In some embodiments, the chemical entities described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the chemical entities described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the compounds described herein or a pharmaceutical composition thereof are suitable for local delivery to the digestive or GI tract by way of oral administration (e.g., solid or liquid dosage forms.).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the chemical entity to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

Dosages

The dosages may be varied depending on the requirement of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/Kg to about 500 mg/Kg (e.g., from about 0.001 mg/Kg to about 200 mg/Kg; from about 0.01 mg/Kg to about 200 mg/Kg; from about 0.01 mg/Kg to about 150 mg/Kg; from about 0.01 mg/Kg to about 100 mg/Kg; from about 0.01 mg/Kg to about 50 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 200 mg/Kg; from about 0.1 mg/Kg to about 150 mg/Kg; from about 0.1 mg/Kg to about 100 mg/Kg; from about 0.1 mg/Kg to about 50 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg).

Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

Methods of Treatment

In some embodiments, methods for treating a subject having condition, disease or disorder in which increased (e.g., excessive)STING activity (e.g., e.g., STING signaling) contributes to the pathology and/or symptoms and/or progression of the condition, disease or disorder (e.g., immune disorders, cancer) are provided.

Indications

In some embodiments, the condition, disease or disorder is cancer. Non-limiting examples of cancer include melanoma, carcinoma, lymphoma, blastoma, sarcoma, and leukemia or lymphoid malignancies. More particular examples of such cancers include breast cancer, colon cancer, rectal cancer, colorectal cancer, kidney or renal cancer, clear cell cancer lung cancer including small-cell lung cancer, non-small cell lung cancer, adenocarcinoma of the lung and squamous carcinoma of the lung, squamous cell cancer (e.g. epithelial squamous cell cancer), cervical cancer, ovarian cancer, prostate cancer, prostatic neoplasms, liver cancer, bladder cancer, cancer of the peritoneum, hepatocellular cancer, gastric or stomach cancer including gastrointestinal cancer, gastrointestinal stromal tumor, pancreatic cancer, head and neck cancer, glioblastoma, retinoblastoma, astrocytoma, thecomas, arrhenoblastomas, hepatoma, hematologic malignancies including non-Hodgkins lymphoma (NHL), multiple myeloma, myelodysplasia disorders, myeloproliferative disorders, chronic myelogenous leukemia, and acute hematologic malignancies, endometrial or uterine carcinoma, endometriosis, endometrial stromal sarcoma, fibrosarcomas, choriocarcinoma, salivary gland carcinoma, vulval cancer, thyroid cancer, esophageal carcinomas, hepatic carcinoma, anal carcinoma, penile carcinoma, nasopharyngeal carcinoma, laryngeal carcinomas, Kaposi's sarcoma, mast cell sarcoma, ovarian sarcoma, uterine sarcoma, melanoma, malignant mesothelioma, skin carcinomas, Schwannoma, oligodendroglioma, neuroblastomas, neuroectodermal tumor, rhabdomyosarcoma, osteogenic sarcoma, leiomyosarcomas, Ewing Sarcoma, peripheral primitive neuroectodermal tumor, urinary tract carcinomas, thyroid carcinomas, Wilm's tumor, as well as abnormal vascular proliferation associated with phakomatoses, edema (such as that associated with brain tumors), and Meigs' syndrome. In some cases, the cancer is melanoma.

In some embodiments, the condition, disease or disorder is a neurological disorder, which includes disorders that involve the central nervous system (brain, brainstem and cerebellum), the peripheral nervous system (including cranial nerves), and the autonomic nervous system (parts of which are located in both central and peripheral nervous system). Non-limiting examples of cancer include acquired epileptiform aphasia; acute disseminated encephalomyelitis; adrenoleukodystrophy; age-related macular degeneration; agenesis of the corpus callosum; agnosia; Aicardi syndrome; Alexander disease; Alpers' disease; alternating hemiplegia; Alzheimer's disease; Vascular dementia; amyotrophic lateral sclerosis; anencephaly; Angelman syndrome; angiomatosis; anoxia; aphasia; apraxia; arachnoid cysts; arachnoiditis; Anronl-Chiari malformation; arteriovenous malformation; Asperger syndrome; ataxia telegiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome; causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy; chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease; cytomegalovirus infection; dancing eyes-dancing feet syndrome; Dandy-Walker syndrome; Dawson disease; De Morsier's syndrome; Dejerine-Klumke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; fronto-temporal dementia and other “tauopathies”; Gaucher's disease; Gerstmann's syndrome; giant cell arteritis; giant cell inclusion disease; globoid cell leukodystrophy; Guillain-Barre syndrome; HTLV-1-associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile phytanic acid storage disease; infantile refsum disease; infantile spasms; inflammatory myopathy; intracranial cyst; intracranial hypertension; Joubert syndrome; Kearns-Sayre syndrome; Kennedy disease Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gustaut syndrome; Lesch-Nyhan syndrome; leukodystrophy; Lewy body dementia; Lissencephaly; locked-in syndrome; Lou Gehrig's disease (i.e., motor neuron disease or amyotrophic lateral sclerosis); lumbar disc disease; Lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neuron disease; Moyamoya disease; mucopolysaccharidoses; milti-infarct dementia; multifocal motor neuropathy; multiple sclerosis and other demyelinating disorders; multiple system atrophy with postural hypotension; p muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenital; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; post-polio syndrome; postherpetic neuralgia; postinfectious encephalomyelitis; postural hypotension; Prader-Willi syndrome; primary lateral sclerosis; prion diseases; progressive hemifacial atrophy; progressive multifocal leukoencephalopathy; progressive sclerosing poliodystrophy; progressive supranuclear palsy; pseudotumor cerebri; Ramsay-Hunt syndrome (types I and II); Rasmussen's encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus dance; Sandhoff disease; Schilder's disease; schizencephaly; septo-optic dysplasia; shaken baby syndrome; shingles; Shy-Drager syndrome; Sjögren's syndrome; sleep apnea; Soto's syndrome; spasticity; spina bifida; spinal cord injury; spinal cord tumors; spinal muscular atrophy; Stiff-Person syndrome; stroke; Sturge-Weber syndrome; subacute sclerosing panencephalitis; subcortical arteriosclerotic encephalopathy; Sydenham chorea; syncope; syringomyelia; tardive dyskinesia; Tay-Sachs disease; temporal arteritis; tethered spinal cord syndrome; Thomsen disease; thoracic outlet syndrome; Tic Douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau disease; Wallenberg's syndrome; Werdnig-Hoffman disease; West syndrome; whiplash; Williams syndrome; Wildon's disease; amyotrophe lateral sclerosis and Zellweger syndrome.

In some embodiments, the condition, disease or disorder is STING-associated conditions, e.g., type I interferonopathies (e.g., STING-associated vasculopathy with onset in infancy (SAVI)), Aicardi-Goutieres Syndrome (AGS), genetic forms of lupus, and inflammation-associated disorders such as systemic lupus erythematosus, and rheumatoid arthritis. In certain embodiments, the condition, disease or disorder is an autoimmune disease (e.g., a cytosolic DNA-triggered autoinflammatory disease). Non-limiting examples include rheumatoid arthritis, systemic lupus erythematosus, multiple sclerosis, inflammatory bowel diseases (IBDs) comprising Crohn disease (CD) and ulcerative colitis (UC), which are chronic inflammatory conditions with polygenic susceptibility. In certain embodiments, the condition is an inflammatory bowel disease. In certain embodiments, the condition is Crohn's disease, autoimmune colitis, iatrogenic autoimmune colitis, ulcerative colitis, colitis induced by one or more chemotherapeutic agents, colitis induced by treatment with adoptive cell therapy, colitis associated by one or more alloimmune diseases (such as graft-vs-host disease, e.g., acute graft vs. host disease and chronic graft vs. host disease), radiation enteritis, collagenous colitis, lymphocytic colitis, microscopic colitis, and radiation enteritis. In certain of these embodiments, the condition is alloimmune disease (such as graft-vs-host disease, e.g., acute graft vs. host disease and chronic graft vs. host disease), celiac disease, irritable bowel syndrome, rheumatoid arthritis, lupus, scleroderma, psoriasis, cutaneous T-cell lymphoma, uveitis, and mucositis (e.g., oral mucositis, esophageal mucositis or intestinal mucositis).

In some embodiments, modulation of the immune system by STING provides for the treatment of diseases, including diseases caused by foreign agents. Exemplary infections by foreign agents which may be treated and/or prevented by the method of the present invention include an infection by a bacterium (e.g., a Gram-positive or Gram-negative bacterium), an infection by a fungus, an infection by a parasite, and an infection by a virus. In one embodiment of the present invention, the infection is a bacterial infection (e.g., infection by E. coli, Klebsiella pneumoniae, Pseudomonas aeruginosa, Salmonella spp., Staphylococcus aureus, Streptococcus spp., or vancomycin-resistant enterococcus), or sepsis. In another embodiment, the infection is a fungal infection (e.g. infection by a mould, a yeast, or a higher fungus). In still another embodiment, the infection is a parasitic infection (e.g., infection by a single-celled or multicellular parasite, including Giardia duodenalis, Cryptosporidium parvum, Cyclospora cayetanensis, and Toxoplasma gondiz). In yet another embodiment, the infection is a viral infection (e.g., infection by a virus associated with AIDS, avian flu, chickenpox, cold sores, common cold, gastroenteritis, glandular fever, influenza, measles, mumps, pharyngitis, pneumonia, rubella, SARS, and lower or upper respiratory tract infection (e.g., respiratory syncytial virus)).

In some embodiments, the condition, disease or disorder is hepatits B (see, e.g., WO 2015/061294).

In some embodiments, the condition, disease or disorder is selected from cardiovascular diseases (including e.g., myocardial infarction).

In some embodiments, the condition, disease or disorder is age-related macular degeneration.

In some embodiments, the condition, disease or disorder is mucositis, also known as stomatitits, which can occur as a result of chemotherapy or radiation therapy, either alone or in combination as well as damage caused by exposure to radiation outside of the context of radiation therapy.

In some embodiments, the condition, disease or disorder is uveitis, which is inflammation of the uvea (e.g., anterior uveitis, e.g., iridocyclitis or iritis; intermediate uveitis (also known as pars planitis); posterior uveitis; or chorioretinitis, e.g., pan-uveitis).

In some embodiments, the condition, disease or disorder is selected from the group consisting of a cancer, a neurological disorder, an autoimmune disease, hepatitis B, uvetitis, a cardiovascular disease, age-related macular degeneration, and mucositis.

Still other examples can include those indications discussed herein and below in contemplated combination therapy regimens.

Combination Therapy

This disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein.

In certain embodiments, the methods described herein can further include administering one or more additional cancer therapies.

The one or more additional cancer therapies can include, without limitation, surgery, radiotherapy, chemotherapy, toxin therapy, immunotherapy, cryotherapy, cancer vaccines (e.g., HPV vaccine, hepatitis B vaccine, Oncophage, Provenge) and gene therapy, as well as combinations thereof. Immunotherapy, including, without limitation, adoptive cell therapy, the derivation of stem cells and/or dendritic cells, blood transfusions, lavages, and/or other treatments, including, without limitation, freezing a tumor.

In some embodiments, the one or more additional cancer therapies is chemotherapy, which can include administering one or more additional chemotherapeutic agents.

In certain embodiments, the additional chemotherapeutic agent is an immunomodulatory moiety, e.g., an immune checkpoint inhibitor. In certain of these embodiments, the immune checkpoint inhibitor targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155; e.g., CTLA-4 or PD1 or PD-L1). See, e.g., Postow, M. J. Clin. Oncol. 2015, 33, 1.

In certain of these embodiments, the immune checkpoint inhibitor is selected from the group consisting of: Urelumab, PF-05082566, MEDI6469, TRX518, Varlilumab, CP-870893, Pembrolizumab (PD1), Nivolumab (PD1), Atezolizumab (formerly MPDL3280A) (PDL1), MEDI4736 (PD-L1), Avelumab (PD-L1), PDR001 (PD1), BMS-986016, MGA271, Lirilumab, IPH2201, Emactuzumab, INCB024360, Galunisertib, Ulocuplumab, BKT140, Bavituximab, CC-90002, Bevacizumab, and MNRP1685A, and MGA271.

In certain embodiments, the additional chemotherapeutic agent is an alkylating agent. Alkylating agents are so named because of their ability to alkylate many nucleophilic functional groups under conditions present in cells, including, but not limited to cancer cells. In a further embodiment, an alkylating agent includes, but is not limited to, Cisplatin, carboplatin, mechlorethamine, cyclophosphamide, chlorambucil, ifosfamide and/or oxaliplatin. In an embodiment, alkylating agents can function by impairing cell function by forming covalent bonds with the amino, carboxyl, sulfhydryl, and phosphate groups in biologically important molecules or they can work by modifying a cell's DNA. In a further embodiment an alkylating agent is a synthetic, semisynthetic or derivative.

In certain embodiments, the additional chemotherapeutic agent is an anti-metabolite. Anti-metabolites masquerade as purines or pyrimidines, the building-blocks of DNA and in general, prevent these substances from becoming incorporated in to DNA during the “S” phase (of the cell cycle), stopping normal development and division. Anti-metabolites can also affect RNA synthesis. In an embodiment, an antimetabolite includes, but is not limited to azathioprine and/or mercaptopurine. In a further embodiment an anti-metabolite is a synthetic, semisynthetic or derivative.

In certain embodiments, the additional chemotherapeutic agent is a plant alkaloid and/or terpenoid. These alkaloids are derived from plants and block cell division by, in general, preventing microtubule function. In an embodiment, a plant alkaloid and/or terpenoid is a vinca alkaloid, a podophyllotoxin and/or a taxane. Vinca alkaloids, in general, bind to specific sites on tubulin, inhibiting the assembly of tubulin into microtubules, generally during the M phase of the cell cycle. In an embodiment, a vinca alkaloid is derived, without limitation, from the Madagascar periwinkle, Catharanthus roseus (formerly known as Vinca rosea). In an embodiment, a vinca alkaloid includes, without limitation, Vincristine, Vinblastine, Vinorelbine and/or Vindesine. In an embodiment, a taxane includes, but is not limited, to Taxol, Paclitaxel and/or Docetaxel. In a further embodiment a plant alkaloid or terpernoid is a synthetic, semisynthetic or derivative. In a further embodiment, a podophyllotoxin is, without limitation, an etoposide and/or teniposide. In an embodiment, a taxane is, without limitation, docetaxel and/or ortataxel. [021] In an embodiment, a cancer therapeutic is a topoisomerase. Topoisomerases are essential enzymes that maintain the topology of DNA. Inhibition of type I or type II topoisomerases interferes with both transcription and replication of DNA by upsetting proper DNA supercoiling. In a further embodiment, a topoisomerase is, without limitation, a type I topoisomerase inhibitor or a type II topoisomerase inhibitor. In an embodiment a type I topoisomerase inhibitor is, without limitation, a camptothecin. In another embodiment, a camptothecin is, without limitation, exatecan, irinotecan, lurtotecan, topotecan, BNP 1350, CKD 602, DB 67 (AR67) and/or ST 1481. In an embodiment, a type II topoisomerase inhibitor is, without limitation, epipodophyllotoxin. In a further embodiment an epipodophyllotoxin is, without limitation, an amsacrine, etoposid, etoposide phosphate and/or teniposide. In a further embodiment a topoisomerase is a synthetic, semisynthetic or derivative, including those found in nature such as, without limitation, epipodophyllotoxins, substances naturally occurring in the root of American Mayapple (Podophyllum peltatum).

In certain embodiments, the additional chemotherapeutic agent is a stilbenoid. In a further embodiment, a stilbenoid includes, but is not limited to, Resveratrol, Piceatannol, Pinosylvin, Pterostilbene, Alpha-Viniferin, Ampelopsin A, Ampelopsin E, Diptoindonesin C, Diptoindonesin F, Epsilon-Vinferin, Flexuosol A, Gnetin H, Hemsleyanol D, Hopeaphenol, Trans-Diptoindonesin B, Astringin, Piceid and Diptoindonesin A. In a further embodiment a stilbenoid is a synthetic, semisynthetic or derivative.

In certain embodiments, the additional chemotherapeutic agent is a cytotoxic antibiotic. In an embodiment, a cytotoxic antibiotic is, without limitation, an actinomycin, an anthracenedione, an anthracycline, thalidomide, dichloroacetic acid, nicotinic acid, 2-deoxyglucose and/or chlofazimine. In an embodiment, an actinomycin is, without limitation, actinomycin D, bacitracin, colistin (polymyxin E) and/or polymyxin B. In another embodiment, an antracenedione is, without limitation, mitoxantrone and/or pixantrone. In a further embodiment, an anthracycline is, without limitation, bleomycin, doxorubicin (Adriamycin), daunorubicin (daunomycin), epirubicin, idarubicin, mitomycin, plicamycin and/or valrubicin. In a further embodiment a cytotoxic antibiotic is a synthetic, semisynthetic or derivative.

In certain embodiments, the additional chemotherapeutic agent is selected from endostatin, angiogenin, angiostatin, chemokines, angioarrestin, angiostatin (plasminogen fragment), basement-membrane collagen-derived anti-angiogenic factors (tumstatin, canstatin, or arrestin), anti-angiogenic antithrombin III, signal transduction inhibitors, cartilage-derived inhibitor (CDI), CD59 complement fragment, fibronectin fragment, gro-beta, heparinases, heparin hexasaccharide fragment, human chorionic gonadotropin (hCG), interferon alpha/beta/gamma, interferon inducible protein (IP-10), interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), various retinoids, tetrahydrocortisol-S, thrombospondin-1 (TSP-1), transforming growth factor-beta (TGF-0), vasculostatin, vasostatin (calreticulin fragment) and the like.

In certain embodiments, the additional chemotherapeutic agent is selected from abiraterone acetate, altretamine, anhydrovinblastine, auristatin, bexarotene, bicalutamide, BMS 184476, 2,3,4,5,6-pentafluoro-N-(3-fluoro-4-methoxyphenyl)benzene sulfonamide, bleomycin, N,N-dimethyl-L-valyl-L-valyl-N-methyl-L-valyl-L-proly-1-Lproline-t-butylamide, cachectin, cemadotin, chlorambucil, cyclophosphamide, 3′,4′-didehydro-4′-deoxy-8′-norvin-caleukoblastine, docetaxol, doxetaxel, cyclophosphamide, carboplatin, carmustine, cisplatin, cryptophycin, cyclophosphamide, cytarabine, dacarbazine (DTIC), dactinomycin, daunorubicin, decitabine dolastatin, doxorubicin (adriamycin), etoposide, 5-fluorouracil, finasteride, flutamide, hydroxyurea and hydroxyureataxanes, ifosfamide, liarozole, lonidamine, lomustine (CCNU), MDV3100, mechlorethamine (nitrogen mustard), melphalan, mivobulin isethionate, rhizoxin, sertenef, streptozocin, mitomycin, methotrexate, taxanes, nilutamide, onapristone, paclitaxel, prednimustine, procarbazine, RPR109881, stramustine phosphate, tamoxifen, tasonermin, taxol, tretinoin, vinblastine, vincristine, vindesine sulfate, and vinflunine.

In certain embodiments, the additional chemotherapeutic agent is platinum, cisplatin, carboplatin, oxaliplatin, mechlorethamine, cyclophosphamide, chlorambucil, azathioprine, mercaptopurine, vincristine, vinblastine, vinorelbine, vindesine, etoposide and teniposide, paclitaxel, docetaxel, irinotecan, topotecan, amsacrine, etoposide, etoposide phosphate, teniposide, 5-fluorouracil, leucovorin, methotrexate, gemcitabine, taxane, leucovorin, mitomycin C, tegafur-uracil, idarubicin, fludarabine, mitoxantrone, ifosfamide and doxorubicin. Additional agents include inhibitors of mTOR (mammalian target of rapamycin), including but not limited to rapamycin, everolimus, temsirolimus and deforolimus.

In still other embodiments, the additional chemotherapeutic agent can be selected from those delineated in U.S. Pat. No. 7,927,613, which is incorporated herein by reference in its entirety.

In some embodiments, the additional therapeutic agent and/or regimen are those that can be used for treating other STING-associated conditions, e.g., type I interferonopathies (e.g., STING-associated vasculopathy with onset in infancy (SAVI)), Aicardi-Goutieres Syndrome (AGS), genetic forms of lupus, and inflammation-associated disorders such as systemic lupus erythematosus, and rheumatoid arthritis and the like.

Non-limiting examples of additional therapeutic agents and/or regimens for treating rheumatoid arthritis include non-steroidal anti-inflammatory drugs (NSAIDs; e.g., ibuprofen and naproxen), corticosteroids (e.g, prednisone), disease-modifying antirheumatic drugs (DMARDs; e.g., methotrexate (Trexall®, Otrexup®, Rasuvo®, Rheumatrex®), leflunomide (Arava®), hydroxychloroquine (Plaquenil), PF-06650833, iguratimod, tofacitinib (Xeljanz®), ABBV-599, evobrutinib, and sulfasalazine (Azulfidine®)), and biologics (e.g., abatacept (Orencia®), adalimumab (Humira®), anakinra (Kineret®), certolizumab (Cimzia®), etanercept (Enbrel®), golimumab (Simponi®), infliximab (Remicade®), rituximab (Rituxan®), tocilizumab (Actemra®), vobarilizumab, sarilumab (Kevzara®), secukinumab, ABP 501, CHS-0214, ABC-3373, and tocilizumab (ACTEMRA®)).

Non-limiting examples of additional therapeutic agents and/or regimens for treating lupus include steroids, topical immunomodulators (e.g., tacrolimus ointment (Protopic®) and pimecrolimus cream (Elidel®)), thalidomide (Thalomid®), non-steroidal anti-inflammatory drugs (NSAIDs; e.g., ibuprofen and naproxen), antimalarial drugs (e.g., Hydroxychloroquine (Plaquenil)), corticosteroids (e.g, prednisone) and immunomodulators (e.g., evobrutinib, iberdomide, voclosporin, cenerimod, azathioprine (Imuran®), cyclophosphamide (Cytoxan®, Neosar®, Endoxan®), and cyclosporine (Neoral, Sandimmune®, Gengraf®), and mycophenolate mofetil) baricitinb, iguratimod, filogotinib, GS-9876, rapamycin, and PF-06650833), and biologics (e.g., belimumab (Benlysta®), anifrolumab, prezalumab, MEDI0700, obinutuzumab, vobarilizumab, lulizumab, atacicept, PF-06823859, and lupizor, rituximab, BT063, BI655064, BIIB059, aldesleukin (Proleukin®), dapirolizumab, edratide, IFN-α-kinoid, OMS721, RC18, RSLV-132, theralizumab, XmAb5871, and ustekinumab (Stelara®)). For example, non-limiting treatments for systemic lupus erythematosus include non-steroidal anti-inflammatory drugs (NSAIDs; e.g., ibuprofen and naproxen), antimalarial drugs (e.g., Hydroxychloroquine (Plaquenil)), corticosteroids (e.g, prednisone) and immunomodulators (e.g., iberdomide, voclosporin, azathioprine (Imuran®), cyclophosphamide (Cytoxan®, Neosar®, Endoxan®), and cyclosporine (Neoral, Sandimmune®, Gengraf®), and mycophenolate mofetil, baricitinb, filogotinib, and PF-06650833), and biologics (e.g., belimumab (Benlysta®), anifrolumab, prezalumab, MEDIO700, vobarilizumab, lulizumab, atacicept, PF-06823859, lupizor, rituximab, BT063, BI655064, BIIB059, aldesleukin (Proleukin®), dapirolizumab, edratide, IFN-α-kinoid, RC18, RSLV-132, theralizumab, XmAb5871, and ustekinumab (Stelara®)). As another example, non-limiting examples of treatments for cutaneous lupus include steroids, immunomodulators (e.g., tacrolimus ointment (Protopic®) and pimecrolimus cream (Elidel®)), GS-9876, filogotinib, and thalidomide (Thalomid®). Agents and regimens for treating drug-induced and/or neonatal lupus can also be administered.

Non-limiting examples of additional therapeutic agents and/or regimens for treating STING-associated vasculopathy with onset in infancy (SAVI) include JAK inhibitors (e.g., tofacitinib, ruxolitinib, filgotinib, and baricitinib).

Non-limiting examples of additional therapeutic agents and/or regimens for treating Aicardi-Goutieres Syndrome (AGS) include physiotherapy, treatment for respiratory complications, anticonvulsant therapies for seizures, tube-feeding, nucleoside reverse transcriptase inhibitors (e.g., emtricitabine (e.g., Emtriva®), tenofovir (e.g., Viread®), emtricitabine/tenofovir (e.g., Truvada®), zidovudine, lamivudine, and abacavir), and JAK inhibitors (e.g., tofacitinib, ruxolitinib, filgotinib, and baricitinib).

Non-limiting examples of additional therapeutic agents and/or regimens for treating IBDs include 6-mercaptopurine, AbGn-168H, ABX464, ABT-494, adalimumab, AJM300, alicaforsen, AMG139, anrukinzumab, apremilast, ATR-107 (PF0530900), autologous CD34-selected peripheral blood stem cells transplant, azathioprine, bertilimumab, BI 655066, BMS-936557, certolizumab pegol (Cimzia®), cobitolimod, corticosteroids (e.g., prednisone, Methylprednisolone, prednisone), CP-690,550, CT-P13, cyclosporine, DIMS0150, E6007, E6011, etrasimod, etrolizumab, fecal microbial transplantation, figlotinib, fingolimod, firategrast (SB-683699) (formerly T-0047), GED0301, GLPG0634, GLPG0974, guselkumab, golimumab, GSK1399686, HMPL-004 (Andrographis paniculata extract), IMU-838, infliximab, Interleukin 2 (IL-2), Janus kinase (JAK) inhibitors, laquinimod, masitinib (AB1010), matrix metalloproteinase 9 (MMP 9) inhibitors (e.g., GS-5745), MEDI2070, mesalamine, methotrexate, mirikizumab (LY3074828), natalizumab, NNC 0142-0000-0002, NNC0114-0006, ozanimod, peficitinib (JNJ-54781532), PF-00547659, PF-04236921, PF-06687234, QAX576, RHB-104, rifaximin, risankizumab, RPC1063, SB012, SHP647, sulfasalazine, TD-1473, thalidomide, tildrakizumab (MK 3222), TJ301, TNF-Kinoid®, tofacitinib, tralokinumab, TRK-170, upadacitinib, ustekinumab, UTTR1147A, V565, vatelizumab, VB-201, vedolizumab, and vidofludimus.

Non-limiting examples of additional therapeutic agents and/or regimens for treating irritable bowel syndrome include alosetron, bile acid sequesterants (e.g., cholestyramine, colestipol, colesevelam), chloride channel activators (e.g., lubiprostone), coated peppermint oil capsules, desipramine, dicyclomine, ebastine, eluxadoline, farnesoid X receptor agonist (e.g., obeticholic acid), fecal microbiota transplantation, fluoxetine, gabapentin, guanylate cyclase-C agonists (e.g., linaclotide, plecanatide), ibodutant, imipramine, JCM-16021, loperamide, lubiprostone, nortriptyline, ondansetron, opioids, paroxetine, pinaverium, polyethylene glycol, pregabalin, probiotics, ramosetron, rifaximin, and tanpanor.

Non-limiting examples of additional therapeutic agents and/or regimens for treating scleroderma include non-steroidal anti-inflammatory drugs (NSAIDs; e.g., ibuprofen and naproxen), corticosteroids (e.g, prednisone), immunomodulators (e.g., azathioprine, methotrexate (Trexall®, Otrexup®, Rasuvo®, Rheumatrex®), cyclophosphamide (Cytoxan®, Neosar®, Endoxan®), and cyclosporine (Neoral®, Sandimmune®, Gengraf®), antithymocyte globulin, mycophenolate mofetil, intravenous immunoglobulin, rituximab, sirolimus, and alefacept), calcium channel blockers (e.g., nifedipine), alpha blockers, serotonin receptor antagonists, angiotensin II receptor inhibitors, statins, local nitrates, iloprost, phosphodiesterase 5 inhibitors (e.g., sildenafil), bosentan, tetracycline antibiotics, endothelin receptor antagonists, prostanoids, and tyrosine kinase inhibitors (e.g., imatinib, nilotinib and dasatinib).

Non-limiting examples of additional therapeutic agents and/or regimens for treating Crohn's Disease (CD) include adalimumab, autologous CD34-selected peripheral blood stem cells transplant, 6-mercaptopurine, azathioprine, certolizumab pegol (Cimzia®), corticosteroids (e.g., prednisone), etrolizumab, E6011, fecal microbial transplantation, figlotinib, guselkumab, infliximab, IL-2, JAK inhibitors, matrix metalloproteinase 9 (MMP 9) inhibitors (e.g., GS-5745), MEDI2070, mesalamine, methotrexate, natalizumab, ozanimod, RHB-104, rifaximin, risankizumab, SHP647, sulfasalazine, thalidomide, upadacitinib, V565, and vedolizumab.

Non-limiting examples of additional therapeutic agents and/or regimens for treating UC include AbGn-168H, ABT-494, ABX464, apremilast, PF-00547659, PF-06687234, 6-mercaptopurine, adalimumab, azathioprine, bertilimumab, brazikumab (MEDJ2070), cobitolimod, certolizumab pegol (Cimzia®), CP-690,550, corticosteroids (e.g., multimax budesonide, Methylprednisolone), cyclosporine, E6007, etrasimod, etrolizumab, fecal microbial transplantation, figlotinib, guselkumab, golimumab, IL-2, IMU-838, infliximab, matrix metalloproteinase 9 (MMP9) inhibitors (e.g., GS-5745), mesalamine, mesalamine, mirikizumab (LY3074828), RPC1063, risankizumab (BI 6555066), SHP647, sulfasalazine, TD-1473, TJ301, tildrakizumab (MK 3222), tofacitinib, tofacitinib, ustekinumab, UTTR1147A, and vedolizumab.

Non-limiting examples of additional therapeutic agents and/or regimens for treating autoimmune colitis include corticosteroids (e.g., budesonide, prednisone, prednisolone, Beclometasone dipropionate), diphenoxylate/atropine, infliximab, loperamide, mesalamine, TIP60 inhibitors (see, e.g., U.S. Patent Application Publication No. 2012/0202848), and vedolizumab.

Non-limiting examples of additional therapeutic agents and/or regimens for treating iatrogenic autoimmune colitis include corticosteroids (e.g., budesonide, prednisone, prednisolone, Beclometasone dipropionate), diphenoxylate/atropine, infliximab, loperamide, TIP60 inhibitors (see, e.g., U.S. Patent Application Publication No. 2012/0202848), and vedolizumab.

Non-limiting examples of additional therapeutic agents and/or regimens for treating colitis induced by one or more chemotherapeutics agents include corticosteroids (e.g., budesonide, prednisone, prednisolone, beclometasone dipropionate), diphenoxylate/atropine, infliximab, loperamide, mesalamine, TIP60 inhibitors (see, e.g., U.S. Patent Application Publication No. 2012/0202848), and vedolizumab.

Non-limiting examples of additional therapeutic agents and/or regimens for treating colitis induced by treatment with adoptive cell therapy include corticosteroids (e.g., budesonide, prednisone, prednisolone, beclometasone dipropionate), diphenoxylate/atropine, infliximab, loperamide, TIP60 inhibitors (see, e.g., U.S. Patent Application Publication No. 2012/0202848), and vedolizumab.

Non-limiting examples of additional therapeutic agents and/or regimens for treating colitis associated with one or more alloimmune diseases include corticosteroids (e.g., budesonide, prednisone, prednisolone, beclometasone dipropionate), sulfasalazine, and eicopentaenoic acid.

Non-limiting examples of additional therapeutic agents and/or regimens for treating radaiation enteritis include teduglutide, amifostine, angiotensin-converting enzyme (ACE) inhibitors (e.g., benazepril, captopril, enalapril, fosinopril, lisinopril, moexipril, perindopril, quinapril, ramipril, and trandolapril), probiotics, selenium supplementation, statins (e.g., atorvastatin, fluvastatin, lovastatin, pravastatin, rosuvastatin, simvastatin, and pitavastatin), sucralfate, and vitamin E.

Non-limiting examples of additional therapeutic agents and/or regimens for treating collagenous colitis include 6-mercaptopurine, azathaioprine, bismuth subsalicate, Boswellia serrata extract, cholestyramine, colestipol, corticosteroids (e.g., budesonide, prednisone, prednisolone, beclometasone dipropionate), loperamide, mesalamine, methotrexate, probiotics, and sulfasalazine.

Non-limiting examples of additional therapeutic agents and/or regimens for treating lyphocytic colitis include 6-mercaptopurine, azathioprine, bismuth subsalicylate, cholestyramine, colestipol, corticosteroids (e.g., budesonide, prednisone, prednisolone, beclometasone dipropionate), loperamide, mesalamine, methotrexate, and sulfasalazine.

Non-limiting examples of additional therapeutic agents and/or regimens for treating microscopic colitis include 6-mercaptopurine, azathioprine, bismuth subsalicylate, Boswellia serrata extract, cholestyramine, colestipol, corticosteroids (e.g., budesonide, prednisone, prednisolone, beclometasone dipropionate), fecal microbial transplantation, loperamide, mesalamine, methotrexate, probiotics, and sulfasalazine.

Non-limiting examples of additional therapeutic agents and/or regimens for treating alloimmune disease include intrauterine platelet transfusions, intravenous immunoglobin, maternal steroids, abatacept, alemtuzumab, alpha1-antitrypsin, AMG592, antithymocyte globulin, barcitinib, basiliximab, bortezomib, brentuximab, cannabidiol, corticosteroids (e.g., methylprednisone, prednisone), cyclosporine, dacilzumab, defribrotide, denileukin diftitox, glasdegib, ibrutinib, IL-2, infliximab, itacitinib, LBH589, maraviroc, mycophenolate mofetil, natalizumab, neihulizumab, pentostatin, pevonedistat, photobiomodulation, photopheresis, ruxolitinib, sirolimus, sonidegib, tacrolimus, tocilizumab, and vismodegib.

Non-limiting examples of additional therapeutic agents and/or regimens for treating multiple sclerosis (MS) include alemtuzumab (Lemtrada®), ALKS 8700, amiloride, ATX-MS-1467, azathioprine, baclofen (Lioresal®), beta interferons (e.g., IFN-β-1a, IFN-β-1b), cladribine, corticosteroids (e.g., methylprednisolone), daclizumab, dimethyl fumarate (Tecfidera®), fingolimod (Gilenya®), fluoxetine, glatiramer acetate (Copaxone®), hydroxychloroquine, ibudilast, idebenone, laquinimod, lipoic acid, losartan, masitinib, MD1003 (biotin), mitoxantrone, montelukast, natalizumab (Tysabri®), NeuroVax™, ocrelizumab, ofatumumab, pioglitazone, and RPC1063.

Non-limiting examples of additional therapeutic agents and/or regimens for treating graft-vs-host disease include abatacept, alemtuzumab, alphal-antitrypsin, AMG592, antithymocyte globulin, barcitinib, basiliximab, bortezomib, brentuximab, cannabidiol, corticosteroids (e.g., methylprednisone, prednisone), cyclosporine, dacilzumab, defribrotide, denileukin diftitox, glasdegib, ibrutinib, IL-2, imatinib, infliximab, itacitinib, LBH589, maraviroc, mycophenolate mofetil, natalizumab, neihulizumab, pentostatin, pevonedistat, photobiomodulation, photopheresis, ruxolitinib, sirolimus, sonidegib, tacrolimus, tocilizumab, and vismodegib.

Non-limiting examples of additional therapeutic agents and/or regimens for treating acute graft-vs-host disease include alemtuzumab, alpha-1 antitrypsin, antithymocyte globulin, basiliximab, brentuximab, corticosteroids (e.g., methylprednisone, prednisone), cyclosporine, dacilzumab, defribrotide, denileukin diftitox, ibrutinib, infliximab, itacitinib, LBH589, mycophenolate mofetil, natalizumab, neihulizumab, pentostatin, photopheresis, ruxolitinib, sirolimus, tacrolimus, and tocilizumab.

Non-limiting examples of additional therapeutic agents and/or regimens for treating chronic graft vs. host disease include abatacept, alemtuzumab, AMG592, antithymocyte globulin, basiliximab, bortezomib, corticosteroids (e.g., methylprednisone, prednisone), cyclosporine, dacilzumab, denileukin diftitox, glasdegib, ibrutinib, IL-2, imatinib, infliximab, mycophenolate mofetil, pentostatin, photobiomodulation, photopheresis, ruxolitinib, sirolimus, sonidegib, tacrolimus, tocilizumab, and vismodegib.

Non-limiting examples of additional therapeutic agents and/or regimens for treating celiac disease include AMG 714, AMY01, Aspergillus niger prolyl endoprotease, BL-7010, CALY-002, GBR 830, Hu-Mik-Beta-1, IM4GX003, KumaMax, Larazotide Acetate, Nexvan2®, pancrelipase, TIP-GLIA, vedolizumab, and ZED1227.

Non-limiting examples of additional therapeutic agents and/or regimens for treating psoriasis include topical corticosteroids, topical crisaborole/AN2728, topical SNA-120, topical SAN021, topical tapinarof, topical tocafinib, topical IDP-118, topical M518101, topical calcipotriene and betamethasone dipropionate (e.g., MC2-01 cream and Taclonex®), topical P-3073, topical LEO 90100 (Enstilar®), topical betamethasone dipropriate (Sernivo®), halobetasol propionate (Ultravate®), vitamin D analogues (e.g., calcipotriene (Dovonex®) and calcitriol (Vectical®)), anthralin (e.g., Dritho-Scalp® and Dritho-Creme®), topical retinoids (e.g., tazarotene (e.g., Tazorac® and Avage®)), calcineurin inhibitors (e.g., tacrolimus (Prograf®) and pimecrolimus (Elidel®)), salicylic acid, coal tar, moisturizers, phototherapy (e.g., exposure to sunlight, UVB phototherapy, narrow band UVB phototherapy, Goeckerman therapy, psoralen plus ultraviolet A (PUVA) therapy, and excimer laser), retinoids (e.g., acitretin (Soriatane®)), methotrexate (Trexall®, Otrexup®, Rasuvo®, Rheumatrex®), Apo805K1, baricitinib, FP187, KD025, prurisol, VTP-43742, XP23829, ZPL-389, CF101 (piclidenoson), LAS41008, VPD-737 (serlopitant), upadacitinib (ABT-494), aprmilast, tofacitibin, cyclosporine (Neoral®, Sandimmune®, Gengraf®), biologics (e.g., etanercept (Enbrel®), entanercept-szzs (Elrezi®), infliximab (Remicade®), adalimumab (Humira®), adalimumab-adbm (Cyltezo®), ustekinumab (Stelara®), golimumab (Simponi®), apremilast (Otezla®), secukinumab (Cosentyx®), certolixumab pegol, secukinumab, tildrakizumab-asmn, infliximab-dyyb, abatacept, ixekizumab (Taltz®), ABP 710, BCD-057, BI695501, bimekizumab (UCB4940), CHS-1420, GP2017, guselkumab (CNTO 1959), HD203, M923, MSB11022, Mirikizumab (LY3074828), PF-06410293, PF-06438179, risankizumab (BI655066), SB2, SB4, SB5, siliq (brodalumab), namilumab (MT203, tildrakizumab (MK-3222), and ixekizumab (Taltz®)), thioguanine, and hydroxyurea (e.g., Droxia® and Hydrea®).

Non-limiting examples of additional therapeutic agents and/or regimens for treating cutaneous T-cell lymphoma include phototherapy (e.g., exposure to sunlight, UVB phototherapy, narrow band UVB phototherapy, Goeckerman therapy, psoralen plus ultraviolet A (PUVA) therapy, and excimer laser), extracorporeal photopheresis, radiation therapy (e.g., spot radiation and total skin body electron beam therapy), stem cell transplant, corticosteroids, imiquimod, bexarotene gel, topical bis-chloroethyl-nitrourea, mechlorethamine gel, vorinostat (Zolinza®), romidepsin (Istodax®), pralatrexate (Folotyn®) biologics (e.g., alemtuzumab (Campath®), brentuximab vedotin (SGN-35), mogamulizumab, and IPH4102).

Non-limiting examples of additional therapeutic agents and/or regimens for treating uveitis include corticosteroids (e.g., intravitreal triamcinolone acetonide injectable suspensions), antibiotics, antivirals (e.g., acyclovir), dexamethasone, immunomodulators (e.g., tacrolimus, leflunomide, cyclophosphamide (Cytoxan®, Neosar®, Endoxan®), and cyclosporine (Neoral®, Sandimmune®, Gengraf®), chlorambucil, azathioprine, methotrexate, and mycophenolate mofetil), biologics (e.g., infliximab (Remicade®), adalimumab (Humira®), etanercept (Enbrel®), golimumab (Simponi®), certolizumab (Cimzia®), rituximab (Rituxan®), abatacept (Orencia®), basiliximab (Simulect®), anakinra (Kineret®), canakinumab (Ilaris®), gevokixumab (XOMA052), tocilizumab (Actemra®), alemtuzumab (Campath®), efalizumab (Raptiva®), LFG316, sirolimus (Santen®), abatacept, sarilumab (Kevzara®), and daclizumab (Zenapax®)), cytotoxic drugs, surgical implant (e.g., fluocinolone insert), and vitrectomy.

on-limiting examples of additional therapeutic agents and/or regimens for treating mucositis include AGO13, SGX942 (dusquetide), amifostine (Ethyol®), cryotherapy, cepacol lonzenges, capsaicin lozenges, mucoadhesives (e.g., MuGard®) oral diphenhydramine (e.g., Benadry® elixir), oral bioadherents (e.g., polyvinylpyrrolidone-sodium hyaluronate gel (Gelclair®)), oral lubricants (e.g., Oral Balance®), caphosol, chamomilla recutita mouthwash, edible grape plant exosome, antiseptic mouthwash (e.g., chlorhexidine gluconate (e.g., Peridex® or Periogard®), topical pain relievers (e.g., lidocaine, benzocaine, dyclonine hydrochloride, xylocaine (e.g., viscous xylocaine 2%), and Ulcerease® (0.6% phenol)), corticosteroids (e.g., prednisone), pain killers (e.g., ibuprofen, naproxen, acetaminophen, and opioids), GC4419, palifermin (keratinocyte growth factor; Kepivance®), ATL-104, clonidine lauriad, IZN-6N4, SGX942, rebamipide, nepidermin, soluble β-1,3/1,6 glucan, P276, LP-0004-09, CR-3294, ALD-518, IZN-6N4, quercetin, granules comprising vaccinium myrtillus extract, macleaya cordata alkaloids and echinacea angustifolia extract (e.g., SAMITAL®), and gastrointestinal cocktail (an acid reducer such aluminum hydroxide and magnesium hydroxide (e.g., Maalox), an antifungal (e.g., nystatin), and an analgesic (e.g., hurricane liquid)). For example, non-limiting examples of treatments for oral mucositis include AGO13, amifostine (Ethyol®), cryotherapy, cepacol lonzenges, mucoadhesives (e.g., MuGard®) oral diphenhydramine (e.g., Benadry® elixir), oral bioadherents (e.g., polyvinylpyrrolidone-sodium hyaluronate gel (Gelclair®)), oral lubricants (e.g., Oral Balance®), caphosol, chamomilla recutita mouthwash, edible grape plant exosome, antiseptic mouthwash (e.g., chlorhexidine gluconate (e.g., Peridex® or Periogard®), topical pain relievers (e.g., lidocaine, benzocaine, dyclonine hydrochloride, xylocaine (e.g., viscous xylocaine 2%), and Ulcerease® (0.6% phenol)), corticosteroids (e.g., prednisone), pain killers (e.g., ibuprofen, naproxen, acetaminophen, and opioids), GC4419, palifermin (keratinocyte growth factor; Kepivance®), ATL-104, clonidine lauriad, IZN-6N4, SGX942, rebamipide, nepidermin, soluble β-1,3/1,6 glucan, P276, LP-0004-09, CR-3294, ALD-518, IZN-6N4, quercetin, and gastrointestinal cocktail (an acid reducer such aluminum hydroxide and magnesium hydroxide (e.g., Maalox), an antifungal (e.g., nystatin), and an analgesic (e.g., hurricane liquid)). As another example, non-limiting examples of treatments for esophageal mucositis include xylocaine (e.g., gel viscous Xylocaine 2%). As another example, treatments for intestinal mucositis, treatments to modify intestinal mucositis, and treatments for intestinal mucositis signs and symptoms include gastrointestinal cocktail (an acid reducer such aluminum hydroxide and magnesium hydroxide (e.g., Maalox), an antifungal (e.g., nystatin), and an analgesic (e.g., hurricane liquid)).

In certain embodiments, the second therapeutic agent or regimen is administered to the subject prior to contacting with or administering the chemical entity (e.g., about one hour prior, or about 6 hours prior, or about 12 hours prior, or about 24 hours prior, or about 48 hours prior, or about 1 week prior, or about 1 month prior).

In other embodiments, the second therapeutic agent or regimen is administered to the subject at about the same time as contacting with or administering the chemical entity. By way of example, the second therapeutic agent or regimen and the chemical entity are provided to the subject simultaneously in the same dosage form. As another example, the second therapeutic agent or regimen and the chemical entity are provided to the subject concurrently in separate dosage forms.

In still other embodiments, the second therapeutic agent or regimen is administered to the subject after contacting with or administering the chemical entity (e.g., about one hour after, or about 6 hours after, or about 12 hours after, or about 24 hours after, or about 48 hours after, or about 1 week after, or about 1 month after).

Patient Selection

In some embodiments, the methods described herein further include the step of identifying a subject (e.g., a patient) in need of such treatment (e.g., by way of biopsy, endoscopy, or other conventional method known in the art). In certain embodiments, the STING protein can serve as a biomarker for certain types of cancer, e.g., colon cancer and prostate cancer. In other embodiments, identifying a subject can include assaying the patient's tumor microenvironment for the absence of T-cells and/or presence of exhausted T-cells, e.g., patients having one or more cold tumors. Such patients can include those that are resistant to treatment with checkpoint inhibitors. In certain embodiments, such patients can be treated with a chemical entity herein, e.g., to recruit T-cells into the tumor, and in some cases, further treated with one or more checkpoint inhibitors, e.g., once the T-cells become exhausted.

In some embodiments, the chemical entities, methods, and compositions described herein can be administered to certain treatment-resistant patient populations (e.g., patients resistant to checkpoint inhibitors; e.g., patients having one or more cold tumors, e.g., tumors lacking T-cells or exhausted T-cells).

Compound Preparation

As can be appreciated by the skilled artisan, methods of synthesizing the compounds of the formulae herein will be evident to those of ordinary skill in the art. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and RGM. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof. The starting materials used in preparing the compounds of the invention are known, made by known methods, or are commercially available. The skilled artisan will also recognize that conditions and reagents described herein that can be interchanged with alternative art-recognized equivalents. For example, in many reactions, triethylamine can be interchanged with other bases, such as non-nucleophilic bases (e.g. diisopropylamine, 1,8-diazabicycloundec-7-ene, 2,6-di-tert-butylpyridine, or tetrabutylphosphazene).

The skilled artisan will recognize a variety of analytical methods that can be used to characterize the compounds described herein, including, for example, ¹H NMR, heteronuclear NMR, mass spectrometry, liquid chromatography, and infrared spectroscopy. The foregoing list is a subset of characterization methods available to a skilled artisan and is not intended to be limiting.

To further illustrate the foregoing, the following non-limiting, exemplary synthetic schemes are included. Variations of these examples within the scope of the claims are within the purview of one skilled in the art and are considered to fall within the scope of the invention as described, and claimed herein. The reader will recognize that the skilled artisan, provided with the present disclosure, and skill in the art is able to prepare and use the invention without exhaustive examples.

The following abbreviations have the indicated meanings:

| | |
|---|---|
| ACN = acetonitrile | PyBOP = Benzotriazol-1-yl- |
| | oxytripyrrolidinophosphonium hexafluorophosphate |
| AcOH = acetic acid | HATU = M-[(Dimethylamino)- 1H-1,2,3-triazolo- |
| | [4,5-b]pyridin-1-ylmethylene]-N- |
| | methylmethanaminium hexafluorophosphate N- |
| | oxide |
| Boc₂O = di-tert-butyl pyrocarbomate | MeOH = methanol |
| Bu = butyl | NaOH = sodium hydroxide |
| DBU = 1,8- diazabicycloundec-7-ene | T3P = 1-propanephosphonic anhydride |
| DCM = dichloromethane | EtOAc = Ethyl acetate |
| DEAD = diethyl | SpeedVac = Savant SC250EXP |
| azodicarboxylate | SpeedVac Concentrator |
| DIEA = N,N- diisopropylethylamine | BTC = (bis(trichloromethyl) carbonate |
| DMF = N,N- dimethylformamide | Dess-Martin = (1,1,1- triacetoxy)-1,1-dihydro-1,2- |
| | benziodoxol-3(1H)-one |
| DMSO = dimethyl sulfoxide | Dioxane = 1,4-dioxane |
| DPPA = diphenyl azidophosphate | DMEDA = N,N′- dimethylethylenediamine |
| FA = formic acid | Et = ethyl |
| HATU = N-[(dimethylamino)- 1H-1,2,3-triazolo- | EtOH = ethanol |
| [4,5-b]pyridin-1- ylmethylene]-N- | |
| methylmethanaminium hexafluorophosphate N- | |
| oxide | |
| HPLC = high-performance liquid chromatography | HATU = N-[(Dimethylamino)- 1H-1,2,3-triazolo- |
| | [4,5-b]pyridin-1- ylmethylene]-N methyl- |
| | methanaminium hexafluorophosphate N-oxide |
| LCMS = liquid chromatography-mass spectrometry | LDA = lithium diisopropylamide |
| Me = methyl | n-Bu = n-butyl |
| NMM = N-methylmorpholine | NBS = N-bromosuccinimide |
| NMR = nuclear magnetic resonance | NCS = N-chlorosuccinimide |
| Pd(dppf)Cl₂ = dichloro [1,1′- | NIS = N-iodosuccinimide |
| bis(diphenyl- phosphino)ferrocene]palladium | |
| Ph = phenyl | Pd(dppf)Cl₂-DCM = 1,1′-Bis(diphenylphosphino) |
| | ferrocene palladium dichloride dichloromethane |
| | complex |
| Py = pyridine | Pd(PPh₃)₄ = |
| | tetrakis(triphenyl- phosplune)Palladium(0) |
| TEA = triethylamine | PTSA = p-toluenesulfonic acid |
| Tf = trifluoromethanesulfonic | RT = room temperature |
| TFA = trifluoroacetic acid | T3P = Propylphosphonic Anhydride Solution |
| TFAA = trifluoroacetic anhydride | TBAF = tetrabutylammonium fluoride |
| THF = tetrahydrofuran | TBDPSC1 = tert- butyldiphenylsilyl chloride |
| T₃P =2,4,6-tripropyl-2,4,6-trioxo- | t-Bu = tert-butyl |
| 1,3,5,2,4,6-trioxatriphosphorinane | |
| XPhos = (2-(2,4,6- | TLC = thin layer |
| triisopropylphenethyl)phenyl)dicyclo- hexylphosphine | chromatography |

### Materials and Methods

The progress of reactions was often monitored by TLC or LC-MS. The identity of the products was often confirmed by LC-MS. The LC-MS was recorded using one of the following methods.

Method A: Ascentis Express C18, 50*3.0 mm, 2.7 μm, 4 μL injection, 1.5 mL/min flowrate, 90-900 amu scan range, 254 nm UV detection. Mobile phase A: Water (water/0.05% TFA) and Mobile Phase B (MPB): ACN/0.05% TFA. 5% MPB to 100% in 0.69 min, hold at 100% MPB for 0.5 min, 100% MPB to 5% in 0.05 min, then equilibration to 5% MPB for 0.15 min.

Method B: Poroshell HPH-C18, 50*3.0 mm, 2.7 μm, 4 μL injection, 1.2 mL/min flowrate, 90-900 amu scan range, 254 nm UV detection. Mobile phase A: Water/0.04% NH₃H₂O and Mobile Phase B (MPB): ACN. 10% MPB to 95% in 1.99 min, hold at 95% MPB for 0.6 min, 95% MPB to 10% in 0.2 min, then equilibration to 10% MPB for 0.5 min.

Method C: EVO C18, 50*3.0 mm, 2.6 μm, 4 μL injection, 1.2 mL/min flowrate, 90-900 amu scan range, 254 nm UV detection. Mobile phase A: Water/5 mM NH₄HCO₃ and Mobile Phase B (MPB): ACN. 10% MPB to 95% in 1.99 min, hold at 95% MPB for 0.6 min, 95% MPB to 10% in 0.05 min, then equilibration to 10% MPB for 0.25 min.

Method D: Titank C18, 50*3.0 mm, 3.0 μm, 0.8 μL injection, 1.5 mL/min flowrate, 90-900 amu scan range, 254 nm UV detection. Mobile phase A: Water/5 mM NH₄HCO₃ and Mobile Phase B (MPB): ACN. 50% MPB to 95% in 1.99 min, hold at 95% MPB for 0.6 min, 95% MPB to 10% in 0.05 min, then equilibration to 10% MPB for 0.25 min.

Method E: Poroshell HPH-C18, 50*3.0 mm, 2.7 μm, 4 μL injection, 1.2 mL/min flowrate, 90-900 amu scan range, 254 nm UV detection. Mobile phase A: Water/5 mM NH₄HCO₃ and Mobile Phase B (MPB): ACN. 10% MPB to 95% in 1.99 min, hold at 95% MPB for 0.6 min, 95% MPB to 10% in 0.2 min, then equilibration to 10% MPB for 0.2 min.

Method F: Agilent LC-MS system equipped with DAD and ELSD detector, Water X-Bridge C18, 50*2.1 mm*5 μm or equivalent, 0.6 mL/min or 0.8 mL/min flowrate, 40° C. or 50° C. column temperature, 220 nm UV detection. Mobile phase A: H₂O (0.04% TFA) and mobile phase B: CH₃CN (0.02% TFA). 4.5 min gradient method, actual gradient varies by compound.

Method G: Agilent LC-MS system equipped with DAD and ELSD detector, Waters X-Bridge ShieldRP18, 50*2.1 mm*5 μm or equivalent, 0.6 mL/min or 0.8 mL/min flowrate, 40° C. column temperature, 220 nm UV detection. Mobile phase A: H₂O (0.05% NH₃H₂O) or 10 mM ammonium bicarbonate and mobile phase B: CH₃CN. 4.5 min gradient method, actual gradient varies by compound.

### Additional LCMS Analysis Conditions

Method AA

Instrument: Agilent LCMS system equipped with DAD and ELSD detector

Ion mode: Positive

Column: Waters X-Bridge C18, 50*2.1 mm*5 m or equivalent

Mobile Phase: A: H₂O (0.04% TFA); B: CH₃CN (0.02% TFA)

Gradient: 4.5 min gradient method, actual method would depend on c log P of compound.

Flow Rate: 0.6 mL/min or 0.8 mL/min

Column Temp: 40° C. or 50° C.

UV: 220 nm

Method AB

Instrument: Agilent LCMS system equipped with DAD and ELSD detector

Ion mode: Positive

Column: Waters X-Bridge ShieldRP18, 50*2.1 mm*5 μm or equivalent

Mobile Phase:A: H₂O (0.05% NH₃H₂O) or 10 mM ammonia bicarbonate; B: CH₃CN

Gradient: 4.5 min gradient method; actual method would depend on the c log P of the compound.

Flow Rate: 0.6 mL/min or 0.8 mL/min

Column Temp: 40° C.

UV: 220 nm

LCMS Method BA:_Shim-pack XR-ODS, 50*3 mm, 3.0 μL injection, 1.2 mL/min flowrate, 90-900 amu scan range, 254 nm UV detection. Mobile Phase A (MPA): Water/0.05% TFA and Mobile Phase B (MPB): Acetonitrile/0.05% TFA. Elution 5% MPB to 100% in 2.00 min, hold at 100% MPB for 0.7 min, 100% MPB to 5% in 0.05 min, then equilibration to 5% MPB for 0.25 min.

LCMS Method BB: EVO C18, 50*3 mm, 2.0 μL injection, 1.2 mL/min flowrate, 90-900 amu scan range, 254 nm UV detection. Mobile Phase A (MPA): Water/5 mM NH4HCO3 and Mobile Phase B (MPB): Acetonitrile. Elution 10% MPB to 95% in 2.00 min, hold at 95% MPB for 0.6 min, 95% MPB to 10% in 0.05 min, then equilibration to 10% MPB for 0.25 min.

LCMS Method BC:_XBridge Shield RP18, 50*4.6 mm, 3.0 μL injection, 1.2 mL/min flowrate, 90-900 amu scan range, 254 nm UV detection. Mobile Phase A (MPA): Water/0.04% NH3.H2O and Mobile Phase B (MPB): Acetonitrile. Elution 40% MPB to 70% in 2.80 min, upto 95% in 0.20 min, hold at 95% MPB for 0.5 min, 95% MPB to 10% in 0.05 min, then equilibration to 10% MPB for 0.25 min.

LCMS Method BD:_Titank C18, 50*3 mm, 2.0 μL injection, 1.2 mL/min flowrate, 90-900 amu scan range, 254 nm UV detection. Mobile Phase A (MPA): Water/5 mM NH4HCO3 and Mobile Phase B (MPB): Acetonitrile. Elution 40% MPB to 70% in 2.80 min, upto 95% in 0.20 min, hold at 95% MPB for 0.5 min, 95% MPB to 10% in 0.05 min, then equilibration to 10% MPB for 0.25 min.

LCMS Method BE: XBridge BEH C18, 50*3 mm, 0.7 μL injection, 1.2 mL/min flowrate, 30-2000 amu scan range, 254 nm UV detection. Mobile Phase A (MPA): Water/5mMNH₄CO₃ and Mobile Phase B (MPB): Acetonitrile. Elution 5% MPB to 95% in 1.29 min, hold at 95% MPB for 0.90 min, 95% MPB to 5% in 0.05 min, then equilibration to 5% MPB for 0.25 min.

LCMS Method CA: Kinetex EVO C18 100A, 30*3 mm, 0.5 μL injection, 1.2 mL/min flowrate, 90-900 amu scan range, 254 nm UV detection. Mobile Phase A (MPA): Water/5 mM NH4HCO3 and Mobile Phase B (MPB): Acetonitrile. Elution 10% MPB to 95% in 2.0 min, hold at 95% MPB for 0.3 min, 95% MPB to 10% in 0.1 min.

LCMS Method CB: Xselect CSH C18, 50*3 mm, 1.0 μL injection, 1.2 mL/min flowrate, 90-900 amu scan range, 254 nm UV detection. Mobile Phase A (MPA): Water/0.1% FA and Mobile Phase B (MPB): Acetonitrile/0.1% FA. Elution 5% MPB to 100% in 2.00 min, hold at 100% MPB for 0.7 min, 100% MPB to 5% in 0.05 min, then equilibration to 5% MPB for 0.15 min.

LCMS Method CC: XBridge Shield RP18, 50*4.6 mm, 0.5 μL injection, 1.2 mL/min flowrate, 90-900 amu scan range, 254 nm UV detection. Mobile Phase A (MPA): Water/0.04% NH₄OH and Mobile Phase B (MPB): Acetonitrile. Elution 10% MPB to 95% in 2.00 min, hold at 95% MPB for 0.79 min, 95% MPB to 10% in 0.06 min, then equilibration to 10% MPB for 0.15 min.

LCMS Method CD: Shim-pack XR-ODS, 50*3 mm, 0.3 μL injection, 1.2 mL/min flowrate, 30-2000 amu scan range, 254 nm UV detection. Mobile Phase A (MPA): Water/0.05 TFA and Mobile Phase B (MPB): Acetonitrile/0.05% TFA. Elution 5% MPB to 100% in 1.10 min, hold at 100% MPB for 0.60 min, 100% MPB to 5% in 0.05 min, then equilibration to 5% MPB for 0.25 min.

LCMS Method CE: EVO C18, 50*3 mm, 0.1 μL injection, 1.2 mL/min flowrate, 30-2000 amu scan range, 254 nm UV detection. Mobile Phase A (MPA): Water/5 mM NH4HCO3 and Mobile Phase B (MPB): Acetonitrile. Elution 10% MPB to 95% in 2.00 min, hold at 95% MPB for 0.60 min, 95% MPB to 10% in 0.15 min, then equilibration to 10% MPB for 0.25 min.

LCMS Method CF: kinetex 2.6 um EVO, 50*3 mm, 0.5 μL injection, 1.2 mL/min flowrate, 30-2000 amu scan range, 254 nm UV detection. Mobile Phase A (MPA): Water/5 mM NH4HCO3 and Mobile Phase B (MPB): Acetonitrile. Elution 10% MPB to 95% in 2.00 min, hold at 95% MPB for 0.70 min, 95% MPB to 10% in 0.05 min, then equilibration to 10% MPB for 0.25 min.

LCMS Method CG: Titank C18, 50*3 mm, 0.5 μL injection, 1.5 mL/min flowrate, 30-2000 amu scan range, 254 nm UV detection. Mobile Phase A (MPA): Water/5 mM NH4HCO3 and Mobile Phase B (MPB): Acetonitrile. Elution 10% MPB to 95% in 1.80 min, hold at 95% MPB for 0.80 min, 95% MPB to 10% in 0.15 min, then equilibration to 10% MPB for 0.25 min.

LCMS Method CH: HALOC18, 30*3 mm, 0.5 μL injection, 1.5 mL/min flowrate, 30-2000 amu scan range, 254 nm UV detection. Mobile Phase A (MPA): Water/0.05% TFA and Mobile Phase B (MPB): Acetonitrile/0.05% TFA. Elution 5% MPB to 100% in 1.20 min, hold at 100% MPB for 0.60 min, 100% MPB to 5% in 0.02 min, then equilibration to 5% MPB for 0.18 min.

LCMS Method CI: Poroshell HPH C18, 50*3 mm, 0.5 μL injection, 1.2 mL/min flowrate, 30-2000 amu scan range, 254 nm UV detection. Mobile Phase A (MPA): Water/5 mM NH4HCO3+5 mM NH₄OH and Mobile Phase B (MPB): Acetonitrile. Elution 10% MPB to 95% in 2.00 min, hold at 95% MPB for 0.70 min, 95% MPB to 5% in 0.05 min, then equilibration to 5% MPB for 0.25 min.

LCMS Method CJ: HALOC18, 30*3 mm, 0.5 μL injection, 1.5 mL/min flowrate, 30-2000 amu scan range, 254 nm UV detection. Mobile Phase A (MPA): Water/0.1% FA and Mobile Phase B (MPB): Acetonitrile/0.1% FA. Elution 5% MPB to 100% in 1.20 min, hold at 100% MPB for 0.60 min, 100% MPB to 5% in 0.02 min, then equilibration to 5% MPB for 0.18 min.

LCMS Method DA: HALOC18, 30*3 mm, 0.5 μL injection, 1.5 mL/min flowrate, 30-2000 amu scan range, 254 nm UV detection. Mobile Phase A (MPA): Water/0.05% TFA and Mobile Phase B (MPB): Acetonitrile/0.05% TFA. Elution 5% MPB to 100% in 1.20 min, hold at 100% MPB for 0.60 min, 100% MPB to 5% in 0.02 min, then equilibration to 5% MPB for 0.18 min.

LCMS Method DB: Shim-pack XR-ODS, 50*3 mm, 0.3 μL injection, 1.2 mL/min flowrate, 30-2000 amu scan range, 254 nm UV detection. Mobile Phase A (MPA): Water/0.05 TFA and Mobile Phase B (MPB): Acetonitrile/0.05% TFA. Elution 5% MPB to 100% in 1.10 min, hold at 100% MPB for 0.60 min, 100% MPB to 5% in 0.05 min, then equilibration to 5% MPB for 0.25 min.

LCMS Method DC: EVO C18, 50*3 mm, 0.1 μL injection, 1.2 mL/min flowrate, 30-2000 amu scan range, 254 nm UV detection. Mobile Phase A (MPA): Water/5 mM NH4HCO3 and Mobile Phase B (MPB): Acetonitrile. Elution 10% MPB to 95% in 2.00 min, hold at 95% MPB for 0.60 min, 95% MPB to 10% in 0.15 min, then equilibration to 10% MPB for 0.25 min.

LCMS Method DD: Titank C18, 50*3 mm, 0.5 μL injection, 1.5 mL/min flowrate, 30-2000 amu scan range, 254 nm UV detection. Mobile Phase A (MPA): Water/5 mM NH4HCO3 and Mobile Phase B (MPB): Acetonitrile. Elution 10% MPB to 95% in 1.80 min, hold at 95% MPB for 0.80 min, 95% MPB to 10% in 0.15 min, then equilibration to 10% MPB for 0.25 min.

LCMS Method DE: Kinetex 2.6 um EVO C18 100A, 50*3 mm, 0.6 μL injection, 1.2 mL/min flowrate, 30-2000 amu scan range, 254 nm UV detection. Mobile Phase A (MPA): Water/5 mM NH4HCO3 and Mobile Phase B (MPB): Acetonitrile. Elution 10% MPB to 95% in 1.20 min, hold at 95% MPB for 0.50 min, 95% MPB to 10% in 0.05 min, then equilibration to 10% MPB for 0.10 min.

LCMS Method DF: Shim-pack Scepter C18, 30*3 mm, 0.5 μL injection, 1.5 mL/min flowrate, 30-2000 amu scan range, 254 nm UV detection. Mobile Phase A (MPA): Water/0.04% NH₄OH and Mobile Phase B (MPB): Acetonitrile. Elution 10% MPB to 95% in 2.00 min, hold at 95% MPB for 0.60 min, 95% MPB to 10% in 0.20 min, then equilibration to 10% MPB for 0.20 min.

Prep-HPLC was carried out using the following methods.

Method H: Prep-HPLC: Column: Xselect CSH OBD Column 30*150 mm, 5 μm; Mobile Phase: Water (w/0.1% FA) and ACN, UV detection 254/210 nm.

Method I: Prep-HPLC: Column: XBridge Prep C18 OBD Column 19*150 mm, 5 μm; Mobile Phase: Water (10 mM NH₄HCO₃+0.1% NH₃.H₂O) and ACN, UV detection 254/210 nm.

Method J: GILSON 281 and Shimadzu LC-MS 2010A, GILSON 215 and Shimadzu LC-20AP, or GILSON 215. Xtimate C18 150*25 mm*5 μm, 25 mL/min or 30 mL/min flowrate, 220 nm and 254 nm UV detection or MS trigger. Mobile phase A: NH₄OH/H₂O=0.05% v/v and mobile phase B: MeCN or mobile phase A: FA/H₂O=0.225% v/v and mobile phase B: MeCN. Gradient varies by compound.

Prep. HPLC Condition (Method AA)

### Instrument:

1. GILSON 281 and Shimadzu LCMS 2010A

2. GILSON 215 and Shimadzu LC-20AP

3. GILSON 215

Mobile Phase:

A: NH₄OH/H₂O=0.05% v/v; B: ACN

A: FA/H₂O=0.225% v/v; B: ACN

Column

Xtimate C18 150*25 mm*5 μm

Flow rate: 25 mL/min or 30 mL/min

Monitor wavelength: 220&254 nm

Gradient: actual method would depend on clog P of compound

Detector: MS Trigger or UV

NMR was recorded on BRUKER NMR 300.03 Mz, DUL-C-H, ULTRASHIELD™ 300, AVANCE II 300 B-ACS™ 120 or BRUKER NMR 400.13 Mz, BBFO, ULTRASHIELD™ 400, AVANCE III 400, B-ACS™ 120.

### EXAMPLES

### Intermediates

### Intermediate 1: 5,6-difluoro-1H-indol-3-amine

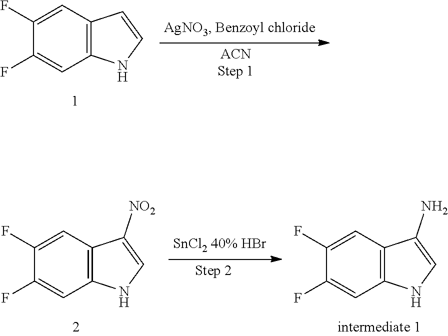 [SMILES: Fc1cc2cc[nH]c2cc1F ; O=[N+]([O-])c1c[nH]c2cc(F)c(F)cc12 ; Nc1c[nH]c2cc(F)c(F)cc12]

Step 1—Synthesis of 5,6-difluoro-3-nitrol-1H-indole: 5,6-Difluoro-1H-indole (5.0 g, 32.7 mmol, 1.0 equiv) was dissolved in CH₃CN (50.0 mL), and AgNO₃ (6.1 g, 36.0 mmol, 1.1 equiv) was added in portions. The resulting solution was then cooled to 0° C., and after 5 minutes, benzoyl chloride (4.1 mL, 36.0 mmol, 1.1 equiv) was added. The resulting solution was allowed to warm to RT for 2 h, and then the pH of the reaction mixture was adjusted to pH 8 by dropwise addition of 1 M aqueous Na₂CO₃ solution. The mixture was extracted with EtOAc (150 mL×3) and the organic layers were combined and concentrated in vacuo. The residue was purified by flash column chromatography on silica gel, eluting with ethyl acetate/petroleum ether (5/95) to give 5,6-difluoro-3-nitro-1H-indole (3.5 g, 17.7 mmol) as a yellow solid. LC-MS Method B, MS-ESI: 199.1 [M+H⁺]. Alternatively, the residue can be purified by flash silica gel chromatography (ISCO®; 24 g SepaFlash® Silica Flash Column, Eluent of 0-100% EtOAc/Petroleum ether gradient @ 30 mL/min) to give 5,6-difluoro-3-nitro-1H-indole (2.9 g, 13.5 mmol) as a yellow solid. MS-ESI, 199.1 [M+H⁺].

Step 2—Synthesis of 5,6-difluoro-1H-indol-3-amine (Intermediate 1): 5,6-Difluoro-3-nitro-1H-indole (3.5 g, 17.7 mmol, 1.0 equiv) was dissolved in 40% HBr/H₂O (40 mL), then SnCl₂ (16.8 g, 88.5 mmol, 5.0 equiv) was added and the reaction mixture was heated to 70° C. for 30 minutes. The reaction mixture was cooled to RT, and the pH was adjusted to pH 8 by dropwise addition of 1 M aqueous NaOH. The mixture was extracted with DCM (150 mL×5) and the combined organic layers were concentrated in vacuo. The residue was used in the next step directly without further purification. LCMS Method B, MS-ESI: 169.1 [M+H⁺].

### Intermediate 2: [[4-(trifluoromethyl)phenyl]carbamoyl]formic acid

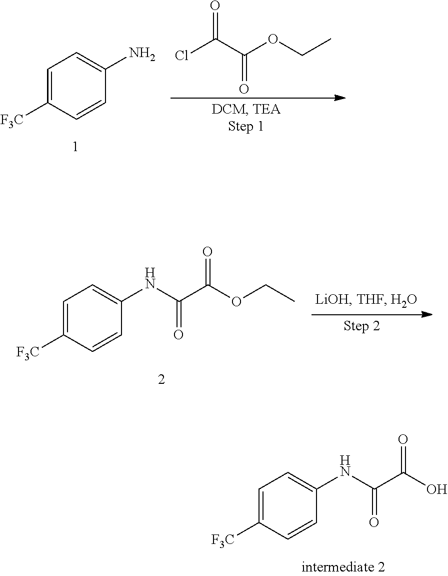

Step 1—Synthesis of ethyl [[4-(trifluoromethyl)phenyl]carbamoyl]formate: p-Trifluoro-methylaniline (500.0 mg, 3.1 mmol, 1.0 equiv) was dissolved in DCM (10.0 mL), then TEA (1.3 mL, 9.3 mmol, 3.0 equiv) and ethyl chloroglyoxylate (508.1 mg, 3.7 mmol, 1.2 equiv) were added. The reaxction mixture was stirred overnight at RT, then concentrated in vacuo. The residue was purified via flash column chromatography on silica gel, eluting with ethyl acetate/petroleum ether (1:1) to give Ethyl [[4-(trifluoromethyl)phenyl]-carbamoyl]formate (600.0 mg, 2.3 mmol) as a yellow oil. LCMS Method A, MS-ESI: 262.2 [M+H⁺].

Step 2—Synthesis of [[4-(trifluoromethyl)phenyl]carbamoyl]formic acid (Intermediate 2): Ethyl [[4-(trifluoro-methyl)phenyl]carbamoyl]formate (600.0 mg, 2.3 mmol, 1.0 equiv) was added to a mixture of THF (15.0 mL) and H₂O (5.0 mL), then LiOH (275.1 mg, 11.5 mmol, 5.0 equiv) was added and the reaction mixture was stirred for 2 h at RT. The reaction mixture was adjusted to pH 5 with concentrated HCl. The aqueous layer was extracted with EtOAc (100 mL×3). The combined organic layers were dried over anhydrous sodium sulfate and concentrated in vacuo to give [[4-(trifluoromethyl)phenyl]carbamoyl]formic acid (500.0 mg, 2.2 mmol) as a yellow oil. LCMS: Method A, MS-ESI: 232.1 [M−H⁻].

### Intermediate 3: [[3-(trifluoromethyl)phenyl]carbamoyl]formic acid

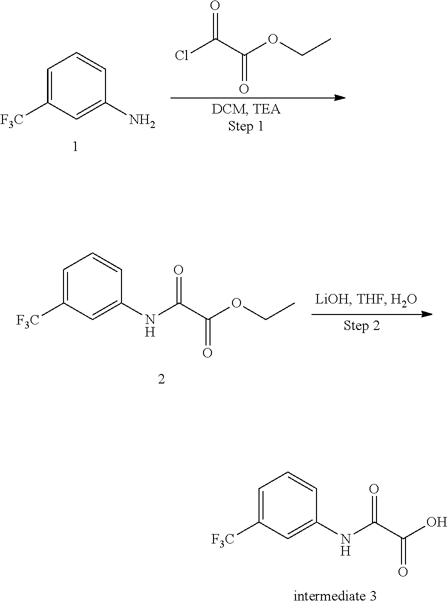

Step 1—Synthesis of ethyl [[3-(trifluoromethyl)phenyl]carbamoyl]formate: The title compound was prepared using the same methods described in Intermediate 2, Step 1. LC-MS: Method A, MS-ESI: 262.1 [M+H⁺].

Step 2—Synthesis of [[3-(trifluoromethyl)phenyl]carbamoyl] formic acid (Intermediate 3): The title compound was prepared using the same methods described in Intermediate 2, Step 2. LC-MS: Method A, MS-ESI: 232.1 [M−H⁻].

### Intermediate 4: [(4-ethylcyclohexyl)carbamoyl]formic acid

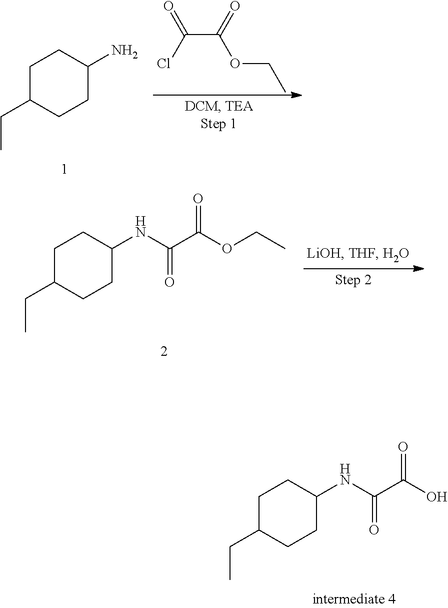

Step 1—Synthesis of ethyl [(4-ethylcyclohexyl)carbamoyl]formate: The title compound was prepared using the same methods described in Intermediate 2, Step 1. LC-MS: Method A, MS-ESI: 228.1 [M+H⁺].

Step 2—Synthesis of [(4-ethylcyclohexyl)carbamoyl]formic acid (Intermediate 4): The title compound was prepared using the same methods described in Intermediate 2, Step 2. LC-MS: Method B, MS-ESI: 198.1 [M−H⁻].

### Intermediate 5: [Methyl[4-(trifluoromethyl)phenyl]carbamoyl]formic acid (Intermediate 5)

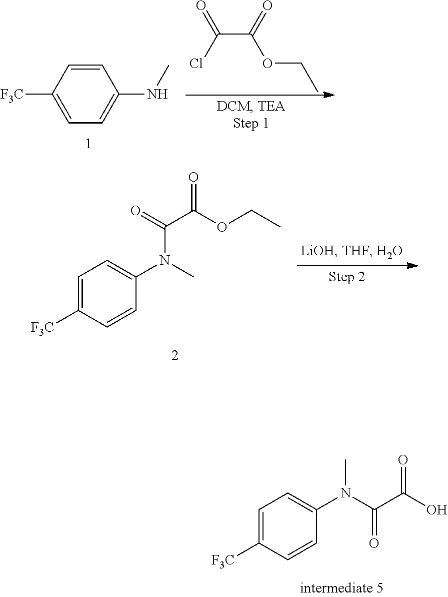

Step 1—Synthesis of ethyl [methyl[4-(trifluoromethyl)phenyl]carbamoyl]formate: The title compound was prepared using the same methods described in Intermediate 2, Step 1. LC-MS: Method B, MS-ESI: 276.1 [M+H⁺].

Step 2—Synthesis of [methyl[4-(trifluoromethyl)phenyl]carbamoyl]formate (Intermediate 5): The title compound was prepared using the same methods described in Intermediate 2, Step 2. LC-MS: Method B, MS-ESI: 246.1 [M−H⁻].

### Intermediate 6: (4-ethylpiperidin-1-yl)(oxo)acetic acid

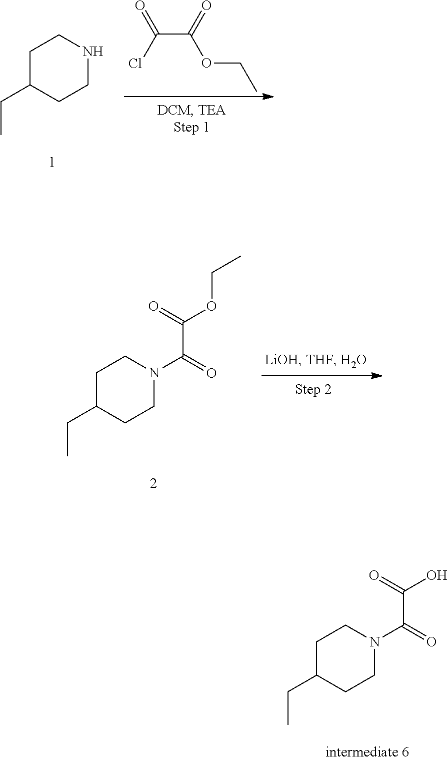

Step 1—Synthesis of ethyl 2-(4-ethylpiperidin-1-yl)-2-oxoacetate: The title compound was prepared using the same methods described in Intermediate 2, Step 1. LC-MS: Method B, MS-ESI: 214.1 [M+H⁺].

Step 2—Synthesis of (4-ethylpiperidin-1-y)(oxo)acetic acid (Intermediate 6): The title compound was prepared using the same methods described in Intermediate 2, Step 2. LC-MS: Method B, MS-ESI: 184.1 [M−H⁻].

### Intermediate 8: 6-cyclohexylpyridin-3-amine

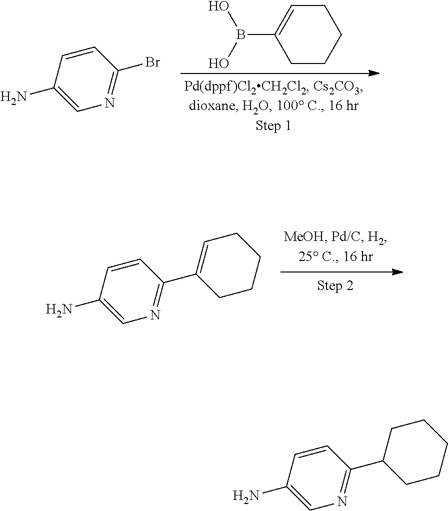 [SMILES: Nc1ccc(Br)nc1 ; OB(O)C1=CCCCC1 ; Nc1ccc(C2=CCCCC2)nc1 ; Nc1ccc(C2CCCCC2)nc1]

Step 1—Synthesis of 6-(cyclohexyl-1-en-1-yl)pyridin-3-amine: To a mixture of 6-bromopyridin-3-amine (10.0 g, 57.8 mmol, 1.0 equiv.) and cyclohexen-1-ylboronic acid (8.7 g, 69.4 mmol, 1.2 equiv.) in dioxane (200 mL) and H₂O (58 mL) was added Cs₂CO₃ (37.7 g, 115.6 mmol, 2.0 equiv.) and Pd(dppf)Cl₂DCM (2.4 g, 2.9 mmol, 0.05 equiv.) under an atmosphere of N₂. The mixture was stirred at 100° C. for 16 hours. The reaction mixture was concentrated in vacuo and the resulting residue was purified by flash silica gel chromatography (ISCO®; 120 g SepaFlash® Silica Flash Column, Eluent of 0˜50% EtOAc/Petroleum ether gradient) to give 6-(cyclohex-1-en-1-yl)pyridin-3-amine (9.0 g, 51.7 mmol) as a brown oil. MS-ESI, 175.2 [M+H⁺].

Step 2—Synthesis of 6-cyclohexylpyridin-3-amine (Intermediate 8): To a mixture of 6-(cyclohexen-1-yl)pyridin-3-amine (3.0 g, 17.2 mmol, 1.0 equiv.) in MeOH (40 mL) under an atmosphere of N₂ was added Pd/C (3.0 g, 10% wt/wt %, 0.28 mmol, 0.02 equiv.) in one portion. The suspension evacuated under vacuum and backfilled with H₂(g) 3 times. The mixture was stirred at 25° C. for 16 hours under an atmosphere of hydrogen (balloon). The reaction mixture was filtered through a pad of Celite and concentrated in vacuo to give 6-cyclohexylpyridin-3-amine (2.1 g, 11.9 mmol) as a white solid. MS-ESI, 176.8 [M+H⁺].

### Intermediate 9: 2-((3-methyl-5-(trifluoromethyl)phenyl)amino)-2-oxoacetic acid

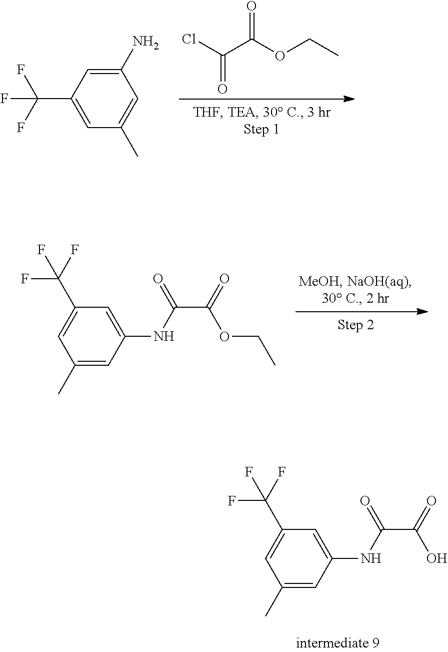

Step 1—Synthesis of ethyl 2-((3-methyl-5-(trifluoromethyl)phenyl)amino)-2-oxoacetate: To a mixture of 3-methyl-5-(trifluoromethyl)aniline (2.0 g, 11.4 mmol, 1.0 equiv.) in THF (80 mL) was added a solution of ethyl 2-chloro-2-oxoacetate (1.7 g, 12.6 mmol, 1.1 equiv.) in THF (20 mL) over 5 minutes at 0° C. Then TEA (4.0 mL, 28.6 mmol, 2.5 equiv.) was added to the reaction mixture. The mixture was stirred at 30° C. for 3 hours. The reaction mixture was filtered and concentrated under reduced pressure to give ethyl 2-((3-methyl-5-(trifluoromethyl)phenyl)amino)-2-oxoacetate (2.7 g, 9.7 mmol) as a yellow oil that was used without additional purification.

Step 2—Synthesis of 2-((3-methyl-5-(trifluoromethyl)phenyl)amino)-2-oxoacetic acid (Intermediate 9): To a mixture of ethyl 2-((3-methyl-5-(trifluoromethyl)phenyl)amino)-2-oxoacetate (2.7 g, 9.7 mmol 1 equiv.) in MeOH (80 mL) was added aqueous NaOH (2 M, 7.3 mL, 14.6 mmol, 1.5 equiv.). The mixture was stirred at 30° C. for 2 hours. The reaction mixture concentrated under reduced pressure to give a residue. Then H₂O (30 mL) was added and the mixture was adjusted to pH 4 by the dropwise addition of 2 M HCl. The resulting solid was collected by filtration and washed with water to give 2-((3-methyl-5-(trifluoromethyl)phenyl)amino)-2-oxoacetic acid (2.0 g, 8.1 mmol) as a white solid. MS-ESI, 248.1 [M+H⁺].

TABLE E1
The intermediates in Table E1 were prepared using the procedure described in
Intermediate 9, starting from the appropriate amine.

| | | | LC-MS |
|---|---|---|---|
| Inter- | | | (ESI, |
| mediate | Structure | IUPAC Name | [M + H⁺] |
| 10 | 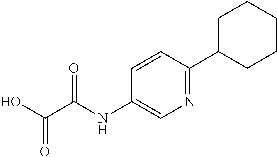 [SMILES: O=C(O)C(=O)Nc1ccc(C2CCCCC2)nc1] | 2-((6-cyclohexylpyridin- 3-yl)amino)-2-oxoacetic acid | 249.2 |
| 11 | 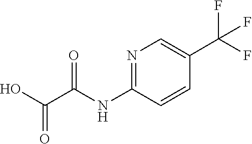 [SMILES: O=C(O)C(=O)Nc1ccc(C(F)(F)F)cn1] | 2-oxo-2-((5- (trifluoromethyl)pyridin- 2-yl)amino)acetic acid | 235.1 |
| 12 | 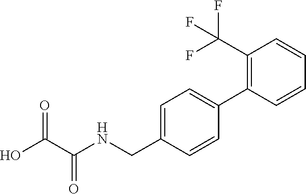 [SMILES: O=C(O)C(=O)NCc1ccc(-c2ccccc2C(F)(F)F)cc1] | 2-oxo-2-(((2′- (trifluoromethyl)-[1,1′- biphenyl]-4- yl)methyl)amino)acetic acid | 324.1 |
| 13 | 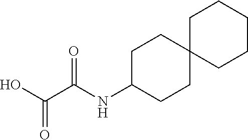 [SMILES: O=C(O)C(=O)NC1CCC2(CCCCC2)CC1] | 2-oxo-2- (spiro[5.5]undecan-3- ylamino)acetic acid | 240.2 |
| 14 | 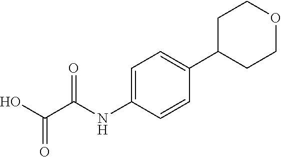 [SMILES: O=C(O)C(=O)Nc1ccc(C2CCOCC2)cc1] | 2-oxo-2-((4-(tetrahydro- 2H-pyran-4- yl)phenyl)amino)acetic acid | 250.2 |
| 15 | 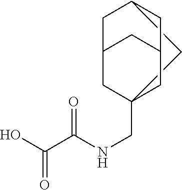 [SMILES: O=C(O)C(=O)NCC12CC3CC(CC(C3)C1)C2] | 2-((adamantan-1- ylmethyl)amino)-2- oxoacetic acid | 238.2 |
| 16 | 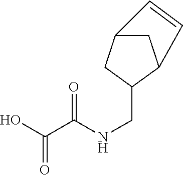 [SMILES: O=C(O)C(=O)NCC1CC2C=CC1C2] | 2-((bicyclo[2.2.1]hept-5- en-2-ylmethyl)amino)-2- oxoacetic acid | 196.2 |
| 17 | 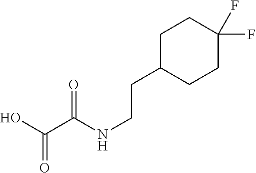 [SMILES: O=C(O)C(=O)NCCC1CCC(F)(F)CC1] | 2-((2-(4,4- difluorocyclohexyl)ethyl) amino)-2-oxoacetic acid | 236.2 |
| 18 | 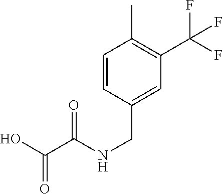 [SMILES: Cc1ccc(CNC(=O)C(=O)O)cc1C(F)(F)F] | 2-((4-methyl-3- (trifluoromethyl)benzyl) amino)-2-oxoacetic acid | 262.1 |
| 19 | 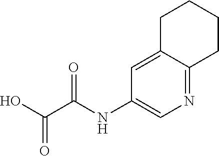 [SMILES: O=C(O)C(=O)Nc1cnc2c(c1)CCCC2] | 2-oxo-2-((5,6,7,8- tetrahydroquinolin-3- yl)amino)acetic acid | 221.2 |
| 20 | 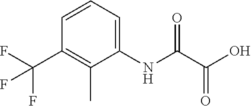 [SMILES: Cc1c(NC(=O)C(=O)O)cccc1C(F)(F)F] | 2-((2-methyl-3- (trifluoromethyl)phenyl) amino)-2-oxoacetic acid | 248.1 |
| 21 | 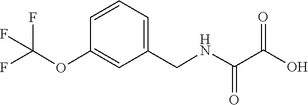 [SMILES: O=C(O)C(=O)NCc1cccc(OC(F)(F)F)c1] | 2-oxo-2-((3- (trifluoromethoxy)benzyl) amino)acetic acid | |
| 22 | 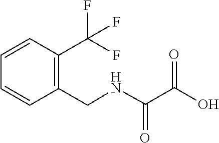 [SMILES: O=C(O)C(=O)NCc1ccccc1C(F)(F)F] | 2-oxo-2-((2- (trifluoromethyl)benzyl) amino)acetic acid | |
| 23 | 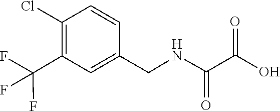 [SMILES: O=C(O)C(=O)NCc1ccc(Cl)c(C(F)(F)F)c1] | 2-((4-chloro-3- (trifluoromethyl)benzyl) amino)-2-oxoacetic acid | |
| 24 | 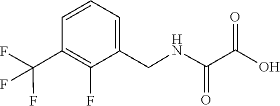 [SMILES: O=C(O)C(=O)NCc1cccc(C(F)(F)F)c1F] | 2-((2-fluoro-3- (trifluoromethyl)benzyl) amino)-2-oxoacetic acid | |
| 25 | 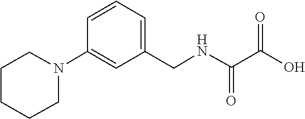 [SMILES: O=C(O)C(=O)NCc1cccc(N2CCCCC2)c1] | 2-oxo-2-((3-(piperidin-1- yl)benzyl)amino)acetic acid | |
| 26 | 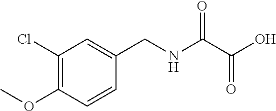 [SMILES: COc1ccc(CNC(=O)C(=O)O)cc1Cl] | 2-((3-chloro-4- methoxybenzyl)amino)-2- oxoacetic acid | |
| 27 | 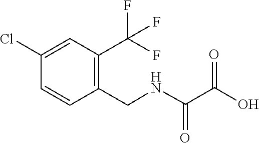 [SMILES: O=C(O)C(=O)NCc1ccc(Cl)cc1C(F)(F)F] | 2-((4-chloro-2- (trifluoromethyl)benzyl) amino)-2-oxoacetic acid | |
| 28 | 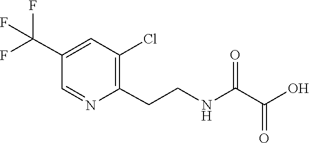 [SMILES: O=C(O)C(=O)NCCc1ncc(C(F)(F)F)cc1Cl] | 2-((2-(3-chloro-5- (trifluoromethyl)pyridin-2- yl)ethyl)amino)-2- oxoacetic acid | |
| 29 | 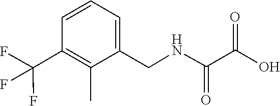 [SMILES: Cc1c(CNC(=O)C(=O)O)cccc1C(F)(F)F] | 2-((2-methyl-3- (trifluoromethyl)benzyl) amino)-2-oxoacetic acid | |
| 30 | 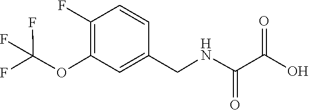 [SMILES: O=C(O)C(=O)NCc1ccc(F)c(OC(F)(F)F)c1] | 2-((4-fluoro-3- (trifluoromethoxy)benzyl) amino)-2-oxoacetic acid | |
| 31 | 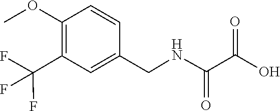 [SMILES: COc1ccc(CNC(=O)C(=O)O)cc1C(F)(F)F] | 2-((4-methoxy-3- (trifluoromethyl)benzyl) amino)-2-oxoacetic acid | |
| 32 | 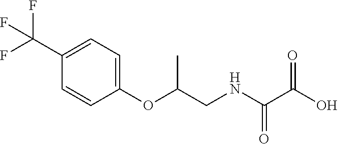 [SMILES: CC(CNC(=O)C(=O)O)Oc1ccc(C(F)(F)F)cc1] | 2-oxo-2-((2-(4- (trifluoromethyl)phenoxy) propyl)amino)acetic acid | |
| 33 | 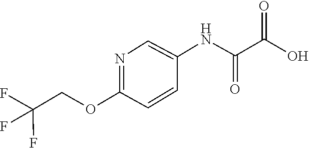 [SMILES: O=C(O)C(=O)Nc1ccc(OCC(F)(F)F)nc1] | 2-oxo-2-((6-(2,2,2- trifluoroethoxy)pyridin-3- yl)amino)acetic acid | |
| 34 | 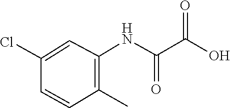 | 2-((5-chloro-2- methylphenyl)amino)-2- oxoacetic acid | |
| 35 | 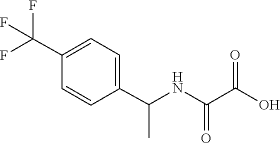 [SMILES: CC(NC(=O)C(=O)O)c1ccc(C(F)(F)F)cc1] | 2-oxo-2-((1-(4- (trifluoromethyl)phenyl) ethyl)amino)acetic acid | |
| 36 | 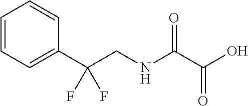 [SMILES: O=C(O)C(=O)NCC(F)(F)c1ccccc1] | 2-((2,2-difluoro-2- phenylethyl)amino)-2- oxoacetic acid | |
| 37 | 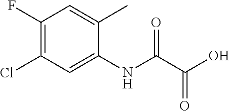 | 2-((5-chloro-4-fluoro-2- methylphenyl)amino)-2- oxoacetic acid | |
| 38 | 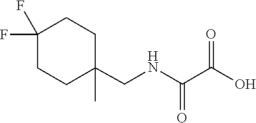 [SMILES: CC1(CNC(=O)C(=O)O)CCC(F)(F)CC1] | 2-(((4,4-difluoro-1- methylcyclohexyl)methyl) amino)-2-oxoacetic acid | |
| 39 | 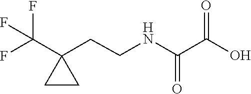 [SMILES: O=C(O)C(=O)NCCC1(C(F)(F)F)CC1] | 2-oxo-2-((2-(1- (trifluoromethyl)cyclopropyl) ethyl)amino)acetic acid | |
| 40 | 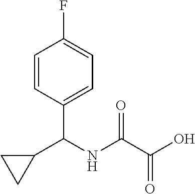 [SMILES: O=C(O)C(=O)NC(c1ccc(F)cc1)C1CC1] | 2-((cyclopropyl(4- fluorophenyl)methyl) amino)-2-oxoacetic acid | |
| 41 | 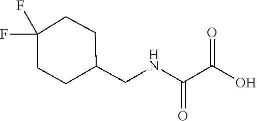 [SMILES: O=C(O)C(=O)NCC1CCC(F)(F)CC1] | 2-(((4,4- difluorocyclohexyl)methyl) amino)-2-oxoacetic acid | |
| 42 | 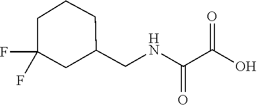 [SMILES: O=C(O)C(=O)NCC1CCCC(F)(F)C1] | 2-(((3,3- difluorocyclohexyl)methyl) amino)-2-oxoacetic acid | |
| 43 | 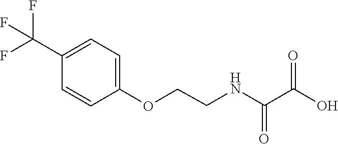 [SMILES: O=C(O)C(=O)NCCOc1ccc(C(F)(F)F)cc1] | 2-oxo-2-((2-(4- (trifluoromethyl)phenoxy) ethyl)amino)acetic acid | |
| 44 | 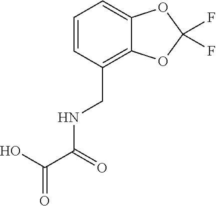 [SMILES: O=C(O)C(=O)NCc1cccc2c1OC(F)(F)O2] | 2-(((2,2- difluorobenzo[d][1,3] dioxol-4-yl)methyl)amino)- 2-oxoacetic acid | |
| 45 | 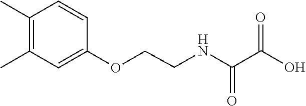 [SMILES: Cc1ccc(OCCNC(=O)C(=O)O)cc1C] | 2-((2-(3,4- dimethylphenoxy)ethyl) amino)-2-oxoacetic acid | |
| 46 | 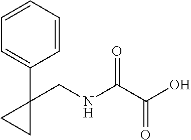 [SMILES: O=C(O)C(=O)NCC1(c2ccccc2)CC1] | 2-oxo-2-(((1- phenylcyclopropyl)methyl) amino)acetic acid | |
| 47 | 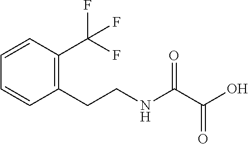 [SMILES: O=C(O)C(=O)NCCc1ccccc1C(F)(F)F] | 2-oxo-2-((2- (trifluoromethyl)phenethyl) amino)acetic acid | |
| 48 | 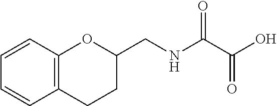 [SMILES: O=C(O)C(=O)NCC1CCc2ccccc2O1] | 2-((chroman-2- ylmethyl)amino)-2- oxoacetic acid | |
| 49 | 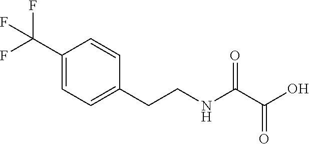 [SMILES: O=C(O)C(=O)NCCc1ccc(C(F)(F)F)cc1] | 2-oxo-2-((4- (trifluoromethyl)phenethyl) amino)acetic acid | |
| 50 | 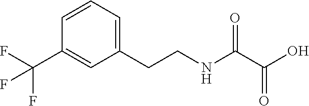 [SMILES: O=C(O)C(=O)NCCc1cccc(C(F)(F)F)c1] | 2-oxo-2-((3- (trifluoromethyl)phenethyl) amino)acetic acid | |

### Synthesis of intermediate BI (5,6-difluoro-1H-indol-3-amine hydrochloride)

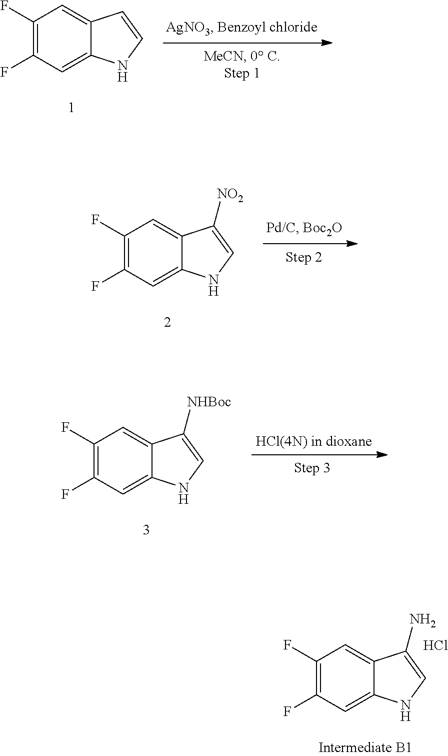 [SMILES: Fc1cc2cc[nH]c2cc1F ; O=[N+]([O-])c1c[nH]c2cc(F)c(F)cc12 ; CC(C)(C)OC(=O)Nc1c[nH]c2cc(F)c(F)cc12 ; Nc1c[nH]c2cc(F)c(F)cc12 ; Cl]

### Step 1: 5,6-difluoro-3-nitro-1H-indole

5,6-Difluoro-1H-indole (25.0 g, 163.3 mmol, 1.0 equiv.) was dissolved in in ACN (300 mL) and cooled to 0° C., then AgNO₃ (33.3 g, 195.9 mmol, 1.2 equiv.) was added. The resulting mixture was stirred for 15 min at 0° C., then benzoyl chloride (27.5 g, 195.9 mmol, 1.2 equiv.) was added batchwise, maintaining the reaction mixture at 0° C. After an additional 3 hours at 0° C. the reaction mixture was quenched by the addition of ice-water. The reaction mixture was adjusted to pH 8 with saturated aqueous NaHCO₃, extracted with DCM, and the combined organic layers were concentrated under vacuum. The residue was purified by flash column chromatography on silica gel, eluting with ethyl acetate/petroleum ether (2:1) to give 5,6-difluoro-3-nitro-1H-indole (24.2 g) as a brown solid. LCMS Method CA: [M+H]⁺=199.

### Step 2: tert-butyl N-(5,6-difluoro-1H-indol-3-yl)carbamate

5,6-Difluoro-3-nitro-1H-indole (24.0 g, 121.1 mmol, 1.0 equiv.) was dissolved in MeOH (300 mL), then Pd/C (10% wt., 2.4 g) and (Boc)₂O (39.7 g, 181.7 mmol, 1.5 equiv.) were added under nitrogen. The mixture was sparged with nitrogen, placed under an atmosphere of hydrogen gas (balloon), then stirred overnight at ambient temperature. The solids were removed by filtration and the filtrate was concentrated under vacuum. The residue was purified by flash column chromatography on silica gel, eluting with ethyl acetate/petroleum ether (1:4) to give tert-butyl N-(5,6-difluoro-1H-indol-3-yl)carbamate (22.4 g) as a yellow solid. LCMS Method CD: [M+H]⁺=269.

### Step 3: 5,6-difluoro-1H-indol-3-amine hydrochloride

tert-Butyl N-(5,6-difluoro-H-indol-3-yl)carbamate (17.0 g, 63.4 mmol, 1.0 equiv.) was dissolved in HCl/1,4-dioxane (4N, 200 mL). The resulting mixture was stirred for 30 min at ambient temperature and then concentrated under vacuum to give 5,6-difluoro-1H-indol-3-amine hydrochloride (12.3 g) as a yellow solid that was used without any additional purification. LCMS Method CD: [M+H]⁺=169.

The following intermediates were prepared using the method described for Intermediate B1.

| Intermediate | Starting Material | Structure | LCMS |
|---|---|---|---|
| Intermediate B2 | 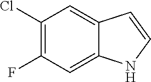 [SMILES: Fc1cc2[nH]ccc2cc1Cl] | 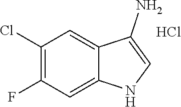 [SMILES: Nc1c[nH]c2cc(F)c(Cl)cc12 ; Cl] | Method CC: MS-ESI: 185 [M + H]⁺ |
| Intermediate B3 | 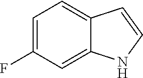 | 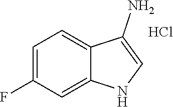 [SMILES: Nc1c[nH]c2cc(F)ccc12 ; Cl] | Method CC: MS-ESI: 151 [M + H]⁺ |
| Intermediate B4 | 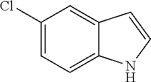 | 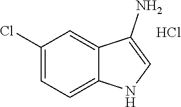 [SMILES: Nc1c[nH]c2ccc(Cl)cc12 ; Cl] | Method CA: MS-ESI: 167 [M + H]⁺ |
| Intermediate B5 | 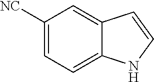 [SMILES: N#Cc1ccc2[nH]ccc2c1] | 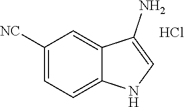 [SMILES: N#Cc1ccc2[nH]cc(N)c2c1 ; Cl] | Method CA: MS-ESI: 158 [M + H]⁺ |

### Synthesis of Intermediate B6 (3-(4-(trifluoromethyl)phenoxy)piperidine)

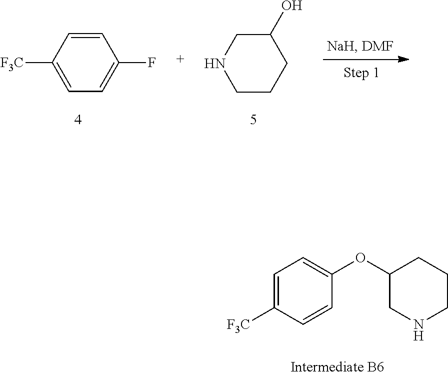 [SMILES: Fc1ccc(C(F)(F)F)cc1 ; OC1CCCNC1 ; FC(F)(F)c1ccc(OC2CCCNC2)cc1]

Piperidin-3-ol (369.8 mg, 3.6 mmol, 1.0 equiv.) was dissolved in DMF (10 mL) and cooled to 0° C., then NaH (60% wt in mineral oil, 292.0 mg, 7.3 mmol, 2.0 equiv.) was added at 0° C. After 20 min at 0° C., 1-fluoro-4-(trifluoromethyl)benzene (600.0 mg, 3.6 mmol, 1.0 equiv.) was added, maintaining the solution at 0° C. The reaction mixture was heated to 60° C. for 3 hours, then cooled to ambient temperature and quenched by the addition of ice water. The resulting solution was extracted with ethyl acetate, dried over anhydrous Na₂SO₄ and concentrated under vacuum. The residue was purified by flash column chromatography on silica gel, eluting with ethyl acetate/petroleum ether (1:1) to give 3-[4-(trifluoromethyl)phenoxy]piperidine (390.0 mg) as a brown oil. LCMS Method CC: [M+H]⁺=246.

The following intermediates were prepared using the method described for Intermediate B6.

| Intermediate | Starting Material A | Starting Material B | Structure | LCMS |
|---|---|---|---|---|
| Intermediate B7 | 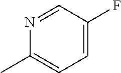 | 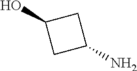 | 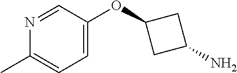 | Method CA: MS-ESI: 179 [M + H]⁺ |
| Intermediate B8 | 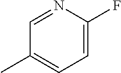 | 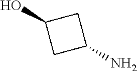 | 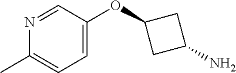 | Method CA: MS-ESI: 179 [M + H]⁺ |
| Intermediate B9 | 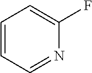 | 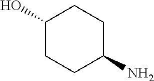 | 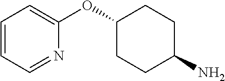 [SMILES: N[C@H]1CC[C@H](Oc2ccccn2)CC1] | Method CC: MS-ESI: 193 [M + H]⁺ |
| Intermediate B10 | 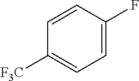 [SMILES: Fc1ccc(C(F)(F)F)cc1] | 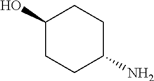 | 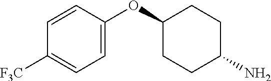 [SMILES: N[C@H]1CC[C@H](Oc2ccc(C(F)(F)F)cc2)CC1] | Method CC: MS-ESI: 260 [M + H]⁺ |
| Intermediate B11 | 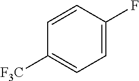 [SMILES: Fc1ccc(C(F)(F)F)cc1] | 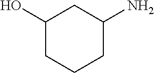 | 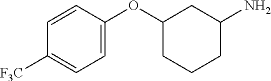 [SMILES: NC1CCCC(Oc2ccc(C(F)(F)F)cc2)C1] | Method CC: MS-ESI: 260 [M + H]⁺ |

### Synthesis of intermediate B12 (cis-3-((6-(trifluoromethyl)pyridin-3-yl)oxy)cyclobutan-1-amine hydrochloride)

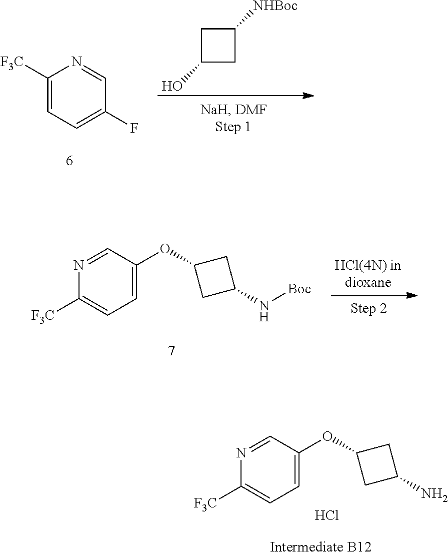 [SMILES: Fc1ccc(C(F)(F)F)nc1 ; CC(C)(C)OC(=O)N[C@H]1C[C@@H](O)C1 ; CC(C)(C)OC(=O)N[C@H]1C[C@@H](Oc2ccc(C(F)(F)F)nc2)C1 ; N[C@H]1C[C@@H](Oc2ccc(C(F)(F)F)nc2)C1 ; Cl]

### Step 1: tert-butyl N-[cis-3-[[6-(trifluoromethyl)pyridin-3-yl]oxy]cyclobutyl]carbamate

tert-Butyl N-[cis-3-hydroxycyclobutyl]carbamate (2.2 g, 12.1 mmol, 2.0 equiv) was dissolved in DMF (10 mL) and cooled to 0° C., then NaH (60% wt in mineral oil, 363.4 mg, 15.1 mmol, 2.5 equiv.) was added in portions, maintaining the solution at 0° C. After 30 min at 0° C., 5-fluoro-2-(trifluoromethyl)pyridine (1.0 g, 6.1 mmol, 1.0 equiv.) was added. The reaction mixture was stirred for 1 hour at ambient temperature and then quenched by the addition of water. The resulting solution was extracted with ethyl acetate, washed with brine, dried over anhydrous Na₂SO₄ and concentrated under vacuum. The residue was purified by flash column chromatography on silica gel, eluting with ethyl acetate/petroleum ether (1:4) to give tert-butyl N-[cis-3-[[6-(trifluoromethyl)pyridin-3-yl]oxy]cyclobutyl]carbamate (1.5 g) as a white solid. LCMS Method CA: [M+H]⁺=333.

### Step 2: cis-3-[[6-(trifluoromethyl)pyridin-3-yl]oxy]cyclobutan-1-amine hygrochloride

tert-Butyl N-[cis-3-[[6-(trifluoromethyl)pyridin-3-yl]oxy]cyclobutyl]carbamate (660.0 mg, 1.9 mmol, 1.0 equiv.) was dissolved in ethyl acetate (8 mL), then HCl/1,4-dioxane (4N, 10 mL) was added. The resulting mixture was stirred for 30 min at ambient temperature and then concentrated under vacuum to give cis-3-[[6-(trifluoromethyl)pyridin-3-yl]oxy]cyclobutan-1-amine hydrochloride (540.0 mg) as an off-white solid that was used without additional purification. LCMS Method CA: [M+H]⁺=233.

The following intermediates were prepared using the method described for Intermediate B12.

| Intermediate | Starting material A | Starting material B | Structure | LCMS data |
|---|---|---|---|---|
| Intermediate B13 | 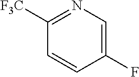 [SMILES: Fc1ccc(C(F)(F)F)nc1] | 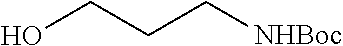 [SMILES: CC(C)(C)OC(=O)NCCCO] | 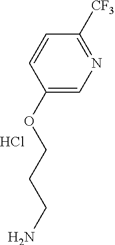 [SMILES: Cl ; NCCCOc1ccc(C(F)(F)F)nc1] | Method CD: MS-ESI: 221 [M + H]⁺ |
| Intermediate B14 | 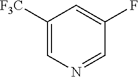 [SMILES: Fc1cncc(C(F)(F)F)c1] | 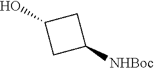 [SMILES: CC(C)(C)OC(=O)N[C@H]1C[C@H](O)C1] | 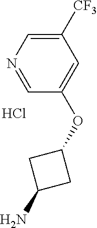 [SMILES: Cl ; N[C@H]1C[C@H](Oc2cncc(C(F)(F)F)c2)C1] | Method CC: MS-ESI: 233 [M + H]⁺ |
| Intermediate B15 | 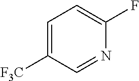 [SMILES: Fc1ccc(C(F)(F)F)cn1] | 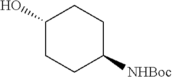 [SMILES: CC(C)(C)OC(=O)N[C@H]1CC[C@H](O)CC1] | 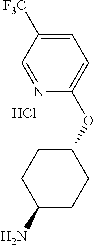 [SMILES: Cl ; N[C@H]1CC[C@H](Oc2ccc(C(F)(F)F)cn2)CC1] | Method CC: MS-ESI: 261 [M + H]⁺ |
| Intermediate B16 | 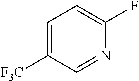 [SMILES: Fc1ccc(C(F)(F)F)cn1] | 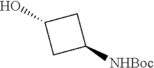 [SMILES: CC(C)(C)OC(=O)N[C@H]1C[C@H](O)C1] | 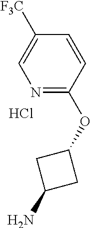 [SMILES: Cl ; N[C@H]1C[C@H](Oc2ccc(C(F)(F)F)cn2)C1] | Method CC: MS-ESI: 233 [M + H]⁺ |
| Intermediate B17 | 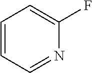 | 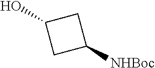 [SMILES: CC(C)(C)OC(=O)N[C@H]1C[C@H](O)C1] | 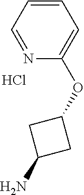 [SMILES: Cl ; N[C@H]1C[C@H](Oc2ccccn2)C1] | Method CD: MS-ESI: 165 [M + H]⁺ |
| Intermediate B18 | 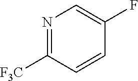 [SMILES: Fc1ccc(C(F)(F)F)nc1] | 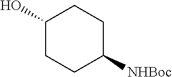 [SMILES: CC(C)(C)OC(=O)N[C@H]1CC[C@H](O)CC1] | 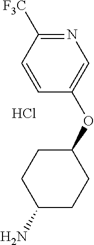 [SMILES: Cl ; N[C@H]1CC[C@H](Oc2ccc(C(F)(F)F)nc2)CC1] | Method CA: MS-ESI: 261 [M + H]⁺ |
| Intermediate B19 | 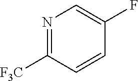 [SMILES: Fc1ccc(C(F)(F)F)nc1] | 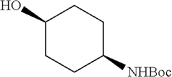 [SMILES: CC(C)(C)OC(=O)N[C@H]1CC[C@@H](O)CC1] | 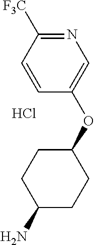 [SMILES: Cl ; N[C@H]1CC[C@@H](Oc2ccc(C(F)(F)F)nc2)CC1] | Method CA: MS-ESI: 261 [M + H]⁺ |
| Intermediate B20 | 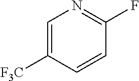 [SMILES: Fc1ccc(C(F)(F)F)cn1] | 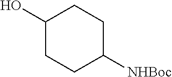 [SMILES: CC(C)(C)OC(=O)NC1CCC(O)CC1] | 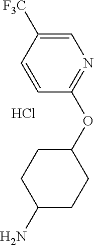 [SMILES: Cl ; NC1CCC(Oc2ccc(C(F)(F)F)cn2)CC1] | Method CC: MS-ESI: 261 [M + H]⁺ |
| Intermediate B21 | 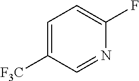 [SMILES: Fc1ccc(C(F)(F)F)cn1] | 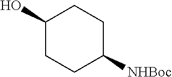 [SMILES: CC(C)(C)OC(=O)N[C@H]1CC[C@@H](O)CC1] | 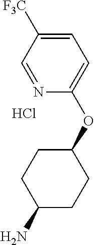 [SMILES: Cl ; N[C@H]1CC[C@@H](Oc2ccc(C(F)(F)F)cn2)CC1] | Method CC: MS-ESI: 261 [M + H]⁺ |
| Intermediate B22 | 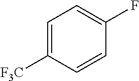 [SMILES: Fc1ccc(C(F)(F)F)cc1] | 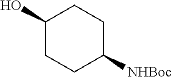 [SMILES: CC(C)(C)OC(=O)N[C@H]1CC[C@@H](O)CC1] | 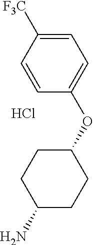 [SMILES: Cl ; N[C@H]1CC[C@@H](Oc2ccc(C(F)(F)F)cc2)CC1] | Method CD: MS-ESI: 260 [M + H]⁺ |
| Intermediate B23 | 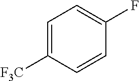 [SMILES: Fc1ccc(C(F)(F)F)cc1] | 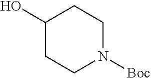 [SMILES: CC(C)(C)OC(=O)N1CCC(O)CC1] | 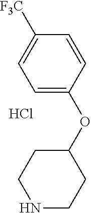 [SMILES: Cl ; FC(F)(F)c1ccc(OC2CCNCC2)cc1] | Method CD: MS-ESI: 246 [M + H]⁺ |

### Synthesis of intermediate B24 (3-(4-(trifluoromethyl)phenoxy)cyclopentan-1-amine hydrochloride)

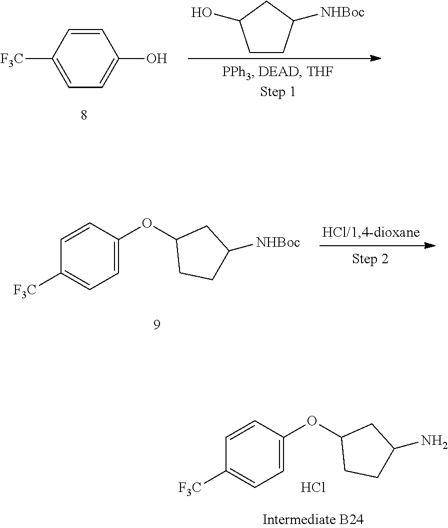 [SMILES: Oc1ccc(C(F)(F)F)cc1 ; CC(C)(C)OC(=O)NC1CCC(O)C1 ; CC(C)(C)OC(=O)NC1CCC(Oc2ccc(C(F)(F)F)cc2)C1 ; NC1CCC(Oc2ccc(C(F)(F)F)cc2)C1 ; Cl]

### Step 1: tert-butyl N-[3-[4-(trifluoromethyl)phenoxy]cyclopentyl]carbamate

tert-Butyl N-(3-hydroxycyclopentyl)carbamate (620.7 mg, 3.0 mmol, 1.0 equiv.), α,α,α-trifluoro-P-cresol (500.0 mg, 3.0 mmol, 1.0 equiv.) and PPh₃ (1.2 g, 4.6 mmol, 1.5 equiv.) were dissolved in THE (20 mL), then DEAD (1.0 g, 6.0 mmol, 2.0 equiv.) was added. The resulting solution was stirred overnight at ambient temperature and then concentrated under vacuum. The residue was diluted with water, extracted with ethyl acetate, dried over anhydrous Na₂SO₄ and concentrated under vacuum. The residue was purified by flash column chromatography on silica gel, eluting with ethyl acetate/petroleum ether (1:4) to give tert-butyl N-[3-[4-(trifluoromethyl)phenoxy]cyclopentyl]carbamate (680.0 mg) as an off-white solid. LCMS Method CB: [M+H]⁺=346.

### Step 2: (3-[4-(trifluoromethyl)phenoxy]cyclopentan-1-amine hydrochloride

The title compound was prepared using the same methods described for Intermediate B12, Step 2. LCMS: Method CA, [M+H]⁺=246.

### Synthesis of Intermediate B25 (trans-N-(5-(trifluoromethyl)pyridin-2-yl)cyclobutane-1,3-diamine hydrochloride)

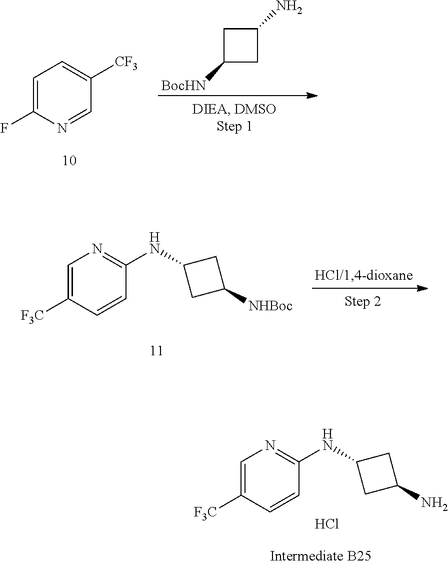 [SMILES: Fc1ccc(C(F)(F)F)cn1 ; CC(C)(C)OC(=O)N[C@H]1C[C@H](N)C1 ; CC(C)(C)OC(=O)N[C@H]1C[C@H](Nc2ccc(C(F)(F)F)cn2)C1 ; N[C@H]1C[C@H](Nc2ccc(C(F)(F)F)cn2)C1 ; Cl]

### Step 1: tert-butyl N-[trans-3-[[5-(trifluoromethyl)pyridin-2-yl]amino]cyclobutyl]carbamate

2-Fluoro-5-(trifluoromethyl)pyridine (316.0 mg, 1.9 mmol, 1.0 equiv.) and tert-butyl N-[trans-3-aminocyclobutyl]carbamate (356.5 mg, 1.9 mmol, 1.0 equiv.) were dissolved in DMSO (15 mL), then DIEA (0.6 mL, 3.8 mmol, 2.0 equiv.) was added. The reaction mixture was heated to 100° C. for 16 hours, then cooled to ambient temperature and quenched by the addition of water. The resulting solution was extracted with ethyl acetate, washed with brine, dried over anhydrous Na₂SO₄ and concentrated under vacuum to give tert-butyl N-[trans-3-[[5-(trifluoromethyl)pyridin-2-yl]amino]cyclobutyl]carbamate (580.0 mg) as a light yellow solid. LCMS Method CA: [M+H]⁺=332.

### Step 2: trans-N-[5-(trifluoromethyl)pyridin-2-yl]cyclobutane-1,3-diamine hydrochloride

The title compound was prepared using the same methods described for Intermediate B12, Step 2. LCMS: Method CA, [M+H]⁺=232.

The following intermediates were prepared using the method described for Intermediate B25.

| Intermediate | Starting Material A | Starting Material B | Structure | LCMS data |
|---|---|---|---|---|
| Intermediate B26 | 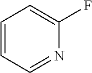 | 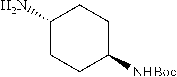 [SMILES: CC(C)(C)OC(=O)N[C@H]1CC[C@H](N)CC1] | 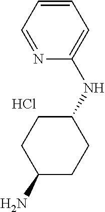 [SMILES: Cl ; N[C@H]1CC[C@H](Nc2ccccn2)CC1] | Method CA: MS-ESI: 192 [M + H]⁺ |
| Intermediate B27 | 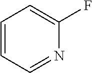 | 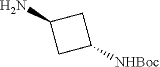 [SMILES: CC(C)(C)OC(=O)N[C@H]1C[C@H](N)C1] | 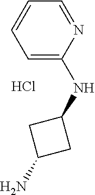 [SMILES: N[C@H]1C[C@H](Nc2ccccn2)C1 ; Cl] | Method CA: MS-ESI: 164 [M + H]⁺ |
| Intermediate B28 | 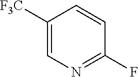 [SMILES: Fc1ccc(C(F)(F)F)cn1] | 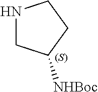 | 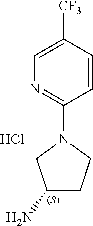 | Method CA: MS-ESI: 232 [M + H]⁺ |
| Intermediate B29 | 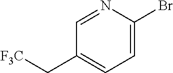 [SMILES: FC(F)(F)Cc1ccc(Br)nc1] | 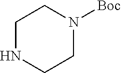 [SMILES: CC(C)(C)OC(=O)N1CCNCC1] | 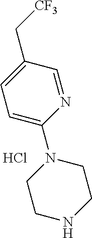 [SMILES: FC(F)(F)Cc1ccc(N2CCNCC2)nc1 ; Cl] | Method CC: MS-ESI: 246 [M + H]+ |

### Synthesis of Intermediate B30 ((1r,4r)-N1-(2,2,2-trifluoroethyl)cyclohexane-1,4-diamine hydrochloride)

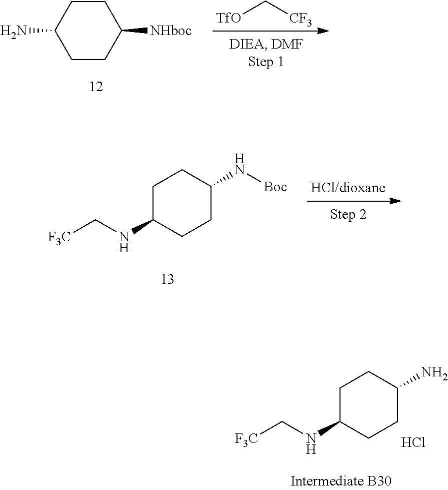

### Step 1: tert-butyl (trans-4-((2,2,2-trifluoroethyl) amino) cyclohexyl) carbamate

tert-Butyl N-[trans-4-aminocyclohexyl] carbamate (500.0 mg, 2.3 mmol, 1.0 equiv.) was dissolved in DMF (8 mL), then 2,2,2-trifluoroethyl trifluoromethanesulfonate (595.7 mg, 2.6 mmol, 1.1 equiv.) and DIEA (1.2 mL, 7.0 mmol, 3.0 equiv.) were added. The reaction mixture was heated to 80° C. for 16 hours, then cooled to ambient temperature and quenched by the addition of water. The resulting solution was extracted with ethyl acetate, dried over anhydrous Na₂SO₄ and concentrated under vacuum to give tert-butyl (trans-4-((2,2,2-trifluoroethyl) amino) cyclohexyl) carbamate (1.1 g) as a brown oil. LCMS Method CA: [M+H]⁺=297.

### Step 2: trans-N-(2,2,2-trifluoroethyl) cyclohexane-1,4-diamine hydrochloride

The title compound was prepared using the same methods described for Intermediate B12, Step 2. LCMS: Method CA, [M+H]⁺=197.

### Synthesis of intermediate B31 (1-(4-(trifluoromethyl)benzyl)piperazine hydrochloride)

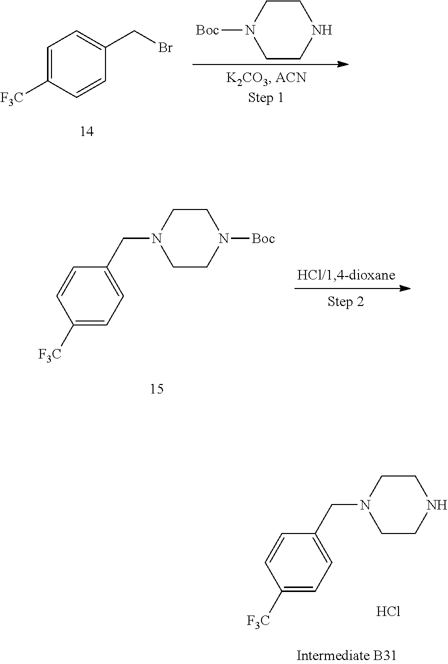 [SMILES: FC(F)(F)c1ccc(CBr)cc1 ; CC(C)(C)OC(=O)N1CCNCC1 ; CC(C)(C)OC(=O)N1CCN(Cc2ccc(C(F)(F)F)cc2)CC1 ; FC(F)(F)c1ccc(CN2CCNCC2)cc1 ; Cl]

### Step 1: tert-butyl 4-[[4-(trifluoromethyl)phenyl]methyl]piperazine-1-carboxylate

tert-Butyl piperazine-1-carboxylate (1.0 g, 5.4 mmol, 1.0 equiv.) was dissolved in ACN (10 mL), then 1-(bromomethyl)-4-(trifluoromethyl)benzene (1.5 g, 6.4 mmol, 1.2 equiv.) and K₂CO₃ (1.5 g, 10.7 mmol, 2.0 equiv.) were added. The reaction mixture was heated to 80° C. for 16 hours then quenched by the addition of water. The resulting solution was extracted with ethyl acetate, washed with brine, dried over anhydrous Na₂SO₄ and concentrated under vacuum to give tert-butyl 4-[[4-(trifluoromethyl)phenyl]methyl]piperazine-1-carboxylate (1.7 g) as a pale yellow oil. LCMS Method CC: [M+H]⁺=345.

### Step 2: 1-[[4-(trifluoromethyl)phenyl]methyl]piperazine hydrochloride

The title compound was prepared using the same methods described for Intermediate B12, Step 2. LCMS: Method CC, [M+H]⁺=245.

The following intermediate was prepared using the method described for Intermediate B31.

| Intermediate | Starting Material A | Starting Material B | Structure | LCMS data |
|---|---|---|---|---|
| Intermediate B32 | 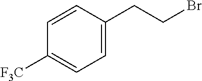 [SMILES: FC(F)(F)c1ccc(CCBr)cc1] | 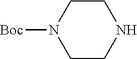 [SMILES: CC(C)(C)OC(=O)N1CCNCC1] | 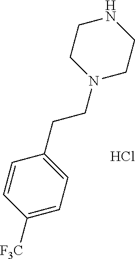 [SMILES: FC(F)(F)c1ccc(CCN2CCNCC2)cc1 ; Cl] | Method CC: MS-ESI: 259 [M + H]⁺ |

### Synthesis of Intermediate B33 (2-amino-1-(2,3-dihydro-1H-pyrrolo[2,3-b]pyridin-1-yl)ethan-1-one hydrochloride)

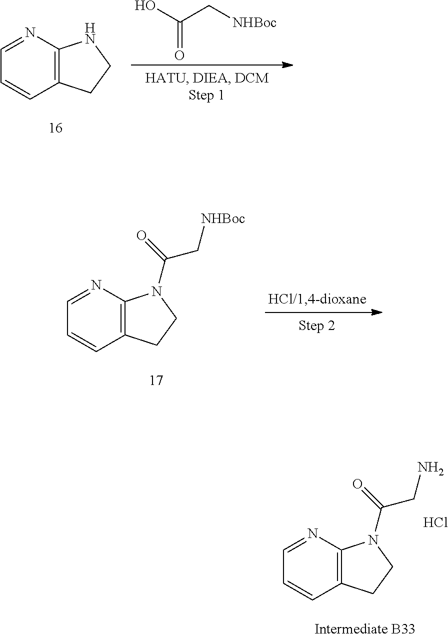 [SMILES: c1cnc2c(c1)CCN2 ; CC(C)(C)OC(=O)NCC(=O)O ; CC(C)(C)OC(=O)NCC(=O)N1CCc2cccnc21 ; NCC(=O)N1CCc2cccnc21 ; Cl]

### Step 1: tert-butyl N-(2-oxo-2-[2H,3H-pyrrolo[2,3-b]pyridin-1-yl]ethyl)carbamate

1H,2H,3H-pyrrolo[2,3-b]pyridine (3.0 g, 25.0 mmol, 1.0 equiv.) was dissolved in DCM (100 mL), then [(tert-butoxycarbonyl)amino]acetic acid (5.3 g, 30.0 mmol, 1.2 equiv.), HATU (14.2 g, 37.5 mmol, 1.5 equiv.) and DIEA (12.4 mL, 74.9 mmol, 3.0 equiv.) were added. The reaction mixture was stirred for 15 hours at ambient temperature and then quenched by the addition of water. The resulting solution was extracted with DCM, washed with brine, dried over anhydrous Na₂SO₄ and concentrated under vacuum. The residue was purified by flash column chromatography on silica gel, eluting with ethyl acetate/petroleum ether (1:2) to give tert-butyl N-(2-oxo-2-[2H,3H-pyrrolo[2,3-b]pyridin-1-yl]ethyl)carbamate (6.5 g) as a pale yellow solid. LCMS Method CA: [M+H]⁺=278.

### Step 2: 2-amino-1-[2H,3H-pyrrolo[2,3-b]pyridin-1-yl]ethanone hydrochloride

The title compound was prepared using the same methods described for Intermediate B12, Step 2. LCMS: Method CC, [M+H]⁺=178.

The following intermediate was prepared using the method described for Intermediate B33.

| Intermediate | Starting Material A | Starting Material | Structure | LCMS data |
|---|---|---|---|---|
| Intermediate B34 | 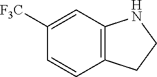 [SMILES: FC(F)(F)c1ccc2c(c1)NCC2] | 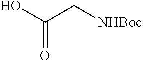 [SMILES: CC(C)(C)OC(=O)NCC(=O)O] | 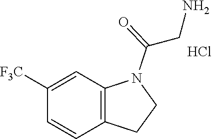 [SMILES: NCC(=O)N1CCc2ccc(C(F)(F)F)cc21 ; Cl] | Method CC: MS-ESI: 178 [M + H]⁺ |

### Synthesis of Intermediate B35 (4-methyl-3,4-dihydro-2H-pyrido[3,2-b][1,4]oxazin-7-amine hydrochloride)

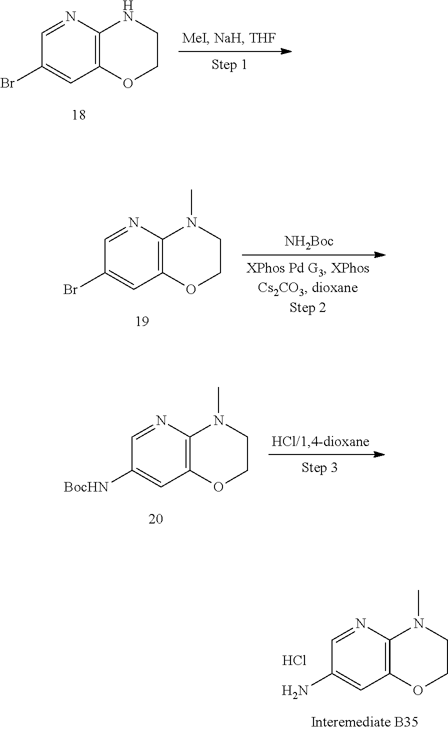 [SMILES: Brc1cnc2c(c1)OCCN2 ; CN1CCOc2cc(Br)cnc21 ; CN1CCOc2cc(NC(=O)OC(C)(C)C)cnc21 ; Cl ; CN1CCOc2cc(N)cnc21 ; "NH2Boc"]

### Step 1: 7-bromo-4-methyl-2H,3H-pyrido[3,2-b][1,4]oxazine

7-Bromo-2H,3H,4H-pyrido[3,2-b][1,4]oxazine (3.0 g, 14.0 mmol, 1.0 equiv.) was dissolved in THE (50 mL) and cooled to 0° C., then NaH (60% wt in mineral oil, 1.1 g, 28.0 mmol, 2.0 equiv.) was added, maintaining the solution at 0° C. After 30 min at 0° C., Mel (1.0 mL, 16.8 mmol, 1.2 equiv.) was added. The reaction mixture was stirred for additional 4 hours at ambient temperature and then quenched by the addition of water. The resulting solution was extracted with ethyl acetate, washed with brine, dried over anhydrous Na₂SO₄ and concentrated under vacuum. The residue was purified by flash column chromatography on silica gel, eluting with ethyl acetate/petroleum ether (1:5) to give 7-bromo-4-methyl-2H,3H-pyrido[3,2-b][1,4]oxazine (2.3 g) as a pale yellow solid. LCMS Method CA: [M+H]⁺=230.

### Step 2: tert-butyl N-[4-methyl-2H,3H-pyrido[3,2-b][1,4]oxazin-7-yl]carbamate

7-Bromo-4-methyl-2H,3H-pyrido[3,2-b][1,4]oxazine (500.0 mg, 2.2 mmol, 1.0 equiv.) was dissolved in 1,4-dioxane (15 mL), then XPhos (208.1 mg, 0.4 mmol, 0.2 equiv.), XPhos Pd G₃ (369.5 mg, 0.4 mmol, 0.2 equiv.), Cs₂CO₃ (1.4 g, 4.4 mmol, 2.0 equiv.) and BocNH₂ (255.7 mg, 2.2 mmol, 1.0 equiv.) were added under an atmosphere of nitrogen. The reaction mixture was heated to 100° C. for 16 hours, then cooled to ambient temperature and quenched by the addition of water. The resulting solution was extracted with ethyl acetate, washed with brine, dried over anhydrous Na₂SO₄ and concentrated under vacuum. The residue was purified by flash column chromatography on silica gel, eluting with ethyl acetate/petroleum ether (1:3) to give tert-butyl N-[4-methyl-2H,3H-pyrido[3,2-b][1,4]oxazin-7-yl]carbamate (333.2 mg) as a pale yellow solid. LCMS Method CA: [M+H]⁺=266.

### Step 3: 4-methyl-2H,3H-pyrido[3,2-b][1,4]oxazin-7-amine hydrochloride

The title compound was prepared using the same methods described for Intermediate B12, Step 2. LCMS: Method CC, [M+H]⁺=166.

### Synthesis of Intermediate B36 (5-amino-2-(pyrrolidin-1-yl)nicotinonitrile)

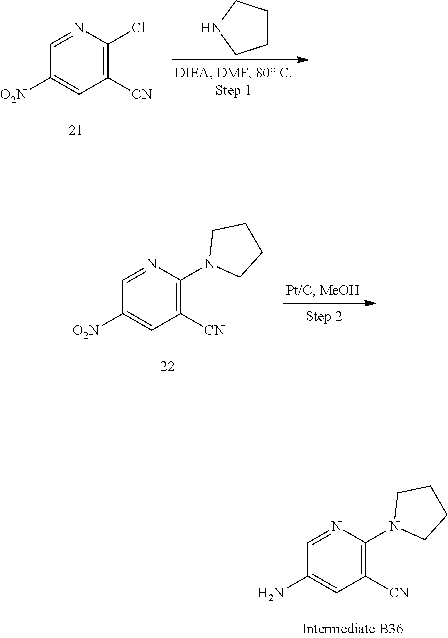 [SMILES: N#Cc1cc([N+](=O)[O-])cnc1Cl ; C1CCNC1 ; N#Cc1cc([N+](=O)[O-])cnc1N1CCCC1 ; N#Cc1cc(N)cnc1N1CCCC1]

### Step 1: 5-nitro-2-(pyrrolidin-1-yl)pyridine-3-carbonitrile

2-Chloro-5-nitropyridine-3-carbonitrile (500.0 mg, 2.7 mmol, 1.0 equiv.) was dissolved in DMF (10 mL), then pyrrolidine (193.7 mg, 2.7 mmol, 1.0 equiv.) and DIEA (0.2 mL, 5.4 mmol, 2.0 equiv.) were added. The resulting solution was heated to 80° C. for 3 hours, then cooled to ambient temperature and quenched by the addition of water. The resulting solution was extracted with ethyl acetate, dried over anhydrous Na₂SO₄ and concentrated under vacuum. The residue was purified by flash column chromatography on silica gel, eluting with ethyl acetate/petroleum ether (1:1) to give crude 5-nitro-2-(pyrrolidin-1-yl)pyridine-3-carbonitrile (600.0 mg) as an off-white solid. LCMS Method CB: [M+H]⁺=219.

### Step 2: 5-amino-2-(pyrrolidin-1-yl)pyridine-3-carbonitrile

5-Nitro-2-(pyrrolidin-1-yl)pyridine-3-carbonitrile (580.0 mg, 2.6 mmol, 1.0 equiv.) was dissolved in MeOH (30 mL), then Pt/C (58.5 mg, wet) was added. The mixture was sparged with nitrogen, placed under an atmosphere of hydrogen gas (balloon), then stirred for 3 hours at ambient temperature. The solids were removed by filtration and the filtrate was concentrated under vacuum. The residue was purified by reverse flash chromatography with the following conditions: column, C18; mobile phase, ACN in water, 0% to 100% gradient in 15 min; detector, UV 254 nm. This gave 5-amino-2-(pyrrolidin-1-yl)pyridine-3-carbonitrile (412.5 mg) as an off-white solid. LCMS Method CB: [M+H]⁺=189.

### Synthesis of Intermediate B37 ((6-(piperidin-1-yl)pyridin-2-yl)methanamine hydrochloride)

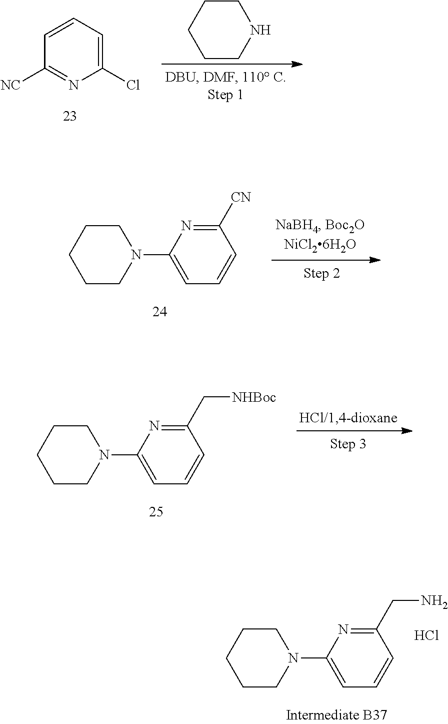 [SMILES: N#Cc1cccc(Cl)n1 ; C1CCNCC1 ; N#Cc1cccc(N2CCCCC2)n1 ; CC(C)(C)OC(=O)NCc1cccc(N2CCCCC2)n1 ; NCc1cccc(N2CCCCC2)n1 ; Cl]

### Step 1: 6-(piperidin-1-yl)pyridine-2-carbonitrile

6-Chloropyridine-2-carbonitrile (100.0 mg, 0.7 mmol, 1.0 equiv.) was dissolved in DMF (15 mL), then piperidine (61.4 mg, 0.7 mmol, 1.0 equiv.) and DBU (329.6 mg, 2.2 mmol, 3.0 equiv.) were added. The reaction mixture was heated to 110° C. for 16 hours, then cooled to ambient temperature and quenched by the addition of water. The resulting solution was extracted with ethyl acetate, washed with brine, dried over anhydrous Na₂SO₄ and concentrated under vacuum to give 6-(piperidin-1-yl)pyridine-2-carbonitrile (105.3 mg) as a yellow oil. LCMS Method CD: [M+H]⁺=188.

### Step 2: tert-butyl N-[[6-(piperidin-1-yl)pyridin-2-yl]methyl]carbamate

6-(Piperidin-1-yl)pyridine-2-carbonitrile (600.0 mg, 3.2 mmol, 1.0 equiv.) and Boc₂O (699.3 mg, 3.2 mmol, 1.0 equiv.) were dissolved in MeOH (100 mL) and cooled to 0° C., then NiCl₂.6H₂O (76.2 mg, 0.3 mmol, 0.1 equiv.), NaBH₄ (969.8 mg, 25.6 mmol, 8.00 equiv.) were added, maintaining the solution at 0° C. The reaction mixture was stirred for 16 hours at ambient temperature, then quenched by the addition of water. The resulting solution was extracted with ethyl acetate, washed with brine, dried over anhydrous Na₂SO₄ and concentrated under vacuum to give tert-butyl N-[[6-(piperidin-1-yl)pyridin-2-yl]methyl]carbamate (411.2 mg) as a pale yellow oil. LCMS Method CC: [M+H]⁺=292.

### Step 3: 1-[6-(piperidin-1-yl)pyridin-2-yl]methanamine hydrochloride

The title compound was prepared using the same methods described for Intermediate B12, Step 2. LCMS: Method CC, [M+H]⁺=192.

### Synthesis of Intermediate B38 (1-(3-(trifluoromethyl)benzyl)pyrrolidin-3-amine hydrochloride)

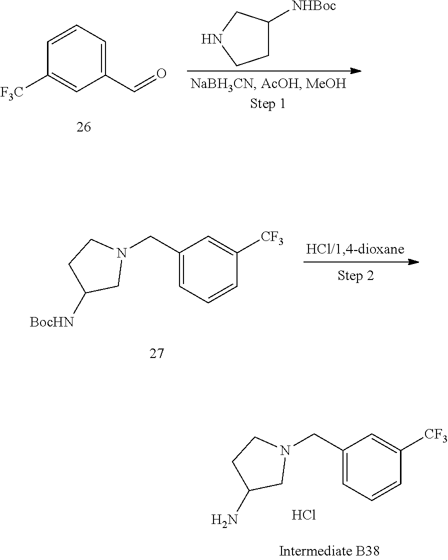 [SMILES: O=Cc1cccc(C(F)(F)F)c1 ; CC(C)(C)OC(=O)NC1CCNC1 ; CC(C)(C)OC(=O)NC1CCN(Cc2cccc(C(F)(F)F)c2)C1 ; NC1CCN(Cc2cccc(C(F)(F)F)c2)C1 ; Cl]

### Step 1: tert-butyl N-(1-[[3-(trifluoromethyl)phenyl]methyl]pyrrolidin-3-yl)carbamate

3-(Trifluoromethyl)benzaldehyde (500.0 mg, 2.9 mmol, 1.0 equiv.) was dissolved in MeOH (50 mL), then tert-butyl N-(pyrrolidin-3-yl)carbamate (534.8 mg, 2.9 mmol, 1.0 equiv.) and AcOH (0.02 mL, 0.3 mmol, 0.1 equiv.) were added. After 10 min, NaBH₃CN (216.5 mg, 3.4 mmol, 1.2 equiv.) was added. The reaction mixture was stirred for 2 hours at ambient temperature and then quenched by the addition of water. The resulting solution was extracted with ethyl acetate, washed with brine, dried over anhydrous Na₂SO₄ and concentrated under vacuum. The residue was purified by flash column chromatography on silica gel, eluting with ethyl acetate/petroleum ether (1:1) to give tert-butyl N-(1-[[3-(trifluoromethyl)phenyl]methyl]pyrrolidin-3-yl)carbamate (751.2 mg) as a white solid. LCMS Method CB: [M+H]⁺=345.

### Step 2: 1-[[3-(trifluoromethyl)phenyl]methyl]pyrrolidin-3-amine hydrochloride

The title compound was prepared using the same methods described for Intermediate B12, Step 2. LCMS: Method CA, [M+H]⁺=245.

### Synthesis of intermediate B39 ((6-(4,4-difluorocyclohexyl)-5-fluoropyridin-3-yl)methanamine hydrochloride)

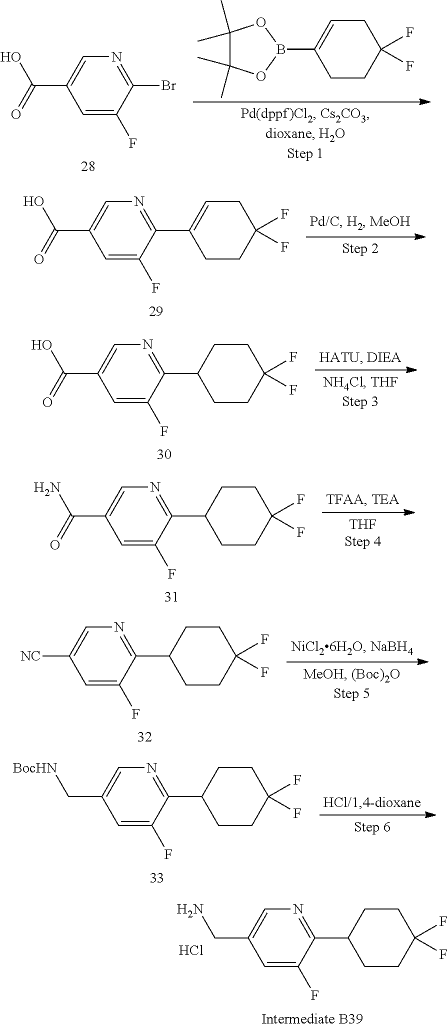

### Step 1: 6-(4,4-difluorocyclohex-1-en-1-yl)-5-fluoronicotinic acid

6-Bromo-5-fluoropyridine-3-carboxylic acid (500.0 mg, 2.2 mmol, 1.0 equiv.) was dissolved in 1,4-dioxane (10 mL) and water (1 mL), then Cs₂CO₃(1.5 g, 4.5 mmol, 2.0 equiv.), Pd(dppf)Cl₂ (166.3 mg, 0.2 mmol, 0.1 equiv.) and 2-(4,4-difluorocyclohex-1-en-1-yl)-4,4,5,5-tetramethyl-1,3,2-dioxaborolane (832.1 mg, 3.4 mmol, 1.5 equiv.) were added under an atmosphere of nitrogen. The reaction mixture was heated to 90° C. for 8 hours, then cooled to ambient temperature and concentrated under vacuum. The residue was diluted with water and then washed with ethyl acetate. The aqueous layer was adjusted to pH 1 with aqueous HCl (2 M). The solids were collected by filtration and dried to give 6-(4,4-difluorocyclohex-1-en-1-yl)-5-fluoronicotinic acid (452.4 mg) as a white solid. LCMS Method CB: [M−H]⁻=256.

### Step 2: 6-(4,4-difluorocyclohexyl)-5-fluoropyridine-3-carboxylic acid

6-(4,4-Difluorocyclohex-1-en-1-yl)-5-fluoronicotinic acid (450.0 mg, 1.7 mmol, 1.0 equiv.) was dissolved in MeOH (10 mL), then Pd/C (45.5 mg, 10% wt.) was added. The mixture was sparged with nitrogen, placed under an atmosphere of hydrogen gas (balloon), then stirred for 5 hours at ambient temperature. The solids were removed by filtration and the filtrate was concentrated under vacuum to give 6-(4,4-difluorocyclohexyl)-5-fluoropyridine-3-carboxylic acid (422.5 mg) as a white solid. LCMS Method CB: [M−H]⁻=258.

### Step 3: 6-(4,4-difluorocyclohexyl)-5-fluoropyridine-3-carboxamide

6-(4,4-Difluorocyclohexyl)-5-fluoropyridine-3-carboxylic acid (380.0 mg, 1.4 mmol, 1.0 equiv.) was dissolved in THE (15 mL), then DIEA (1.2 mL, 7.3 mmol, 5.0 equiv.), HATU (836.0 mg, 2.2 mmol, 1.5 equiv.) and NH₄Cl (392.0 mg, 7.3 mmol, 5.0 equiv.) were added. The resulting solution was stirred for 5 hours at ambient temperature, and then concentrated under vacuum. The residue was purified by flash column chromatography on silica gel, eluting with ethyl acetate/petroleum ether (1:1) to give 6-(4,4-difluorocyclohexyl)-5-fluoropyridine-3-carboxamide (322.5 mg) as a white solid. LCMS Method CC: [M+H]⁺=259.

### Step 4: 6-(4,4-difluorocyclohexyl)-5-fluoropyridine-3-carbonitrile

6-(4,4-Difluorocyclohexyl)-5-fluoropyridine-3-carboxamide (210.0 mg, 0.8 mmol, 1.0 equiv.) and TEA (0.2 mL, 1.6 mmol, 2.0 equiv.) were dissolved in THE (10 mL), then TFAA (341.6 mg, 1.6 mmol, 2.0 equiv.) was added. The resulting solution was stirred for 2 hours at ambient temperature, then quenched by the addition of water. The mixture was extracted with ethyl acetate, washed with brine, dried over anhydrous Na₂SO₄ and concentrated under vacuum to give 6-(4,4-difluorocyclohexyl)-5-fluoropyridine-3-carbonitrile (181.2 mg) as a white solid. LCMS Method CH: [M+H]⁺=241.

### Step 5: tert-butyl N-[[6-(4,4-difluorocyclohexyl)-5-fluoropyridin-3-yl]methyl]carbamate

6-(4,4-Difluorocyclohexyl)-5-fluoropyridine-3-carbonitrile (180.0 mg, 0.7 mmol, 1.0 equiv.) was dissolved in MeOH (10 mL), then NiCl₂.6H₂O (356.2 mg, 1.5 mmol, 2.0 equiv.) and Boc₂O (327.0 mg, 1.5 mmol, 2.0 equiv.) were added. This was followed by the addition of NaBH₄ (56.7 mg, 1.5 mmol, 2.0 equiv.). The reaction mixture was stirred for 3 hours at ambient temperature and then quenched by the addition of water. The solids were removed by filtration and the filtrate was concentrated under vacuum. The residue was purified by flash column chromatography on silica gel, eluting with ethyl acetate/petroleum ether (1:2) to give tert-butyl N-[[6-(4,4-difluorocyclohexyl)-5-fluoropyridin-3-yl]methyl]carbamate (121.3 mg) as a white solid. LCMS Method CA: [M+H]⁺=345.

### Step 6: 1-[6-(4,4-difluorocyclohexyl)-5-fluoropyridin-3-yl]methanamine hydrochloride

The title compound was prepared using the same methods described for Intermediate B12, Step 2. LCMS: Method CA, [M+H]⁺=245.

### Synthesis of intermediate B40 (5-bromo-6-fluoro-1H-indol-3-amine hydrochloride)

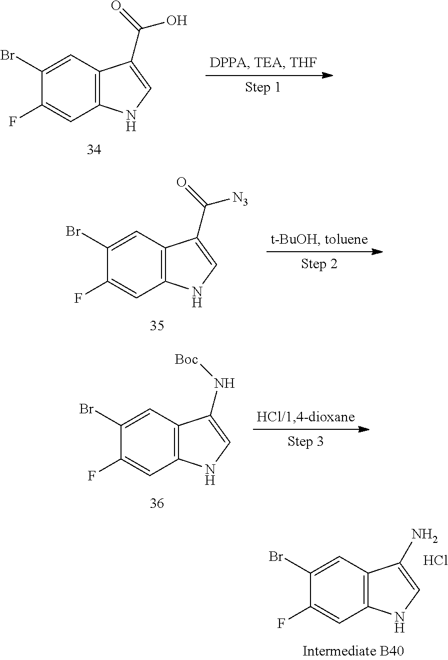 [SMILES: O=C(O)c1c[nH]c2cc(F)c(Br)cc12 ; [N-]=[N+]=NC(=O)c1c[nH]c2cc(F)c(Br)cc12 ; CC(C)(C)OC(=O)Nc1c[nH]c2cc(F)c(Br)cc12 ; Nc1c[nH]c2cc(F)c(Br)cc12 ; Cl]

### Step 1: 5-bromo-6-fluoro-1H-indole-3-carbonyl azide

5-Bromo-6-fluoro-1H-indole-3-carboxylic acid (1.0 g, 3.8 mmol, 1.0 equiv.) was dissolved in THE (20 mL), then TEA (1.1 mL, 7.7 mmol, 2.0 equiv.) and DPPA (1.6 g, 5.8 mmol, 1.5 equiv.) were added. The reaction mixture was stirred for 12 hours at ambient temperature and then quenched by the addition of water. The resulting solution was extracted with of ethyl acetate, washed with brine, dried over anhydrous Na₂SO₄ and concentrated under vacuum to give 5-bromo-6-fluoro-1H-indole-3-carbonyl azide (805.3 mg) of as a white solid. LCMS Method CC: [M+H]⁺=283.

### Step 2: tert-butyl N-(5-bromo-6-fluoro-1H-indol-3-yl)carbamate

5-Bromo-6-fluoro-1H-indole-3-carbonyl azide (800.0 mg, 2.8 mmol, 1.0 equiv.) was dissolved in t-BuOH (15 mL). The resulting solution was heated to 90° C. for 12 hours, then cooled to ambient temperature and concentrated under vacuum. The residue was purified by flash column chromatography on silica gel, eluting with ethyl acetate/petroleum ether (1:2) to give tert-butyl N-(5-bromo-6-fluoro-1H-indol-3-yl)carbamate (661.5 mg) as a white solid. LCMS Method CA: [M+H]⁺=329.

### Step 3: 5-bromo-6-fluoro-1H-indol-3-amine hydrochloride

The title compound was prepared using the same methods described for Intermediate B12, Step 2. LCMS: Method CA, [M+H]⁺=229.

The following intermediates were prepared using the method described for Intermediate B40.

| Intermediate | Starting material | Structure | LCMS data |
|---|---|---|---|
| Intermediate B41 | 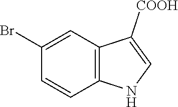 [SMILES: O=C(O)c1c[nH]c2ccc(Br)cc12] | 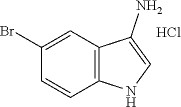 [SMILES: Nc1c[nH]c2ccc(Br)cc12 ; Cl] | Method CA: MS- ESI: 211 [M + H]⁺ |
| Intermediate B42 | 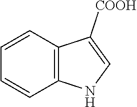 [SMILES: O=C(O)c1c[nH]c2ccccc12] | 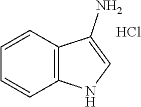 | Method CA: MS- ESI: 169 [M + H]⁺ |

### Synthesis of Intermediate B43 (6-(trans-4-aminocyclohexyl)oxy)nicotinonitrile hydrochloride)

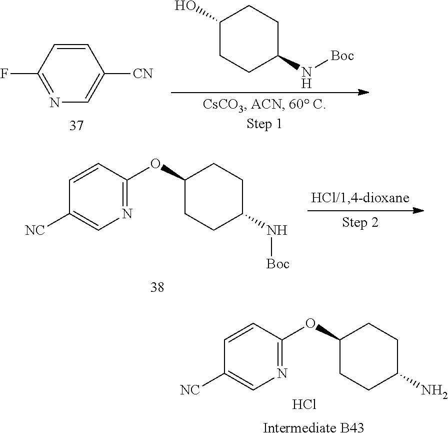 [SMILES: N#Cc1ccc(F)nc1 ; CC(C)(C)OC(=O)N[C@H]1CC[C@H](O)CC1 ; CC(C)(C)OC(=O)N[C@H]1CC[C@H](Oc2ccc(C#N)cn2)CC1 ; N#Cc1ccc(O[C@H]2CC[C@H](N)CC2)nc1 ; Cl]

### Step 1: tert-butyl N-[trans-4-[(5-cyanopyridin-2-yl)oxy]cyclohexyl]carbamate

6-fluoropyridine-3-carbonitrile (1.0 g, 8.1 mmol, 1.0 equiv.) was dissolved in ACN (20 mL), then tert-butyl N-[trans-4-hydroxycyclohexyl]carbamate (1.7 g, 8.1 mmol, 1.0 equiv.) and Cs₂CO₃ (5.3 g, 16.3 mmol, 2.0 equiv.) were added. The reaction mixture was heated to 60° C. overnight, then cooled to ambient temperature and quenched by the addition of water. The resulting solution was extracted with ethyl acetate, dried over anhydrous Na₂SO₄ and concentrated under vacuum. The residue was purified by flash column chromatography on silica gel, eluting with ethyl acetate/petroleum ether (1:1) to give tert-butyl N-[trans-4-[(5-cyanopyridin-2-yl)oxy]cyclohexyl]carbamate (1.7 g) as an off-white solid. LCMS Method CA: [M+H]⁺=318.

### Step 2: 6-((trans-4-aminocyclohexyl)oxy)nicotinonitrile hydrochloride

The title compound was prepared using the same methods described for Intermediate B12, Step 2. LCMS: Method CA, [M+H]⁺=218.

### Synthesis of Intermediate B44 (trans-3-(pyridin-3-yloxy)cyclobutan-1-amine hydrochloride)

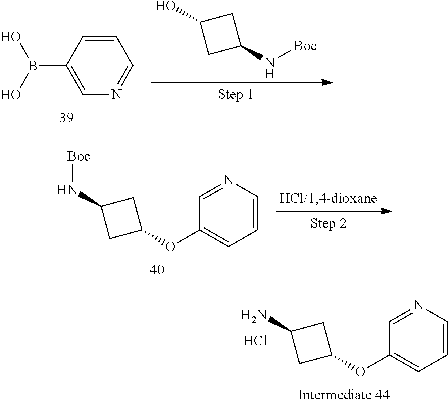 [SMILES: OB(O)c1cccnc1 ; CC(C)(C)OC(=O)N[C@H]1C[C@H](O)C1 ; CC(C)(C)OC(=O)N[C@H]1C[C@H](Oc2cccnc2)C1 ; Cl ; N[C@H]1C[C@H](Oc2cccnc2)C1]

### Step 1: tert-butyl N-[trans-3-(pyridin-3-yloxy)cyclobutyl]carbamate

Pyridin-3-ylboronic acid (2.0 g, 16.2 mmol, 1.0 equiv.) was dissolved in THE (50 mL), then tert-butyl N-[trans-3-hydroxycyclobutyl]carbamate (3.0 g, 16.2 mmol, 1.0 equiv.), Cu(AcO)₂ (591.0 mg, 3.2 mmol, 0.2 equiv.) and TEA (4.5 mL, 32.5 mmol, 2.0 equiv.) were added under an atmosphere of nitrogen. The reaction mixture was stirred overnight at ambient temperature and then concentrated under vacuum. The residue was diluted with water, extracted with ethyl acetate, dried over anhydrous Na₂SO₄ and concentrated under vacuum. The residue was purified by flash column chromatography on silica gel, eluting with ethyl acetate/petroleum ether (1:1) to give tert-butyl N-[trans-3-(pyridin-3-yloxy)cyclobutyl]carbamate (1.5 g) as a brown oil. LCMS Method CA: [M+H]⁺=265.

### Step 2: trans-3-(pyridin-3-yloxy)cyclobutan-1-amine hydrochloride

The title compound was prepared using the same methods described for Intermediate B12, Step 2. LCMS: Method CA, [M+H]⁺=165.

### Example 1: N1-(5,6-difluoro-1H-indol-3-yl)-N₂-(4-(trifluoromethyl)phenyl) oxalamide (Compound 115)

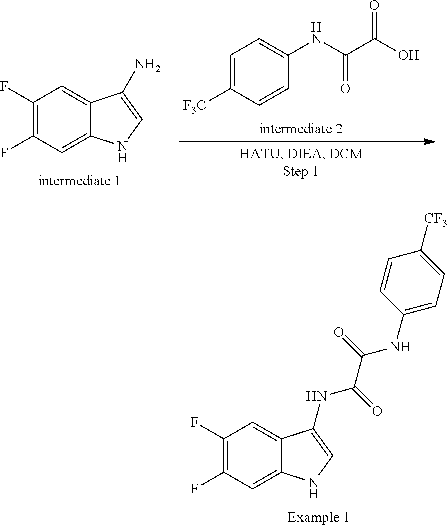 [SMILES: Nc1c[nH]c2cc(F)c(F)cc12 ; O=C(O)C(=O)Nc1ccc(C(F)(F)F)cc1 ; O=C(Nc1ccc(C(F)(F)F)cc1)C(=O)Nc1c[nH]c2cc(F)c(F)cc12]

[[4-(trifluoromethyl)phenyl]carbamoyl]formic acid (50.0 mg, 0.2 mmol, 1.0 equiv) was dissolved in DCM (10.0 mL), then 5,6-difluoro-1H-indol-3-amine (32.5 mg, 0.2 mmol, 0.9 equiv), HATU (163.1 mg, 0.4 mmol, 2.0 equiv) and DIEA (0.069 mL, 0.4 mmol, 2.0 equiv) were added. The reaction mixture was allowed to stir overnight at RT, then concentrated in vacuo. The residue was purified by Prep-HPLC using Method G, to give N₁-(5,6-difluoro-1H-indol-3-yl)-N₂-(4-(trifluoromethyl)phenyl) oxalamide (5.0 mg, 0.01 mmol) as a white solid. LCMS: Method B, MS-ESI: 382.1 [M−H⁻]. ¹HNMR (400 MHz, DMSO-d₆): δ 11.24 (s, 1H), 11.18 (s, 1H), 11.00 (s, 1H), 8.12 (d, 2H), 8.04-7.95 (m, 1H), 7.88 (m, 1H), 7.78 (d, 2H), 7.43 (m, 1H).

TABLE E2
The examples in Table E2 were prepared using the procedure described in
Example 1, starting from the appropriate amine.

| Exam- | Com- | | | LC-MS |
|---|---|---|---|---|
| ple | pound # | Starting Materials | Structure | Data |
| 2 | 102 | Int. 1: 5,6-difluoro-1H- indol-3-amine; Int. 3: [[3- (trifluoromethyl)phenyl]- carbamoyl]formic acid | 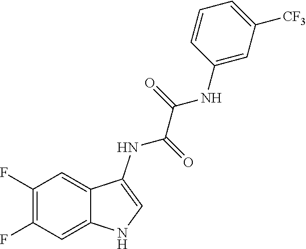 [SMILES: O=C(Nc1cccc(C(F)(F)F)c1)C(=O)Nc1c[nH]c2cc(F)c(F)cc12] | Method C: MS-ESI: 381.9 [M − H⁻]. |
| 3 | 113 | Int. 1: 5,6-difluoro-1H- indol-3-amine; Int. 4: [(4- ethylcyclohexyl)- carbamoyl]formic acid | 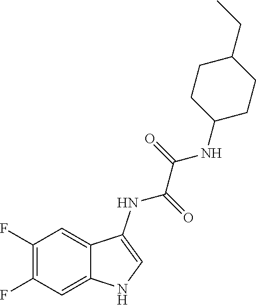 [SMILES: CCC1CCC(NC(=O)C(=O)Nc2c[nH]c3cc(F)c(F)cc23)CC1] | Method D: MS-ESI: 350.2 [M + H⁺]. |
| 5 | 114 | Int. 1: 5,6-difluoro-1H- indol-3-amine; Int. 5: [methyl[4- (trifluoromethyl)phenyl]- carbamoyl]formic acid | 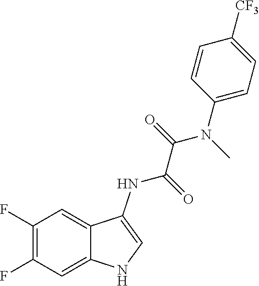 [SMILES: CN(C(=O)C(=O)Nc1c[nH]c2cc(F)c(F)cc12)c1ccc(C(F)(F)F)cc1] | Method C: MS-ESI: 398.2 [M + H⁺]. |
| 6 | 117 | Int. 1: 5,6-difluoro-1H- indol-3-amine; Int. 6: (4-ethylpiperidin- 1-yl)(oxo)acetic acid | 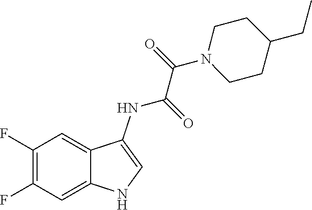 [SMILES: CCC1CCN(C(=O)C(=O)Nc2c[nH]c3cc(F)c(F)cc23)CC1] | Method D: MS-ESI: 336.2 [M + H⁺]. |

### Example 7: N1-(1H-indol-3-yl)-N₂-(4-(trifluoromethyl)phenyl)oxalamide (Compound 116)

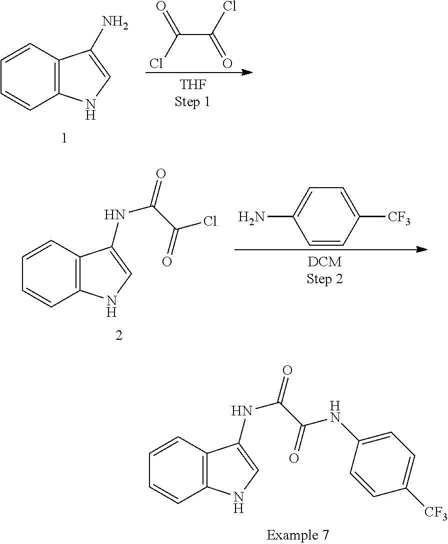 [SMILES: Nc1c[nH]c2ccccc12 ; O=C(Cl)C(=O)Cl ; O=C(Cl)C(=O)Nc1c[nH]c2ccccc12 ; Nc1ccc(C(F)(F)F)cc1 ; O=C(Nc1ccc(C(F)(F)F)cc1)C(=O)Nc1c[nH]c2ccccc12]

Step 1—Synthesis of 2-((1H-indol-3-yl)amino)-2-oxoacetyl chloride: 1H-indol-3-amine (100.0 mg, 0.7 mmol, 1.0 equiv) was dissolved in THE (10.0 mL) and the reaction mixture was cooled to 0° C. Oxalyl chloride (0.07 mL, 0.7 mmol, 1.0 equiv) was added and the reaction mixture was stirred for 2 h at 0° C. The reaction mixture was concentrated in vacuo and the residue was used directly in the next step without additional purification.

Step 2—Synthesis of N₁-(1H-indol-3-yl)-N₂-(4-(trifluoromethyl)phenyl)oxalamide (Example 2): 4-Trifluoromethylaniline (146.4 mg, 0.9 mmol, 1.2 equiv) was dissolved in THE (10.0 mL) then 2-((1H-indol-3-yl)amino)-2-oxoacetyl chloride (100 mg) from Step 1 was added and the reaction mixture was stirred for 2 h at 0° C. The reaction mixture was concentrated in vacuo and the residue was purified by Prep-HPLC using Method G to give N₁-(1H-indol-3-yl)-N₂-(4-(trifluoromethyl)phenyl)oxalamide (18.3 mg, 0.05 mmol) as a white solid. LCMS: Method C, MS-ESI: 346.0 [M−H⁻]. ¹HNMR: (400 MHz, DMSO-d₆) δ 11.18 (s, 1H), 11.07 (s, 1H), 10.81 (s, 1H), 8.13-8.11 (m, 2H), 7.89 (d, 1H), 7.79-7.76 (m, 3H), 7.40 (d, 1H), 7.15-7.11 (m, 1H), 7.04-7.00 (m, 1H).

### Example 8: N -(5,6-difluoro-1H-indol-3-yl)-N²-(3-methyl-5-(trifluoromethyl)phenyl)-oxalamide (Compound 103)

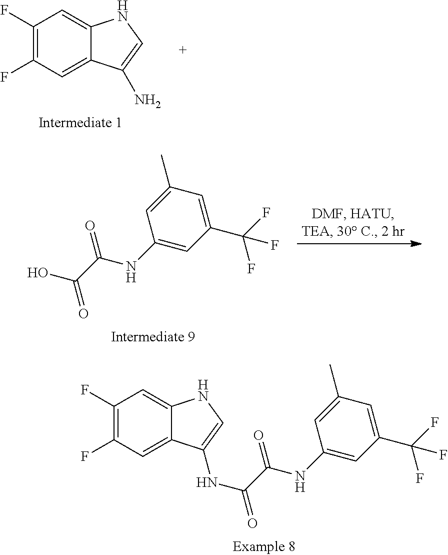 [SMILES: Nc1c[nH]c2cc(F)c(F)cc12 ; Cc1cc(NC(=O)C(=O)O)cc(C(F)(F)F)c1 ; Cc1cc(NC(=O)C(=O)Nc2c[nH]c3cc(F)c(F)cc23)cc(C(F)(F)F)c1]

To a mixture of 2-((3-methyl-5-(trifluoromethyl)phenyl)amino)-2-oxoacetic acid (61.8 mg, 0.25 mmol, 1.0 equiv.) and 5,6-difluoro-1H-indol-3-amine (42.0 mg, 0.25 mmol, 1.0 equiv.) in DMF (3 mL) was added HATU (95.0 mg, 0.25 mmol, 1.0 equiv.) and TEA (70 μl, 0.5 mmol, 2.0 equiv.). The mixture was stirred at 30° C. for 2 hours. The solvent was removed in vacuo and the residue was purified by prep. HIPLC to give N¹-(5,6-difluoro-1H-indol-3-yl)-N²-(3-methyl-5-(trifluoromethyl)phenyl)oxalamide (12.1 mg, 0.03 mmol) as a purple powder. Analysis Condition: Method F MS-ESI, 398.0 [M+H⁺]. ¹H NMR (400 MHz, DMSO-d₆) δ=11.24 (br s, 1H), 11.08 (s, 1H), 10.95 (s, 1H), 8.17 (s, 1H), 8.01-7.95 (m, 2H), 7.88 (d, 1H), 7.40 (dd, 1H), 7.35 (s, 1H), 2.40 (s, 3H).

TABLE E3
The examples in Table E3 were prepared using the procedure described in
Example 8, starting from the appropriate acid.

| | | | | LC- |
|---|---|---|---|---|
| | Compound | Starting | | MS |
| Example | # | Materials | Structure | Data |
| 9 | 104 | Int. 10 | 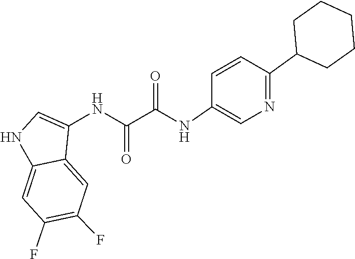 [SMILES: O=C(Nc1ccc(C2CCCCC2)nc1)C(=O)Nc1c[nH]c2cc(F)c(F)cc12] | 399.1 |
| 10 | 101 | Int. 11 | 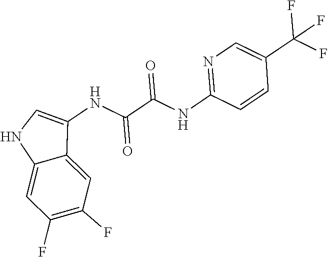 [SMILES: O=C(Nc1ccc(C(F)(F)F)cn1)C(=O)Nc1c[nH]c2cc(F)c(F)cc12] | 385.0 |
| 11 | 108 | Int. 12 | 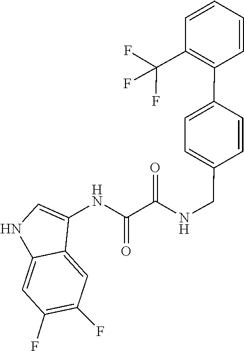 [SMILES: O=C(NCc1ccc(-c2ccccc2C(F)(F)F)cc1)C(=O)Nc1c[nH]c2cc(F)c(F)cc12] | 474.0 |
| 12 | 109 | Int. 13 | 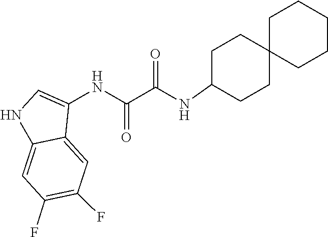 [SMILES: O=C(Nc1c[nH]c2cc(F)c(F)cc12)C(=O)NC1CCC2(CCCCC2)CC1] | 390.1 |
| 13 | 105 | Int. 14 | 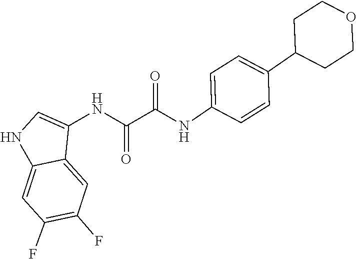 [SMILES: O=C(Nc1ccc(C2CCOCC2)cc1)C(=O)Nc1c[nH]c2cc(F)c(F)cc12] | 400.0 |
| 14 | 112 | Int. 15 | 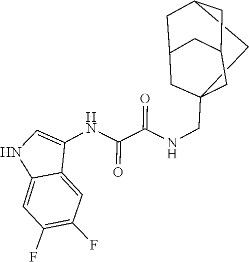 [SMILES: O=C(NCC12CC3CC(CC(C3)C1)C2)C(=O)Nc1c[nH]c2cc(F)c(F)cc12] | 388.1 |
| 15 | 111 | Int. 16 | 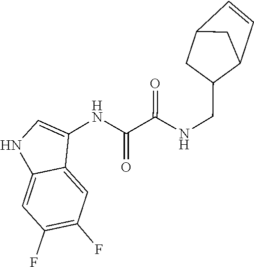 [SMILES: O=C(NCC1CC2C=CC1C2)C(=O)Nc1c[nH]c2cc(F)c(F)cc12] | 346.1 |
| 16 | 110 | Int. 17 | 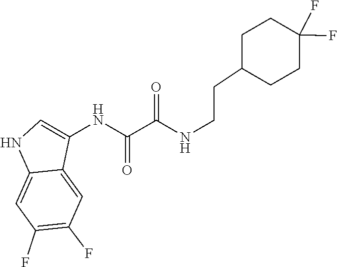 [SMILES: O=C(NCCC1CCC(F)(F)CC1)C(=O)Nc1c[nH]c2cc(F)c(F)cc12] | 386.1 |
| 17 | 107 | Int. 18 | 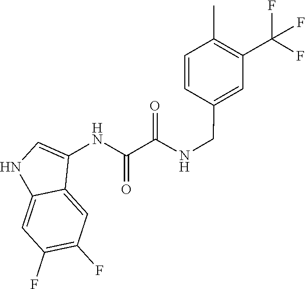 [SMILES: Cc1ccc(CNC(=O)C(=O)Nc2c[nH]c3cc(F)c(F)cc23)cc1C(F)(F)F] | 412.0 |
| 18 | 106 | Int. 19 | 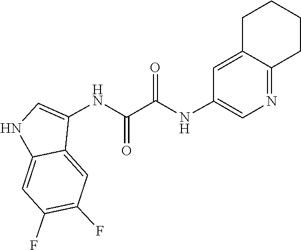 [SMILES: O=C(Nc1cnc2c(c1)CCCC2)C(=O)Nc1c[nH]c2cc(F)c(F)cc12] | 371.1 |

### Example 19. N-(5,6-difluoro-1H-indol-3-yl)-N′-[[5-(trifluoromethyl)pyridin-2-yl]methyl]ethanediamide (Compound 119)

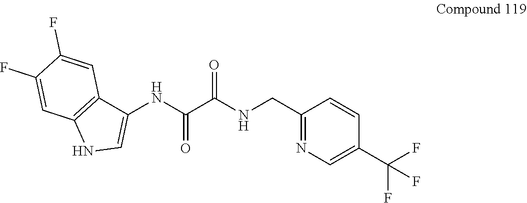 [SMILES: O=C(NCc1ccc(C(F)(F)F)cn1)C(=O)Nc1c[nH]c2cc(F)c(F)cc12]

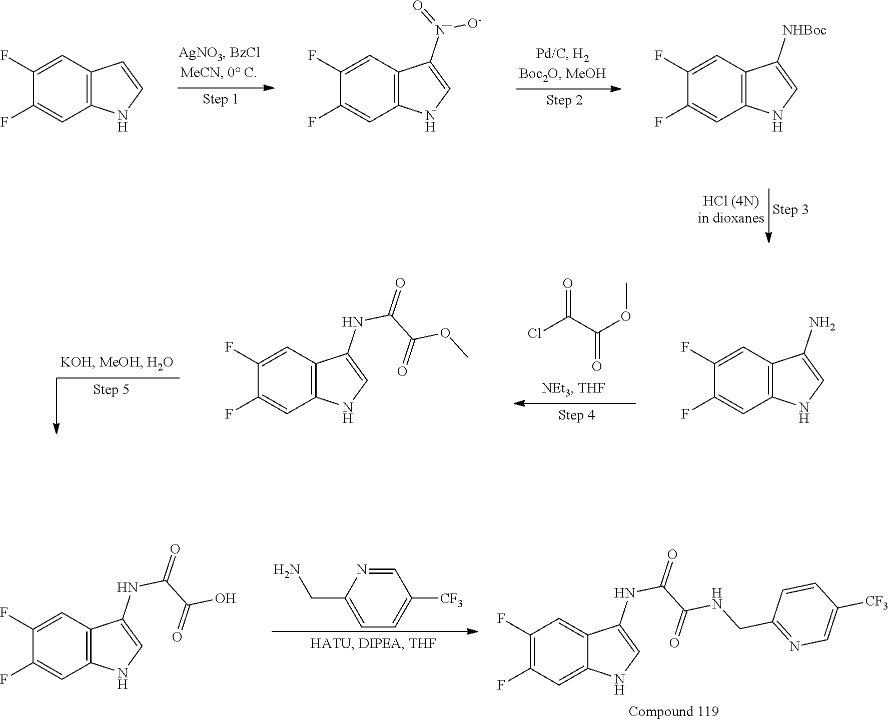

### Step 1: 5,6-difluoro-3-nitro-1H-indole

5,6-difluoro-1H-indole (25.0 g, 163 mmol, 1.0 eq.) was dissolved in in ACN (300 mL) and cooled to 0° C. AgNO₃ (33.3 g, 196. mmol, 1.2 eq.) was then added. After 15 min, benzoyl chloride (27.5 g, 196. mmol, 1.2 eq.) was added. The resulting solution was stirred for 3 hours at 0° C. The pH of the solution was adjusted to 8 by the dropwise addition of saturated aqueous NaHCO₃. The solids were removed by filtration, then filtrate was extracted with DCM, and the combined organic layers were concentrated under vacuum. The residue was purified by flash column chromatography on silica gel, eluting with EtOAc/petroleum ether (2:1) to give 5,6-difluoro-3-nitro-1H-indole (24 g) as a brown solid. MS-ESI: [M+H]⁺=199.

### Step 2: tert-butyl N-(5,6-difluoro-1H-indol-3-yl)carbamate

5,6-difluoro-3-nitro-1H-indole (24.0 g, 121 mmol, 1.0 eq.) was dissolved in MeOH (300 mL). Pd/C (2.4 g, 10% wt, 2 mmol, 0.02 eq.) and (Boc)₂O (39.7 g, 182 mmol, 1.5 eq.) were then added. The reaction vessel was evacuated then back filled with hydrogen three times. The reaction mixture was then stirred for 16 h under an atmosphere of hydrogen. After filtration and concentration, the resulting residue was purified by flash column chromatography on silica gel, eluting with ethyl acetate/petroleum ether (1:4) to give tert-butyl N-(5,6-difluoro-1H-indol-3-yl)carbamate (22 g) as a yellow solid. LCMS Method BA: [M+H]⁺=269.

### Step 3: 5,6-difluoro-1H-indol-3-amine hydrochloride

Tert-butyl-N-(5,6-difluoro-1H-indol-3-yl)carbamate (17.0 g, 63 mmol, 1.0 eq.) was added to 4 N HCl in 1,4-dioxane (200.0 mL). The resulting mixture was stirred for 30 min and then concentrated to give 5,6-difluoro-1H-indol-3-amine hydrochloride (12 g) as a yellow solid that was used without additional purification. MS-ESI: [M+H]⁺=169.

### Step 4: methyl [(5,6-difluoro-1H-indol-3-yl)carbamoyl]formate

5,6-difluoro-1H-indol-3-amine hydrochloride (16.0 g, 95.2 mmol, 1.0 eq.) was dissolved in THE (250 mL). TEA (9.6 g, 95.2 mmol, 1.0 eq.) and methyl oxalochloridate (11.7 g, 95.2 mmol, 1.0 eq.) were then added. The resulting solution was stirred for 2 h at 0° C. The resulting solids were collected by filtration to give methyl [(5,6-difluoro-1H-indol-3-yl)carbamoyl]formate (16.3 g) as a yellow solid. LCMS Method BE: [M+H]⁺=255.

### Step 5: [(5,6-difluoro-1H-indol-3-yl)carbamoyl]formic acid

Methyl [(5,6-difluoro-1H-indol-3-yl)carbamoyl]formate (5.0 g, 19.6 mmol, 1.0 eq) was dissolved in MeOH (60 mL)/H₂O (12 mL). KOH (2.2 g, 39.4 mmol, 2.0 eq.) was then added. The resulting solution was stirred for 3 hr. The pH of the resulting solution was adjusted to 3 with 6 M HCl. The resulting solids were collected by filtration to give 2.5 g of [(5,6-difluoro-1H-indol-3-yl)carbamoyl]formic acid as a yellow solid. MS-ESI: [M−H]⁻=239.

### Step 6: N-(5,6-difluoro-1H-indol-3-yl)-N′-[[5-(trifluoromethyl)pyridin-2-yl]methyl]ethanediamide

[(5,6-difluoro-1H-indol-3-yl)carbamoyl]formic acid (0.8 g, 3.3 mmol, 1.0 eq.) was dissolved in THE (100.0 mL). DIEA (0.8 g, 6.7 mmol, 2.0 eq.), HATU (2.5 g, 6.7 mmol, 2.0 eq.) and 1-[5-(trifluoromethyl)pyridin-2-yl]methanamine (0.6 g, 3.3 mmol, 1.0 eq.) were then added. The resulting solution was stirred for 6 h and then concentrated under vacuum. The residue was purified by flash column chromatography on silica gel, eluting with EtOAc/petroleum ether (1:2). The resulting solid was washed with MeOH to give N-(5,6-difluoro-1H-indol-3-yl)-N′-[[5-(trifluoromethyl)pyridin-2-yl]methyl]ethanediamide (511 mg) as an off-white solid. LCMS Method BB: [M+H]⁺=399. ¹H NMR (400 MHz, DMSO-d₆) δ 11.21 (s, 1H), 10.79 (s, 1H), 9.60 (t, 1H), 8.94 (s, 1H), 8.19 (d, 1H), 7.97 (dd, 1H), 7.87 (d, 1H), 7.58 (d, 1H), 7.37 (dd, 1H), 4.65 (d, 2H).

TABLE E4
The examples in Table E4 were prepared using the procedure described in
Example 8, starting from the appropriate acid.

| | | | | LC-MS, | |
|---|---|---|---|---|---|
| | | | | MS- | |
| Example | Compound | | | ESI,-- | LCMS |
| # | # | Structure | IUPAC Name | [M + H⁺]. | Method |
| 20 | 167 | 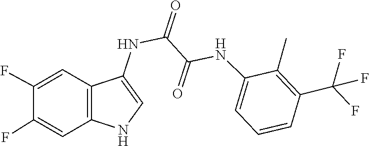 [SMILES: Cc1c(NC(=O)C(=O)Nc2c[nH]c3cc(F)c(F)cc23)cccc1C(F)(F)F] | N1-(5,6- difluoro-1H- indol-3-yl)-N2- (2-methyl-3- (trifluoromethyl) phenyl)oxalamide | 398.1 | |
| 21 | 166 | 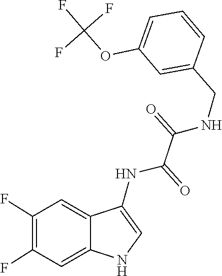 [SMILES: O=C(NCc1cccc(OC(F)(F)F)c1)C(=O)Nc1c[nH]c2cc(F)c(F)cc12] | N1-(5,6- difluoro-1H- indol-3-yl)-N2-(3- (trifluoromethoxy) benzyl)oxalamide | 414.2 | Method AA |
| 22 | 165 | 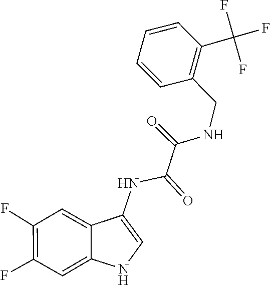 [SMILES: O=C(NCc1ccccc1C(F)(F)F)C(=O)Nc1c[nH]c2cc(F)c(F)cc12] | N1-(5,6- difluoro-1H- indol-3-yl)-N2-(2- (trifluoromethyl) benzyl)oxalamide | 398.2 | Method AA |
| 23 | 164 | 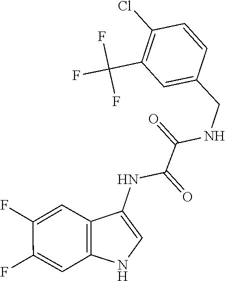 [SMILES: O=C(NCc1ccc(Cl)c(C(F)(F)F)c1)C(=O)Nc1c[nH]c2cc(F)c(F)cc12] | N1-(4-chloro-3- (trifluoromethyl) benzyl)-N2- (5,6-difluoro- 1H-indol-3- yl)oxalamide | 432.1 | Method AA |
| 24 | 163 | 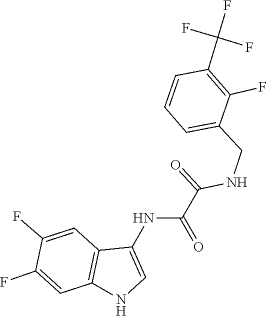 [SMILES: O=C(NCc1cccc(C(F)(F)F)c1F)C(=O)Nc1c[nH]c2cc(F)c(F)cc12] | N1-(5,6- difluoro-1H- indol-3-yl)-N2- (2-fluoro-3- (trifluoromethyl) benzyl)oxalamide | 416.2 | Method AA |
| 25 | 162 | 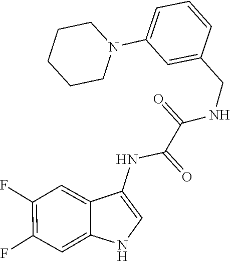 [SMILES: O=C(NCc1cccc(N2CCCCC2)c1)C(=O)Nc1c[nH]c2cc(F)c(F)cc12] | N1-(5,6- difluoro-1H- indol-3-yl)-N2- (3-(piperidin-1- yl)benzyl) oxalamide | 413.2 | Method AA |
| 26 | 161 | 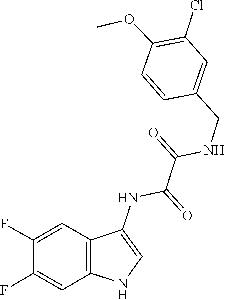 [SMILES: COc1ccc(CNC(=O)C(=O)Nc2c[nH]c3cc(F)c(F)cc23)cc1Cl] | N1-(3-chloro-4- methoxybenzyl) N2-(5,6- difluoro-1H- indol-3- yl)oxalamide | 394.1 | Method AA |
| 27 | 160 | 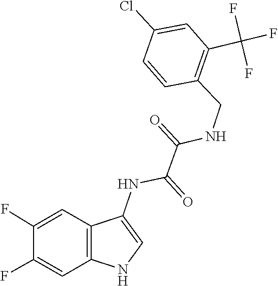 [SMILES: O=C(NCc1ccc(Cl)cc1C(F)(F)F)C(=O)Nc1c[nH]c2cc(F)c(F)cc12] | N1-(4-chloro-2- (trifluoromethyl) benzyl)-N2- (5,6-difluoro- 1H-indol-3- yl)oxalamide | 432.1 | Method AA |
| 28 | 192 | 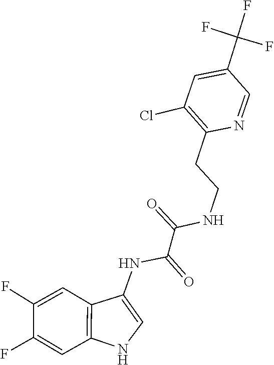 [SMILES: O=C(NCCc1ncc(C(F)(F)F)cc1Cl)C(=O)Nc1c[nH]c2cc(F)c(F)cc12] | N1-(2-(3-chloro-5- (trifluoromethyl) pyridin-2- yl)ethyl)-N2- (5,6-difluoro- 1H-indol-3- yl)oxalamide | 447.1 | Method AA |
| 29 | 132 | 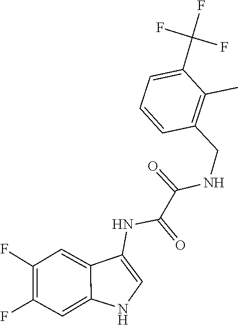 [SMILES: Cc1c(CNC(=O)C(=O)Nc2c[nH]c3cc(F)c(F)cc23)cccc1C(F)(F)F] | N1-(5,6- difluoro-1H- indol-3-yl)-N2- (2-methyl-3- (trifluoromethyl) benzyl)oxalamide | 412.1 | Method AA |
| 30 | 131 | 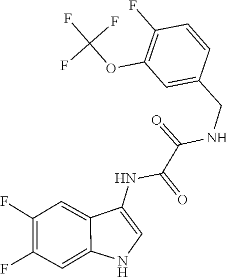 [SMILES: O=C(NCc1ccc(F)c(OC(F)(F)F)c1)C(=O)Nc1c[nH]c2cc(F)c(F)cc12] | N1-(5,6- difluoro-1H- indol-3-yl)-N2- (4-fluoro-3- (trifluoromethoxy) benzyl) oxalamide | 432.0 | Method AA |
| 31 | 159 | 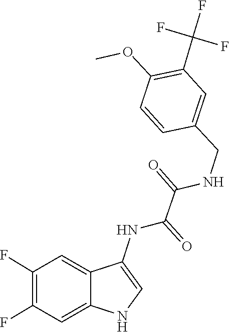 [SMILES: COc1ccc(CNC(=O)C(=O)Nc2c[nH]c3cc(F)c(F)cc23)cc1C(F)(F)F] | N1-(5,6- difluoro-1H- indol-3-yl)-N2- (4-methoxy-3- (trifluoromethyl) benzyl)oxalamide | 428.2 | Method AA |
| 32 | 158 | 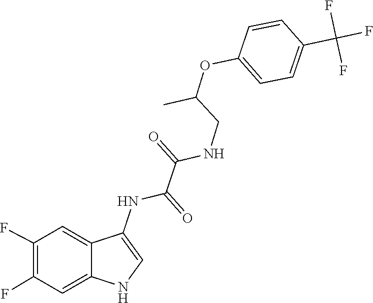 [SMILES: CC(CNC(=O)C(=O)Nc1c[nH]c2cc(F)c(F)cc12)Oc1ccc(C(F)(F)F)cc1] | N1-(5,6- difluoro-1H- indol-3-yl)-N2- (2-(4- (trifluoromethyl) phenoxy)propyl) oxalamide | 442.2 | Method AA |
| 33 | 157 | 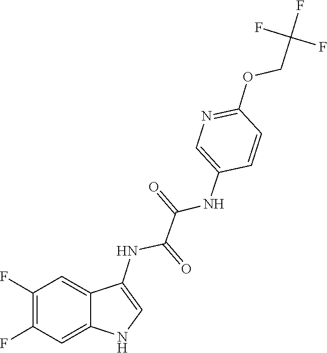 [SMILES: O=C(Nc1ccc(OCC(F)(F)F)nc1)C(=O)Nc1c[nH]c2cc(F)c(F)cc12] | N1-(5,6- difluoro-1H- indol-3-yl)-N2- (6-(2,2,2- trifluoroethoxy) pyridin-3-yl) oxalamide | 415.1 | Method AA |
| 34 | 130 | 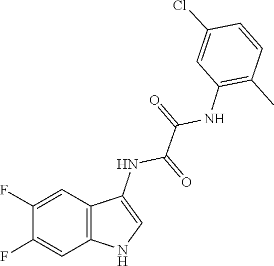 [SMILES: Cc1ccc(Cl)cc1NC(=O)C(=O)Nc1c[nH]c2cc(F)c(F)cc12] | N1-(5-chloro-2- methylphenyl)- N2-(5,6- difluoro-1H- indol-3- yl)oxalamide | 364.1 | Method AB |
| 35 | 156 | 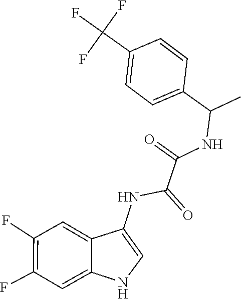 [SMILES: CC(NC(=O)C(=O)Nc1c[nH]c2cc(F)c(F)cc12)c1ccc(C(F)(F)F)cc1] | N1-(5,6- difluoro-1H- indol-3-yl)-N2- (1-(4- (trifluoromethyl) phenyl)ethyl) oxalamide | 412.2 | Method AA |
| 36 | 155 | 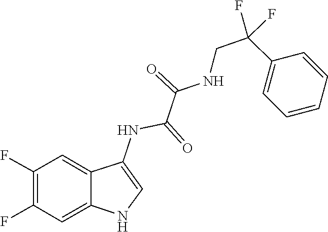 [SMILES: O=C(NCC(F)(F)c1ccccc1)C(=O)Nc1c[nH]c2cc(F)c(F)cc12] | N1-(5,6- difluoro-1H- indol-3-yl)-N2- (2,2-difluoro-2- phenylethyl) oxalamide | 380.1 | Method AA |
| 37 | 154 | 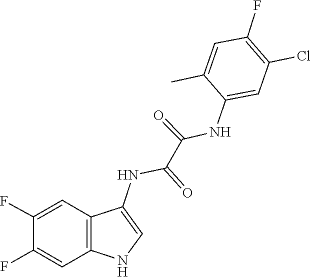 [SMILES: Cc1cc(F)c(Cl)cc1NC(=O)C(=O)Nc1c[nH]c2cc(F)c(F)cc12] | N1-(5-chloro-4- fluoro-2- methylphenyl)- N2-(5,6- difluoro-1H- indol-3- yl)oxalamide | 382.1 | Method AA |
| 38 | 153 | 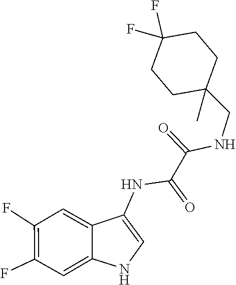 [SMILES: CC1(CNC(=O)C(=O)Nc2c[nH]c3cc(F)c(F)cc23)CCC(F)(F)CC1] | N1-((4,4- difluoro-1- methylcyclohexyl) methyl)-N2- (5,6-difluoro- 1H-indol-3- yl)oxalamide | 386.2 | Method AA |
| 39 | 152 | 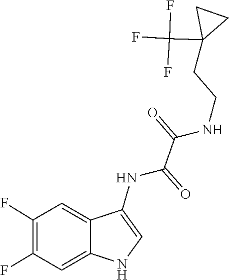 [SMILES: O=C(NCCC1(C(F)(F)F)CC1)C(=O)Nc1c[nH]c2cc(F)c(F)cc12] | N1-(5,6- difluoro-1H- indol-3-yl)-N2- (2-(1- (trifluoromethyl) cyclopropyl) ethyl)oxalamide | 376.2 | Method AA |
| 40 | 193 | 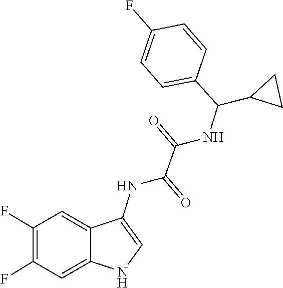 [SMILES: O=C(Nc1c[nH]c2cc(F)c(F)cc12)C(=O)NC(c1ccc(F)cc1)C1CC1] | N1- (cyclopropyl(4- fluorophenyl) methyl)-N2-(5, 6-difluoro-1H- indol-3- yl)oxalamide | 388.2 | Method AB |
| 41 | 151 | 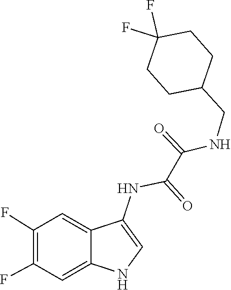 [SMILES: O=C(NCC1CCC(F)(F)CC1)C(=O)Nc1c[nH]c2cc(F)c(F)cc12] | N1-(5,6- difluoro-1H- indol-3-yl)-N2- ((4,4- difluorocyclohexyl) methyl) oxalamide | 372.2 | Method AA |
| 42 | 150 | 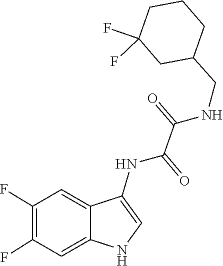 [SMILES: O=C(NCC1CCCC(F)(F)C1)C(=O)Nc1c[nH]c2cc(F)c(F)cc12] | N1-(5,6- difluoro-1H- indol-3-yl)-N2- ((3,3- difluorocyclohexyl) methyl)oxalamide | 372.2 | Method AA |
| 43 | 149 | 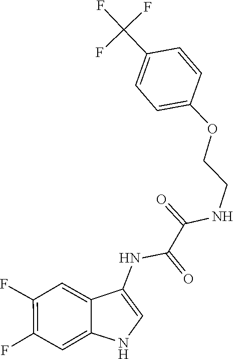 [SMILES: O=C(NCCOc1ccc(C(F)(F)F)cc1)C(=O)Nc1c[nH]c2cc(F)c(F)cc12] | N1-(5,6- difluoro-1H- indol-3-yl)-N2- (2-(4- (trifluoromethyl) phenoxy)ethyl) oxalamide | 428.1 | Method AA |
| 44 | 148 | 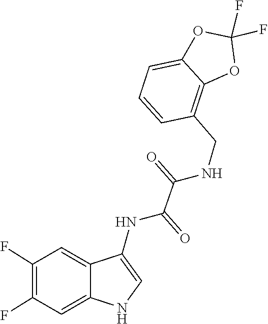 [SMILES: O=C(NCc1cccc2c1OC(F)(F)O2)C(=O)Nc1c[nH]c2cc(F)c(F)cc12] | N1-(5,6- difluoro-1H- indol-3-yl)-N2- ((2,2- difluorobenzo[d] [1,3]dioxol-4- yl)methyl) oxalamide | 410.1 | Method AA |
| 45 | 147 | 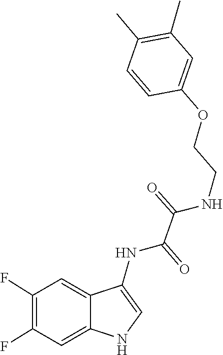 [SMILES: Cc1ccc(OCCNC(=O)C(=O)Nc2c[nH]c3cc(F)c(F)cc23)cc1C] | N1-(5,6- difluoro-1H- indol-3-yl)-N2- (2-(3,4- dimethylphenoxy) ethyl)oxalamide | 388.2 | Method AA |
| 46 | 146 | 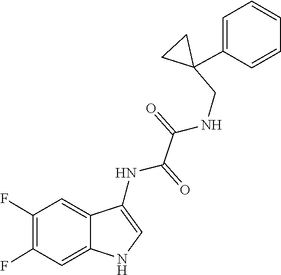 [SMILES: O=C(NCC1(c2ccccc2)CC1)C(=O)Nc1c[nH]c2cc(F)c(F)cc12] | N1-(5,6- difluoro-1H- indol-3-yl)-N2- ((1- phenylcyclopropyl) methyl)oxalamide | 370.2 | Method AA |
| 47 | 138 | 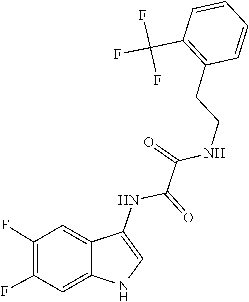 [SMILES: O=C(NCCc1ccccc1C(F)(F)F)C(=O)Nc1c[nH]c2cc(F)c(F)cc12] | N1-(5,6- difluoro-1H- indol-3-yl)-N2- (2- (trifluoromethyl) phenethyl) oxalamide | 412.2 | Method AA |
| 48 | 145 | 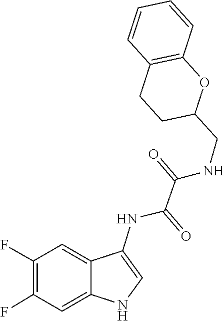 [SMILES: O=C(NCC1CCc2ccccc2O1)C(=O)Nc1c[nH]c2cc(F)c(F)cc12] | N-(chroman-2- ylmethyl)-N2- (5,6-difluoro- 1H-indol-3- yl)oxalamide | 386.2 | Method AA |
| 49 | 144 | 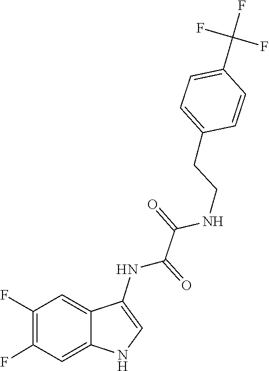 [SMILES: O=C(NCCc1ccc(C(F)(F)F)cc1)C(=O)Nc1c[nH]c2cc(F)c(F)cc12] | N1-(5,6- difluoro-1H- indol-3-yl)-N2- (4- (trifluoromethyl) phenethyl) oxalamide | 412.2 | Method AA |
| 50 | 194 | 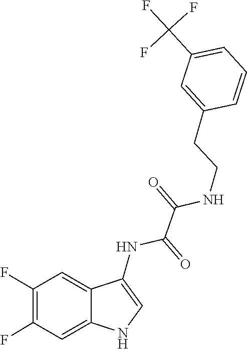 [SMILES: O=C(NCCc1cccc(C(F)(F)F)c1)C(=O)Nc1c[nH]c2cc(F)c(F)cc12] | N1-(5,6- difluoro-1H- indol-3-yl)-N2- (3- (trifluoromethyl) phenethyl) oxalamide | 412.0 | Method AA |

The following compounds were synthesized using methods similar to those described herein from the appropriate starting materials.

| Example | | | Mol. Weight | | LC/MS |
|---|---|---|---|---|---|
| # | Compound # | Structure | (Calc.) | LC/MS* | Method |
| 51 | 124 | 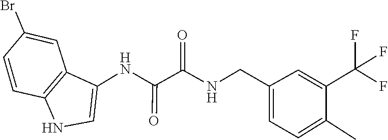 [SMILES: Cc1ccc(CNC(=O)C(=O)Nc2c[nH]c3ccc(Br)cc23)cc1C(F)(F)F] | 454.247 | 454.0 | BD |
| 52 | 125 | 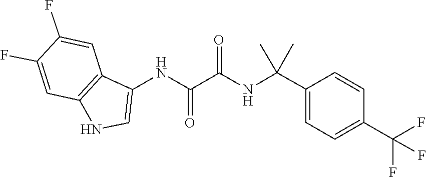 [SMILES: CC(C)(NC(=O)C(=O)Nc1c[nH]c2cc(F)c(F)cc12)c1ccc(C(F)(F)F)cc1] | 425.359 | 424.1 | BD |
| 53 | 126 | 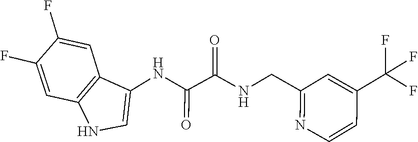 [SMILES: O=C(NCc1cc(C(F)(F)F)ccn1)C(=O)Nc1c[nH]c2cc(F)c(F)cc12] | 398.293 | 399.1 | BB |
| 54 | 127 | 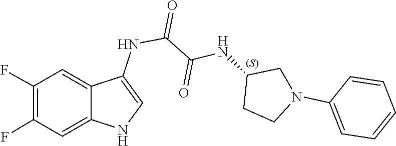 | 384.387 | 385.0 | BB |
| 55 | 128 | 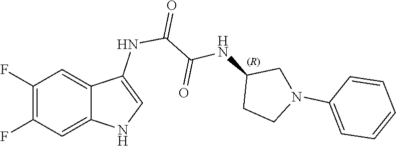 | 384.387 | 385.1 | BB |
| 56 | 129 | 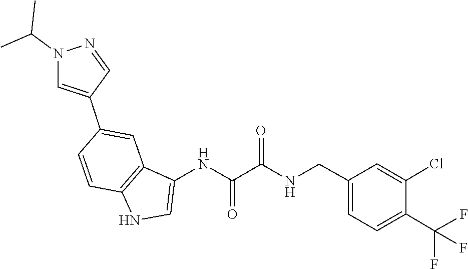 [SMILES: CC(C)n1cc(-c2ccc3[nH]cc(NC(=O)C(=O)NCc4ccc(C(F)(F)F)c(Cl)c4)c3c2)cn1] | 503.91 | 504.1 | BB |
| 57 | 133 | 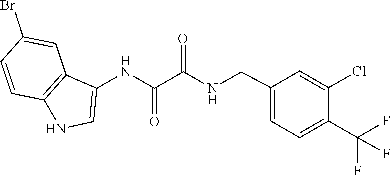 [SMILES: O=C(NCc1ccc(C(F)(F)F)c(Cl)c1)C(=O)Nc1c[nH]c2ccc(Br)cc12] | 474.66 | 473.95 | BD |
| 58 | 134 | 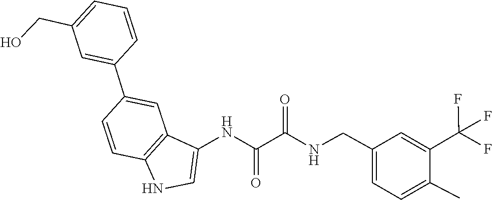 [SMILES: Cc1ccc(CNC(=O)C(=O)Nc2c[nH]c3ccc(-c4cccc(CO)c4)cc23)cc1C(F)(F)F] | 481.475 | 480.2 | BC |
| 59 | 135 | 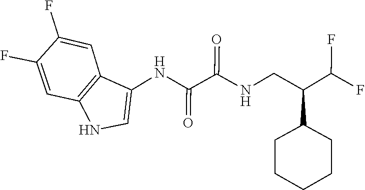 [SMILES: O=C(NC[C@@H](C(F)F)C1CCCCC1)C(=O)Nc1c[nH]c2cc(F)c(F)cc12] | 399.39 | 398.05 | BB |
| 60 | 136 | 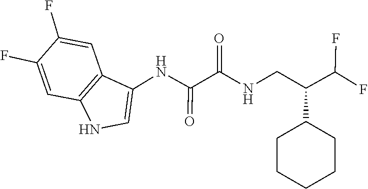 [SMILES: O=C(NC[C@H](C(F)F)C1CCCCC1)C(=O)Nc1c[nH]c2cc(F)c(F)cc12] | 399.39 | 398.1 | BB |
| 61 | 137 | 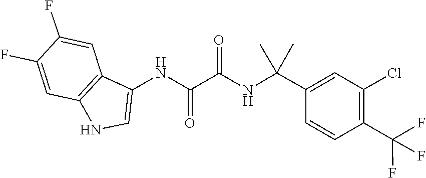 [SMILES: CC(C)(NC(=O)C(=O)Nc1c[nH]c2cc(F)c(F)cc12)c1ccc(C(F)(F)F)c(Cl)c1] | 459.8 | 458.05 | BB |
| 62 | 139 | 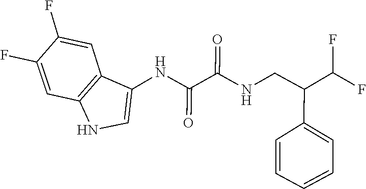 [SMILES: O=C(NCC(c1ccccc1)C(F)F)C(=O)Nc1c[nH]c2cc(F)c(F)cc12] | 393.342 | 394.1 | BB |
| 63 | 140 | 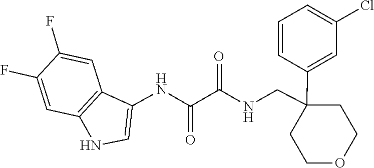 [SMILES: O=C(NCC1(c2cccc(Cl)c2)CCOCC1)C(=O)Nc1c[nH]c2cc(F)c(F)cc12] | 447.87 | 448.0 | BA |
| 64 | 141 | 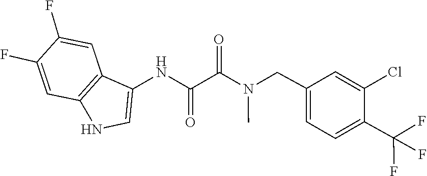 [SMILES: CN(Cc1ccc(C(F)(F)F)c(Cl)c1)C(=O)C(=O)Nc1c[nH]c2cc(F)c(F)cc12] | 445.77 | 444.0 | BB |
| 65 | 142 | 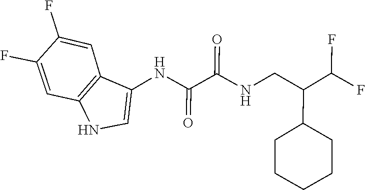 [SMILES: O=C(NCC(C(F)F)C1CCCCC1)C(=O)Nc1c[nH]c2cc(F)c(F)cc12] | 399.39 | 398.1 | BB |
| 66 | 143 | 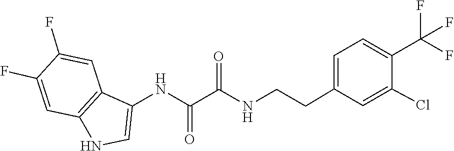 [SMILES: O=C(NCCc1ccc(C(F)(F)F)c(Cl)c1)C(=O)Nc1c[nH]c2cc(F)c(F)cc12] | 445.77 | 444.0 | BB |
| 67 | 168 | 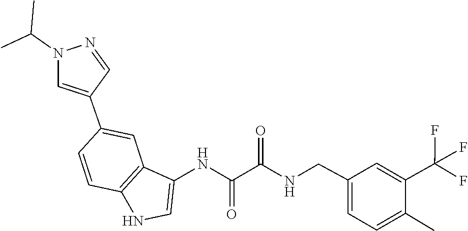 [SMILES: Cc1ccc(CNC(=O)C(=O)Nc2c[nH]c3ccc(-c4cnn(C(C)C)c4)cc23)cc1C(F)(F)F] | 483.495 | 484.1 | BA |
| 68 | 169 | 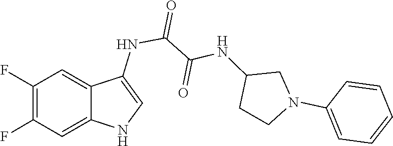 [SMILES: O=C(Nc1c[nH]c2cc(F)c(F)cc12)C(=O)NC1CCN(c2ccccc2)C1] | 384.387 | 385.0 | BB |
| 69 | 170 | 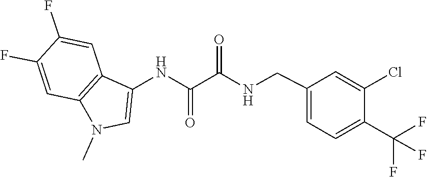 [SMILES: Cn1cc(NC(=O)C(=O)NCc2ccc(C(F)(F)F)c(Cl)c2)c2cc(F)c(F)cc21] | 445.77 | 446.1 | BB |
| 70 | 171 | 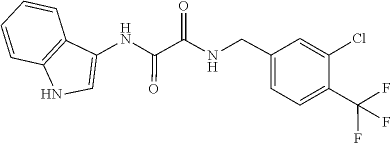 [SMILES: O=C(NCc1ccc(C(F)(F)F)c(Cl)c1)C(=O)Nc1c[nH]c2ccccc12] | 395.77 | 394.0 | BD |
| 71 | 172 | 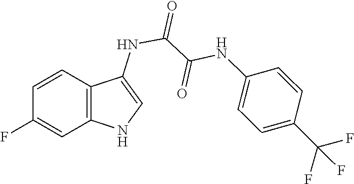 [SMILES: O=C(Nc1ccc(C(F)(F)F)cc1)C(=O)Nc1c[nH]c2cc(F)ccc12] | 365.288 | 364.0 | BB |
| 72 | 173 | 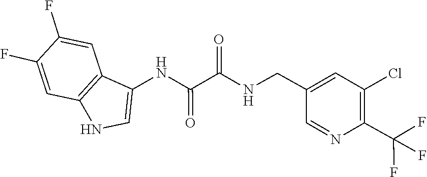 [SMILES: O=C(NCc1cnc(C(F)(F)F)c(Cl)c1)C(=O)Nc1c[nH]c2cc(F)c(F)cc12] | 432.74 | 431.0 | BB |
| 73 | 174 | 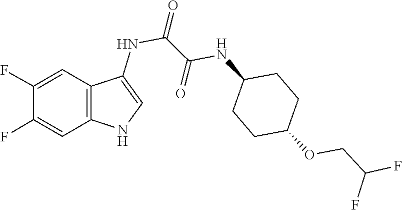 [SMILES: O=C(Nc1c[nH]c2cc(F)c(F)cc12)C(=O)N[C@H]1CC[C@H](OCC(F)F)CC1] | 401.362 | 400.0 | BB |
| 74 | 175 | 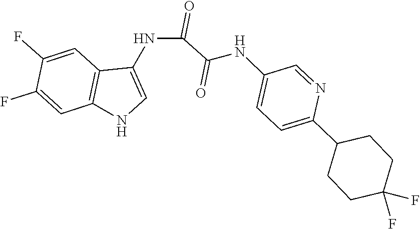 [SMILES: O=C(Nc1ccc(C2CCC(F)(F)CC2)nc1)C(=O)Nc1c[nH]c2cc(F)c(F)cc12] | 434.395 | 435.1 | BB |
| 75 | 176 | 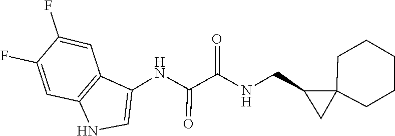 [SMILES: O=C(NC[C@@H]1CC12CCCCC2)C(=O)Nc1c[nH]c2cc(F)c(F)cc12] | 361.393 | 360.1 | BD |
| 76 | 177 | 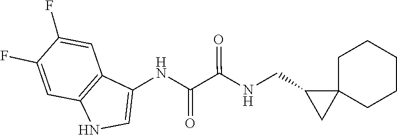 [SMILES: O=C(NC[C@H]1CC12CCCCC2)C(=O)Nc1c[nH]c2cc(F)c(F)cc12] | 361.393 | 360.0 | BD |
| 77 | 178 | 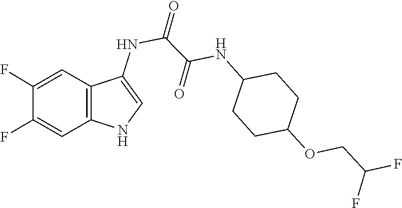 [SMILES: O=C(Nc1c[nH]c2cc(F)c(F)cc12)C(=O)NC1CCC(OCC(F)F)CC1] | 401.362 | 400.5 | BB |
| 78 | 179 | 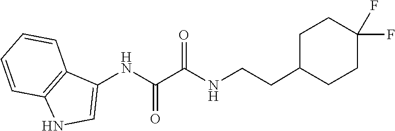 [SMILES: O=C(NCCC1CCC(F)(F)CC1)C(=O)Nc1c[nH]c2ccccc12] | 349.382 | 350.2 | BB |
| 79 | 180 | 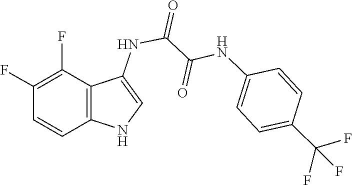 [SMILES: O=C(Nc1ccc(C(F)(F)F)cc1)C(=O)Nc1c[nH]c2ccc(F)c(F)c12] | 383.278 | 382.0 | BD |
| 80 | 181 | 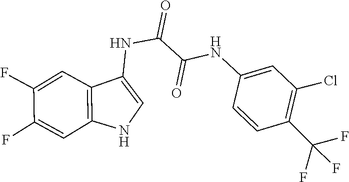 [SMILES: O=C(Nc1ccc(C(F)(F)F)c(Cl)c1)C(=O)Nc1c[nH]c2cc(F)c(F)cc12] | 417.72 | 416.0 | |
| 81 | 182 | 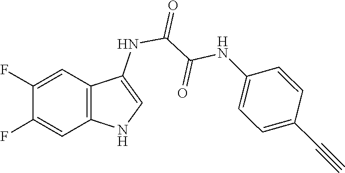 [SMILES: C#Cc1ccc(NC(=O)C(=O)Nc2c[nH]c3cc(F)c(F)cc23)cc1] | 339.302 | 340.0 | |
| 82 | 183 | 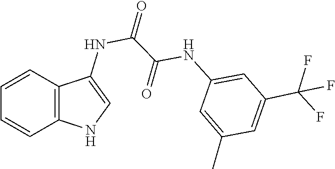 [SMILES: Cc1cc(NC(=O)C(=O)Nc2c[nH]c3ccccc23)cc(C(F)(F)F)c1] | 361.324 | 362.1 | |
| 83 | 184 | 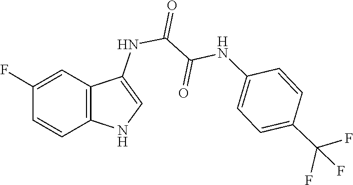 [SMILES: O=C(Nc1ccc(C(F)(F)F)cc1)C(=O)Nc1c[nH]c2ccc(F)cc12] | 365.288 | 364.0 | |
| 84 | 185 | 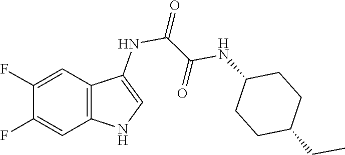 [SMILES: CC[C@H]1CC[C@@H](NC(=O)C(=O)Nc2c[nH]c3cc(F)c(F)cc23)CC1] | 349.382 | 350.1 | |
| 85 | 186 | 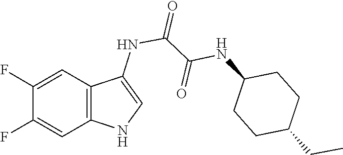 [SMILES: CC[C@H]1CC[C@H](NC(=O)C(=O)Nc2c[nH]c3cc(F)c(F)cc23)CC1] | 349.382 | 350.2 | |
| 86 | 187 | 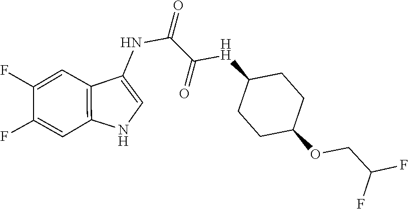 | 401.362 | 402.2 | |
| 87 | 188 | 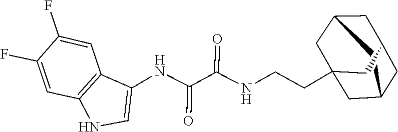 [SMILES: O=C(NCCC12C[C@H]3C[C@@H](C1)C[C@@H](C2)C3)C(=O)Nc1c[nH]c2cc(F)c(F)cc12] | 401.458 | 402.2 | |
| 88 | 189 | 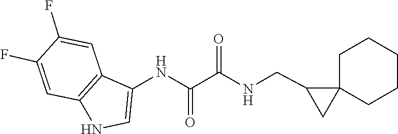 [SMILES: O=C(NCC1CC12CCCCC2)C(=O)Nc1c[nH]c2cc(F)c(F)cc12] | 361.393 | 362.3 | |
| 89 | 190 | 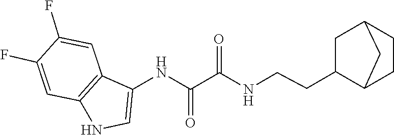 [SMILES: O=C(NCCC1CC2CCC1C2)C(=O)Nc1c[nH]c2cc(F)c(F)cc12] | 361.393 | 362.3 | |
| 90 | 191 | 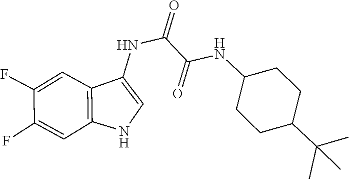 [SMILES: CC(C)(C)C1CCC(NC(=O)C(=O)Nc2c[nH]c3cc(F)c(F)cc23)CC1] | 377.436 | 378.2 | |
| 91 | 122 | 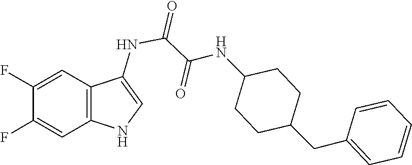 [SMILES: O=C(Nc1c[nH]c2cc(F)c(F)cc12)C(=O)NC1CCC(Cc2ccccc2)CC1] | 411.453 | 412.2 | |
| 92 | 123 | 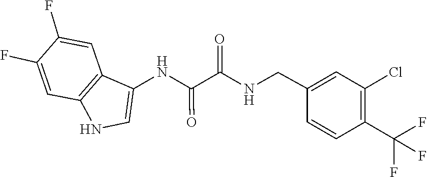 [SMILES: O=C(NCc1ccc(C(F)(F)F)c(Cl)c1)C(=O)Nc1c[nH]c2cc(F)c(F)cc12] | 431.75 | 430.1 | |
| 93 | 118 | 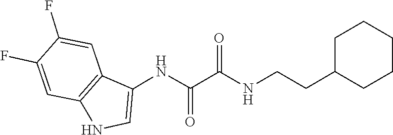 [SMILES: O=C(NCCC1CCCCC1)C(=O)Nc1c[nH]c2cc(F)c(F)cc12] | 349.382 | 348.1 | |

### Example 94: N-(5,6-difluoro-1H-indol-3-yl)-N′-trans-3-((5-methylpyridin-2-yl)oxy)cyclobutyl)oxalamide (Compound 199)

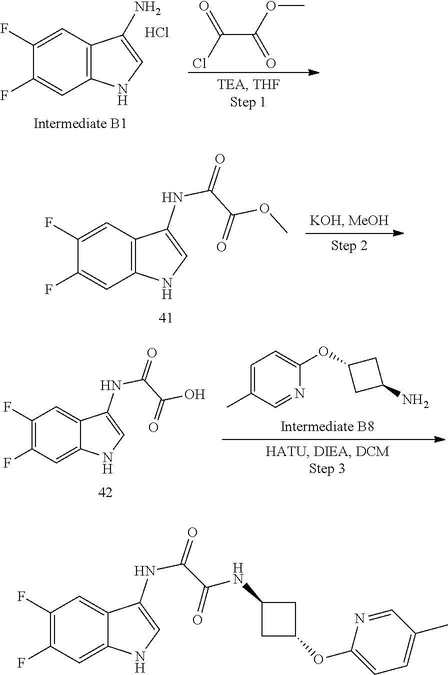

### Step 1: methyl [(5,6-difluoro-1H-indol-3-yl)carbamoyl]formate

5,6-difluoro-1H-indol-3-amine hydrochloride (16.0 g, 95.2 mmol, 1.0 equiv.) and TEA (13.1 mL, 95.2 mmol, 1.0 equiv.) were dissolved in THE (250 mL) and the solution was cooled to 0° C. Then methyl oxalyl chloride (8.6 mL, 95.2 mmol, 1.0 equiv.) was added dropwise, maintaining the solution at 0° C. The reaction mixture was stirred for 2 hours at 0° C. The resulting solids were collected by filtration to give methyl [(5,6-difluoro-1Hindol-3-yl)carbamoyl]formate (16.3 g) as a yellow solid. LCMS Method CB: [M+H]⁺=255.

### Step 2: [(5,6-difluoro-1H-indol-3-yl)carbamoyl]formic acid

Methyl [(5,6-difluoro-1H-indol-3-yl)carbamoyl]formate (5.0 g, 19.6 mmol, 1.0 equiv.) was dissolved in MeOH (60 mL) and water (12 mL), then KOH (2.2 g, 39.4 mmol, 2.0 equiv.) was added. The reaction mixture was stirred for 3 hours at ambient temperature and concentrated under vacuum. The residue was diluted with water, then adjusted to pH 3 with aqueous HCl (6M). The resulting solids were collected by filtration and dried to give [(5,6-difluoro-1H-indol-3-yl)carbamoyl]formic acid (2.5 g) as a yellow solid. LCMS Method CB: [M−H]⁻=239.

### Step 3: N-(5,6-difluoro-1H-indol-3-yl)-N′-[trans-3-[(5-methylpyridin-2-yl)oxy]cyclobutyl]ethanediamide

[(5,6-difluoro-1H-indol-3-yl)carbamoyl]formic acid (200.0 mg, 0.8 mmol, 1.0 equiv.) was dissolved in DCM (20.0 mL), then trans-3-[(5-methylpyridin-2-yl)oxy]cyclobutan-1-amine (148.4 mg, 0.8 mmol, 1.0 equiv.), HATU (474.9 mg, 1.2 mmol, 1.5 equiv.) and DIEA (0.3 mL, 1.7 mmol, 2.0 equiv.) were added. The reaction mixture was stirred for 16 hours at ambient temperature and then quenched by the addition of water. The resulting mixture was extracted with ethyl acetate, washed with brine, dried over anhydrous Na₂SO₄ and concentrated under vacuum. The residue was purified by Prep-HPLC with the following conditions: Column: XBridge Shield RP18 OBD Column, 30*150 mm, 5 m; Mobile Phase A: Water/10 mM NH₄HCO₃+0.1% NH₄OH, Mobile Phase B: Acetonitrile; Flow rate: 60 mL/min; Gradient: 40 B to 65 B in 7 min; 254 nm. This gave N-(5,6-difluoro-1H-indol-3-yl)-N′-[trans-3-[(5-methylpyridin-2-yl)oxy]cyclobutyl]ethanediamide (154.1 mg) as a white solid. LCMS Method CF: [M+H]⁺=401. ¹H NMR (400 MHz, DMSO-d₆) δ 11.17 (s, 1H), 10.67 (s, 1H), 9.35 (d, 1H), 7.96-7.91 (m, 2H), 7.81 (d, 1H), 7.56-7.53 (m, 1H), 7.40-7.36 (m, 1H), 6.74 (d, 1H), 5.30-5.26 (m, 1H), 4.51-4.46 (m, 1H), 2.69-2.61 (m, 2H), 2.42-2.37 (m, 2H), 2.21 (s, 3H).

The following compounds were prepared using the method described for Example 94.

| Example | Compound # | Starting material | Structure | LCMS |
|---|---|---|---|---|
| 95 | 195 | Intermediate B1: 5.6-difluoro-1H-indol-3- amine hydrochloride; Intermediate B6: 3-(4- (trifluoromethyl)phenoxy) piperidine | 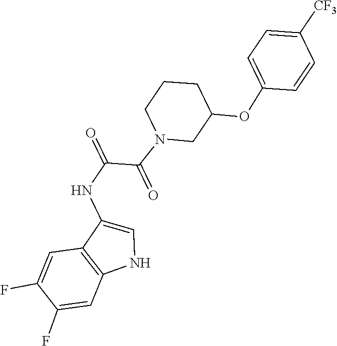 [SMILES: O=C(Nc1c[nH]c2cc(F)c(F)cc12)C(=O)N1CCCC(Oc2ccc(C(F)(F)F)cc2)C1] | Method CH: MS- ESI: 468 [M + H]⁺. |
| 96 | 196 | Intermediate B1: 5,6-difluoro-1H-indol-3- amine hydrochloride; Intermediate B7: trans-3-((6-methylpyridin- 3-yl)oxy)cyclobutan-1- amine | 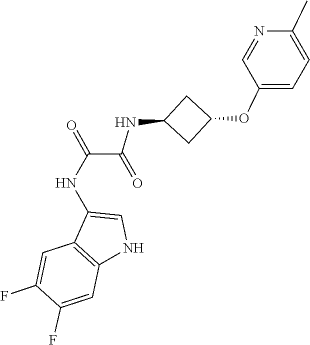 [SMILES: Cc1ccc(O[C@H]2C[C@H](NC(=O)C(=O)Nc3c[nH]c4cc(F)c(F)cc34)C2)cn1] | Method CF: MS- ESI: 401 [M + H]⁺. |
| 97 | 197 | Intermediate B1: 5,6-difluoro-1H-indol-3- amine hydrochloride; Intermediate B25: trans-N-(6- (trifluoromethyl)pyridin-3- yl)cyclobutane-1,3-diamine hydrochloride | 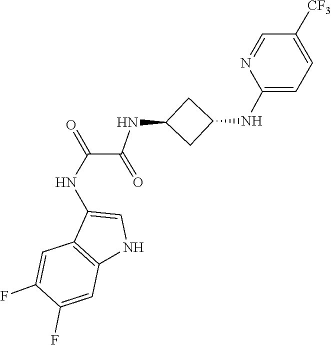 [SMILES: O=C(Nc1c[nH]c2cc(F)c(F)cc12)C(=O)N[C@H]1C[C@H](Nc2ccc(C(F)(F)F)cn2)C1] | Method CH: MS- ESI: 454 [M + H]⁺. |
| 98 | 198 | Intermediate B1: 5,6-difluoro-1H-indol-3- amine hydrochloride; Intermediate B12: cis-3-((6- (trifluoromethyl)pyridin-3- yl)oxy)cyclobutan-1-amine hydrochloride | 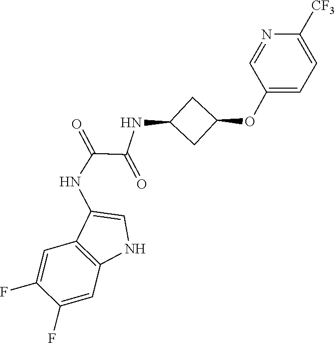 [SMILES: O=C(Nc1c[nH]c2cc(F)c(F)cc12)C(=O)N[C@H]1C[C@@H](Oc2ccc(C(F)(F)F)nc2)C1] | Method CI: MS- ESI: 455 [M + H]⁺. |
| 99 | 200 | Intermediate B1: 5,6-difluoro-1H-indol-3- amine hydrochloride; Intermediate B13: 3-((6- (trifluoromethyl)pyridin-3- yl)oxy)propan-1-amine hydrochloride | 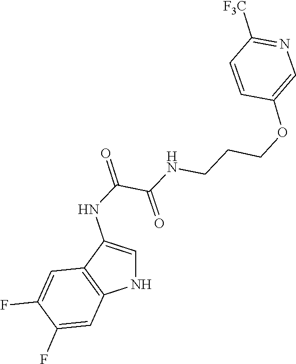 [SMILES: O=C(NCCCOc1ccc(C(F)(F)F)nc1)C(=O)Nc1c[nH]c2cc(F)c(F)cc12] | Method CI: MS- ESI: 443 [M + H]⁺. |
| 100 | 201 | Intermediate B1: 5,6-difluoro-1H-indol-3- amine hydrochloride; Intermediate B14: trans-3-((5- (trifluoromethyl)pyridin-3- yl)oxy)cyclobutan-1-amine hydrochloride | 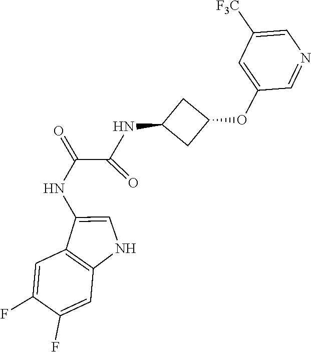 [SMILES: O=C(Nc1c[nH]c2cc(F)c(F)cc12)C(=O)N[C@H]1C[C@H](Oc2cncc(C(F)(F)F)c2)C1] | Method CJ: MS- ESI: 455 [M + H]⁺. |
| 101 | 207 | Intermediate B4: 5-chloro-1H-indol-3-amine hydrochloride; Intermediate B9: trans-4-(pyridin-2- yloxy)cyclohexan-1-amine | 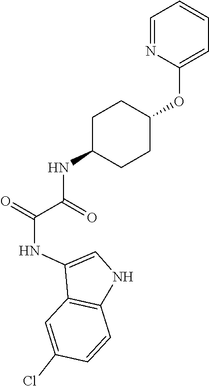 [SMILES: O=C(Nc1c[nH]c2ccc(Cl)cc12)C(=O)N[C@H]1CC[C@H](Oc2ccccn2)CC1] | Method CF: MS- ESI: 413 [M + H]⁺. |
| 102 | 208 | Intermediate B4: 5-chloro-1H-indol-3-amine hydrochloride; Intermediate B27: trans-N-(pyridin-2- yl)cyclobutane-1,3-diamine hydrochloride | 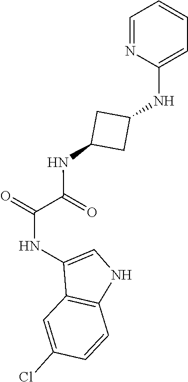 [SMILES: O=C(Nc1c[nH]c2ccc(Cl)cc12)C(=O)N[C@H]1C[C@H](Nc2ccccn2)C1] | Method CF: MS- ESI: 384 [M + H]⁺. |
| 103 | 210 | Intermediate B1: 5,6-difluoro-1H-indol-3- amine hydrochloride; Intermediate B16: trans-3-((5- (trifluoromethyl)pyridin-2- yl)oxy)cyclobutan-1-amine hydrochloride | 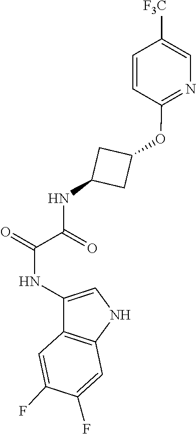 [SMILES: O=C(Nc1c[nH]c2cc(F)c(F)cc12)C(=O)N[C@H]1C[C@H](Oc2ccc(C(F)(F)F)cn2)C1] | Method CF: MS- ESI: 455 [M + H]⁺. |
| 104 | 212 | Intermediate B4: 5-chloro-1H-indol-3-amine hydrochloride; Intermediate B17: trans-3-(pyridin-2- yloxy)cyclobutan-1-amine hydrochloride | 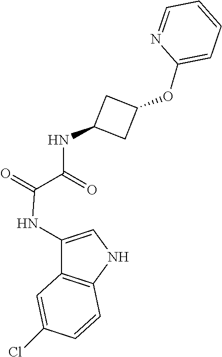 [SMILES: O=C(Nc1c[nH]c2ccc(Cl)cc12)C(=O)N[C@H]1C[C@H](Oc2ccccn2)C1] | Method CH: MS- ESI: 385 [M + H]⁺. |
| 105 | 241 | Intermediate B1: 5,6-difluoro-1H-indol-3- amine hydrochloride; Intermediate B29: 1-(5-(2,2,2- trifluoroethyl)pyridin-2- yl)piperazine hydrochloride | 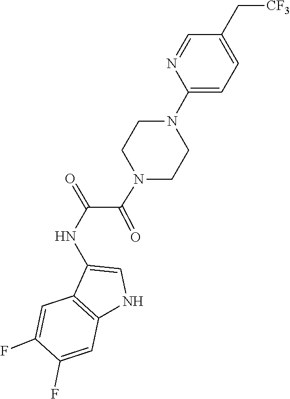 [SMILES: O=C(Nc1c[nH]c2cc(F)c(F)cc12)C(=O)N1CCN(c2ccc(CC(F)(F)F)cn2)CC1] | Method CG: MS- ESI: 468 [M + H]⁺. |
| 106 | 253 | Intermediate B1: 5,6-difluoro-1H-indol-3- amine hydrochloride; Intermediate B31: 1-(4- (trifluoromethyl)benzyl) piperazine hydrochloride | 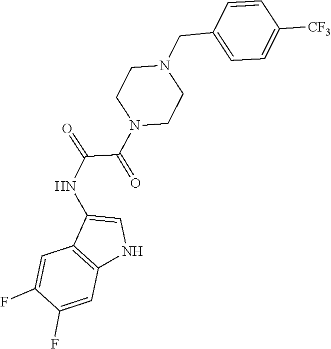 [SMILES: O=C(Nc1c[nH]c2cc(F)c(F)cc12)C(=O)N1CCN(Cc2ccc(C(F)(F)F)cc2)CC1] | Method CH: MS- ESI: 467 [M + H]⁺. |
| 107 | 251 | Intermediate B1: 5,6-difluoro-1H-indol-3- amine hydrochloride; 4-(4- (trifluoromethyl)benzyl) piperidine | 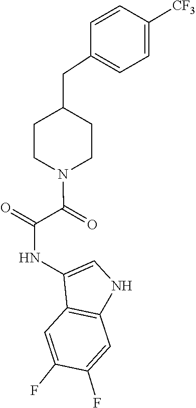 [SMILES: O=C(Nc1c[nH]c2cc(F)c(F)cc12)C(=O)N1CCC(Cc2ccc(C(F)(F)F)cc2)CC1] | Method CJ: MS- ESI: 466 [M + H]⁺. |
| 108 | 317 | Intermediate B1: 5,6-difluoro-1H-indol-3- amine hydrochloride; Intermediate B23: 4-(4- (trifluoromethyl)phenoxy) piperidine hydrochloride | 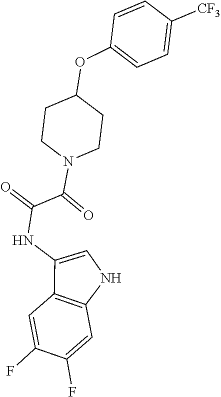 [SMILES: O=C(Nc1c[nH]c2cc(F)c(F)cc12)C(=O)N1CCC(Oc2ccc(C(F)(F)F)cc2)CC1] | Method CE: MS- ESI: 468 [M + H]⁺. |
| 109 | 333 | Intermediate B1: 5,6-difluoro-1H-indol-3- amine hydrochloride; Intermediate B39: (6-(4,4- difluorocyclohexyl)-5- fluoropyridin-3- yl)methanamine hydrochloride | 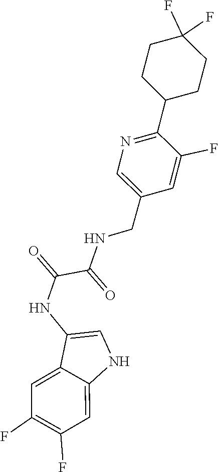 [SMILES: O=C(NCc1cnc(C2CCC(F)(F)CC2)c(F)c1)C(=O)Nc1c[nH]c2cc(F)c(F)cc12] | Method CG: MS- ESI: 467 [M + H]⁺. |
| 110 | 336 | Intermediate B1: 5,6-difluoro-1H-indol-3- amine hydrochloride; Intermediate B38: 1-(3- (trifluoromethyl)benzyl) pyrrolidin-3-amine hydrochloride | 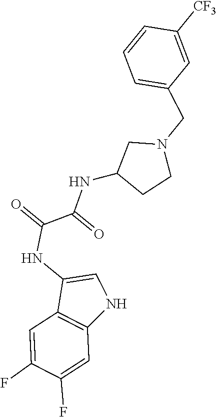 [SMILES: O=C(Nc1c[nH]c2cc(F)c(F)cc12)C(=O)NC1CCN(Cc2cccc(C(F)(F)F)c2)C1] | Method CG: MS- ESI: 467 [M + H]⁺. |
| 111 | 352 | Intermediate B1: 5,6-difluoro-1H-indol-3- amine hydrochloride; Intermediate B44: trans-3-(pyridin-3- yloxy)cyclobutan-1-amine hydrochloride | 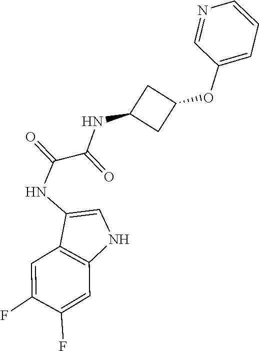 [SMILES: O=C(Nc1c[nH]c2cc(F)c(F)cc12)C(=O)N[C@H]1C[C@H](Oc2cccnc2)C1] | Method CH: MS- ESI: 387 [M + H]⁺. |

### Example 112: N-(5,6-difluoro-1H-indol-3-yl)-N′-(trans-4-(4-(trifluoromethyl)phenoxy)cyclohexyl)oxalamide (Compound 250)

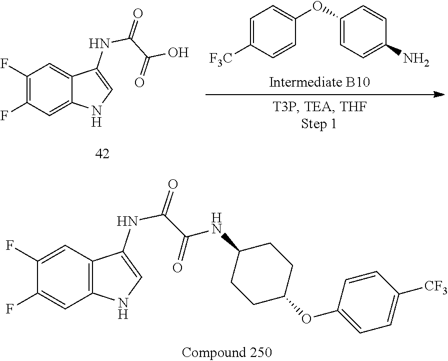

[(5,6-difluoro-1H-indol-3-yl)carbamoyl]formic acid (150.0 mg, 0.6 mmol, 1.0 equiv.) was dissolved in THE (15 mL), then trans-4-[4-(trifluoromethyl)phenoxy]cyclohexan-1-amine (161.9 mg, 0.6 mmol, 1.0 equiv.), T3P (wt. 50% in ethyl acetate, 1.1 mL, 0.9 mmol, 1.5 equiv.) and TEA (0.2 mL, 1.2 mmol, 2.0 equiv.) were added. The reaction mixture was stirred for 16 hours at ambient temperature and then quenched by the addition of water. The resulting mixture was extracted with ethyl acetate, washed with brine, dried over anhydrous Na₂SO₄ and concentrated under vacuum. The residue was purified by Prep-HPLC with the following conditions: Column: XBridge Prep OBD C18 Column, 30*150 mm, 5 m; Mobile Phase A: Water (10 mM NH₄HCO₃), Mobile Phase B: ACN; Flow rate: 50 mL/min; Gradient: 52 B to 72 B in 7 min; 254 nm. This gave N-(5,6-difluoro-1H-indol-3-yl)-N′-[trans-4-[4-(trifluoromethyl)phenoxy]cyclohexyl]ethanediamide (107.4 mg) as a white solid. LCMS Method CG: [M−H]⁻=480.

¹HNMR (400 MHz, DMSO-d₆) δ 11.16 (s, 1H), 10.67 (s, 1H), 8.79 (d, J=7.6 Hz, 1H), 7.95-7.90 (m, 1H), 7.80 (d, J=3.2 Hz, 1H), 7.63 (d, J=8.4 Hz, 2H), 7.40-7.36 (m, 1H), 7.15 (d, J=8.8 Hz, 2H), 4.46-4.40 (m, 1H), 3.79-3.73 (m, 1H), 2.15-2.13 (m, 2H), 1.90-1.86 (m, 2H), 1.66-1.62 (m, 2H), 1.53-1.49 (m, 2H).

The following compounds were prepared using the method described for Example 112.

| Example | Compound # | Starting Material | Structure | LCMS data |
|---|---|---|---|---|
| 113 | 249 | Intermediate B1: 5,6-difluoro-1H-indol- 3-amine hydrochloride; Intermediate B22: cis-4-(4- (trifluoromethyl) phenoxy) cyclohexan-1-amine hydrochloride | 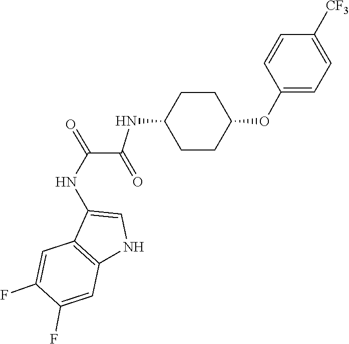 [SMILES: O=C(Nc1c[nH]c2cc(F)c(F)cc12)C(=O)N[C@H]1CC[C@@H](Oc2ccc(C(F)(F)F)cc2)CC1] | Method CG: MS- ESI: 480 [M − H]⁻. |
| 114 | 211 | Intermediate B1: 5,6-difluoro-1H-indol- 3-amine hydrochloride; Intermediate B30: trans-N-(2,2,2- trifluoroethyl) cyclohexane- 1,4-diamine hydrochloride | 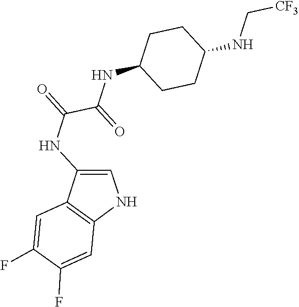 [SMILES: O=C(Nc1c[nH]c2cc(F)c(F)cc12)C(=O)N[C@H]1CC[C@H](NCC(F)(F)F)CC1] | Method CF: MS- ESI: 419 [M + H]⁺. |
| 115 | 216 | Intermediate B1: 5,6-difluoro-1H-indol- 3-amine hydrochloride; Intermediate B32: 1-(4- (trifluoromethyl) phenethyl)piperazine hydrochloride | 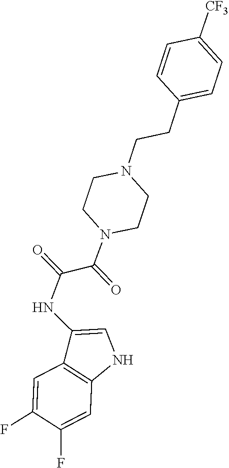 [SMILES: O=C(Nc1c[nH]c2cc(F)c(F)cc12)C(=O)N1CCN(CCc2ccc(C(F)(F)F)cc2)CC1] | Method CF: MS- ESI: 481 [M + H]⁺. |
| 116 | 217 | Intermediate B1: 5,6-difluoro-1H-indol- 3-amine hydrochloride; Intermediate B24: 3-(4-(trifluoromethyl) phenoxy) cyclopentan-1-amine hydrochloride | 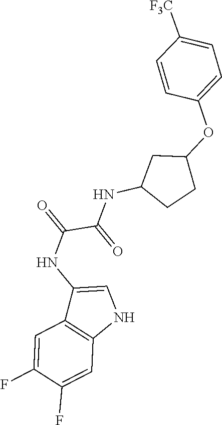 [SMILES: O=C(Nc1c[nH]c2cc(F)c(F)cc12)C(=O)NC1CCC(Oc2ccc(C(F)(F)F)cc2)C1] | Method CH: MS- ESI: 468 [M + H]⁺. |
| 117 | 219 | Intermediate B1: 5,6-difluoro-1H-indol- 3-amine hydrochloride; Intermediate B35: 4-methyl-3,4-dihydro- 2H-pyrido[3.2- b][1,4]oxazin-7-amine hydrochloride | 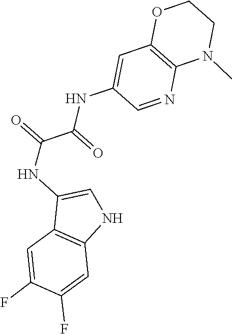 [SMILES: CN1CCOc2cc(NC(=O)C(=O)Nc3c[nH]c4cc(F)c(F)cc34)cnc21] | Method CD: MS- ESI: 388 [M + H]⁺. |
| 118 | 231 | Intermediate B1: 5,6-difluoro-1H-indol- 3-amine hydrochloride; Intermediate B36: 5-amino-2-(pyrrolidin- 1-yl)nicotinonitrile | 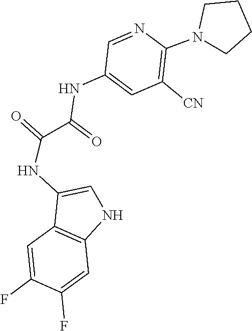 [SMILES: N#Cc1cc(NC(=O)C(=O)Nc2c[nH]c3cc(F)c(F)cc23)cnc1N1CCCC1] | Method CD: MS- ESI: 411 [M + H]⁺. |
| 119 | 326 | Intermediate B1: 5,6-difluoro-1H-indol- 3-amine hydrochloride; Intermediate 37: (6-(piperidin-1- yl)pyridin-2- yl)methanamine hydrochloride | 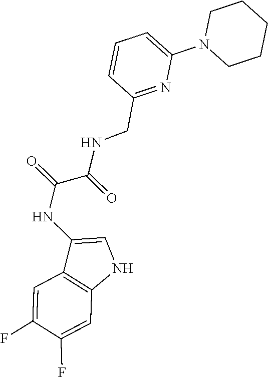 [SMILES: O=C(NCc1cccc(N2CCCCC2)n1)C(=O)Nc1c[nH]c2cc(F)c(F)cc12] | Method CG: MS- ESI: 414 [M + H]⁺. |

### Example 120: N-(5,6-difluoro-1H-indol-3-yl)-N′-[trans-4-(pyridin-2-ylamino)cyclohexyl]ethanediamide (Compound 202)

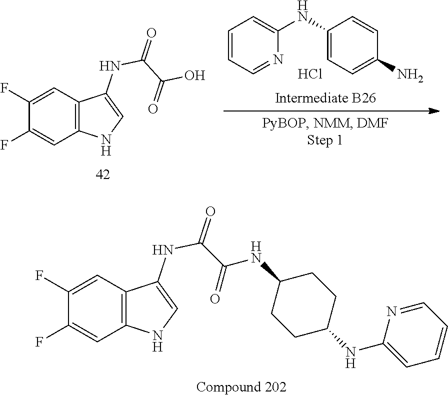

[(5,6-Difluoro-1H-indol-3-yl)carbamoyl]formic acid (376.6 mg, 1.6 mmol, 1.0 equiv.) was dissolved in DMF (15 mL), then trans-N-(pyridin-2-yl)cyclohexane-1,4-diamine hydrochloride (236.5 mg, 1.5 mmol, 1.0 equiv.), PyBOP (816.1 mg, 1.5 mmol, 1.0 equiv.) and NMM (951.8 mg, 9.4 mmol, 6.0 equiv.) were added. The reaction mixture was stirred for 4 hours at ambient temperature and then quenched by the addition of water. The resulting mixture was extracted with ethyl acetate, dried over anhydrous Na₂SO₄ and concentrated under vacuum. The residue was purified by reverse flash chromatography with the following conditions: column, C18; Mobile Phase A: Water (0.1% NH₄HCO₃), Mobile Phase B: ACN, 0% B increasing to 100% gradient in 15 min. The resulting crude product was further purified by Prep-HPLC with the following conditions: Column: XBridge Shield RP18 OBD Column, 30*150 mm, 5 m; Mobile Phase A: Water (10 mM NH₄HCO₃+0.1% NH₄OH), Mobile Phase B: ACN; Flow rate: 60 mL/min; Gradient: 35 B to 55 B in 7 min; 254 nm. This gave N-(5,6-difluoro-1H-indol-3-yl)-N′-[trans-4-(pyridin-2-ylamino)cyclohexyl]ethanediamide (12.6 mg) as a pink solid. LCMS Method CF: [M+H]⁺=414. ¹H NMR (400 MHz, DMSO-d₆) δ 11.17 (s, 1H), 10.66 (s, 1H), 8.75 (d, 1H), 7.95-7.90 (m, 2H), 7.81 (s, 1H), 7.41-7.31 (m, 2H), 6.44-6.41 (m, 2H), 6.32 (d, 1H), 3.75-3.65 (m, 2H), 2.02-2.00 (m, 2H), 1.88-1.80 (m, 2H), 1.62-1.53 (m, 2H), 1.32-1.24 (m, 2H).

The following compounds were prepared using the method described for Example 120.

| Example | Compound # | Starting Material | Structure | LCMS Data |
|---|---|---|---|---|
| 121 | 203 | Intermediate B1: 5,6-difluoro-1H-indol- 3-amine hydrochloride; Intermediate B33: 2-amino-1-(2,3- dihydro-1H- pyrrolo[2,3-b]pyridin- 1-yl)ethan-1-one hydrochloride | 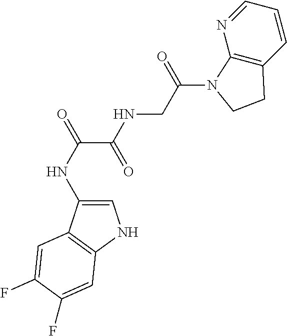 [SMILES: O=C(NCC(=O)N1CCc2cccnc21)C(=O)Nc1c[nH]c2cc(F)c(F)cc12] | Method CI: MS-ESI: 400 [M + H]⁺. |
| 122 | 204 | Intermediate B1: 5,6-difluoro-1H-indol- 3-amine hydrochloride Intermediate B34: 6-(trifluoromethyl) indoline hydrochloride; | 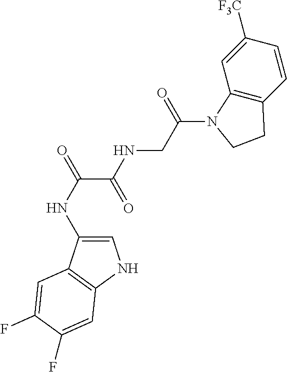 [SMILES: O=C(NCC(=O)N1CCc2ccc(C(F)(F)F)cc21)C(=O)Nc1c[nH]c2cc(F)c(F)cc12] | Method CH: MS-ESI: 467 [M + H]⁺. |
| 123 | 321 | Intermediate B5: (3-chloro-4- (trifluoromethyl) phenyl)methanamine hydrochloride; (3-chloro-4- (trifluoromethyl)phenyl) methanamine | 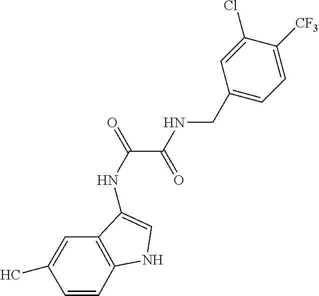 | Method CE: MS-ESI: 421 [M + H]⁺. |
| 124 | 353 | Intermediate B4: 5-chloro-1H-indol-3- amine hydrochloride; Intermediate B8: trans-3-((5- methylpyridin-2- yl)oxy)cyclobutan-1- amine | 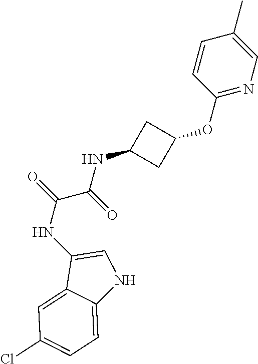 [SMILES: Cc1ccc(O[C@H]2C[C@H](NC(=O)C(=O)Nc3c[nH]c4ccc(Cl)cc34)C2)nc1] | Method CF: MS-ESI: 399 [M + H]⁺. |
| 125 | 354 | Intermediate B42: 1H-indol-3-amine hydrochloride; Intermediate B8: trans-3-((5- methylpyridin-2- yl)oxy)cyclobutan-1- amine | 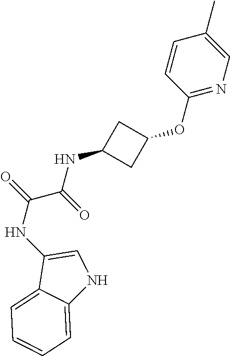 [SMILES: Cc1ccc(O[C@H]2C[C@H](NC(=O)C(=O)Nc3c[nH]c4ccccc34)C2)nc1] | Method CF: MS-ESI: 365 [M + H]⁺. |
| 126 | 355 | Intermediate B4: 5-chloro-1H-indol-3- amine hydrochloride; Intermediate B43: 6-(trans-4- aminocyclohexyl)oxy) nicotinonitrile hydrochloride | 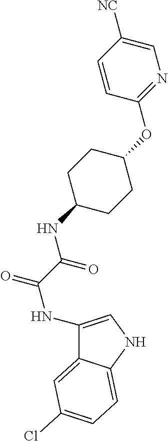 [SMILES: N#Cc1ccc(O[C@H]2CC[C@H](NC(=O)C(=O)Nc3c[nH]c4ccc(Cl)cc34)CC2)nc1] | Method CF: MS-ESI: 438 [M + H]⁺. |

### Example 127: N-(5-chloro-6-fluoro-1H-indol-3-yl)-N′-(trans-4-((5-(trifluoromethyl)pyridin-2-yl)oxy)cyclohexyl)oxalamide (Compound 209)

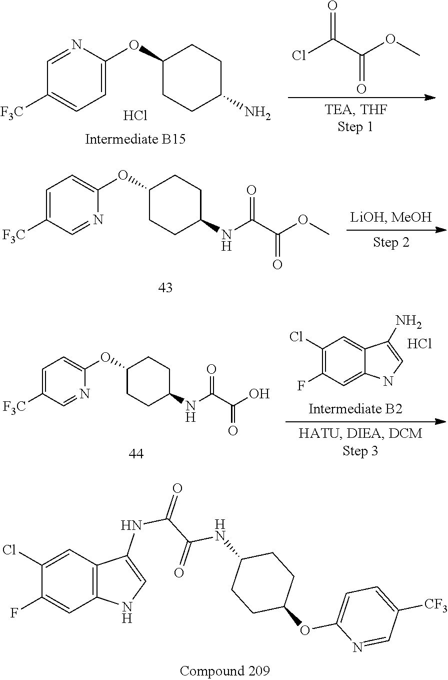

### Step 1: methyl [[trans-4-[[5-(trifluoromethyl)pyridin-2-yl]oxy]cyclohexyl]carbamoyl]formate

trans-4-[[5-(trifluoromethyl)pyridin-2-yl]oxy]cyclohexan-1-amine hydrochloride (550.0 mg, 2.1 mmol, 1.0 equiv.) and TEA (0.6 mL, 4.2 mmol, 2.0 equiv.) were dissolved in THE (20 mL) and cooled to 0° C., then methyl oxalyl chloride (0.2 mL, 2.1 mmol, 1.0 equiv.) was added dropwise, maintaining the solution at 0° C. The reaction mixture was stirred for 2 hours at ambient temperature and quenched by the addition of water. The resulting solution was extracted with ethyl acetate, washed with brine, dried over anhydrous Na₂SO₄ and concentrated under vacuum to give methyl [[trans-4-[[5-(trifluoromethyl)pyridin-2-yl]oxy]cyclohexyl]carbamoyl]formate (650.0 mg) as a white solid. LCMS Method CA: [M+H]⁺=347.

### Step 2: [[trans-4-[[5-(trifluoromethyl)pyridin-2-yl]oxy]cyclohexyl]carbamoyl]formic acid

Methyl [[trans-4-[[5-(trifluoromethyl)pyridin-2-yl]oxy]cyclohexyl]carbamoyl]formate (550.0 mg, 1.5 mmol, 1.0 equiv.) was dissolved in MeOH (15 mL) and water (5 mL), then LiGH (76.0 mg, 3.1 mmol, 2.00 equiv.) was added. The reaction mixture was stirred for 2 hours at ambient temperature and concentrated under vacuum. The residue was diluted with water, then adjusted to pH 3 with aqueous HCl (2N). The resulting solution was extracted with ethyl acetate, washed with brine, dried over anhydrous Na₂SO₄ and concentrated under vacuum to give [[trans-4-[[5-(trifluoromethyl)pyridin-2-yl]oxy]cyclohexyl]carbamoyl]formic acid (312.5 mg) as a white solid. LCMS Method CB: [M+H]⁺=332.

### Step 3: N-(5-chloro-6-fluoro-1H-indol-3-yl)-N′-[trans-4-[[5-(trifluoromethyl)pyridin-2-yl]oxy]cyclohexyl]ethanediamide

[[trans-4-[[5-(trifluoromethyl)pyridin-2-yl]oxy]cyclohexyl]carbamoyl]formic acid (200.0 mg, 0.6 mmol, 1.0 equiv.) was dissolved in DCM (20 mL), then DIEA (0.3 mL, 1.8 mmol, 3.0 equiv.), HATU (343.0 mg, 0.9 mmol, 1.5 equiv.) and N-(5-chloro-6-fluoro-1H-indol-3-yl)chloranamine hydrochloride (131.0 mg, 0.6 mmol, 1.0 equiv.) were added. The reaction mixture was stirred for 2 hours at ambient temperature and quenched by the addition of water. The resulting solution was extracted with ethyl acetate, washed with brine, dried over anhydrous Na₂SO₄ and concentrated under vacuum. The residue was purified by Prep-HPLC with following conditions: Column: XBridge Prep OBD C18 Column, 30×150 mm, 5 m; Mobile Phase A: Water (10 mM NH₄HCO₃+0.1% NH₄OH, Mobile Phase B: ACN; Flow rate: 60 mL/min; Gradient: 55 B to 85 B in 7 min; 220 nm. This gave N-(5-chloro-6-fluoro-1H-indol-3-yl)-N′-[trans-4-[[5-(trifluoromethyl)pyridin-2-yl]oxy]cyclohexyl]ethanediamide (87.1 mg) as a white solid. LCMS Method CI: [M+H]⁺=499. ¹H NMR (400 MHz, DMSO-d₆) δ 11.24 (brs, 1H), 10.76 (brs, 1H), 8.79 (d, 1H), 8.60 (s, 1H), 8.15 (d, 1H), 8.07-8.04 (m, 1H), 7.79 (s, 1H), 7.40-7.37 (m, 1H), 6.98 (d, 1H), 5.06-5.00 (m, 1H), 3.82-3.73 (m, 1H), 2.16-2.09 (m, 2H), 1.88-1.85 (m, 2H), 1.69-1.52 (m, 4H).

### Example 128: N-(5-chloro-1H-indol-3-yl)-N′-(trans-4-(4-(trifluoromethyl)phenoxy)cyclohexyl)oxalamide (Compound 213)

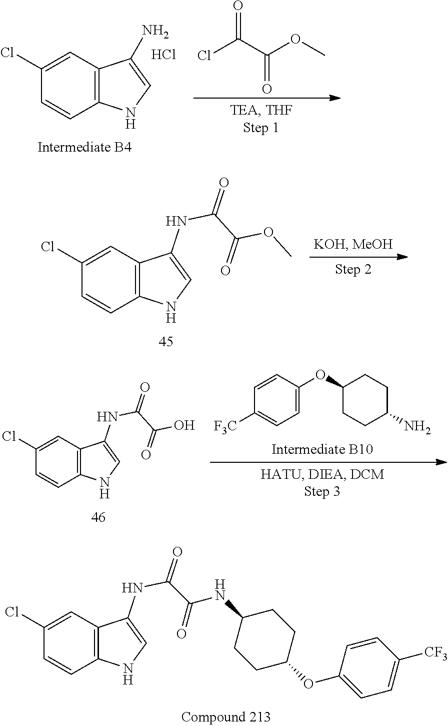

### Step 1: methyl [(5-chloro-1H-indol-3-yl)carbamoyl]formate

5-Chloro-1H-indol-3-amine hydrochloride (9.0 g, 44.3 mmol, 1.0 equiv.) and TEA (7.2 mL, 53.1 mmol, 1.2 equiv.) were dissolved in THE (100 mL) and cooled to 0° C., then methyl oxalyl chloride (4.0 mL, 44.3 mmol, 1.0 equiv.) was added dropwise, maintaining the solution at 0° C. The reaction mixture was stirred for 2 hours at ambient temperature and concentrated under vacuum. The residue was slurried in ethyl acetate (50 mL) for 1 hour at ambient temperature and the solids were collected by filtration to give methyl [(5-chloro-1H-indol-3-yl)carbamoyl]formate (6.5 g) as a light brown solid. LCMS Method CB: [M+H]⁺=253.

### Step 2: [(5-chloro-1H-indol-3-yl)carbamoyl]formic acid

Methyl [(5-chloro-1H-indol-3-yl)carbamoyl]formate (6.5 g, 25.7 mmol, 1.0 equiv.) was dissolved in MeOH (50 mL) and water (10 mL), then KOH (2.1 g, 38.5 mmol, 1.5 equiv.) was added in portions. The resulting mixture was stirred for 2 hours at ambient temperature and concentrated under vacuum. The residue was diluted with of water, then adjusted to pH 3 with aqueous HCl (2M). The solids were collected by filtration and dried to give [(5-chloro-1H-indol-3-yl)carbamoyl]formic acid (5.1 g) as a light brown solid. LCMS Method CB: [M−H]⁻=237.

### Step 3: N-(5-chloro-1H-indol-3-yl)-N′-[trans-4-[4-(trifluoromethyl)phenoxy]cyclohexyl]ethanediamide

[(5-chloro-1H-indol-3-yl)carbamoyl]formic acid (5.2 g, 21.7 mmol, 1.0 equiv.) was dissolved in DMF (100 mL), then trans-4-[4-(trifluoromethyl)phenoxy]cyclohexan-1-amine (5.6 g, 21.7 mmol, 1.0 equiv.), NMM (13.2 g, 130.7 mmol, 6.0 equiv.) and PyBOP (13.6 g, 26.1 mmol, 1.2 equiv.) were added. The reaction mixture was stirred for 2 hours at ambient temperature. The resulting solution was poured into water (1.5 L) and stirred for 30 min. The solids were collected by filtration and purified by flash column chromatography on silica gel, eluting with ethyl acetate/petroleum ether (1:1). The resulting solid was slurried with ethyl acetate for 16 hours at ambient temperature. After filtration, the resulting solid was further slurried with ACN for 2 hours at 80° C. then filtered o give N-(5-chloro-1H-indol-3-yl)-N′-[trans-4-[4-(trifluoromethyl)phenoxy]cyclohexyl]ethanediamide (4.3 g) as a pale pink solid. LCMS Method CF: [M+H]⁺=480. ¹H NMR (400 MHz, DMSO-d₆) δ 11.22 (brs, 1H), 10.65 (brs, 1H), 8.81 (d, 1H), 7.96 (d, 1H), 7.78 (s, 1H), 7.64-7.61 (m, 2H), 7.40-7.38 (m, 1H), 7.16-7.09 (m, 3H), 4.45-4.38 (m, 1H), 3.80-3.72 (m, 1H), 2.15-2.12 (m, 2H), 1.89-1.86 (m, 2H), 1.69-1.63 (m, 2H), 1.60-1.53 (m, 2H).

### Example 129: N-(5,6-difluoro-1H-indol-3-yl)-N′-[trans-4-[[6-(trifluoromethyl)pyridin-3-yl]oxy]cyclohexyl]ethanediamide (Compound 223)

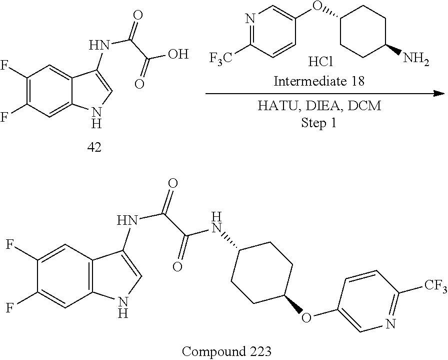 [SMILES: O=C(O)C(=O)Nc1c[nH]c2cc(F)c(F)cc12 ; N[C@H]1CC[C@H](Oc2ccc(C(F)(F)F)nc2)CC1 ; Cl ; O=C(Nc1c[nH]c2cc(F)c(F)cc12)C(=O)N[C@H]1CC[C@H](Oc2ccc(C(F)(F)F)nc2)CC1]

[(5,6-difluoro-1H-indol-3-yl)carbamoyl]formic acid (184.5 mg, 0.7 mmol, 1.0 equiv.) was dissolved in DCM (20 mL), then DIEA (0.4 mL, 2.3 mmol, 3.0 equiv.), HATU (438.2 mg, 1.1 mmol, 1.5 equiv.) and trans-4-[[6-(trifluoromethyl)pyridin-3-yl]oxy]cyclohexan-1-amine hydrochloride (200.0 mg, 0.7 mmol, 1.0 equiv.) were added. The reaction mixture was stirred for 2 hours at ambient temperature, then concentrated under vacuum. The residue was diluted with water, extracted with ethyl acetate, washed with brine, dried over anhydrous Na₂SO₄ and concentrated under vacuum. The residue was purified by Chiral-Prep-HPLC with the following conditions: Column: XBridge Prep OBD C18 Column, 30*150 mm 5 m; Mobile Phase A: Water (10 mM NH₄HCO₃), Mobile Phase B: ACN; Flow rate: 50 mL/min; Gradient: 40 B to 75 B in 7 min; 254 nm. This gave N-(5,6-difluoro-1H-indol-3-yl)-N′-[trans-4-[[6-(trifluoromethyl)pyridin-3-yl]oxy]cyclohexyl]ethanediamide (111.0 mg) as a white solid. LCMS Method CD: [M+H]⁺=483. ¹H NMR (400 MHz, DMSO-d₆) δ 11.17 (d, 1H), 10.70 (s, 1H), 8.85 (d, 1H), 8.44 (d, 1H), 7.96-7.91 (m, 1H), 7.81 (d, 2H), 7.70-7.67 (m, 1H), 7.40-7.36 (m, 1H), 4.53-4.51 (m, 1H), 3.77-3.75 (m, 1H), 2.16-2.13 (m, 2H), 1.88-1.85 (m, 2H), 1.67-1.52 (m, 4H).

The following compound was prepared using the method described for Example 129.

| | | | LCMS |
|---|---|---|---|
| Example | Starting material | Structure | data |
| 130 (Compound 222) | Intermediate B1: 5,6-difluoro-1H-indol-3-amine hydrochloride; Intermediate B19: cis-4-[[6- (trifluoromethyl)pyridin-3- yl]oxy]cyclohexan-1-amine hydrochloride | 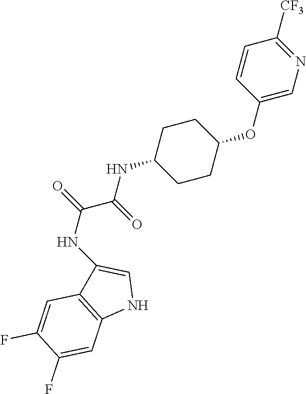 [SMILES: O=C(Nc1c[nH]c2cc(F)c(F)cc12)C(=O)N[C@H]1CC[C@@H](Oc2ccc(C(F)(F)F)nc2)CC1] | Method CD: MS- ESI: 483 [M + H]⁺. |

### Example 131: N-(5,6-difluoro-1H-indol-3-yl)-N′-[trans-4-[[6-(trifluoromethyl)pyridin-3-yl]oxy]cyclohexyl]ethanediamide (Compound 230)

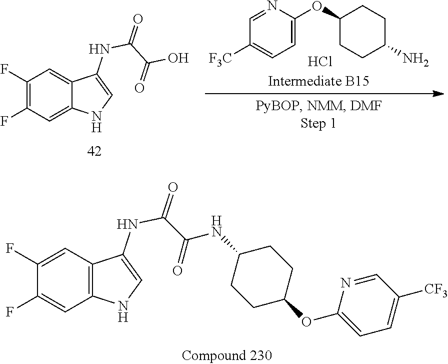 [SMILES: O=C(O)C(=O)Nc1c[nH]c2cc(F)c(F)cc12 ; N[C@H]1CC[C@H](Oc2ccc(C(F)(F)F)cn2)CC1 ; Cl ; O=C(Nc1c[nH]c2cc(F)c(F)cc12)C(=O)N[C@H]1CC[C@H](Oc2ccc(C(F)(F)F)cn2)CC1]

[(5,6-difluoro-1H-indol-3-yl)carbamoyl]formic acid (4.0 g, 16.6 mmol, 1.0 equiv.) was dissolved in DMF (60 mL), trans-4-[[5-(trifluoromethyl)pyridin-2-yl]oxy]cyclohexan-1-amine hydrochloride (4.9 g, 16.6 mmol, 1.0 equiv.), PyBOP (10.4 g, 19.9 mmol, 1.2 equiv.) and NMM (10.1 g, 99.9 mmol, 6.0 equiv.) were added. The reaction mixture was stirred for 2 hours at ambient temperature, then poured into water (1.5 L) and stirred for 30 min. The solids were collected by filtration and purified by flash column chromatography on silica gel, eluting with ethyl acetate/petroleum ether (1:1). The resulting material was further purified by Prep-achiral-SFC with the following conditions: Column: GreenSep Basic, 30*150 mm 5 μm; Mobile Phase A: CO₂, Mobile Phase B: MeOH (0.1% 2M NH₃-MeOH); Flow rate: 60 mL/min; Gradient: holding 30% B 10 min; 254 nm. This gave N-(5,6-difluoro-1H-indol-3-yl)-N′-[trans-4-[[5-(trifluoromethyl)pyridin-2-yl]oxy]cyclohexyl]ethanediamide (3.5 g) as an off-white solid. LCMS Method CF: [M+H]⁺=483.

¹HNMR (400 MHz, DMSO-d₆) δ 11.17 (s, 1H), 10.69 (s, 1H), 8.78 (d, J=8.4 Hz, 1H), 8.60 (s, 1H), 8.07-8.04 (m, 1H), 7.96-7.90 (m, 1H), 7.80 (d, J=2.4 Hz, 1H), 7.40-7.36 (m, 1H), 6.98 (d, J=8.8 Hz, 1H), 5.06-5.00 (m, 1H), 3.81-3.74 (m, 1H), 2.16-2.13 (m, 2H), 1.89-1.85 (m, 2H), 1.66-1.54 (m, 4H).

The following compound was prepared using the method described for Example 131.

| | | | LCMS |
|---|---|---|---|
| Example | Starting material | Structure | data |
| 132 (Compound 229) | Intermediate B1: 5,6-difluoro-1H-indol- 3-amine hydrochloride; Intermediate B21: cis-4-((5- (trifluoromethyl)pyridin- 2-yl)oxy)cyclohexan- 1-amine hydrochloride | 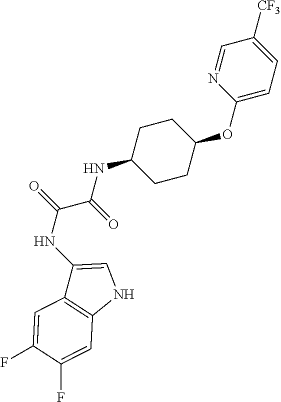 [SMILES: O=C(Nc1c[nH]c2cc(F)c(F)cc12)C(=O)N[C@H]1CC[C@@H](Oc2ccc(C(F)(F)F)cn2)CC1] | Method CD: MS- ESI: 483 [M + H]⁺. |

### Example 133: (S)-N-(5,6-difluoro-1H-indol-3-yl)-N′-(1-(5-(trifluoromethyl)pyridin-2-yl)pyrrolidin-3-yl)oxalamide (Compound 264)

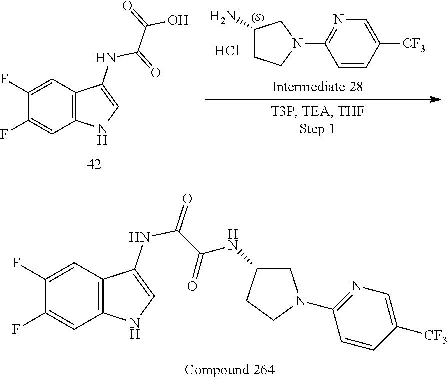

[(5,6-Difluoro-1H-indol-3-yl)carbamoyl]formic acid (207.7 mg, 0.8 mmol, 1.0 equiv.) was dissolved in THE (30 mL), (S)-1-(5-(trifluoromethyl)pyridin-2-yl)pyrrolidin-3-amine hydrochloride (200.0 mg, 0.8 mmol, 1.0 equiv.), TEA (0.4 mL, 2.6 mmol, 3.0 equiv.), T3P (wt. 50% in ethyl acetate, 1.3 mL, 1.3 mmol, 1.5 equiv.) were added. The resulting solution was stirred for 3 hours at ambient temperature and then quenched by the addition of water. The resulting solution was extracted with ethyl acetate, dried over anhydrous Na₂SO₄ and concentrated under vacuum. The residue was purified by flash column chromatography on silica gel, eluting with ethyl acetate/petroleum ether (1:1) to give material that was further purified by Prep-HPLC with the following conditions: Column: XBridge Prep OBD C18 Column, 30*150 mm 5 m; Mobile Phase A: Water (0.05% NH₄OH), Mobile Phase B: ACN; Flow rate: 50 mL/min; Gradient: 35 B to 70 B in 11 min; 254 nm. This gave (S)-N-(5,6-difluoro-1H-indol-3-yl)-N′-(1-(5-(trifluoromethyl)pyridin-2-yl)pyrrolidin-3-yl)oxalamide (43.7 mg) as a white solid. LCMS Method CD: [M+H]⁺=454. ¹H NMR (400 MHz, DMSO-d₆) δ 11.17 (s, 1H), 10.74 (s, 1H), 9.26 (d, 1H), 8.40 (s, 1H), 7.98-7.90 (m, 1H), 7.81-7.78 (m, 2H), 7.40-7.36 (m, 1H), 6.59 (d, 1H), 4.57-4.52 (m, 1H), 3.81-3.65 (m, 2H), 3.51-3.46 (m, 2H), 2.25-2.14 (m, 2H).

### Example 134: N-(6-fluoro-1H-indol-3-yl)-N′-(4-((5-(trifluoromethyl)pyridin-2-yl)oxy)cyclohexyl)oxalamide (Compound 225)

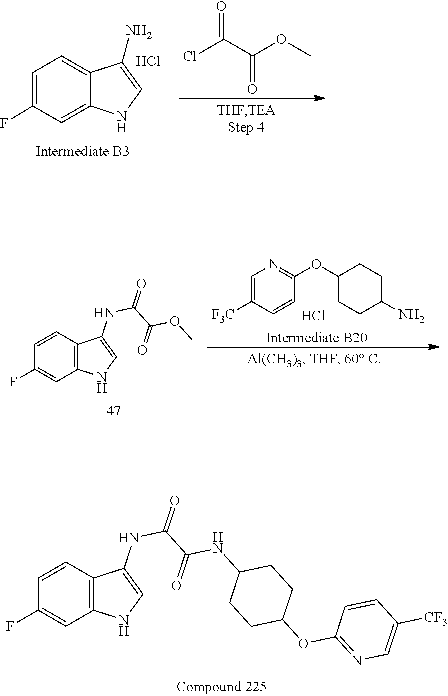

### Step 1: methyl [(6-fluoro-1H-indol-3-yl)carbamoyl]formate

6-Fluoro-1H-indol-3-amine hydrochloride (434.2 mg, 2.3 mmol, 1.0 equiv.) and TEA (0.3 mL, 2.3 mmol, 1.0 equiv.) were dissolved in THF (20 mL) and cooled to 0° C., then methyl oxalyl chloride (0.2 mL, 2.3 mmol, 1.0 equiv.) was added dropwise, maintaining the solution at 0° C. The reaction mixture was stirred for 2 hours at ambient temperature and then quenched by the addition of MeOH. The reaction mixture was concentrated under vacuum, diluted with water, extracted with ethyl acetate, dried over anhydrous Na₂SO₄ and concentrated under vacuum. The residue was purified by flash column chromatography on silica gel, eluting with ethyl acetate/petroleum ether (1:1) to give methyl [(6-fluoro-1H-indol-3-yl)carbamoyl]formate (361.5 mg) as a pale yellow solid. LCMS Method CD: [M+H]⁺=237.

### Step 2: N-(6-fluoro-1H-indol-3-yl)-N′-(4-[[5-(trifluoromethyl)pyridin-2-yl]oxy]cyclohexyl)ethanediamide

Methyl [(6-fluoro-1H-indol-3-yl)carbamoyl]formate (130.0 mg, 0.5 mmol, 1.0 equiv.), 4-[[5-(trifluoromethyl)pyridin-2-yl]oxy]cyclohexan-1-amine hydrochloride (166.4 mg, 0.5 mmol, 1.0 equiv.) were dissolved in THE (10 mL) and cooled to 0° C., then trimethylaluminium (2 N in toluene, 0.8 mL, 1.6 mmol, 3.0 equiv.) was added dropwise, maintaining the solution at 0° C. The reaction mixture was heated to 60° C. for 1 hour, then cooled to ambient temperature and concentrated under vacuum. The residue was purified by Pre-HPLC with the following conditions: Column: XBridge Prep OBD C18 Column, 30*150 mm 5 m; Mobile Phase A: Water (0.05% FA), Mobile Phase B: ACN; Flow rate: 50 mL/min; Gradient: 35 B to 60 B in 7 min; 254 nm. This gave N-(6-fluoro-1H-indol-3-yl)-N′-(4-[[5-(trifluoromethyl)pyridin-2-yl]oxy]cyclohexyl)ethanediamide (75.6 mg) as an off-white solid. LCMS Method CD: [M+H]⁺=465. ¹H NMR (400 MHz, DMSO-d₆) δ 11.08 (s, 1H), 10.60 (s, 1H), 8.81 (d, 1H), 8.59 (s, 1H), 8.07-8.04 (m, 1H), 7.87-7.83 (m, 1H), 7.72 (d, 1H), 7.16-7.13 (m, 1H), 6.98 (d, 1H), 6.89-6.84 (m, 1H), 5.06-5.00 (m, 1H), 3.81-3.73 (m, 1H), 2.15-2.13 (m, 2H), 1.88-1.85 (m, 2H), 1.69-1.60 (m, 4H).
Example 135: N-(5,6-difluoro-1H-indol-3-yl)-N′-((1R,3S)-3-(4-(trifluoromethyl)phenoxy)cyclohexyl)oxalamide, peak one, absolute stereochemistry unconfirmed] (Compound 220),
Example 136: N-(5,6-difluoro-1H-indol-3-yl)-N′-((1S,3R)-3-(4-(trifluoromethyl)phenoxy)cyclohexyl)oxalamide, peak two, absolute stereochemistry unconfirmed] (Compound 356),
Example 137: N-(5,6-difluoro-1H-indol-3-yl)-N′-((1R,3R)-3-(4-(trifluoromethyl)phenoxy)cyclohexyl)oxalamide, peak three, absolute stereochemistry unconfirmed] (Compound 228) and
Example 138: N-(5,6-difluoro-1H-indol-3-yl)-N′-((1S,3S)-3-(4-(trifluoromethyl)phenoxy)cyclohexyl)oxalamide, peak four, absolute stereochemistry unconfirmed] (Compound 227)

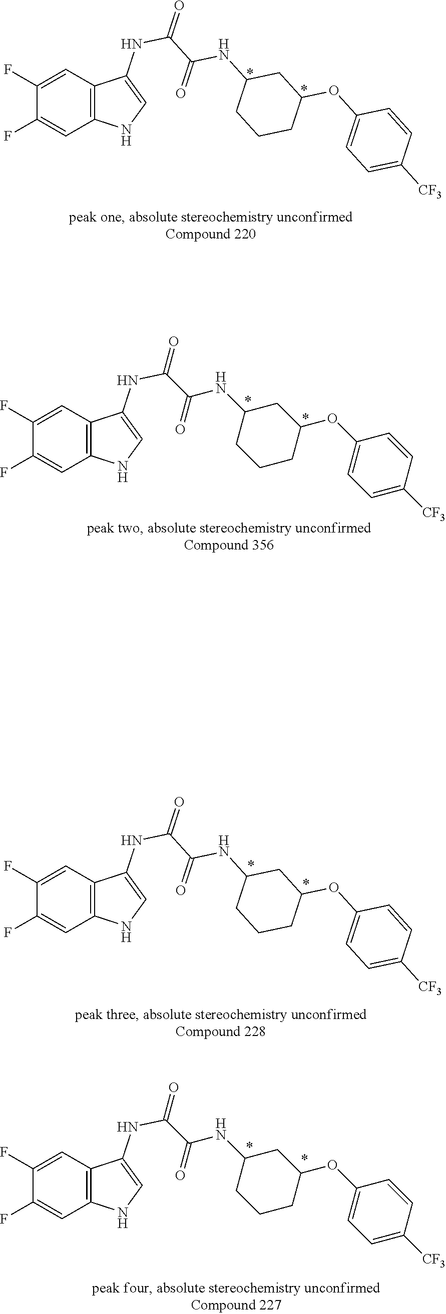 [SMILES: O=C(Nc1c[nH]c2cc(F)c(F)cc12)C(=O)NC1CCCC(Oc2ccc(C(F)(F)F)cc2)C1 ; O=C(Nc1c[nH]c2cc(F)c(F)cc12)C(=O)NC1CCCC(Oc2ccc(C(F)(F)F)cc2)C1 ; O=C(Nc1c[nH]c2cc(F)c(F)cc12)C(=O)NC1CCCC(Oc2ccc(C(F)(F)F)cc2)C1 ; O=C(Nc1c[nH]c2cc(F)c(F)cc12)C(=O)NC1CCCC(Oc2ccc(C(F)(F)F)cc2)C1]

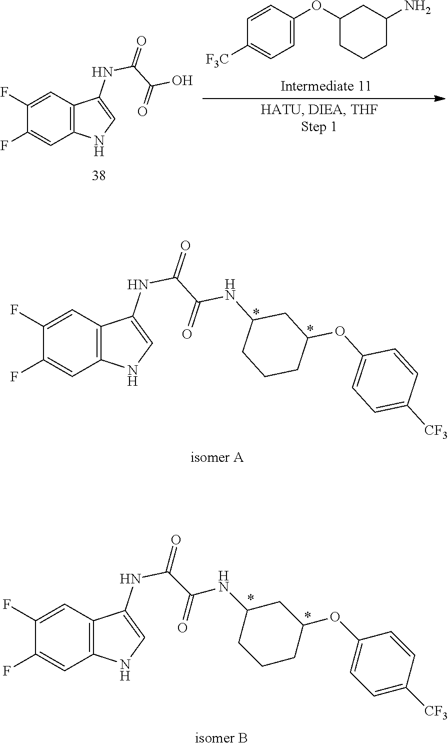 [SMILES: O=C(O)C(=O)Nc1c[nH]c2cc(F)c(F)cc12 ; NC1CCCC(Oc2ccc(C(F)(F)F)cc2)C1 ; O=C(Nc1c[nH]c2cc(F)c(F)cc12)C(=O)NC1CCCC(Oc2ccc(C(F)(F)F)cc2)C1 ; O=C(Nc1c[nH]c2cc(F)c(F)cc12)C(=O)NC1CCCC(Oc2ccc(C(F)(F)F)cc2)C1]

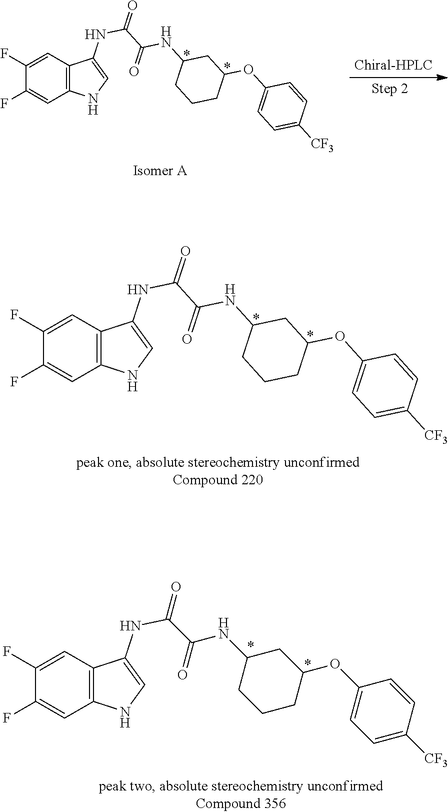 [SMILES: O=C(Nc1c[nH]c2cc(F)c(F)cc12)C(=O)NC1CCCC(Oc2ccc(C(F)(F)F)cc2)C1 ; O=C(Nc1c[nH]c2cc(F)c(F)cc12)C(=O)NC1CCCC(Oc2ccc(C(F)(F)F)cc2)C1 ; O=C(Nc1c[nH]c2cc(F)c(F)cc12)C(=O)NC1CCCC(Oc2ccc(C(F)(F)F)cc2)C1]

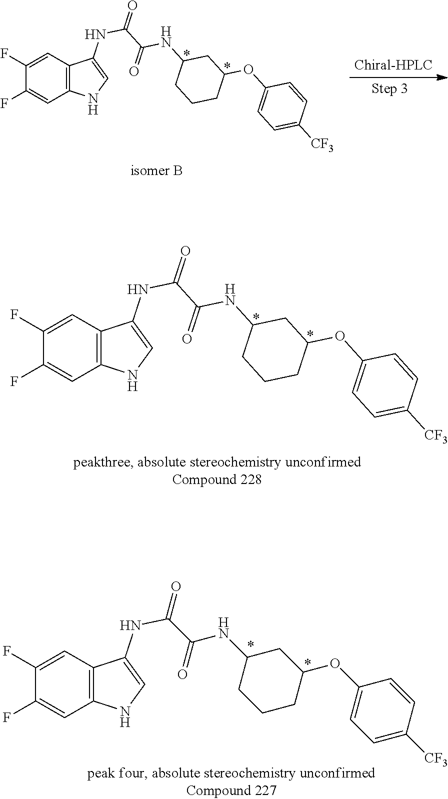 [SMILES: O=C(Nc1c[nH]c2cc(F)c(F)cc12)C(=O)NC1CCCC(Oc2ccc(C(F)(F)F)cc2)C1 ; O=C(Nc1c[nH]c2cc(F)c(F)cc12)C(=O)NC1CCCC(Oc2ccc(C(F)(F)F)cc2)C1 ; O=C(Nc1c[nH]c2cc(F)c(F)cc12)C(=O)NC1CCCC(Oc2ccc(C(F)(F)F)cc2)C1]

### Step 1: Isomer A and Isomer B

[(5,6-Difluoro-1H-indol-3-yl)carbamoyl]formic acid (463.1 mg, 1.9 mmol, 1.0 equiv.) was dissolved in THE (30 mL), then 3-[4-(trifluoromethyl)phenoxy]cyclohexan-1-amine (500.0 mg, 1.9 mmol, 1.0 equiv.), HATU (1.4 g, 3.8 mmol, 2.0 equiv.) and DIEA (0.6 mL, 3.8 mmol, 2.0 equiv.) were added. The resulting mixture was stirred overnight at ambient temperature and then quenched by the addition of water. The resulting mixture was extracted with ethyl acetate, dried over anhydrous Na₂SO₄ and concentrated under vacuum. The residue was purified by flash column chromatography on silica gel, eluting with ethyl acetate/petroleum ether (1:1) to give material that was further purified via prep-HPLC with the following conditions: Column: XBridge Prep OBD C18 Column, 30*150 mm 5 m; Mobile Phase A: Water (10 mM NH₄HCO₃), Mobile Phase B: ACN; Flow rate: 80 mL/min; Gradient: 70 B to 70 B in 19 min; 254 nm. This gave isomer A (front peak, 90 mg) as a white solid and isomer B (second peak, 150 mg). LCMS method CA: [M+H]⁺=482.

### Step 2: Compound 220 and Compound 356

Isomer A (90 mg, 0.2 mmol, 1.0 equiv.) was separated by Pre-Chiral-HPLC with the following conditions: Column: CHIRALPAK IG, 20*250 mm, 5 m; Mobile Phase A: Hex (0.5% 2M NH₃-MeOH)-HPLC, Mobile Phase B: EtOH-HPLC; Flow rate: 16 mL/min; Gradient: 50 B to 50 B in 15 min; 220/254 nm; RT1: 7.214; RT2: 12.204. This gave Compound 220 (peak one, 15.9 mg) as an off-white solid and Compound 356 (peak two, 14.6 mg).

Compound 220: LCMS Method CD: [M+H]⁺=482. ¹H NMR (400 MHz, DMSO-d₆) δ 11.18 (s, 1H), 10.70 (s, 1H), 8.92 (d, 1H), 7.95-7.90 (m, 1H), 7.80 (d, 1H), 7.64 (d, 2H), 7.40-7.35 (m, 1H), 7.16 (d, 2H), 4.64-4.59 (m, 1H), 3.96-3.90 (m, 1H), 2.18-2.15 (m, 1H), 2.06-2.03 (m, 1H), 2.78-2.75 (m, 2H), 1.48-1.39 (m, 2H), 1.35-1.25 (m, 2H).

Compound 356: LCMS Method CD: [M+H]⁺=482. ¹H NMR (400 MHz, DMSO-d₆) δ 11.18 (s, 1H), 10.70 (s, 1H), 8.92 (d, 1H), 7.95-7.90 (m, 1H), 7.80 (d, 1H), 7.64 (d, 2H), 7.40-7.35 (m, 1H), 7.16 (d, 2H), 4.64-4.59 (m, 1H), 3.96-3.90 (m, 1H), 2.18-2.15 (m, 1H), 2.06-2.03 (m, 1H), 2.78-2.75 (m, 2H), 1.48-1.39 (m, 2H), 1.35-1.25 (m, 2H).

### Step 3: Compound 228 and Compound 227

Isomer B (150 mg, 0.3 mmol, 1.0 equiv.) was separated by Pre-Chiral-HPLC with the following conditions: Column: Chiralpak IA, 2*25 cm, 5 m; Mobile Phase A: Hex (0.5% 2M NH₃-MeOH)-HPLC, Mobile Phase B: EtOH-HPLC; Flow rate: 20 mL/min; Gradient: 50 B to 50 B in 20 min; 220/254 nm; RT1: 11.595; RT2: 16.289. This gave Compound 228 (peak three, 30.6 mg) as a white solid and Compound 227 (peak four, 37.7 mg).

Compound 228: LCMS Method CD: [M+H]⁺=482. ¹H NMR (400 MHz, DMSO-d₆) δ 11.16 (brs, 1H), 10.63 (s, 1H), 8.82 (d, 1H), 7.93-7.88 (m, 1H), 7.79 (d, 1H), 7.65 (d, 2H), 7.39-7.35 (m, 1H), 7.17 (d, 2H), 4.97-4.93 (m, 1H), 4.10-4.06 (m, 1H), 2.04-2.01 (m, 1H), 1.87-1.78 (m, 3H), 1.70-1.51 (m, 4H).

Compound 227: LCMS Method CD: [M+H]⁺=482. ¹H NMR (400 MHz, DMSO-d₆) δ 11.16 (brs, 1H), 10.64 (s, 1H), 8.82 (d, 1H), 7.93-7.88 (m, 1H), 7.79 (d, 1H), 7.65 (d, 2H), 7.39-7.35 (m, 1H), 7.17 (d, 2H), 4.97-4.93 (m, 1H), 4.10-4.05 (m, 1H), 2.04-2.01 (m, 1H), 1.87-1.78 (m, 3H), 1.74-1.52 (m, 4H).

### Example 139-140: (First Peak)-N-(5,6-difluoro-1H-indol-3-yl)-N′-(2,2,2-trifluoro-1-phenylethyl)oxalamide (Compound 240) and (Second Peak)-N-(5,6-difluoro-1H-indol-3-yl)-N′-(2,2,2-trifluoro-1-phenylethyl)oxalamide (Compound 239)

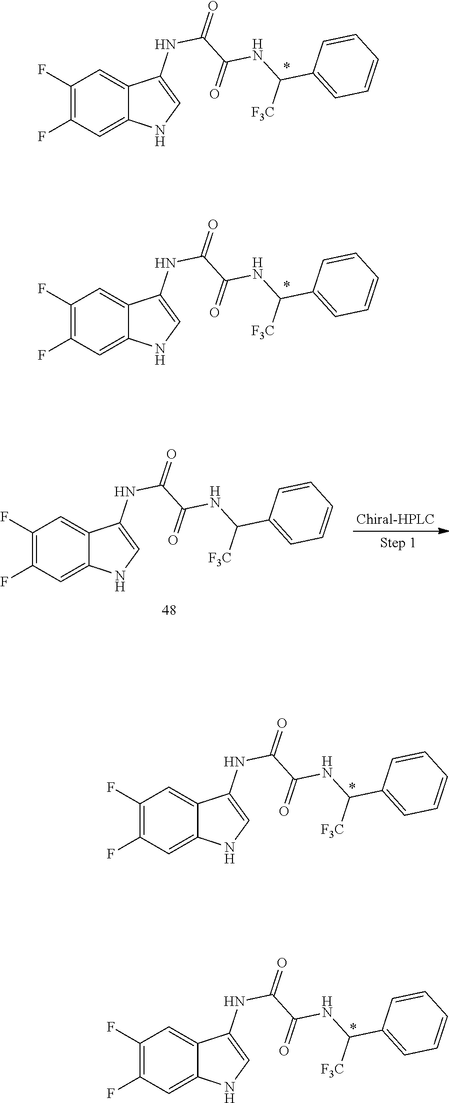 [SMILES: O=C(Nc1c[nH]c2cc(F)c(F)cc12)C(=O)NC(c1ccccc1)C(F)(F)F ; O=C(Nc1c[nH]c2cc(F)c(F)cc12)C(=O)NC(c1ccccc1)C(F)(F)F ; O=C(Nc1c[nH]c2cc(F)c(F)cc12)C(=O)NC(c1ccccc1)C(F)(F)F ; O=C(Nc1c[nH]c2cc(F)c(F)cc12)C(=O)NC(c1ccccc1)C(F)(F)F ; O=C(Nc1c[nH]c2cc(F)c(F)cc12)C(=O)NC(c1ccccc1)C(F)(F)F]

Compound 48 was prepared using the same method described for Example 94 with Intermediate B1 (5,6-difluoro-1H-indol-3-amine hydrochloride) and 2,2,2-trifluoro-1-phenylethan-1-amine.

The racemic N-(5,6-difluoro-1H-indol-3-yl)-N′-(2,2,2-trifluoro-1-phenylethyl)ethanediamide (18.9 mg) was separated by Prep-Chrial-HPLC with the following conditions: Column: CHIRALPAK IA, 2*25 cm, 5 m; Mobile Phase A: Hex(0.5% 2M NH₃-MeOH)-HPLC, Mobile Phase B: EtOH-HPLC; Flow rate: 20 mL/min; Gradient: 25 B to 25 B in 14 min; 220/254 nm; RT1: 7.205, RT2: 11.682. This gave Compound 240 (peak 1, 6.3 mg) as a white solid and Compound 239 (peak 2, 4.9 mg) as a white solid.

Compound 240: LCMS Method CG: [M−H]⁻=396. ¹H NMR (400 MHz, DMSO-d₆): δ 11.20 (s, 1H), 10.86 (s, 1H), 10.08 (d, 1H), 7.96-7.92 (m, 1H), 7.83 (d, 1H), 7.77-7.74 (m, 2H), 7.46-7.44 (m, 2H), 7.41-7.38 (m, 2H), 5.90-5.86 (m, 1H).

Compound 239: LCMS Method CG: [M−H]⁻=396. ¹H NMR (400 MHz, DMSO-d₆): δ 11.19 (s, 1H), 10.86 (s, 1H), 10.06 (d, 1H), 7.96-7.91 (m, 1H), 7.83 (d, 1H), 7.76-7.74 (m, 2H), 7.47-7.44 (m, 2H), 7.41-7.36 (m, 2H), 5.93-5.88 (m, 1H).

The following compounds were obtained from Compound 195 (Example 95) using the method described for Examples 139-140, above.

| Example | Racemate | Structure | LCMS/Chiral data |
|---|---|---|---|
| 141 (Compound 357) | 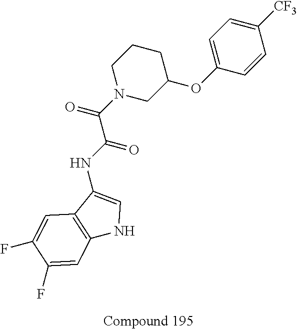 [SMILES: O=C(Nc1c[nH]c2cc(F)c(F)cc12)C(=O)N1CCCC(Oc2ccc(C(F)(F)F)cc2)C1] | 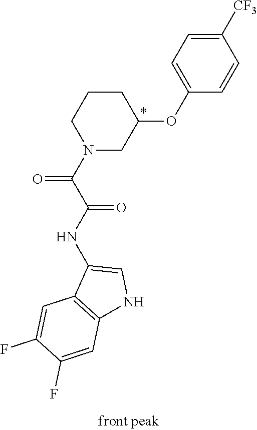 [SMILES: O=C(Nc1c[nH]c2cc(F)c(F)cc12)C(=O)N1CCCC(Oc2ccc(C(F)(F)F)cc2)C1] | Method CH: MS-ESI: 468 [M + H]⁺. Column: CHIRALPAK IG, 2 * 25 cm, 5 μm; Mobile Phase A: Hex(0.2% DEA)--HPLC, Mobile Phase B: IPA-- HPLC; Flow rate: 20 mL/min; Gradient: 15 B to 15 B in 16 min; 220/254 nm; RT1: 11.554 |
| 142 (Compound 358) | 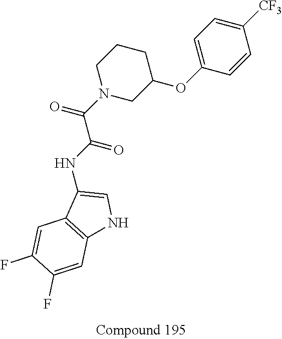 [SMILES: O=C(Nc1c[nH]c2cc(F)c(F)cc12)C(=O)N1CCCC(Oc2ccc(C(F)(F)F)cc2)C1] | 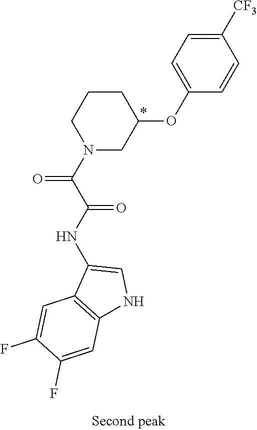 [SMILES: O=C(Nc1c[nH]c2cc(F)c(F)cc12)C(=O)N1CCCC(Oc2ccc(C(F)(F)F)cc2)C1] | Method CH: MS-ESI: 468 [M + H]⁺. Column: CHIRALPAK IG, 2 * 25 cm, 5 μm; Mobile Phase A: Hex(0.2% DEA)--HPLC, Mobile Phase B: IPA-- HPLC; Flow rate: 20 mL/min; Gradient: 15 B to 15 B in 16 min; 220/254 nm; RT2: 14.52 |

### Example 143: N-(3-chloro-4-(trifluoromethyl)benzyl)-N′-(5-(3-(hydroxymethyl)phenyl)-1H-indol-3-yl)oxalamide (Compound 349)

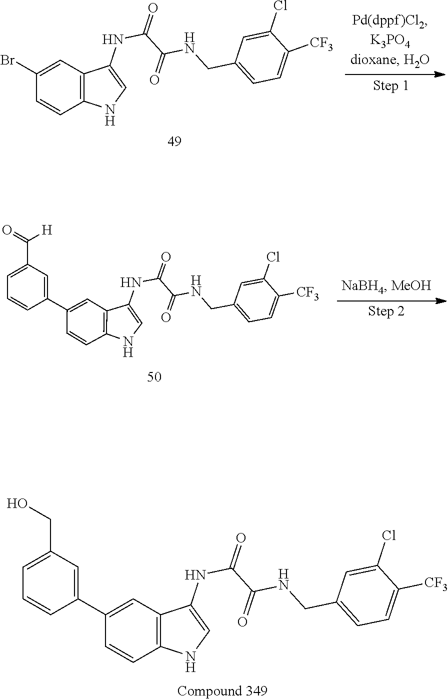 [SMILES: O=C(NCc1ccc(C(F)(F)F)c(Cl)c1)C(=O)Nc1c[nH]c2ccc(Br)cc12 ; O=Cc1cccc(-c2ccc3[nH]cc(NC(=O)C(=O)NCc4ccc(C(F)(F)F)c(Cl)c4)c3c2)c1 ; O=C(NCc1ccc(C(F)(F)F)c(Cl)c1)C(=O)Nc1c[nH]c2ccc(-c3cccc(CO)c3)cc12]

Compound 49 was prepared using the same method for Example 94 with Intermediate B41 (5-bromo-1H-indol-3-amine hydrochloride) and (3-chloro-4-(trifluoromethyl)phenyl)methanamine.

### Step 1: N-[[3-chloro-4-(trifluoromethyl)phenyl]methyl]-N′-[5-(3-formylphenyl)-1H-indol-3-yl]ethanediamide

N-(5-bromo-1H-indol-3-yl)-N′-(3-chloro-4-(trifluoromethyl)benzyl)oxalamide (150.0 mg, 0.3 mmol, 1.0 equiv.) was dissolved in dioxane (4 mL) and water (1 mL), then 3-(4,4,5,5-tetramethyl-1,3,2-dioxaborolan-2-yl)benzaldehyde (146.6 mg, 0.6 mmol, 2.0 equiv.), K₃PO₄ (134.1 mg, 0.6 mmol, 2.0 equiv.) and Pd(dppf)Cl₂ (23.1 mg, 0.03 mmol, 0.1 equiv.) were added under an atmosphere of nitrogen. The reaction mixture was stirred for 4 hours at ambient temperature and then concentrated under vacuum. The residue was purified by flash column chromatography on silica gel, eluting with ethyl acetate/petroleum ether (1:3) to give N-[[3-chloro-4-(trifluoromethyl)phenyl]methyl]-N′-[5-(3-formylphenyl)-1H-indol-3-yl]ethanediamide (105.2 mg) as a pale yellow solid. LCMS Method CC: [M+H]⁺=500.

### Step 2: N-[[3-chloro-4-(trifluoromethyl)phenyl]methyl]-N′-[5-[3-(hydroxymethyl)phenyl]-1H-indol-3-yl]ethanediamideas

N-[[3-chloro-4-(trifluoromethyl)phenyl]methyl]-N′-[5-(3-formylphenyl)-1H-indol-3-yl]ethanediamide (100.0 mg, 0.2 mmol, 1.0 equiv.) was dissolved in MeOH (20 mL), then NaBH₄ (11.4 mg, 0.3 mmol, 1.5 equiv.) was added. The reaction mixture was heated to 60° C. for 2 hours, then cooled to ambient temperature and quenched by the addition of water. The resulting solution was extracted with ethyl acetate, dried over anhydrous Na₂SO₄ and concentrated under vacuum. The residue was purified by flash column chromatography on silica gel, eluting with ethyl acetate/petroleum ether (1:3) to give material that was further purified by Prep-HPLC using the following conditions: Column, XBridge Shield RP18 OBD Column, 5 m, 19*150 mm; mobile phase A, Water (10 mM NH₄HCO₃+0.1% NH₄OH), mobile phase B, ACN (42% Phase B up to 72% in 7 min); Detector, uv 254 nm. This gave N-[[3-chloro-4-(trifluoromethyl)phenyl]methyl]-N′-[5-[3-(hydroxymethyl)phenyl]-1H-indol-3-yl]ethanediamideas (17.5 mg) as an off-white solid. LCMS Method CD: [M+H]⁺=502. ¹H NMR (400 MHz, DMSO-d₆) δ 11.12 (s, 1H), 10.76 (s, 1H), 9.66-9.64 (m, 1H), 8.27 (s, 1H), 7.86-7.82 (m, 2H), 7.68-7.64 (m, 2H), 7.56-7.50 (m, 2H), 7.44-7.39 (m, 3H), 7.25 (d, 1H), 5.22 (t, 1H), 4.58 (d, 2H), 4.52-4.49 (m, 2H).

### Example 144: N-(3-chloro-4-(trifluoromethyl)benzyl)-N′-(5-(2-hydroxyethyl)-1H-indol-3-yl)oxalamide (Compound 339

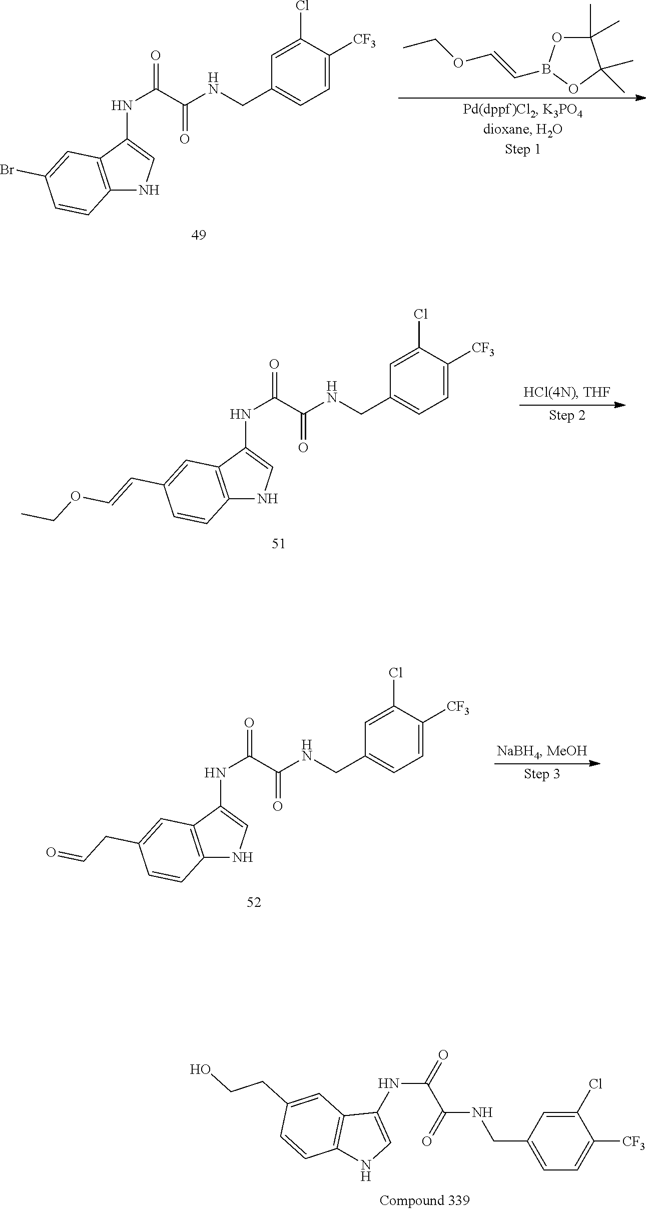

### Step 1: N-[[3-chloro-4-(trifluoromethyl)phenyl]methyl]-N′-[5-[(E)-2-ethoxyethenyl]-1H-indol-3-yl]ethanediamide

N-(5-bromo-1H-indol-3-yl)-N′-(3-chloro-4-(trifluoromethyl)benzyl)oxalamide (300.0 mg, 0.6 mmol, 1.0 equiv.) was dissolved in dioxane (5 mL) and water (1 mL), then 2-[(E)-2-ethoxyethenyl]-4,4,5,5-tetramethyl-1,3,2-dioxaborolane (250.3 mg, 1.2 mmol, 2.0 equiv.), K₃PO₄ (268.3 mg, 1.2 mmol, 2.0 equiv.) and Pd(dppf)Cl₂ (46.2 mg, 0.06 mmol, 0.1 equiv.) were added under an atmosphere of nitrogen. The reaction mixture was heated to 90° C. for 3 hours, then cooled to ambient temperature and concentrated under vacuum. The residue was purified by flash column chromatography on silica gel, eluting with ethyl acetate/petroleum ether (1:3) to give N-[[3-chloro-4-(trifluoromethyl)phenyl]methyl]-N′-[5-[(E)-2-ethoxyethenyl]-1H-indol-3-yl]ethanediamide (240.0 mg) as a pale yellow solid. LCMS Method CA: [M+H]⁺=466.

### Step 2: N-[[3-chloro-4-(trifluoromethyl)phenyl]methyl]-N′-[5-(2-oxoethyl)-1H-indol-3-yl]ethanediamide

N-[[3-chloro-4-(trifluoromethyl)phenyl]methyl]-N′-[5-[(E)-2-ethoxyethenyl]-1H-indol-3-yl]ethanediamide (270.0 mg, 0.6 mmol, 1.0 equiv.) was dissolved in THE (20 mL), then HCl/1,4-dioxane (4N, 2 mL) was added. The reaction mixture was stirred for 30 min at ambient temperature and then quenched by the addition of water. The resulting solution was extracted with ethyl acetate, dried over anhydrous Na₂SO₄ and concentrated under vacuum to give N-[[3-chloro-4-(trifluoromethyl)phenyl]methyl]-N′-[5-(2-oxoethyl)-1H-indol-3-yl]ethanediamide (230.1 mg) as a pale yellow solid. LCMS Method CA: [M+H]⁺=438.

### Step 3: N-[[3-chloro-4-(trifluoromethyl)phenyl]methyl]-N′-[5-(2-hydroxyethyl)-1H-indol-3-yl]ethanediamide

N-[[3-chloro-4-(trifluoromethyl)phenyl]methyl]-N′-[5-(2-oxoethyl)-1H-indol-3-yl]ethanediamide (180.0 mg, 0.4 mmol, 1.0 equiv.) was dissolved in MeOH (15 mL), then NaBH₄ (31.1 mg, 0.8 mmol, 2.0 equiv.) was added. The reaction mixture was stirred for 2 hours at ambient temperature and then quenched by the addition of water. The resulting solution was extracted with ethyl acetate, dried over anhydrous Na₂SO₄ and concentrated under vacuum. The residue was purified by Prep-HPLC with the following conditions: Column: XBridge Shield RP18 OBD Column, 5 m, 19*150 mm; Mobile Phase A: Water (10 mM NH₄HCO₃+0.1% NH₄OH), Mobile Phase B: ACN; Flow rate: 60 mL/min; Gradient: 60 B to 75 B in 7 min; 210/254 nm. This gave N-[[3-chloro-4-(trifluoromethyl)phenyl]methyl]-N′-[5-(2-hydroxyethyl)-1H-indol-3-yl]ethanediamide (15.9 mg) as a white solid. LCMS Method CH: [M+H]⁺=440. ¹HNMR (400 MHz, DMSO-d₆) δ 10.90 (s, 1H), 10.46 (s, 1H), 9.62 (t, 1H), 7.85 (d, 1H), 7.69-7.64 (m, 3H), 7.49 (d, 1H), 7.26 (d, 1H), 6.98 (d, 1H), 4.60 (t, 1H), 4.48 (d, 2H), 3.63-3.58 (m, 2H), 2.78 (t, 2H).

### Example 145: N-(6-fluoro-5-(1-isopropyl-1H-pyrazol-4-yl)-1H-indol-3-yl)-N′-(4-(trifluoromethyl)phenyl)oxalamide (Compound 341)

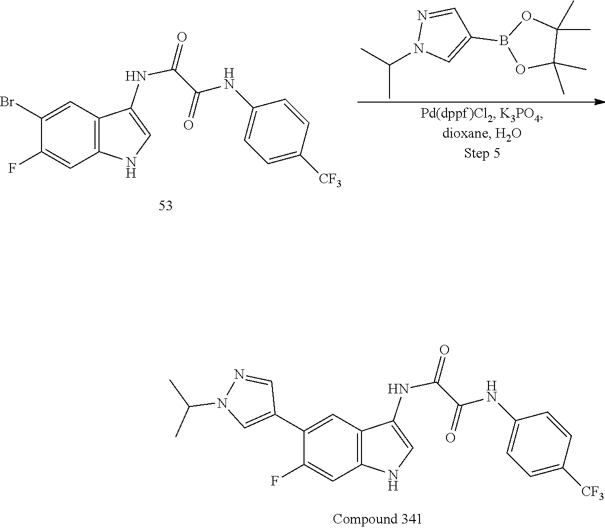 [SMILES: O=C(Nc1ccc(C(F)(F)F)cc1)C(=O)Nc1c[nH]c2cc(F)c(Br)cc12 ; CC(C)n1cc(B2OC(C)(C)C(C)(C)O2)cn1 ; CC(C)n1cc(-c2cc3c(NC(=O)C(=O)Nc4ccc(C(F)(F)F)cc4)c[nH]c3cc2F)cn1]

Compound 53 was prepared using the method described for Example 112 with Intermediate B40 (5-bromo-6-fluoro-1H-indol-3-amine hydrochloride) and 4-(trifluoromethyl)aniline.

N-(5-bromo-6-fluoro-1H-indol-3-yl)-N′-[4-(trifluoromethyl)phenyl]ethanediamide (580.0 mg, 1.3 mmol, 1.0 equiv.) was dissolved in dioxane (10 mL) and water (1 mL), then K₃PO₄ (554.3 mg, 2.6 mmol, 2.0 equiv.), Pd(dppf)Cl₂ (95.5 mg, 0.1 mmol, 0.1 equiv.) and 1-isopropyl-4-(4,4,5,5-tetramethyl-1,3,2-dioxaborolan-2-yl)pyrazole (616.6 mg, 2.6 mmol, 2.0 equiv.) were added under an atmosphere of nitrogen. The reaction mixture was heated to 80° C. for 2 hours, then cooled to ambient temperature and quenched by the addition of water. The resulting solution was extracted with ethyl acetate, dried over anhydrous Na₂SO₄ and concentrated under vacuum. The residue was purified by Prep-HPLC with the following conditions: Column: YMC-Actus Triart C18, 30*250, 5 m; Mobile Phase A: Water (10 mM NH₄HCO₃+0.1% NH₄OH), Mobile Phase B: ACN; Flow rate: 60 mL/min; Gradient: 67 B to 78 B in 10 min; 254/210 nm. This gave N-[6-fluoro-5-(1-isopropylpyrazol-4-yl)-1H-indol-3-yl]-N′-[4-(trifluoromethyl)phenyl]ethanediamide (252.3 mg) as a white solid. LCMS Method CG: [M+H]⁺=474. ¹H NMR (400 MHz, DMSO-d₆): δ 11.31 (brs, 1H), 11.08 (brs, 1H), 10.90 (brs, 1H), 8.35-8.30 (m, 1H), 8.20-8.13 (m, 3H), 7.84-7.78 (m, 4H), 7.24-7.21 (m, 1H), 4.58-4.56 (m, 1H), 1.47 (d, 6H).

### Example 146: Synthesis of Ni-(5,6-difluoro-1H-indol-3-yl)-N₂-(2,2,2-trifluoro-1-phenylethyl)oxalamide (Compound 312)

### Step 1: Synthesis of 2-((5,6-difluoro-1H-indol-3-yl)amino)-2-oxoacetic acid

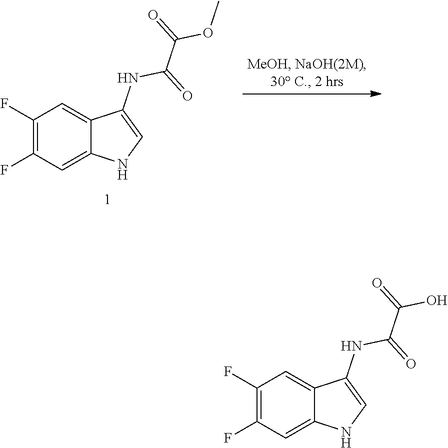 [SMILES: COC(=O)C(=O)Nc1c[nH]c2cc(F)c(F)cc12 ; O=C(O)C(=O)Nc1c[nH]c2cc(F)c(F)cc12]

Methyl 2-((5,6-difluoro-1H-indol-3-yl)amino)-2-oxoacetate (4.0 g, 15.75 mmol, 1 equiv.) was dissolved in MeOH (100 mL) and then aqueous NaOH (2 M, 11.8 mL, 23.60 mmol, 1.5 equiv.) was added. The reaction mixture was heated at 30° C. for 2 hours, then concentrated in vacuo. Then H₂O (30 mL) was added to the residue and the mixture was adjusted to pH 4 by the dropwise addition of 2 M HCl. The resulting solid was collected by filtration and washed with water to give 2-((5,6-difluoro-1H-indol-3-yl)amino)-2-oxoacetic acid (3.5 g, 14.58 mmol) as a light yellow solid. MS-ESI, 241.1 [M+H⁺].

### Step 2: Synthesis of Ni-(5,6-difluoro-1H-indol-3-yl)-N₂-(2,2,2-trifluoro-1-phenylethyl)oxalamide (Compound 312)

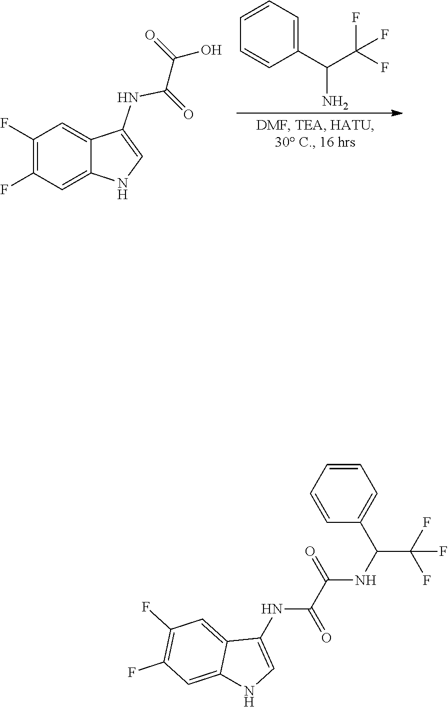

2-((5,6-difluoro-1H-indol-3-yl)amino)-2-oxoacetic acid (60.0 mg, 0.25 mmol, 1.0 equiv.) and 2,2,2-trifluoro-1-phenylethanamine (43.8 mg, 0.25 mmol, 1.0 equiv.) were dissolved in DMF (3 mL). Then HATU (95.0 mg, 0.25 mmol, 1.0 equiv.) and TEA (70 μl, 0.5 mmol, 2.0 equiv.) were added. The mixture was heated at 30° C. for 16 hours. The solvent was concentrated in vacuo and the residue was purified by prep HPLC to give N1-(5,6-difluoro-1H-indol-3-yl)-N₂-(2,2,2-trifluoro-1-phenylethyl)oxalamide (27.4 mg, 69.0 μmol) as a white powder. MS-ESI, 398.1 [M+H⁺]. ¹H NMR (400 MHz, DMSO-d₆) δ ppm 11.19 (s, 1H), 10.87 (s, 1H), 10.08 (d, 1H), 7.93 (dd, 1H), 7.83 (d, 1H), 7.78-7.70 (m, 2H), 7.48-7.36 (m, 4H), 5.90-5.85 (m, 1H)

The following examples were synthesized following the method above.

| | | | | LC-MS, |
|---|---|---|---|---|
| | | | | MS-ESI, -- |
| | | | | [M + H⁺]. |
| Exam- | Compound | | | Methods |
| ple # | # | Structure | IUPAC Name | AA or AB |
| 147 | 313 | 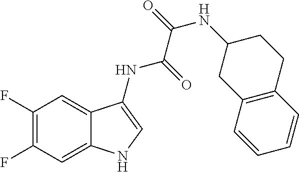 [SMILES: O=C(Nc1c[nH]c2cc(F)c(F)cc12)C(=O)NC1CCc2ccccc2C1] | N-(5,6-difluoro-1H- indol-3-yl)-N′-(1,2,3,4- tetrahydronaphthalen- 2-yl)ethanediamide | 370.0 |
| 148 | 301 | 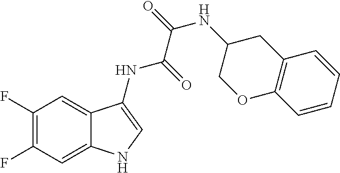 [SMILES: O=C(Nc1c[nH]c2cc(F)c(F)cc12)C(=O)NC1COc2ccccc2C1] | N-(5,6-difluoro-1H- indol-3-yl)-N′-(3,4- dihydro-2H-1- benzopyran-3- yl)ethanediamide | 372.2 |
| 149 | 300 | 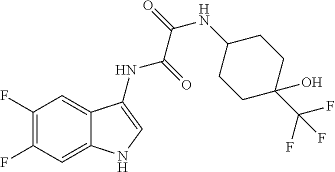 [SMILES: O=C(Nc1c[nH]c2cc(F)c(F)cc12)C(=O)NC1CCC(O)(C(F)(F)F)CC1] | N-(5,6-difluoro-1H- indol-3-yl)-N′-[4- hydroxy-4- (trifluoromethyl) cyclohexyl] ethanediamide | 406.2 |
| 150 | 299 | 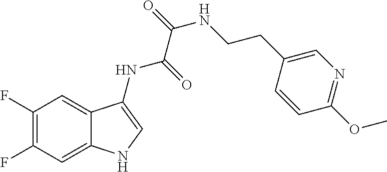 [SMILES: COc1ccc(CCNC(=O)C(=O)Nc2c[nH]c3cc(F)c(F)cc23)cn1] | N-(5,6-difluoro-1H- indol-3-yl)-N′-[2-(6- methoxypyridin-3- yl)ethyl]ethanediamide | 375.1 |
| 151 | 298 | 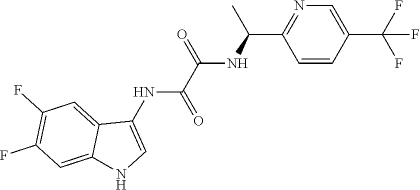 [SMILES: C[C@H](NC(=O)C(=O)Nc1c[nH]c2cc(F)c(F)cc12)c1ccc(C(F)(F)F)cn1] | N-(5,6-difluoro-1H- indol-3-yl)-N′-[(1S)-1- [5-(trifluoromethyl) pyridin-2- yl]ethyl]ethanediamide | 413.2 |
| 152 | 277 | 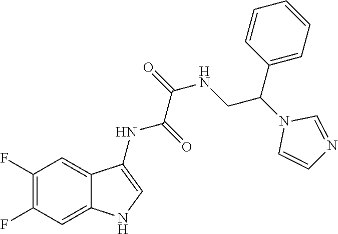 [SMILES: O=C(NCC(c1ccccc1)n1ccnc1)C(=O)Nc1c[nH]c2cc(F)c(F)cc12] | N-(5,6-difluoro-1H- indol-3-yl)-N′-[2-(1H- imidazol-1-yl)-2- phenylethyl] ethanediamide | 410.1 |
| 153 | 276 | 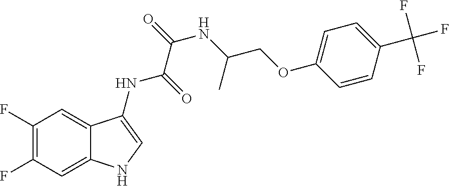 [SMILES: CC(COc1ccc(C(F)(F)F)cc1)NC(=O)C(=O)Nc1c[nH]c2cc(F)c(F)cc12] | N-(5,6-difluoro-1H- indol-3-yl)-N′-{1-[4- (trifluoromethyl) phenoxy]propan-2- yl}ethanediamide | 441.8 |
| 154 | 275 | 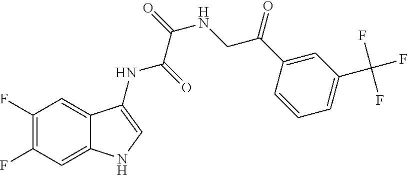 [SMILES: O=C(NCC(=O)c1cccc(C(F)(F)F)c1)C(=O)Nc1c[nH]c2cc(F)c(F)cc12] | N-(5,6-difluoro-1H- indol-3-yl)-N′-{2-oxo- 2-[3-(trifluoromethyl) phenyl]ethyl} ethanediamide | 426.1 |
| 155 | 297 | 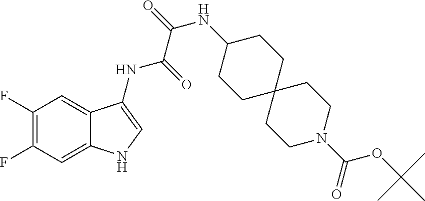 [SMILES: CC(C)(C)OC(=O)N1CCC2(CCC(NC(=O)C(=O)Nc3c[nH]c4cc(F)c(F)cc34)CC2)CC1] | tert-butyl 9-{[(5,6- difluoro-1H-indol-3- yl)carbamoyl] formamido}-3- azaspiro[5.5]undecane- 3-carboxylate | 435.1 |
| 156 | 311 | 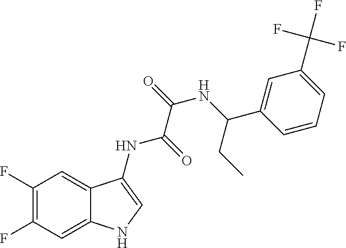 [SMILES: CCC(NC(=O)C(=O)Nc1c[nH]c2cc(F)c(F)cc12)c1cccc(C(F)(F)F)c1] | N-(5,6-difluoro-1H- indol-3-yl)-N′-{1-[3- (trifluoromethyl) phenyl]propyl} ethanediamide | 426.1 |
| 157 | 310 | 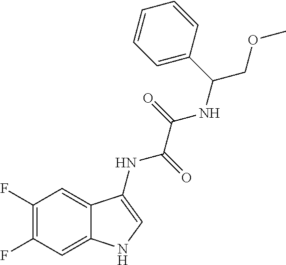 [SMILES: COCC(NC(=O)C(=O)Nc1c[nH]c2cc(F)c(F)cc12)c1ccccc1] | N-(5,6-difluoro-1H- indol-3-yl)-N′-(2- methoxy-1- phenylethyl) ethanediamide | 374.1 |
| 158 | 271 | 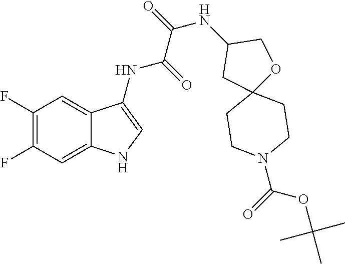 [SMILES: CC(C)(C)OC(=O)N1CCC2(CC1)CC(NC(=O)C(=O)Nc1c[nH]c3cc(F)c(F)cc13)CO2] | tert-butyl 3-{[(5,6- difluoro-1H-indol-3- yl)carbamoyl] formamido}-1-oxa-8- azaspiro[4.5]decane- 8-carboxylate | 379.1 |
| 159 | 270 | 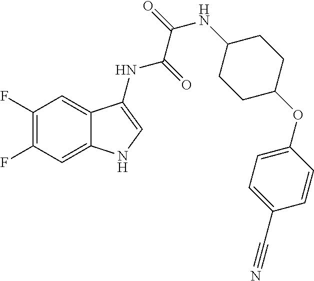 [SMILES: N#Cc1ccc(OC2CCC(NC(=O)C(=O)Nc3c[nH]c4cc(F)c(F)cc34)CC2)cc1] | N′-[4-(4- cyanophenoxy) cyclohexyl]-N-(5,6- difluoro-1H-indol-3- yl)ethanediamide | 439.2 |
| 160 | 269 | 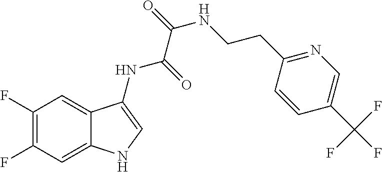 [SMILES: O=C(NCCc1ccc(C(F)(F)F)cn1)C(=O)Nc1c[nH]c2cc(F)c(F)cc12] | N-(5,6-difluoro-1H- indol-3-yl)-N′-{2-[5- (trifluoromethyl) pyridin-2- yl]ethyl}ethanediamide | 412.8 |
| 161 | 294 | 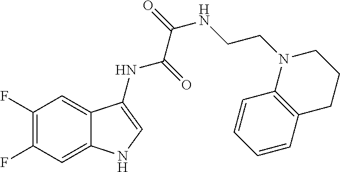 [SMILES: O=C(NCCN1CCCc2ccccc21)C(=O)Nc1c[nH]c2cc(F)c(F)cc12] | N-(5,6-difluoro-1H- indol-3-yl)-N′-[2- (1,2,3,4- tetrahydroquinolin-1- yl)ethyl]ethanediamide | 399.1 |
| 162 | 309 | 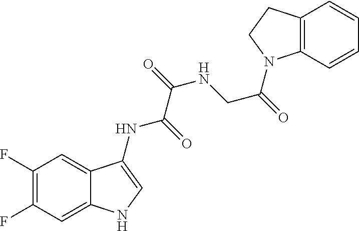 [SMILES: O=C(NCC(=O)N1CCc2ccccc21)C(=O)Nc1c[nH]c2cc(F)c(F)cc12] | N-(5,6-difluoro-1H- indol-3-yl)-N′-[2-(2,3- dihydro-1H-indol-1- yl)-2-oxoethyl] ethanediamide | 399.1 |
| 163 | 308 | 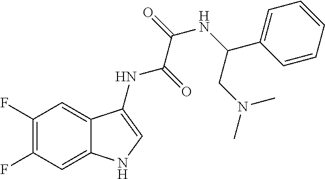 [SMILES: CN(C)CC(NC(=O)C(=O)Nc1c[nH]c2cc(F)c(F)cc12)c1ccccc1] | N-(5,6-difluoro-1H- indol-3-yl)-N′-[2- (dimethylamino)-1- phenylethyl] ethanediamide | 387.1 |
| 164 | 291 | 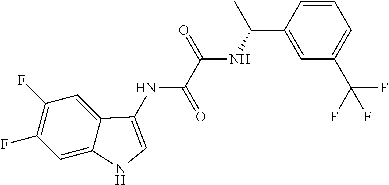 [SMILES: C[C@@H](NC(=O)C(=O)Nc1c[nH]c2cc(F)c(F)cc12)c1cccc(C(F)(F)F)c1] | N-(5,6-difluoro-1H- indol-3-yl)-N′-[(1R)-1- [3-(trifluoromethyl) phenyl]ethyl] ethanediamide | 412.1 |
| 165 | 307 | 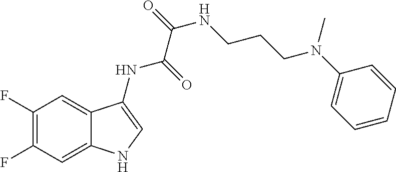 [SMILES: CN(CCCNC(=O)C(=O)Nc1c[nH]c2cc(F)c(F)cc12)c1ccccc1] | N-(5,6-difluoro-1H- indol-3-yl)-N′-{3- [methyl(phenyl)amino] propyl}ethanediamide | 387.2 |
| 166 | 306 | 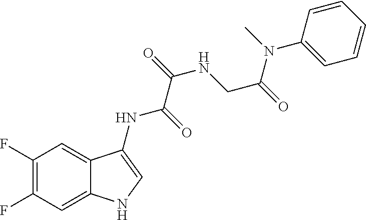 [SMILES: CN(C(=O)CNC(=O)C(=O)Nc1c[nH]c2cc(F)c(F)cc12)c1ccccc1] | N-(5,6-difluoro-1H- indol-3-yl)-N′- {[methyl(phenyl) carbamoyl]methyl} ethanediamide | 387.2 |
| 167 | 290 | 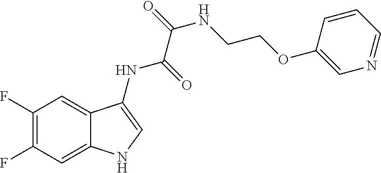 [SMILES: O=C(NCCOc1cccnc1)C(=O)Nc1c[nH]c2cc(F)c(F)cc12] | N-(5,6-difluoro-1H- indol-3-yl)-N′-[2- (pyridin-3-yloxy)ethyl] ethanediamide | 361.1 |
| 168 | 305 | 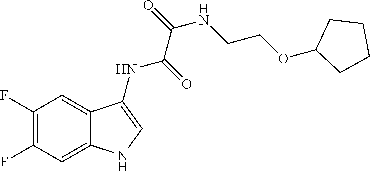 [SMILES: O=C(NCCOC1CCCC1)C(=O)Nc1c[nH]c2cc(F)c(F)cc12] | N'-[2- (cyclopentyloxy)ethyl]- N-(5,6-difluoro-1H- indol-3- yl)ethanediamide | 352.2 |
| 169 | 304 | 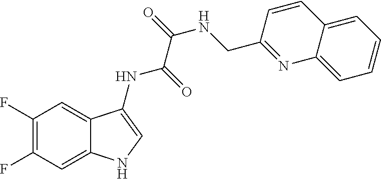 [SMILES: O=C(NCc1ccc2ccccc2n1)C(=O)Nc1c[nH]c2cc(F)c(F)cc12] | N-(5,6-difluoro-1H- indol-3-yl)-N′- [(quinolin-2- yl)methyl] ethanediamide | 381.1 |
| 170 | 288 | 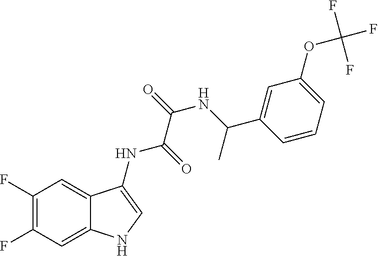 [SMILES: CC(NC(=O)C(=O)Nc1c[nH]c2cc(F)c(F)cc12)c1cccc(OC(F)(F)F)c1] | N-(5,6-difluoro-1H- indol-3-yl)-N′-{1-[3- (trifluoromethoxy) phenyl]ethyl} ethanediamide | 428.1 |
| 171 | 267 | 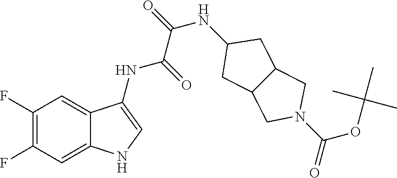 [SMILES: CC(C)(C)OC(=O)N1CC2CC(NC(=O)C(=O)Nc3c[nH]c4cc(F)c(F)cc34)CC2C1] | tert-butyl 5-{[(5,6- difluoro-1H-indol-3- yl)carbamoyl] formamido}- octahydrocyclopenta[c] pyrrole-2-carboxylate | 393.2 |
| 172 | 261 | 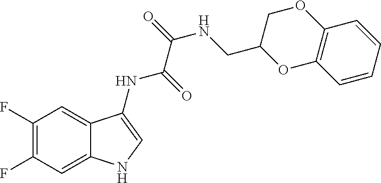 [SMILES: O=C(NCC1COc2ccccc2O1)C(=O)Nc1c[nH]c2cc(F)c(F)cc12] | N-(5,6-difluoro-1H- indol-3-yl)-N′-[(2,3- dihydro-1,4- benzodioxin-2- yl)methyl] ethanediamide | 388.2 |

### Example 173: Synthesis of N1-(5,6-difluoro-1H-indol-3-yl)-N₂-(1-oxo-2,3-dihydro-1H-inden-4-yl)oxalamide

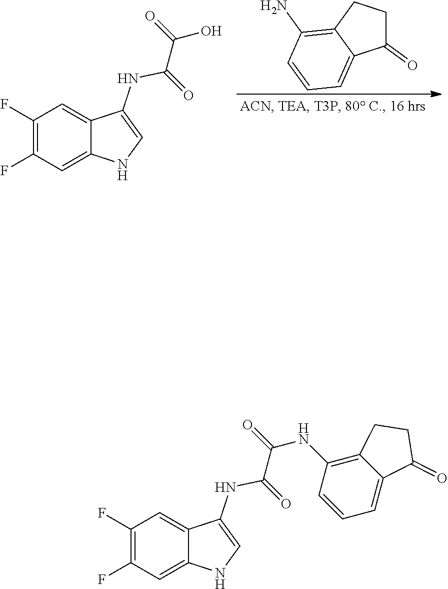 [SMILES: O=C(O)C(=O)Nc1c[nH]c2cc(F)c(F)cc12 ; Nc1cccc2c1CCC2=O ; O=C(Nc1cccc2c1CCC2=O)C(=O)Nc1c[nH]c2cc(F)c(F)cc12]

2-((5,6-difluoro-1H-indol-3-yl)amino)-2-oxoacetic acid 60.0 mg, 0.25 mmol, 1.0 equiv.) and 4-amino-2,3-dihydro-1H-inden-1-one (36.8 mg, 0.25 mmol, 1.0 equiv.) was dissolved in ACN (3 mL). Then T3P (50 wt. % in EtOAc, 300 μl, 0.52 mmol, 2.0 equiv.) and TEA (70 μl, 0.5 mmol, 2.0 equiv.) were added. The mixture was heated at 80° C. for 16 hours. The solvent was removed in vacuo and the residue was purified by prep HPLC to give N1-(5,6-difluoro-1H-indol-3-yl)-N₂-(1-oxo-2,3-dihydro-1H-inden-4-yl)oxalamide (4.4 mg, 11.9 mol) as a white powder. MS-ESI, 370.2 [M+H⁺]. ¹H NMR (400 MHz, DMSO-d₆) δ ppm 11.24 (s, 1H), 11.00 (s, 1H), 10.60 (s, 1H), 8.00 (dd, 1H), 7.96-7.86 (m, 2H), 7.58-7.47 (m, 2H), 7.40 (dd, 1H), 3.17-3.10 (m, 2H), 2.76-2.61 (m, 2H)

The following compounds were synthesized using the method described above for Example 173.

| | | | | LC-MS, |
|---|---|---|---|---|
| | | | | MS-ESI, -- |
| | | | | [M + H+]. |
| Example | Compound | | | Methods |
| # | No. | Structure | IUPAC Name | AA or AB |
| 174 | 296 | 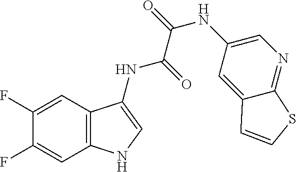 [SMILES: O=C(Nc1cnc2sccc2c1)C(=O)Nc1c[nH]c2cc(F)c(F)cc12] | N′-(5,6-difluoro- 1H-indol-3-yl)-N- {thieno[2,3- b]pyridin-5- yl}ethanediamide | 373.0 |
| 175 | 262 | 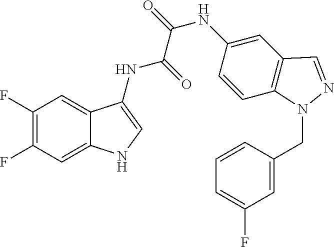 [SMILES: O=C(Nc1ccc2c(cnn2Cc2cccc(F)c2)c1)C(=O)Nc1c[nH]c2cc(F)c(F)cc12] | N-(5,6-difluoro- 1H-indol-3-yl)-N′- {1-[(3- fluorophenyl) methyl]-1H-indazol- 5-yl}ethanediamide | 464.2 |
| 176 | 274 | 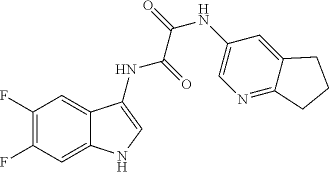 [SMILES: O=C(Nc1cnc2c(c1)CCC2)C(=O)Nc1c[nH]c2cc(F)c(F)cc12] | N′-{5H,6H,7H- cyclopenta[b] pyridin-3-yl}-N- (5,6-difluoro-1H- indol-3- yl)ethanediamide | 357.1 |
| 177 | 273 | 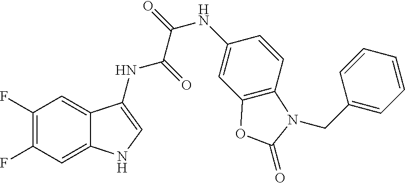 [SMILES: O=C(Nc1ccc2c(c1)oc(=O)n2Cc1ccccc1)C(=O)Nc1c[nH]c2cc(F)c(F)cc12] | N′-(3-benzyl-2- oxo-2,3-dihydro- 1,3-benzoxazol-6- yl)-N-(5,6- difluoro-1H-indol- 3-yl)ethanediamide | 462.8 |
| 178 | 295 | 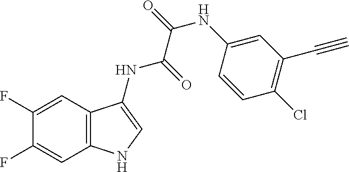 [SMILES: C#Cc1cc(NC(=O)C(=O)Nc2c[nH]c3cc(F)c(F)cc23)ccc1Cl] | N′-(4-chloro-3- ethynylphenyl)-N- (5,6-difluoro-1H- indol-3- yl)ethanediamide | 374.0 |
| 179 | 272 | 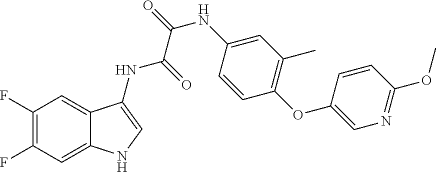 [SMILES: COc1ccc(Oc2ccc(NC(=O)C(=O)Nc3c[nH]c4cc(F)c(F)cc34)cc2C)cn1] | N-(5,6-difluoro- 1H-indol-3-yl)-N′- {4-[(6- methoxypyridin-3- yl)oxy]-3- methylphenyl} ethanediamide | 453.2 |
| 180 | 293 | 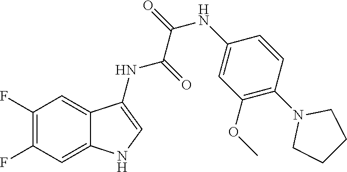 [SMILES: COc1cc(NC(=O)C(=O)Nc2c[nH]c3cc(F)c(F)cc23)ccc1N1CCCC1] | N-(5,6-difluoro- 1H-indol-3-yl)-N′- [3-methoxy-4- (pyrrolidin-1- yl)phenyl] ethanediamide | 415.1 |
| 181 | 292 | 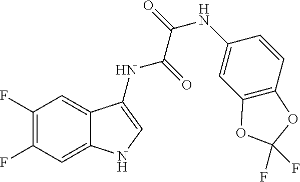 [SMILES: O=C(Nc1ccc2c(c1)OC(F)(F)O2)C(=O)Nc1c[nH]c2cc(F)c(F)cc12] | N-(5,6-difluoro- 1H-indol-3-yl)-N′- (2,2-difluoro-2H- 1,3-benzodioxol-5- yl)ethanediamide | 396.1 |
| 182 | 268 | 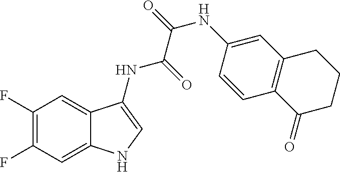 [SMILES: O=C(Nc1ccc2c(c1)CCCC2=O)C(=O)Nc1c[nH]c2cc(F)c(F)cc12] | N-(5,6-difluoro- 1H-indol-3-yl)-N′- (5-oxo-5,6,7,8- tetrahydro- naphthalen-2- yl)ethanediamide | 384.2 |
| 183 | 289 | 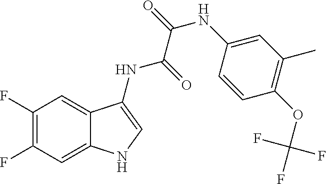 [SMILES: Cc1cc(NC(=O)C(=O)Nc2c[nH]c3cc(F)c(F)cc23)ccc1OC(F)(F)F] | N-(5,6-difluoro- 1H-indol-3-yl)-N′- [3-methyl-4- (trifluoromethoxy) phenyl] ethanediamide | 414.1 |
| 184 | 287 | 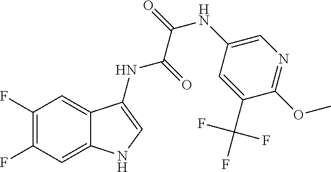 [SMILES: COc1ncc(NC(=O)C(=O)Nc2c[nH]c3cc(F)c(F)cc23)cc1C(F)(F)F] | N-(5,6-difluoro- 1H-indol-3-yl)-N′- [6-methoxy-5- (trifluoromethyl) pyridin-3- yl]ethanediamide | 415.1 |
| 185 | 266 | 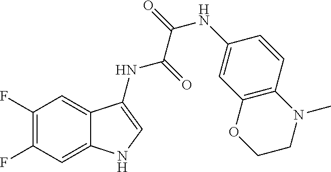 [SMILES: CN1CCOc2cc(NC(=O)C(=O)Nc3c[nH]c4cc(F)c(F)cc34)ccc21] | N-(5,6-difluoro- 1H-indol-3-yl)-N′- (4-methyl-3,4- dihydro-2H-1,4- benzoxazin-7- yl)ethanediamide | 386.9 |
| 186 | 265 | 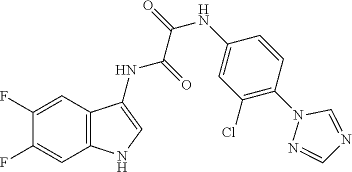 [SMILES: O=C(Nc1ccc(-n2cncn2)c(Cl)c1)C(=O)Nc1c[nH]c2cc(F)c(F)cc12] | N′-[3-chloro-4- (1H-1,2,4-triazol- 1-yl)phenyl]-N- (5,6-difluoro-1H- indol-3- yl)ethanediamide | 417.1 |
| 187 | 286 | 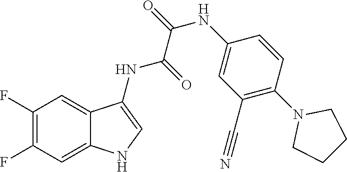 [SMILES: N#Cc1cc(NC(=O)C(=O)Nc2c[nH]c3cc(F)c(F)cc23)ccc1N1CCCC1] | N′-[3-cyano-4- (pyrrolidin-1- yl)phenyl]-N-(5,6- difluoro-1H-indol- 3-yl)ethanediamide | 410.1 |

### Example 188: Synthesis of N1-(6-(2H-1,2,3-triazol-2-yl)-5-(trifluoromethyl)pyridin-3-yl)-N₂-(5,6-difluoro-1H-indol-3-yl)oxalamide (Compound 238)

### Step 1: Synthesis of ethyl 2-((6-(2H-1,2,3-triazol-2-yl)-5-(trifluoromethyl)pyridin-3-yl)amino)-2-oxoacetate

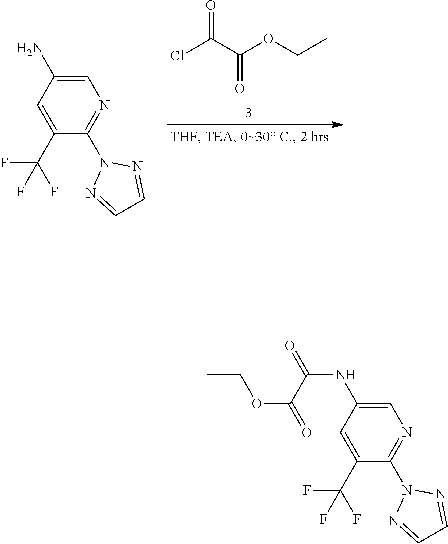

6-(2H-1,2,3-triazol-2-yl)-5-(trifluoromethyl)pyridin-3-amine (100.0 mg, 0.44 mmol, 1.0 equiv.) was dissolved in THF (3 mL) and cooled to 0° C. Then a solution of ethyl 2-chloro-2-oxoacetate (71.8 mg, 0.53 mmol, 1.2 equiv.) in THF (2 mL) was added over 5 minutes, maintaining the reaction mixture at 0° C. Then TEA (0.16 mL, 1.1 mmol, 2.5 equiv.) was added to the reaction mixture. The mixture was heated at 30° C. for 2 hours. The reaction mixture was filtered and the filtrate was concentrated in vacuo to give ethyl 2-((6-(2H-1,2,3-triazol-2-yl)-5-(trifluoromethyl)pyridin-3-yl)amino)-2-oxoacetate (120 mg, 0.36 mmol) as a yellow oil that was used without additional purification.

### Step 2: Synthesis of 2-((6-(2H-1,2,3-triazol-2-yl)-5-(trifluoromethyl)pyridin-3-yl)amino)-2-oxoacetic acid

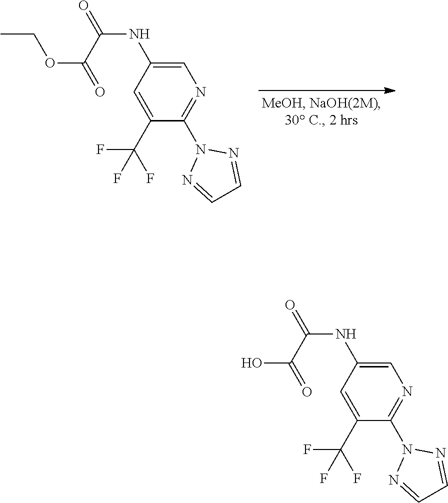 [SMILES: CCOC(=O)C(=O)Nc1cnc(-n2nccn2)c(C(F)(F)F)c1 ; O=C(O)C(=O)Nc1cnc(-n2nccn2)c(C(F)(F)F)c1]

Ethyl 2-((6-(2H-1,2,3-triazol-2-yl)-5-(trifluoromethyl)pyridin-3-yl)amino)-2-oxoacetate (120.0 mg, 0.36 mmol, 1 equiv.) was dissolved in MeOH (5 mL) and then aqueous NaOH (2 M, 0.27 mL, 0.54 mmol, 1.5 equiv.) was added. The mixture was heated at 30° C. for 2 hours, then concentrated in vacuo. Then H₂O (30 mL) was added to the residue and the mixture was adjusted to pH 4 by the dropwise addition of 2 M HCl. The resulting solid was collected by filtration and washed with water to give 2-((6-(2H-1,2,3-triazol-2-yl)-5-(trifluoromethyl)pyridin-3-yl)amino)-2-oxoacetic acid (80 mg, 0.27 mmol) as a light yellow solid.

### Step 3: N1-(6-(2H-1,2,3-triazol-2-yl)-5-(trifluoromethyl)pyridin-3-yl)-N₂-(5,6-difluoro-1H-indol-3-yl)oxalamide (Compound 238)

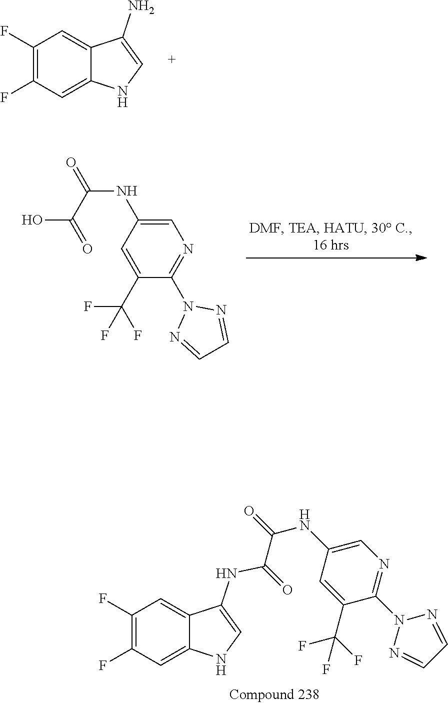 [SMILES: Nc1c[nH]c2cc(F)c(F)cc12 ; O=C(O)C(=O)Nc1cnc(-n2nccn2)c(C(F)(F)F)c1 ; O=C(Nc1cnc(-n2nccn2)c(C(F)(F)F)c1)C(=O)Nc1c[nH]c2cc(F)c(F)cc12]

5,6-difluoro-1H-indol-3-amine (42.0 mg, 0.25 mmol, 1.0 equiv.) and 2-((6-(2H-1,2,3-triazol-2-yl)-5-(trifluoromethyl)pyridin-3-yl)amino)-2-oxoacetic acid (75.0 mg, 0.25 mmol, 1.0 equiv.) was dissolved in DMF (3 mL). Then HATU (95.0 mg, 0.25 mmol, 1.0 equiv.) and TEA (70 μl, 0.5 mmol, 2.0 equiv.) were added. The mixture was heated at 30° C. for 16 hours. The solvent was concentrated in vacuo and the residue was purified by prep HPLC to give N1-(6-(2H-1,2,3-triazol-2-yl)-5-(trifluoromethyl)pyridin-3-yl)-N₂-(5, 6-difluoro-1H-indol-3-yl)oxalamide (69.8 mg, 0.15 mmol) as a white powder. MS-ESI, 452.1 [M+H⁺]. ¹H NM/R (400 MHz, DMSO-d₆) δ ppm 11.72 (s, 1H), 11.27 (s, 1H), 11.11 (s, 1H), 9.39 (d, 1H), 9.03 (d, 1H), 8.28-8.15 (m, 2H), 8.01 (dd, 1H), 7.91 (d, 1H), 7.41 (dd, 1H) The following compounds were prepared using the method described for Example 94.

| | Compound | | |
|---|---|---|---|
| Example | # | Structure | LCMS data |
| 189 | 205 | 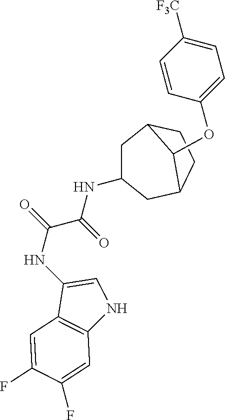 [SMILES: O=C(Nc1c[nH]c2cc(F)c(F)cc12)C(=O)NC1CC2CCC(C1)C2Oc1ccc(C(F)(F)F)cc1] | Method DA: MS-ESI: 508 [M + H]⁺. |
| 190 | 206 | 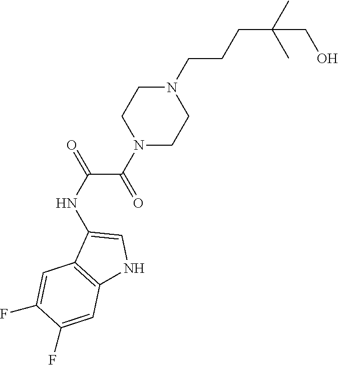 [SMILES: CC(C)(CO)CCCN1CCN(C(=O)C(=O)Nc2c[nH]c3cc(F)c(F)cc23)CC1] | Method DA: MS-ESI: 423 [M + H]⁺. |
| 191 | 215 | 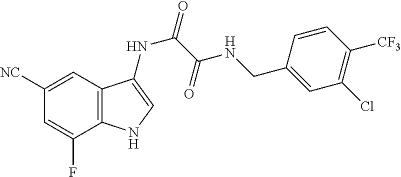 [SMILES: N#Cc1cc(F)c2[nH]cc(NC(=O)C(=O)NCc3ccc(C(F)(F)F)c(Cl)c3)c2c1] | Method DA: MS-ESI: 439 [M + H]⁺. |
| 192 | 235 | 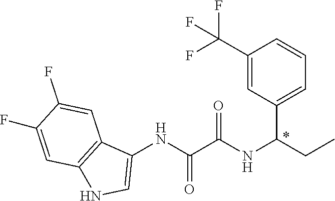 [SMILES: CCC(NC(=O)C(=O)Nc1c[nH]c2cc(F)c(F)cc12)c1cccc(C(F)(F)F)c1] | Method DB: MS-ESI: 426 [M + H]⁺. Column: CHIRAL ART Cellulose-SB, 2 * 25 cm, 5 um; Mobile Phase A: Hex(0.5% 2M NH3— MeOH)--HPLC, Mobile Phase B: EtOH--HPLC; Flow rate: 20 mL/min; Gradient: 10 B to 10 B in 14 min; 220/254 nm; RT2: 11.514; Injection Volumn: 0.5 ml |
| 193 | 236 | 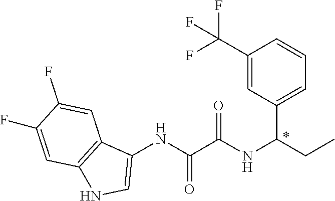 [SMILES: CCC(NC(=O)C(=O)Nc1c[nH]c2cc(F)c(F)cc12)c1cccc(C(F)(F)F)c1] | Method DB: MS-ESI: 426 [M + H]⁺. Column: CHIRAL ART Cellulose-SB, 2 * 25 cm, 5 um; Mobile Phase A: Hex(0.5% 2M NH3— MeOH)--HPLC, Mobile Phase B: EtOH--HPLC; Flow rate: 20 mL/min; Gradient: 10 B to 10 B in 14 min; 220/254 nm; RT1: 9.839; Injection Volumn: 0.5 ml |
| 194 | 243 | 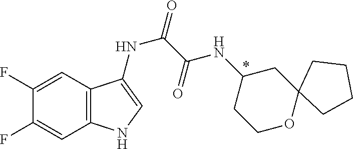 [SMILES: O=C(Nc1c[nH]c2cc(F)c(F)cc12)C(=O)NC1CCOC2(CCCC2)C1] | Method DD: MS-ESI: 376 [M − H]⁻. Column: CHIRALPAK IA, 2 * 25 cm, 5 um; Mobile Phase A: Hex(0.5% 2M NH3—MeOH)--HPLC, Mobile Phase B: EtOH-- HPLC; Flow rate: 18 mL/min; Gradient: 50 B to 50 B in 21 min; |
| | | | 220/254 nm; RT2: |
| | | | 16.919; Injection |
| | | | Volumn: 1.5 ml |
| 195 | 244 | 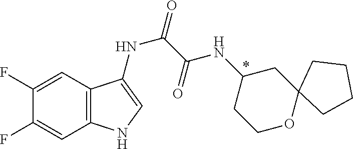 [SMILES: O=C(Nc1c[nH]c2cc(F)c(F)cc12)C(=O)NC1CCOC2(CCCC2)C1] | Method DD: MS-ESI: 376 [M − H]⁻. Column: CHIRALPAK IA, 2 * 25 cm, 5 um; Mobile Phase A: Hex(0.5% 2M NH3—MeOH)--HPLC, Mobile Phase B: EtOH-- HPLC; Flow rate: 18 mL/min; Gradient: 50 B to 50 B in 21 min; |
| | | | 220/254 nm; RTl: |
| | | | 11.799; Injection |
| | | | Volumn: 1.5 ml |
| 196 | 245 | 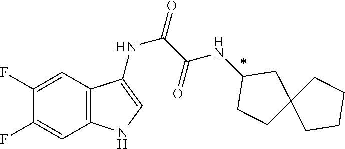 [SMILES: O=C(Nc1c[nH]c2cc(F)c(F)cc12)C(=O)NC1CCC2(CCCC2)C1] | Method DD: MS-ESI: 360 [M − H]⁻. Column: LUX 5 um Cellulose-2, 2.12 * 25 cm, 5 um; Mobile Phase A: Hex(0.5% 2M NH3— MeOH)--HPLC, Mobile Phase B: IPA--HPLC; Flow rate: 23 mL/min; Gradient: 10 B to 10 B in |
| | | | 25 min; 220/254 nm; |
| | | | RT2: 20.353; Injection |
| | | | Volumn: 0.5 ml |
| 197 | 246 | 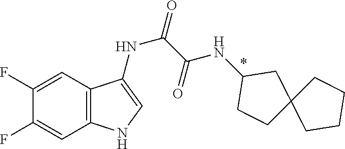 [SMILES: O=C(Nc1c[nH]c2cc(F)c(F)cc12)C(=O)NC1CCC2(CCCC2)C1] | Method DD: MS-ESI: 360 [M − H]⁻. Column: LUX 5 um Cellulose-2, 2.12 * 25 cm, 5 um; Mobile Phase A: Hex(0.5% 2M NH3— MeOH)--HPLC, Mobile Phase B: IPA--HPLC; Flow rate: 23 mL/min; Gradient: 10 B to 10 B in |
| | | | 25 min; 220/254 nm; |
| | | | RT1: 15.344; Injection |
| | | | Volumn: 0.5 ml |
| 198 | 247 | 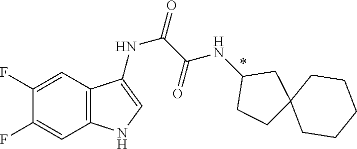 [SMILES: O=C(Nc1c[nH]c2cc(F)c(F)cc12)C(=O)NC1CCC2(CCCCC2)C1] | Method DC: MS-ESI: 376 [M + H]⁺. Column: CHIRALPAK IA, 2 * 25 cm, 5 um; Mobile Phase A: Hex(0.5% 2M NH3—MeOH)--HPLC, Mobile Phase B: EtOH-- HPLC; Flow rate: 18 mL/min; Gradient: 50 B to 50 B in 13 min; |
| | | | 220/254 nm; RT2: 9.97; |
| | | | Injection Volumn: 0.5 ml |
| 199 | 248 | 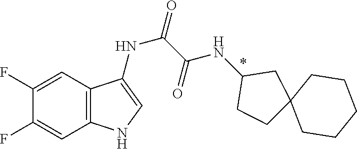 [SMILES: O=C(Nc1c[nH]c2cc(F)c(F)cc12)C(=O)NC1CCC2(CCCCC2)C1] | Method DC: MS-ESI: 376 [M + H]⁺. Column: CHIRALPAK IA, 2 * 25 cm, 5 um; Mobile Phase A: Hex(0.5% 2M NH3—MeOH)--HPLC, Mobile Phase B: EtOH-- HPLC; Flow rate: 18 mL/min; Gradient: 50 B to 50 B in 13 min; |
| | | | 220/254 nm; RT1: 7.796; |
| | | | Injection Volumn: 0.5 ml |
| 200 | 252 | 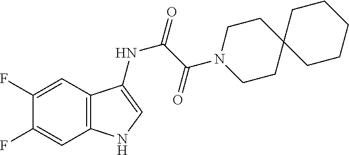 [SMILES: O=C(Nc1c[nH]c2cc(F)c(F)cc12)C(=O)N1CCC2(CCCCC2)CC1] | Method DB: MS-ESI: 376 [M + H]⁺. |
| 201 | 254 | 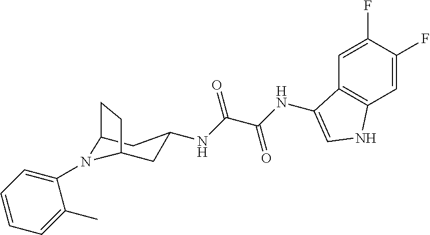 [SMILES: Cc1ccccc1N1C2CCC1CC(NC(=O)C(=O)Nc1c[nH]c3cc(F)c(F)cc13)C2] | Method DD: MS-ESI: 439 [M + H]⁺. |
| 202 | 256 | 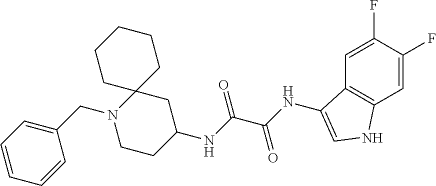 [SMILES: O=C(Nc1c[nH]c2cc(F)c(F)cc12)C(=O)NC1CCN(Cc2ccccc2)C2(CCCCC2)C1] | Method DE: MS-ESI: 481 [M + H]⁺. |
| 203 | 257 | 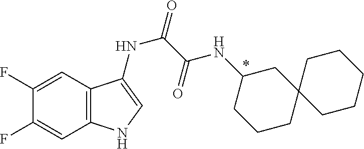 [SMILES: O=C(Nc1c[nH]c2cc(F)c(F)cc12)C(=O)NC1CCCC2(CCCCC2)C1] | Method DD: MS-ESI: 388 [M − H]⁻. Column: CHIRALPAK IA, 2 * 25 cm, 5 um; Mobile Phase A: Hex(0.5% 2M NH3—MeOH)--HPLC, Mobile Phase B: EtOH-- HPLC; Flow rate: 18 mL/min; Gradient: 50 B to 50 B in 19 min; |
| | | | 220/254 nm; RT2: |
| | | | 12.034; Injection |
| | | | Volumn: 2 ml |
| 204 | 258 | 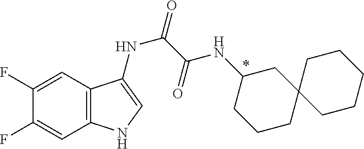 [SMILES: O=C(Nc1c[nH]c2cc(F)c(F)cc12)C(=O)NC1CCCC2(CCCCC2)C1] | Method DD: MS-ESI: 388 [M − H]⁻. Column: CHIRALPAK IA, 2 * 25 cm, 5 um; Mobile Phase A: Hex(0.5% 2M NH3—MeOH)--HPLC, Mobile Phase B: EtOH-- HPLC; Flow rate: 18 mL/min; Gradient: 50 B to 50 B in 19 min; |
| | | | 220/254 nm; RTl: 7.448; |
| | | | Injection Volumn: 2 ml |
| 205 | 259 | 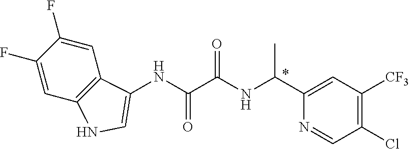 [SMILES: CC(NC(=O)C(=O)Nc1c[nH]c2cc(F)c(F)cc12)c1cc(C(F)(F)F)c(Cl)cn1] | Method DE: MS-ESI: 447 [M + H]⁺. Column: CHIRAL ART Cellulose-SB, 3 * 25 cm, 5 um; Mobile Phase A: Hex(0.1% FA)--HPLC, Mobile Phase B: EtOH-- HPLC; Flow rate: 45 mL/min; Gradient: 20 B to 20 B in 15 min; |
| | | | 220/254 nm; RT2: 13; |
| | | | Injection Volumn: 1 ml |
| 206 | 260 | 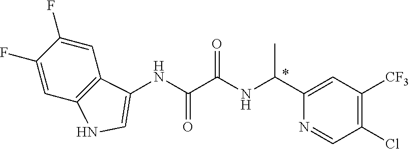 [SMILES: CC(NC(=O)C(=O)Nc1c[nH]c2cc(F)c(F)cc12)c1cc(C(F)(F)F)c(Cl)cn1] | Method DE: MS-ESI: 447 [M + H]⁺. Column: CHIRAL ART Cellulose-SB, 3 * 25 cm, 5 um; Mobile Phase A: Hex(0.1% FA)--HPLC, Mobile Phase B: EtOH-- HPLC; Flow rate: 45 mL/min; Gradient: 20 B to 20 B in 15 min; |
| | | | 220/254 nm; RT1: 9.8; |
| | | | Injection Volumn: 1 ml |
| 207 | 263 | 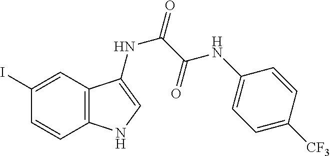 [SMILES: O=C(Nc1ccc(C(F)(F)F)cc1)C(=O)Nc1c[nH]c2ccc(I)cc12] | Method DD: MS-ESI: 472[M − H]⁻. |
| 208 | 282 | 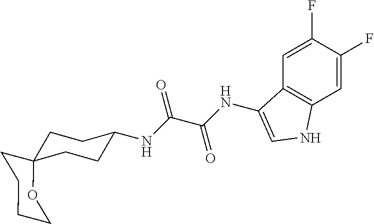 [SMILES: O=C(Nc1c[nH]c2cc(F)c(F)cc12)C(=O)NC1CCC2(CCCCO2)CC1] | Method DF: MS-ESI: 392 [M + H]⁺. |
| 209 | 284 | 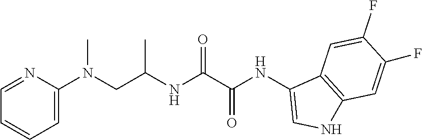 [SMILES: CC(CN(C)c1ccccn1)NC(=O)C(=O)Nc1c[nH]c2cc(F)c(F)cc12] | Method DE: MS-ESI: 388 [M + H]⁺. |
| 210 | 285 | 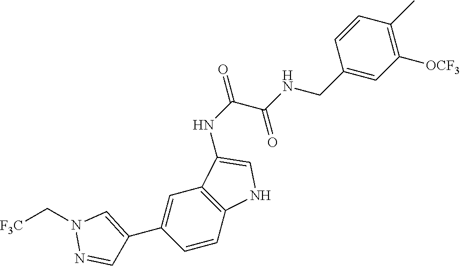 [SMILES: Cc1ccc(CNC(=O)C(=O)Nc2c[nH]c3ccc(-c4cnn(CC(F)(F)F)c4)cc23)cc1OC(F)(F)F] | Method DE: MS-ESI: 540 [M + H]⁺. |
| 211 | 302 | 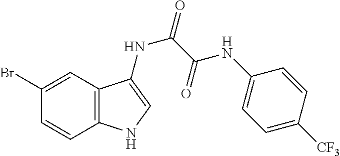 [SMILES: O=C(Nc1ccc(C(F)(F)F)cc1)C(=O)Nc1c[nH]c2ccc(Br)cc12] | Method DC: MS-ESI: 424 [M − H]⁻. |
| 212 | 314 | 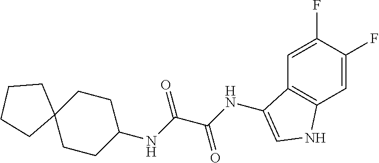 [SMILES: O=C(Nc1c[nH]c2cc(F)c(F)cc12)C(=O)NC1CCC2(CCCC2)CC1] | Method DF: MS-ESI: 374 [M − H]⁻. |
| 213 | 318 | 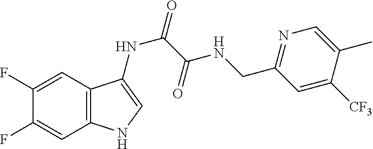 [SMILES: Cc1cnc(CNC(=O)C(=O)Nc2c[nH]c3cc(F)c(F)cc23)cc1C(F)(F)F] | Method DB: MS-ESI: 413 [M + H]⁺. |
| 214 | 319 | 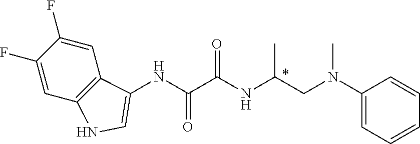 [SMILES: CC(CN(C)c1ccccc1)NC(=O)C(=O)Nc1c[nH]c2cc(F)c(F)cc12] | Method DA: MS-ESI: 387 [M + H]⁺. Column: CHIRALPAK IA, 2 * 25 cm, 5 um; Mobile Phase A: Hex(0.5% 2M NH3—MeOH)--HPLC, Mobile Phase B: EtOH-- HPLC; Flow rate: 20 mL/min; Gradient: 30 B to 30 B in 13 min; |
| | | | 220/254 nm; RT2: |
| | | | 9.116; Injection |
| | | | Volumn: 0.8 ml |
| 215 | 320 | 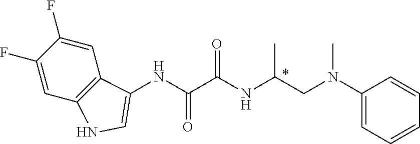 [SMILES: CC(CN(C)c1ccccc1)NC(=O)C(=O)Nc1c[nH]c2cc(F)c(F)cc12] | Method DA: MS-ESI: 387 [M + H]⁺. Column: CHIRALPAK IA, 2 * 25 cm, 5 um; Mobile Phase A: Hex(0.5% 2M NH3—MeOH)--HPLC, Mobile Phase B: EtOH-- HPLC; Flow rate: 20 mL/min; Gradient: 30 B to 30 B in 13 min; |
| | | | 220/254 nm; RTl: 8.624; |
| | | | Injection Volumn: 0.8 ml |
| 216 | 322 | 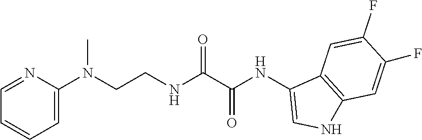 [SMILES: CN(CCNC(=O)C(=O)Nc1c[nH]c2cc(F)c(F)cc12)c1ccccn1] | Method DD: MS-ESI: 374 [M + H]⁺. |
| 217 | 323 | 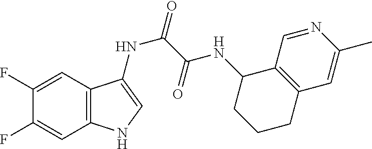 [SMILES: Cc1cc2c(cn1)C(NC(=O)C(=O)Nc1c[nH]c3cc(F)c(F)cc13)CCC2] | Method DF: MS-ESI: 385 [M + H]⁺. |
| 218 | 324 | 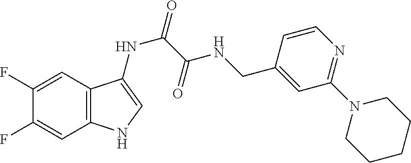 [SMILES: O=C(NCc1ccnc(N2CCCCC2)c1)C(=O)Nc1c[nH]c2cc(F)c(F)cc12] | Method DC: MS-ESI: 414 [M + H]⁺. |
| 219 | 325 | 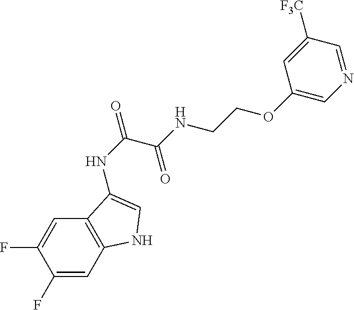 [SMILES: O=C(NCCOc1cncc(C(F)(F)F)c1)C(=O)Nc1c[nH]c2cc(F)c(F)cc12] | Method DD: MS-ESI: 427 [M − H]⁻. |
| 220 | 327 | 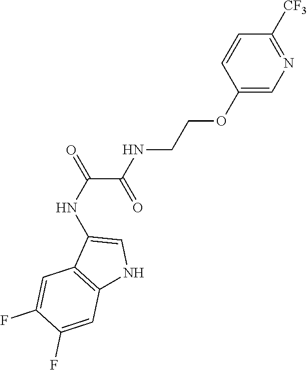 [SMILES: O=C(NCCOc1ccc(C(F)(F)F)nc1)C(=O)Nc1c[nH]c2cc(F)c(F)cc12] | Method DC: MS-ESI: 429 [M + H]⁺. |
| 221 | 329 | 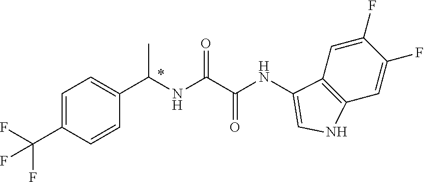 [SMILES: CC(NC(=O)C(=O)Nc1c[nH]c2cc(F)c(F)cc12)c1ccc(C(F)(F)F)cc1] | Method DD: MS-ESI: 410 [M − H]⁻. Column: CHIRALPAK IA, 2 * 25 cm, 5 um; Mobile Phase A: Hex(0.5% 2M NH3—MeOH)--HPLC, Mobile Phase B: EtOH-- HPLC; Flow rate: 20 mL/min; Gradient: 50 B to 50 B in 8 min; 254/220 nm; RT2: 6.733; Injection Volumn: 1.2 ml |
| 222 | 330 | 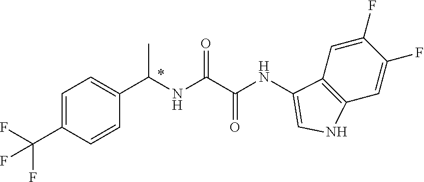 [SMILES: CC(NC(=O)C(=O)Nc1c[nH]c2cc(F)c(F)cc12)c1ccc(C(F)(F)F)cc1] | Method DD: MS-ESI: 410 [M − H]⁻. Column: CHIRALPAK IA, 2 * 25 cm, 5 um; Mobile Phase A: Hex(0.5% 2M NH3—MeOH)--HPLC, Mobile Phase B: EtOH-- HPLC; Flow rate: 20 mL/min; Gradient: 50 B to 50 B in 8 min; 254/220 nm; RT1: 5.381; Injection Volumn: 1.2 ml |
| 223 | 331 | 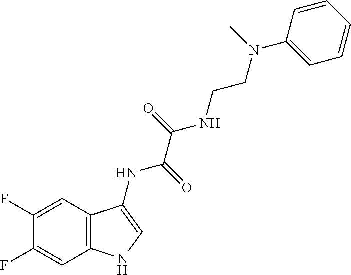 [SMILES: CN(CCNC(=O)C(=O)Nc1c[nH]c2cc(F)c(F)cc12)c1ccccc1] | Method DE: MS-ESI: 373 [M + H]⁺. |
| 224 | 334 | 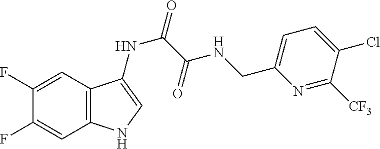 [SMILES: O=C(NCc1ccc(Cl)c(C(F)(F)F)n1)C(=O)Nc1c[nH]c2cc(F)c(F)cc12] | Method DC: MS-ESI: 433 [M + H]⁺. |
| 225 | 335 | 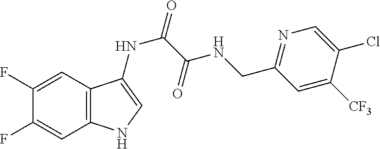 [SMILES: O=C(NCc1cc(C(F)(F)F)c(Cl)cn1)C(=O)Nc1c[nH]c2cc(F)c(F)cc12] | Method DD: MS-ESI: 433 [M + H]⁺. |
| 226 | 338 | 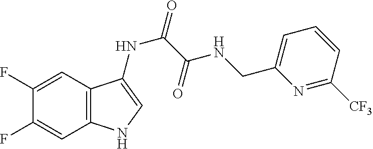 [SMILES: O=C(NCc1cccc(C(F)(F)F)n1)C(=O)Nc1c[nH]c2cc(F)c(F)cc12] | Method DD: MS-ESI: 399 [M + H]⁺. |
| 227 | 340 | 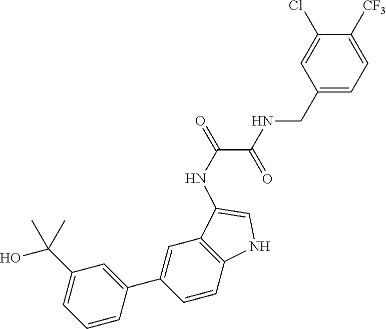 [SMILES: CC(C)(O)c1cccc(-c2ccc3[nH]cc(NC(=O)C(=O)NCc4ccc(C(F)(F)F)c(Cl)c4)c3c2)c1] | Method DC: MS-ESI: 528 [M − H]⁻. |
| 228 | 342 | 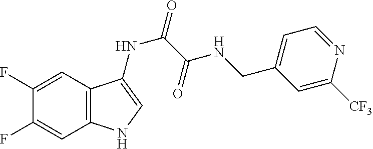 [SMILES: O=C(NCc1ccnc(C(F)(F)F)c1)C(=O)Nc1c[nH]c2cc(F)c(F)cc12] | Method DD: MS-ESI: 399 [M + H]⁺. |
| 229 | 343 | 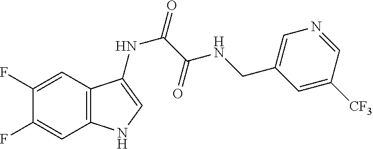 [SMILES: O=C(NCc1cncc(C(F)(F)F)c1)C(=O)Nc1c[nH]c2cc(F)c(F)cc12] | Method DC: MS-ESI: 399 [M + H]⁺. |
| 230 | 344 | 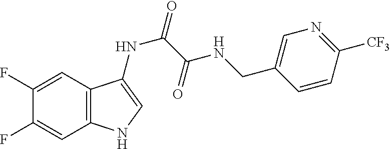 [SMILES: O=C(NCc1ccc(C(F)(F)F)nc1)C(=O)Nc1c[nH]c2cc(F)c(F)cc12] | Method DC: MS-ESI: 399 [M + H]⁺. |
| 231 | 346 | 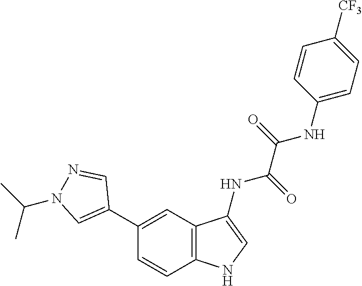 [SMILES: CC(C)n1cc(-c2ccc3[nH]cc(NC(=O)C(=O)Nc4ccc(C(F)(F)F)cc4)c3c2)cn1] | Method DA: MS-ESI: 456 [M + H]⁺. |
| 232 | 347 | 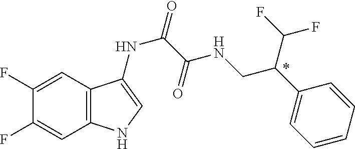 [SMILES: O=C(NCC(c1ccccc1)C(F)F)C(=O)Nc1c[nH]c2cc(F)c(F)cc12] | Method DC: MS-ESI: 392 [M − H]⁻. Column: CHIRAL ART Cellulose-SB, 2 * 25 cm, 5 um; Mobile Phase A: Hex(8 mmol/L NH3•MeOH)--HPLC, Mobile Phase B: EtOH-- HPLC; Flow rate: 45 mL/min; Gradient: 30 B |
| | | | to 30 B in 10 min; |
| | | | 220/254 nm; RT2: 9.116; |
| | | | Injection Volumn: 1 ml |
| 233 | 350 | 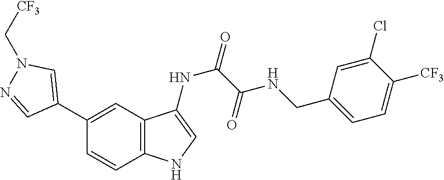 [SMILES: O=C(NCc1ccc(C(F)(F)F)c(Cl)c1)C(=O)Nc1c[nH]c2ccc(-c3cnn(CC(F)(F)F)c3)cc12] | Method DC: MS-ESI: 544 [M + H]⁺. |

### Biological Assays

STING pathway activation by the compounds described herein was measured using THIP1-Dua1™ cells (KO-IFNAR2).

TTIP1-Dual™ KO-IFNAR2 Cells (obtained from invivogen) were maintained in RPMI, 10% FCS, 5 ml P/S, 2 mM L-glut, 10 mM Hepes, and 1 mM sodium pyruvate. Compounds were spotted in empty 384 well tissue culture plates (Greiner 781182) by Echo for a final concentration of 0.0017-100 μM. Cells were plated into the TC plates at 40 μL per well, 2×10E6 cells/mL. For activation with STING ligand, 2′3′cGAMP (MW 718.38, obtained from Invivogen), was prepared in Optimem media.

The following solutions were prepared for each 1×384 plate:
Solution A: 2 mL Optimem with one of the following stimuli:
   60 uL of 10 mM 2′3′cGAMP->150 μM stock
Solution B: 2 mL Optimem with 60 μL Lipofectamine 2000->Incubate 5 min at RT

2 mL of solution A and 2 ml Solution B was mixed and incubated for 20 min at room temperature (RT). 20 uL of transfection solution (A+B) was added on top of the plated cells, with a final 2′3′cGAMP concentration of 15 μM. The plates were then centrifuged immediately at 340 g for 1 minute, after which they were incubated at 37° C., 5% C02, >98% humidity for 24h. Luciferase reporter activity was then measured. EC₅₀ values were calculated by using standard methods known in the art.

Luciferase reporter assay: 10 μL of supernatant from the assay was transferred to white 384-plate with flat bottom and squared wells. One pouch of QUANTI-Luc™ Plus was dissolved in 25 mL of water. 100 μL of QLC Stabilizer per 25 mL of QUANTI-Luc™ Plus solution was added. 50 μL of QUANTI-Luc™ Plus/QLC solution per well was then added. Luminescence was measured on a Platereader (e.g., Spectramax I3X (Molecular Devices GF3637001)).

Luciferase reporter activity was then measured. EC₅₀ values were calculated by using standard methods known in the art.

Table BA shows the activity of compounds in STING reporter assay: <0.008 μM=“++++++”; >0.008 and <0.04 μM=“+++++”; >0.04 and <0.2 μM=“++++”; >0.2 and <1 μM=“+++” >1 and <5 μM=“++”; >5 and <100 μM=“+”.

TABLE BA

| Compound | hSTING: EC₅₀ |
|---|---|
| No. | (μM) |
| 101 | +++ |
| 102 | +++ |
| 103 | ++++ |
| 104 | +++ |
| 105 | ++++ |
| 107 | ++++ |
| 108 | +++ |
| 109 | +++ |
| 110 | +++ |
| 111 | +++ |
| 112 | ++ |
| 113 | +++ |
| 114 | ++ |
| 115 | ++++ |
| 116 | +++ |
| 117 | ++ |
| 118 | ++++ |
| 119 | ++++ |
| 122 | +++ |
| 123 | ++++ |
| 124 | +++ |
| 125 | +++ |
| 126 | ++++ |
| 127 | ++++ |
| 128 | ++ |
| 129 | +++ |
| 130 | +++ |
| 131 | +++ |
| 132 | +++ |
| 133 | ++++ |
| 134 | +++ |
| 135 | +++ |
| 136 | ++++ |
| 137 | +++ |
| 138 | +++ |
| 139 | +++ |
| 140 | ++ |
| 141 | + |
| 142 | +++ |
| 143 | +++ |
| 144 | +++ |
| 145 | +++ |
| 146 | +++ |
| 147 | +++ |
| 148 | ++++ |
| 149 | +++ |
| 150 | +++ |
| 151 | +++ |
| 152 | +++ |
| 153 | ++ |
| 154 | +++ |
| 155 | +++ |
| 156 | +++ |
| 157 | +++ |
| 158 | +++ |
| 159 | +++ |
| 160 | +++ |
| 161 | +++ |
| 162 | +++ |
| 163 | +++ |
| 164 | ++++ |
| 165 | ++++ |
| 166 | +++ |
| 167 | +++ |
| 168 | +++ |
| 169 | +++ |
| 170 | + |
| 171 | +++ |
| 172 | +++ |
| 173 | +++ |
| 174 | +++ |
| 175 | +++ |
| 176 | +++ |
| 177 | ++++ |
| 178 | +++ |
| 179 | ++ |
| 180 | +++ |
| 181 | +++ |
| 182 | +++ |
| 183 | +++ |
| 184 | +++ |
| 185 | +++ |
| 186 | ++++ |
| 187 | ++ |
| 188 | +++ |
| 189 | +++ |
| 190 | +++ |
| 191 | +++ |
| 195 | +++ |
| 196 | +++ |
| 197 | +++ |
| 198 | +++ |
| 199 | ++++ |
| 200 | +++ |
| 201 | +++ |
| 202 | + |
| 203 | +++ |
| 204 | ++++ |
| 205 | ++ |
| 206 | + |
| 207 | +++ |
| 208 | ++ |
| 209 | +++ |
| 210 | +++ |
| 211 | ++ |
| 212 | +++ |
| 213 | ++++ |
| 215 | +++ |
| 216 | ++ |
| 217 | +++ |
| 218 | +++ |
| 219 | ++ |
| 220 | +++ |
| 221 | +++ |
| 222 | +++ |
| 223 | +++ |
| 224 | ++++ |
| 225 | ++++ |
| 226 | ++++ |
| 227 | ++++ |
| 228 | +++ |
| 229 | ++ |
| 230 | ++++ |
| 231 | +++ |
| 232 | ++ |
| 233 | +++ |
| 234 | +++ |
| 235 | +++ |
| 236 | +++ |
| 238 | ++ |
| 239 | +++ |
| 240 | +++ |
| 241 | +++ |
| 242 | + |
| 243 | ++ |
| 244 | ++ |
| 245 | +++ |
| 246 | +++ |
| 247 | +++ |
| 248 | ++++ |
| 249 | ++ |
| 250 | ++++ |
| 251 | ++ |
| 252 | ++ |
| 253 | +++ |
| 254 | +++ |
| 255 | + |
| 256 | ++ |
| 257 | +++ |
| 258 | +++ |
| 259 | ++++ |
| 260 | ++ |
| 261 | +++ |
| 262 | +++ |
| 263 | +++ |
| 264 | +++ |
| 265 | ++ |
| 266 | +++ |
| 267 | +++ |
| 268 | +++ |
| 269 | +++ |
| 270 | +++ |
| 271 | ++ |
| 272 | +++ |
| 273 | +++ |
| 274 | ++ |
| 275 | +++ |
| 276 | +++ |
| 277 | + |
| 278 | ++ |
| 279 | +++ |
| 280 | +++ |
| 281 | +++ |
| 282 | +++ |
| 284 | ++ |
| 285 | ++++ |
| 286 | +++ |
| 287 | ++ |
| 288 | +++ |
| 289 | ++++ |
| 290 | + |
| 291 | +++ |
| 292 | +++ |
| 293 | +++ |
| 294 | +++ |
| 295 | +++ |
| 296 | +++ |
| 297 | +++ |
| 298 | +++ |
| 299 | ++ |
| 300 | + |
| 301 | +++ |
| 302 | ++++ |
| 303 | +++ |
| 304 | +++ |
| 305 | +++ |
| 306 | ++ |
| 307 | +++ |
| 308 | + |
| 309 | +++ |
| 310 | ++ |
| 311 | +++ |
| 312 | +++ |
| 313 | +++ |
| 314 | +++ |
| 315 | ++++ |
| 316 | +++ |
| 317 | +++ |
| 318 | +++ |
| 319 | +++ |
| 320 | +++ |
| 321 | +++ |
| 322 | +++ |
| 323 | +++ |
| 324 | +++ |
| 325 | +++ |
| 326 | +++ |
| 327 | +++ |
| 328 | +++ |
| 329 | ++++ |
| 330 | ++++ |
| 331 | +++ |
| 332 | ++ |
| 333 | +++ |
| 334 | +++ |
| 335 | ++++ |
| 336 | +++ |
| 337 | +++ |
| 338 | +++ |
| 339 | +++ |
| 340 | ++ |
| 341 | +++ |
| 342 | +++ |
| 343 | +++ |
| 344 | +++ |
| 346 | +++ |
| 347 | +++ |
| 348 | +++ |
| 349 | ++ |
| 350 | +++ |
| 351 | +++ |

### Numbered Clauses

The compounds, compositions, methods, and other subject matter described herein are further described in the following numbered clauses:

1. A compound of Formula I:

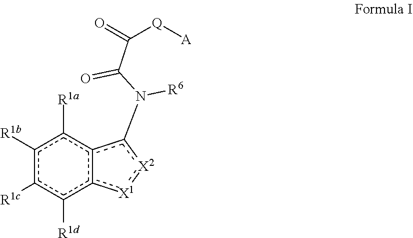

or a pharmaceutically acceptable salt thereof or a tautomer thereof, wherein:

X¹ is selected from the group consisting of O, S, N, NR², and CR⁵;

X² is selected from the group consisting of O, S, N, NR⁴, and CR⁵;

each [symbol] is independently a single bond or a double bond, provided that the five-membered ring comprising X¹ and X² is heteroaryl; and

the 6-membered ring

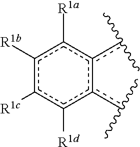

is aromatic;

Q-A is defined according to (A) or (B) below:
(A)

Q is selected from the group consisting of: NH and N(C₁₋₆ alkyl) wherein the C₁₋₆ alkyl is optionally substituted with 1-2 independently selected R^{a}; and

A is:

(i) —(Y^{A1})ₙ—Y^{A2}, wherein:
n is 0 or 1;
Y^{A1} is C₁₋₆ alkylene, which is optionally substituted with 1-6 substituents each independently selected from the group consisting of:
   oxo;
   R^{a};
   C₆₋₁₀ aryl optionally substituted with 1-4 independently selected C₁₋₄ alkyl; and
   heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl ring is optionally substituted with 1-4 independently selected C₁₋₄ alkyl; and
Y^{A1} is —Y^{A3}—Y^{A4}—Y^{A5} which is connected to Q via Y^{A3} wherein:
   Y^{A3} is a C₁₋₃ alkylene optionally substituted with 1-2 substituents each independently selected from the group consisting of oxo and R^{a};
   Y^{A4} is —O—, —NH—, —N(C₁₋₆ alkyl)-, or —S—; and
   Y^{A5} is a bond or C₁₋₃ alkylene which is optionally substituted with 1-2 independently selected R^{a}; or
Y^{A2} is:
   (a) C₃₋₂₀ cycloalkyl or C₃₋₂₀ cycloalkenyl, each of which is optionally substituted with 1-4 R^{b},
   (b) C₆₋₂₀ aryl, which is optionally substituted with 1-4 R^{c};
   (c) heteroaryl of 5-20 ring atoms, wherein 1-3 ring atoms are heteroatoms, each independently selected from the group consisting of N, N(H), N(R^{d}), O, and S(O)₀₋₂, and wherein the heteroaryl ring is optionally substituted with 1-4 independently selected R^{c}; or
   (d) heterocyclyl or heterocycloalkenyl of 3-16 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl ring is optionally substituted with 1-4 independently selected R^{b},

or

(ii) —Z¹—Z²—Z³, wherein:
Z¹ is C₁₋₃ alkylene, which is optionally substituted with 1-4 R^{a};
Z² is —N(H)—, —N(R^{d})—, —O—, or —S—; and
Z³ is C₂₋₇ alkyl, which is optionally substituted with 1-4 R^{a};

or

(iii) C₁₋₂₀ alkyl, which is optionally substituted with 1-6 independently selected R^{a}, or

Q and A, taken together, form:

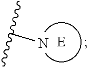

and

E is a ring of 3-16 ring atoms, wherein 0-3 ring atoms are heteroatoms (in addition to the nitrogen atom this is already present), each independently selected from the group consisting of N, N(H), N(R^{d}), O, and S(O)₀₋₂, and wherein the ring is optionally substituted with 1-4 independently selected R^{b}, each of R^{1a}, R^{1b}, R^{1c}, and R^{1d} is independently selected from the group consisting of H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; -L³-L⁴-Rⁱ; —S(O)₁₋₂(C₁₋₄ alkyl); —S(O)(═NH)(C₁₋₄ alkyl); SF₅; —NR^{e}R^{f}; —OH; oxo; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″); or

R^{1a} and R^{1b}, R^{1b} and R^{1c}, or R^{1c} and R^{1d}, taken together with the atoms connecting them, form a ring of 3-10 ring atoms, wherein 0-2 ring atoms are heteroatoms each independently selected from the group consisting of N, N(H), N(R^{d}), 0, and S(O)₀₋₂; and wherein the ring is optionally substituted with 1-4 substituents each independently selected from the group consisting of C₁₋₆ alkyl, halo, C₁₋₆ haloalkyl, —OH, NR^{e}R^{f}, C₁₋₆ alkoxy, and C₁₋₆ haloalkoxy,

each occurrence of R² is independently selected from the group consisting of:
(i) C₁₋₆ alkyl, which is optionally substituted with 1-2 independently selected R^{a};
(ii) C₃₋₆ cycloalkyl or C₃₋₆ cycloalkenyl;
(iii) heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂;
(iv) C₆₋₁₀ aryl;
(v) heteroaryl of 5-10 ring atoms, wherein 1-3 ring atoms are heteroatoms, each independently selected from the group consisting of N, N(H), N(R^{d}), 0, and S(O)₀₋₂;
(vi) —C(O)(C₁₋₄ alkyl);
(vii) —C(O)O(C₁₋₄ alkyl);
(viii) —CON(R′)(R″);
(ix) —S(O)₁₋₂(NR′R″);
(x) —S(O)₁₋₂(C₁₋₄ alkyl);
(xi) —OH;
(xii) C₁₋₄ alkoxy; and
(xiii) H;

R⁴ is selected from the group consisting of H and C₁₋₆ alkyl optionally substituted with 1-3 independently selected R^{a};

R⁵ is selected from the group consisting of H; halo; —OH; —C₁₋₄ alkyl; —C₁₋₄ haloalkyl; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano, and C₃₋₆ cycloalkyl or C₃₋₆ cycloalkenyl, each optionally substituted with 1-4 independently selected C₁₋₄ alkyl;

R⁶ is selected from the group consisting of H; C₁₋₆ alkyl optionally substituted with 1-3 independently selected R^{a}; —OH; C₁₋₄ alkoxy; C(═O)H; C(═O)(C₁₋₄ alkyl); C₆₋₁₀ aryl optionally substituted with 1-4 independently selected C₁₋₄ alkyl; and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 independently selected C₁₋₄ alkyl;

each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano, and C₃₋₆ cycloalkyl or C₃₋₆ cycloalkenyl, each optionally substituted with 1-4 independently selected C₁₋₄ alkyl;

each occurrence of R^{b} is independently selected from the group consisting of: C₁₋₁₀ alkyl optionally substituted with 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₁₀ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};

each occurrence of R^{c} is independently selected from the group consisting of:

halo; cyano; C₁₋₁₀ alkyl which is optionally substituted with 1-6 independently selected R^{a}; C₂₋₆ alkenyl; C₂₋₆ alkynyl; oxo; C₁₋₄ alkoxy optionally substituted with 1-2 independently selected R^{a}; C₁₋₄ haloalkoxy; —S(O)₁₋₂(C₁₋₄ alkyl) or —S(O)₁₋₂(C₁₋₄ haloalkyl); —NR^{e}R^{f}; —OH; —S(O)₁₋₂(NR′R″); —C₁₋₄ thioalkoxy or —C₁₋₄ thiohaloalkoxy; —NO₂; —SF₅; —C(═O)(C₁₋₁₀ alkyl); —C(═O)O(C₁₋₄ alkyl); —C(═O)OH; —C(═O)N(R′)(R″); and -L¹-L²-R^{h};

R^{d} is selected from the group consisting of: C₁₋₆ alkyl optionally substituted with 1-3 substituents each independently selected from the group consisting of halo and OH; C₃₋₆ cycloalkyl or C₃₋₆ cycloalkenyl, each optionally substituted with 1-3 substituents each independently selected from the group consisting of halo and OH; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;

each occurrence of R^{e} and R^{f} is independently selected from the group consisting of: H; C1.6 alkyl; C1.6 haloalkyl; C3-6 cycloalkyl or C3-6 cycloalkenyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or R^{e} and R^{f} together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R^{e} and R^{f}), which are each independently selected from the group consisting of N(R^{d}), NH, 0, and S;

-L¹ is a bond or C₁₋₃ alkylene;

-L² is —O—, —N(H)—, —N(C₁₋₃ alkyl)-, —S(O)₀₋₂—, or a bond;

R^{h} is selected from the group consisting of:
C₃₋₈ cycloalkyl or C₃₋₈ cycloalkenyl, each optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy (in certain embodiments, it is provided that when R^{h} is C₃₋₆ cycloalkyl or C₃₋₆ cycloalkenyl, each optionally substituted with 1-4 substituents independently selected C₁₋₄ alkyl, -L¹ is a bond, or -L² is —O—, —N(H)—, or —S—);
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;

-L³ is a bond or C₁₋₃ alkylene;

-L⁴ is —O—, —N(H)—, —N(C₁₋₃ alkyl)-, —S(O)₀₋₂-, or a bond;

Rⁱ is selected from the group consisting of:
C₃₋₈ cycloalkyl or C₃₋₈ cycloalkenyl, each optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy (in certain embodiments, it is provided that when R is C₃₋₆ cycloalkyl or C₃₋₆ cycloalkenyl, each optionally substituted with 1-4 substituents independently selected C₁₋₄ alkyl, -L¹ is a bond, or -L² is —O—, —N(H)—, or —S—);
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy; and

each occurrence of R′ and R″ is independently selected from the group consisting of: H, C₁₋₄ alkyl, C₆₋₁₀ aryl optionally substituted with 1-2 substituents selected from the group consisting of halo, C₁₋₄ alkyl, and C₁₋₄ haloalkyl, and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo, —OH, NH₂, NH(C₁₋₄ alkyl), N(C₁₋₄ alkyl)₂, C₁₋₄ alkyl, and C₁₋₄ haloalkyl; or R′ and R″ together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R′ and R″), which are each independently selected from the group consisting of N(H), N(C₁₋₆ alkyl), O, and S.

2. The compound of clause 1, wherein X¹ is NR².

3. The compound of any one of clauses 1-2, wherein X¹ is NH.

4. The compound of any one of clauses 1-3, wherein X² is CR⁵.

5. The compound of any one of clauses 1-4, wherein X² is CH.

6. The compound of any one of clauses 1-5, wherein X¹ is NH; and X² is CH.

7. The compound of any one of clauses 1-6, wherein the

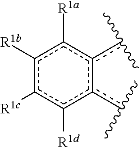

moiety is

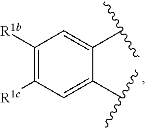

optionally wherein R^{1b} and R^{1c} are each independently selected substituents that are other than hydrogen, such as wherein R^{1b} and R^{1c} are independently selected halo, such as —F or —Cl.

8. The compound of any one of clauses 1-6, wherein the

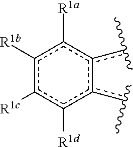

moiety is

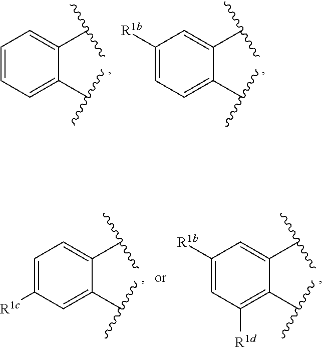

optionally wherein R^{1b}, R^{1c}, and R^{1d} are each independently selected substituents that are other than hydrogen, such as wherein R^{1b} and R^{1c} are independently selected halo, such as —F or —Cl.

9. The compound of any one of clauses 1-2, wherein the compound is a compound of Formula (I-a):

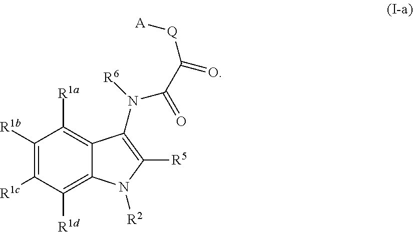

10. The compound of clause 9, wherein the compound has formula (I-a1):

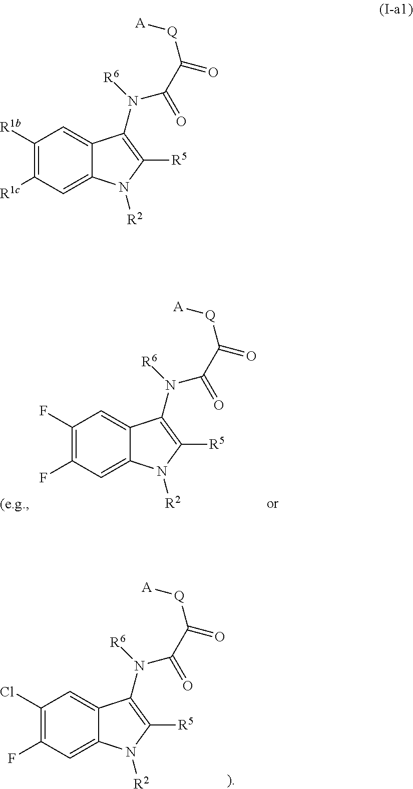

11. The compound of clause 9, wherein the compound has formula (I-a2):

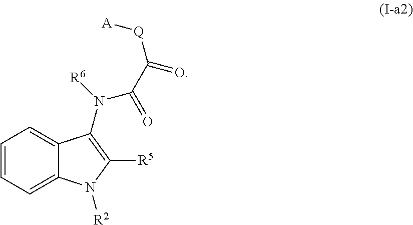

12. The compound of clause 9, wherein the compound has formula (I-a3) or (I-a4):

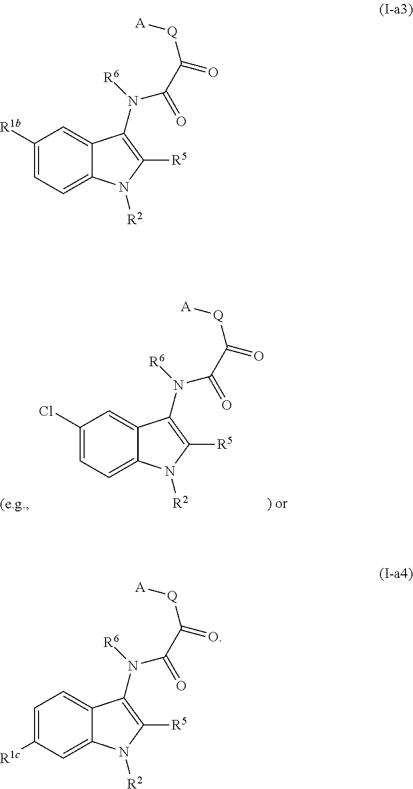

13. The compound of any one of clauses 1-12, wherein R² is H; and R⁵ is H.

14. The compound of any one of clauses 1-13, wherein each of R^{1a}, R^{1b}, R^{1c}, and R^{1d} is independently selected from the group consisting of: H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; -L3-L4-Rⁱ; —S(O)₁₋₂(C₁₋₄ alkyl); —S(O)(═NH)(C₁₋₄ alkyl); SF₅; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″).

15. The compound of any one of clauses 1-14, wherein 1-2 of R^{1a}, R^{1b}, R^{1c}, and R^{1d} is other than H; and each of the remaining of R^{1a}, R^{1b}, R^{1c}, and R^{1d} is H.

16. The compound of any one of clauses 1-15, wherein each of R^{1a}, R^{1b}, R^{1c}, and R^{1d} is H.

17. The compound of any one of clauses 1-15, wherein 1-2 occurrences of R^{1a}, R^{1b}, R^{1c}, and R^{1d} is other than H (e.g., R^{1b} and/or R^{1c} is other than H)

18. The compound of clause 17, wherein two of R^{1a}, R^{1b}, R^{1c}, and R^{1d} are other than H (e.g., R^{1b} and R^{1c} are other than H).

19. The compound of any one of clauses 1-15 and 17-18, wherein 1-2 of R^{1a}, R^{1b}, R^{1c}, and R^{1d} is selected from the group consisting of: halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; —S(O)₁₋₂(C₁₋₄ alkyl); —S(O)₁₋₂(NR′R″); —NO₂; —C(═O)(C₁₋₄ alkyl); —C(═O)O(C₁₋₄ alkyl); —C(═O)OH; and —C(═O)N(R′)(R″).

20. The compound of any one of clauses 1-15 and 17-19, wherein 1-2 occurrence of R^{1a}, R^{1b}, R^{1c}, and R^{1d} is halo (e.g., F or Cl (e.g., F)).

21. The compound of any one of clauses 1-15 and 17-18, wherein one occurrence of R^{1a}, R^{1b}, R^{1c}, and R^{1d} is -L³-L⁴-Rⁱ, such as R^{1b} is -L³-L⁴-Rⁱ; and each remaining occurrences of R^{1a}, R^{1b}, R^{1c}, and R^{1d} is H.

22. The compound of clause 21, wherein -L³ is a bond.

23. The compound of any one of clauses 21-22, wherein -L⁴ is a bond.

24. The compound of any one of clauses 21-23, wherein —Rⁱ is selected from the group consisting of:
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy.

25. The compound of clause 24, wherein —Rⁱ is selected from the group consisting of:
heteroaryl of 5-6 ring atoms (e.g., pyrazolyl), wherein 1-4 ring atoms are heteroatoms, each independently selected from the group consisting of N, N(H), N(R^{d}), O, and S(O)₀₋₂ and wherein the heteroaryl ring is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy; and
phenyl, which is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy.

26. The compound of any one of clauses 21-25, wherein one of R^{1a}, R^{1b}, R^{1c}, and R^{1d} (such as R^{1b}) is selected from the group consisting of:
heteroaryl of 5-6 ring atoms (such as pyrazolyl), wherein 1-4 ring atoms are heteroatoms, each independently selected from the group consisting of N, N(H), N(R^{d}), O, and S(O)₀₋₂ and wherein the heteroaryl ring is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy (e.g.,

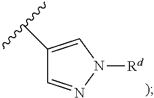

and
phenyl, which is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy (e.g.,

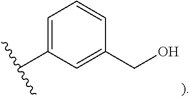 [SMILES: *c1cccc(CO)c1]

27. The compound of any one of clauses 1-13, wherein R^{1a} is H; and R^{1d} is H or halo, such as: wherein R^{1a} is H, and R^{1d} is H; or wherein R^{1a} is H, and R^{1d} is halo such as —F or —Cl.

28. The compound of any one of clauses 1-13 or 27, wherein R^{1b} and R^{1c} are independently selected halo, such as: wherein R^{1b} is —F, and R^{1c} is —F; or wherein R^{1b} is —Cl, and R^{1c} is —F; or wherein R^{1b} is —F, and R^{1c} is —Cl; or wherein R^{1b} is —Cl, and R^{1c} is —Cl.

29. The compound of any one of clauses 1-13 or 27, wherein R^{1b} is halo; and R^{1c} is H, such as: wherein R^{1b} is —Cl, and R^{1c} is H; or wherein R^{1b} is —F, and R^{1c} is H.

30. The compound of any one of clauses 1-13 or 27, wherein R^{1b} is H; and R^{1c} is halo, such as: wherein R^{1b} is H, and R^{1c} is —F; or wherein R^{1b} is H, and R^{1c} is —Cl.

31. The compound of any one of clauses 1-13 or 27, wherein R^{1b} is Rⁱ; and R^{1c} is H or halo, such as H; such as: wherein R^{1b} is selected from the group consisting of:
heteroaryl of 5-6 ring atoms, such as pyrazolyl, wherein 1-4 ring atoms are heteroatoms, each independently selected from the group consisting of N, N(H), N(R^{d}), O, and S(O)₀₋₂ and wherein the heteroaryl ring is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy (e.g.,

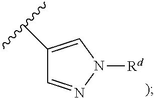

and
phenyl, which is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy (e.g.,

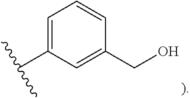 [SMILES: *c1cccc(CO)c1]

32. The compound of any one of clauses 1-13 or 27, wherein one of R^{1b} and R^{1c} is selected from the group consisting of: cyano, C₁₋₃ alkyl optionally substituted with R^{a}, and C₁₋₃ haloalkyl; and the other of R^{1b} and R^{1c} is H or halo, such as —H, —F, or —Cl.

33. The compound of any one of clauses 28-32, wherein R^{1a} is H; and R^{1d} is H.

34. The compound of any one of clauses 28-32, wherein R^{1a} is H; and R^{1d} is halo, such as —F or —Cl.

35. The compound of any one of claims 1-34, wherein Q-A is defined according to (A).

36. The compound of any one of clauses 1-35, wherein Q is NH.

37. The compound of any one of clauses 1-35, wherein Q is N(C₁₋₃ alkyl) (e.g., NMe or NEt).

38. The compound of any one of clauses 1-37, wherein A is —(Y^{A1})ₙ—Y^{A2}

39. The compound of any one of clauses 1-38, wherein n is 0.

40. The compound of any one of clauses 1-38, wherein n is 1.

41. The compound of any one of clauses 1-38 or 40, wherein Y^{A1} is C₁₋₆ alkylene, which is optionally substituted with 1-4 R^{a} and further optionally substituted with oxo.

42. The compound of clauses 1-38 or 40-41, wherein Y^{A1} is C1-6 alkylene which is optionally substituted with 1-4 R^{a}.

43. The compound of clause 42, wherein each R^{a} is independently selected from the group consisting of: —F, —Cl, —Br, —C₃₋₅ cycloalkyl, and —OH.

44. The compound of any one of clauses 1-38 or 40-42, wherein Y^{A1} is —CH₂—, —CH₂CH₂—, —CH₂CH₂CH₂—, —CH(CF₃)—, —CH₂CH(OH)—,

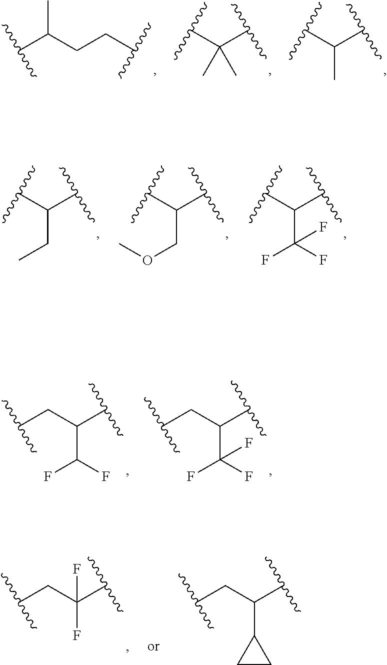

(e.g., Y^{A1} is CH₂ or CH₂CH₂).

45. The compound of clause 44, wherein Y^{A1} is —CH₂—, —CH₂CH₂—, or

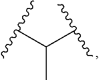 [SMILES: *C(*)C]

such as

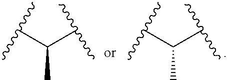

46. The compound of clauses 1-38 or 40-41, wherein Y^{A1} is C₁₋₆ alkylene that is substituted with oxo, wherein Y^{A1} is further optionally substituted with 1-2 R^{a}.

47. The compound of clause 46, wherein Y^{A1} is C₂₋₃ alkylene substituted with oxo, such as wherein Y^{A1} is

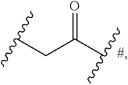 [SMILES: *CC(*)=O]

wherein # represents point of attachment to Y^{A2}.

48. The compound of any one of clauses 1-38 or 40, wherein Y^{A1} is —Y^{A3}—Y^{A4}—Y^{A5} which is connected to Q via Y^{A3}.

49. The compound of clause 48, wherein Y^{A5} is a bond.

50. The compound of clauses 48 or 49, wherein Y^{A4} is —O—.

51. The compound of clauses 48 or 49, wherein Y^{A4} is —NH— or —N(C₁₋₃ alkyl)-, such as wherein Y^{A4} is -N(Me)-.

52. The compound of any one of clauses 48-51, wherein Y^{A3} is C₂₋₃ alkylene optionally substituted with 1-2 R^{a}, such as wherein Y^{A3} is

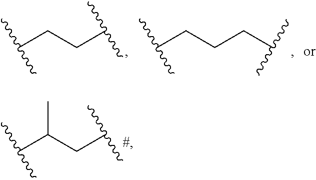

wherein # represents point of attachment to Y^{A4}.

53. The compound of any one of clauses 48-51, wherein Y^{A3} is C₂₋₃ alkylene substituted with oxo and further optionally substituted with R^{a}, such as wherein Y^{A3} is

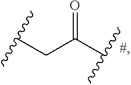 [SMILES: *CC(*)=O]

wherein # represents point of attachment to Y^{A4}.

54. The compound of any one of clauses 1-38, 40, or 48-49, wherein Y^{A1} is —Y^{A3}—O—; and Y^{A3} is C₂₋₃ alkylene optionally substituted with 1-2 R^{a}, such as wherein Y^{A3} is

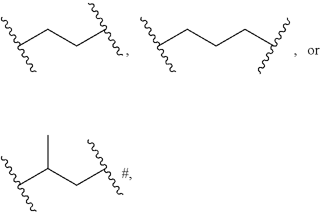

wherein # represents point of attachment to —O—.

55. The compound of any one of clauses 40 or 48-49, wherein Y^{A1} is —Y^{A3}—N(C₁₋₃ alkyl)-, such as —Y^{A3}—N(Me)-; and Y^{A3} is C₂₋₃ alkylene optionally substituted with 1-2 R^{a}, such as wherein Y^{A3} is

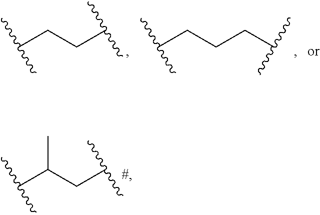

wherein # represents point of attachment to —N(C₁₋₃ alkyl)-.

56. The compound of any one of clauses 38-55, wherein Y^{A2} is C₆₋₁₀ aryl, which is optionally substituted with 1-3 R^{c}.

57. The compound of any one of clauses 38-56, wherein Y^{A2} is C₆ aryl, which is optionally substituted with 1-3 R^{c}.

58. The compound of any one of clauses 38-57, wherein Y^{A2} is C₆ aryl, which is substituted with 1-3 R^{c}.

59. The compound of any one of clauses 38-58, wherein Y^{A2} is phenyl substituted with 1-3 R^{c}, wherein one R^{c} is at the ring carbon para to the point of attachment to Y^{A1}.

60. The compound of any one of clauses 38-58, wherein Y^{A2} is phenyl substituted with 1-3 R^{c}, wherein 1-2 R^{c} is at the ring carbons meta to the point of attachment to Y^{A1}.

61. The compound of any one of clauses 38-58, wherein Y^{A2} is phenyl substituted with 1-3 R^{c}, wherein 1-2 R^{c} is at the ring carbons ortho to the point of attachment to Y^{A1}.

62. The compound of any one of clauses 38-57, wherein Y^{A2} is unsubstituted phenyl.

63. The compound of any one of clauses 38-56, wherein Y^{A2} is C₇₋₁₀ bicyclic aryl, which is optionally substituted with 1-3 R^{c} (e.g., Y^{A2} is naphthyl (e.g.,

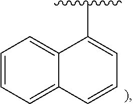 [SMILES: *c1cccc2ccccc12]

indanyl (e.g.,

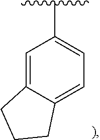 [SMILES: *c1ccc2c(c1)CCC2]

or tetrahydronapthyl (e.g.,

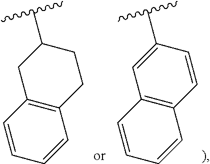 [SMILES: *C1CCc2ccccc2C1 ; *c1ccc2ccccc2c1]

each of which is optionally substituted with 1-3 R^{c}.

64. The compound of any one of clauses 38-55, wherein Y^{A2} is heteroaryl of 5-14 ring atoms, wherein 1-3 ring atoms are heteroatoms, each independently selected from the group consisting of N, N(H), N(R^{d}), O, and S(O)₀₋₂, and wherein the heteroaryl ring is optionally substituted with 1-4 independently selected R^{c}.

65. The compound of any one of clauses 38-55 and 64, wherein Y^{A2} is heteroaryl of 6 ring atoms (e.g., pyridyl or pyrimidinyl (e.g., pyridyl)), wherein 1-2 ring atoms are ring nitrogen atoms, and wherein the heteroaryl ring is optionally substituted with 1-3 independently selected R^{c}.

66. The compound of clause 65, wherein Y^{A2} is substituted with 1-3 independently selected R^{c}; and one occurrence of R^{c} is at the ring carbon atom para to the point of attachment to Y^{A1}, such as wherein Y^{A2} is

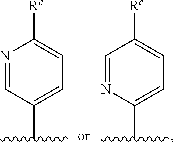

each of which is further optionally substituted with 1-2 independently selected R^{c}.

67. The compound of clause 65, wherein Y^{A2} is substituted with 1-3 independently selected R^{c}; and one occurrence of R^{c} is at the ring carbon atom meta to the point of attachment to Y^{A1}, such as wherein Y^{A2} is

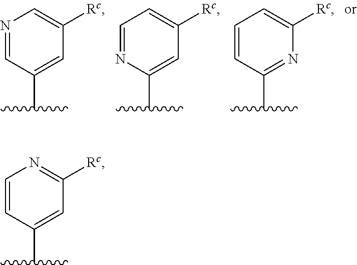

each of which is further optionally substituted with 1-2 independently selected R^{c}.

68. The compound of any one of clauses 38-55 or 64, wherein Y^{A2} is bicyclic or tricyclic heteroaryl of 7-14 (e.g., 9-12 (e.g., 9, 10, 11, or 12)) ring atoms, wherein 1-3 ring atoms are heteroatoms, each independently selected from the group consisting of N, N(H), N(R^{d}), O, and S(O)₀₋₂, and wherein the heteroaryl ring is optionally substituted with 1-4 independently selected R^{c}.

69. The compound of clause 68, wherein Y^{A2} is bicyclic heteroaryl of 9-10 ring atoms, wherein 1-3 ring atoms are heteroatoms, each independently selected from the group consisting of N, N(H), N(R^{d}), O, and S(O)₀₋₂, and wherein the heteroaryl ring is optionally substituted with 1-4 independently selected R^{c}.

70. The compound of clause 69, wherein Y^{A2} is bicyclic heteroaryl of 9-10 ring atoms, wherein 1-3 ring atoms are heteroatoms, each independently selected from the group consisting of N, N(H), N(R^{d}), O, and S(O)₀₋₂ and wherein the heteroaryl ring is optionally substituted with 1-3 independently selected R^{c}.

71. The compound of clause 70, wherein Y^{A2} is selected from the group consisting of:

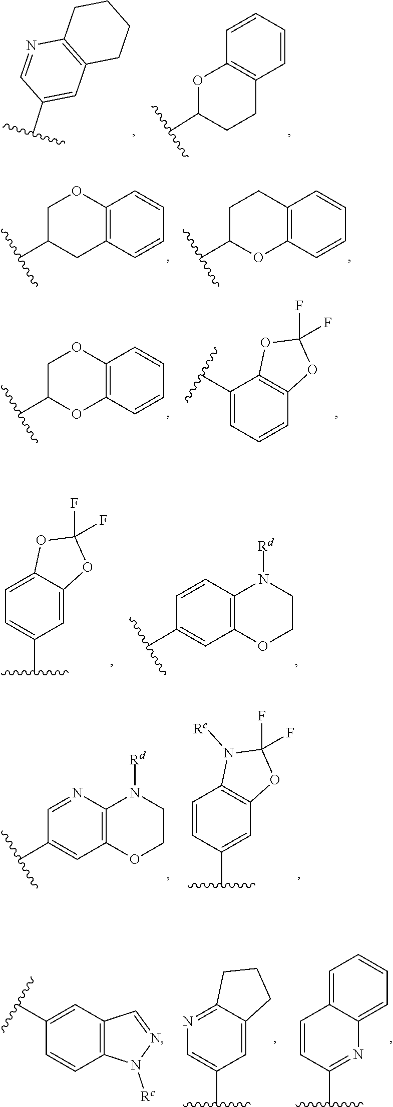

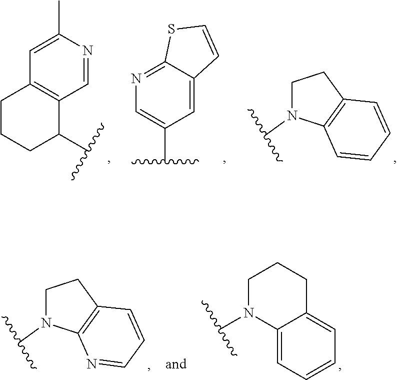 [SMILES: *C1CCCc2cc(C)ncc21 ; *c1cnc2sccc2c1 ; *N1CCc2ccccc21 ; *N1CCc2cccnc21 ; *N1CCCc2ccccc21]

each of which is further optionally substituted with 1-2 independently selected R^{c}.

72. The compound of any one of clauses 56-71, wherein each occurrence of R^{c} is independently selected from the group consisting of: halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —NR^{e}R^{f}; —C₁₋₄ thioalkoxy; —C(═O)(C₁₋₁₀ alkyl); —C(═O)(OH); —C(═O)O(C1.4 alkyl); and -L¹-L²-R^{h}.

73. The compound of any one of clauses 56-72, wherein one occurrence of R^{c} is halo (e.g., F or C1 (e.g., C1)); or wherein one occurrence of R is C₂₋₆ alkynyl (e.g.,

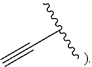 [SMILES: *C#C]

74. The compound of any one of clauses 56-72, wherein one occurrence of R^{c} is C₁₋₁₀ alkyl which is optionally substituted with 1-6 independently selected R^{a}.

75. The compound of any one of clauses 56-72 or 74, wherein one occurrence of R^{c} is unsubstituted C₁₋₁₀ alkyl (e.g., C₂, C₃, C₄, C₅, C₆, or C₇₋₁₀).

76. The compound of clause 75, wherein one occurrence of R^{c} is ethyl, propyl (e.g., n-propyl), butyl (e.g., n-butyl, iso-butyl, sec-butyl, tert-butyl), or octyl (e.g., n-octyl).

77. The compound of clause 74, wherein one occurrence of R^{c} is C₁₋₁₀ alkyl which is substituted with 1-6 independently selected R^{a}.

78. The compound of clause 77, wherein each occurrence of R^{a} is independently selected from —F, —Cl, —Br, OH, C₁₋₄ alkoxy, and C₁₋₄ haloalkoxy.

79. The compound of clause 78, wherein each occurrence of R^{a} is —F or —Cl, such as —F.

80. The compound of clause 77, wherein one occurrence of R^{c} is C₁₋₃ alkyl substituted with 1-6-F, such as wherein R^{c} is CF₃.

81. The compound of any one of clauses 56-72, wherein one occurrence of R^{c} is -L¹-L²-R^{h}.

82. The compound of clause 81, wherein L¹ is a bond.

83. The compound of clause 81, wherein L¹ is CH₂.

84. The compound of any one of clauses 81-83, wherein L² is —O—.

85. The compound of any one of clauses 81-83, wherein L² is —N(H)— or —NH(C₁₋₃ alkyl)-, such as —N(H)—.

86. The compound of any one of clauses 81-83, wherein L² is a bond.

87. The compound of clause 81, wherein one occurrence of R^{c} is R^{h}.

88. The compound of clause 81, wherein one occurrence of R^{c} is —CH₂—R^{h}.

89. The compound of clause 81, wherein one occurrence of R^{c} is —O—R^{h}, —N(H)—R^{h}, or —N(C₁₋₃ alkyl)-R^{h}.

90. The compound of any one of clauses 81-89, wherein R^{h} is C₆₋₁₀ aryl, which is optionally substituted with 1-4 substituents independently selected from the group consisting of halo, C₁₋₄ alkyl, and C₁₋₄ haloalkyl.

91. The compound of any one of clauses 81-90, wherein R^{h} is C₆ aryl, which is optionally substituted with 1-2 substituents independently selected from the group consisting of halo, C₁₋₄ alkyl, and C₁₋₄ haloalkyl (e.g., R^{h} can be

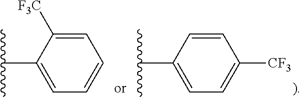

92. The compound of any one of clauses 81-89, wherein R^{h} is heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy.

93. The compound of any one of clauses 81-89 or 92, wherein R^{h} is heteroaryl of 5-6 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy.

94. The compound of any one of clauses 81-89, wherein R^{h} is heterocyclyl or heterocycloalkenyl, wherein the heterocyclyl or heterocycloalkenyl has 3-10 (e.g., 5-6) ring atoms, wherein 1-3 (e.g., 1-2) ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo, C₁₋₄ alkyl, and C₁₋₄ haloalkyl.

95. The compound of clause 94, wherein R^{h} is

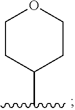 [SMILES: *C1CCOCC1]

or wherein R^{h} is

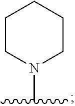 [SMILES: *N1CCCCC1]

or wherein R^{h} is

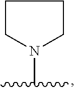 [SMILES: *N1CCCC1]

each of which is optionally substituted with 1-4 substituents independently selected from the group consisting of halo, C₁₋₄ alkyl, and C₁₋₄ haloalkyl.

96. The compound of any one of clauses 81-89, wherein R^{h} is C₃₋₈ cycloalkyl or C₃₋₈ cycloalkenyl, each optionally substituted with 1-4 substituents independently selected from the group consisting of halo, C₁₋₄ alkyl, and C₁₋₄ haloalkyl.

97. The compound of clause 96, wherein R^{h} is C₃₋₆ cycloalkyl or C₃₋₆ cycloalkenyl, each optionally substituted with 1-4 substituents independently selected from the group consisting of halo, C₁₋₄ alkyl, and C₁₋₄ haloalkyl.

98. The compound of clause 97, wherein R^{h} is selected from the group consisting of:

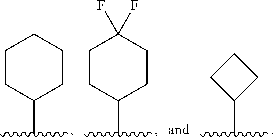

99. The compound of any one of clauses 73-98, wherein each of the remaining occurrences of R^{c} is C1.6 alkyl or halo.

100. The compound of any one of clauses 1-55, wherein Y^{A2} is monocyclic C₃₋₁₀ cycloalkyl or C₃₋₁₀ cycloalkenyl, each of which is optionally substituted with 1-4 R^{b}.

101. The compound of any one of clauses 1-55 or 100, wherein Y^{A2} is C₃₋₆(e.g., C₃, C₅, or C₆) cycloalkyl or C₃₋₆(e.g., C₃, C₅, or C₆) cycloalkenyl, each of which is substituted with 1-4 (e.g., 1-2) R^{b}, such as wherein Y^{A2} is C₃₋₆(e.g., C₃, C₅, or C₆) cycloalkyl which is optionally substituted with 1-4 (e.g., 1-2) R^{b}.

102. The compound of any one of clauses 1-55 or 100-101, wherein Y^{A2} is cyclopropyl, cyclobutyl, cyclopentyl, or cyclohexyl, each of which is optionally substituted with 1-2 R^{b}.

103. The compound of clause 102, wherein Y^{A2} is cyclohexyl which is optionally substituted with 1-2 R^{b}.

104. The compound of clause 103, wherein one occurrence of R^{b} is at the ring carbon atom para to the point of attachment to Y^{A1}; or one occurrence of R^{b} is at the ring carbon atom meta to the point of attachment to Y^{A1}, such as wherein Y^{A2} is

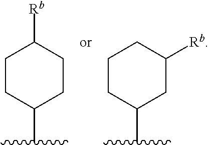

105. The compound of clause 103, wherein two occurrences of R^{b} are at the ring carbon atom para to the point of attachment to Y^{A1}; or two occurrences of R^{b} are at the ring carbon atom meta to the point of attachment to Y^{A1}.

106. The compound of clause 103, wherein Y^{A2} is unsubstituted cyclohexyl.

107. The compound of clause 102, wherein Y^{A2} is cyclobutyl which is substituted with 1-2 R^{b}, such as wherein Y^{A2} is

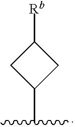

108. The compound of clause 102, wherein Y^{A2} is cyclopentyl which is optionally substituted with 1-2 R^{b}, such as wherein Y^{A2} is unsubstituted cyclopentyl; or wherein Y^{A2} is

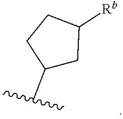

109. The compound of clause 102, wherein Y^{A2} is cyclopropyl which is substituted with 1-2 R^{b}, such as cyclopropyl substituted with -L¹-L²-R^{h} (e.g.,

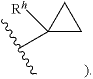

110. The compound of any one of clauses 1-55 or 100, wherein Y^{A2} is

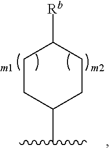

wherein m1 and m2 are independently 0, 1, or 2.

111. The compound of any one of clauses 1-55, wherein Y^{A2} is bicyclic, tricyclic, or polycyclic C₇₋₂₀ (e.g., C₇₋₁₂) cycloalkyl or C7-20 (e.g., C₇₋₁₂) cycloalkenyl, each optionally substituted with 1-2 R^{b}.

112. The compound of any one of clauses 1-55 or 111, wherein Y^{A2} is a spirocyclic bicyclic C₇₋₁₂ cycloalkyl optionally substituted with 1-2 R^{b}, such as wherein Y^{A2} is: spiro[5.5]undecanyl (e.g.,

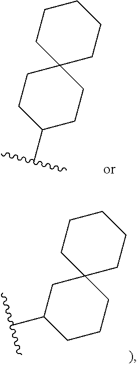 [SMILES: *C1CCC2(CCCCC2)CC1 ; *C1CCCC2(CCCCC2)C1]

spiro[4.4]nonanyl (e.g.,

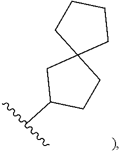 [SMILES: *C1CCC2(CCCC2)C1]

spiro[4.5]decanyl (e.g.,

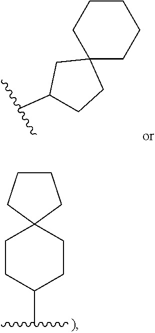 [SMILES: *C1CCC2(CCCCC2)C1 ; *C1CCC2(CCCC2)CC1]

or spiro[2.5]octanyl (e.g.,

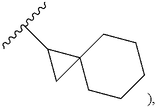 [SMILES: *C1CC12CCCCC2]

each of which is optionally substituted with R^{b}.

113. The compound of any one of clauses 1-55 or 111, wherein Y^{A2} is a bridged bicyclic C₇₋₁₂ cycloalkyl or C₇₋₁₂ cycloalkenyl optionally substituted with 1-2 R^{b}, such as wherein Y^{A2} is adamantly (e.g.,

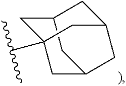 [SMILES: *C12CC3CC(CC(C3)C1)C2]

bicyclo[2.2.1]heptanyl (e.g.,

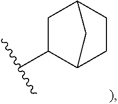 [SMILES: *C1CC2CCC1C2]

and bicyclo[2.2.1]hept-2-enyl (e.g.,

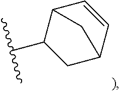 [SMILES: *C1CC2C=CC1C2]

or bicyclo[3.2.1]octan (e.g.,

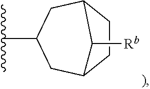

each of which is optionally substituted with R^{b}.

114. The compound of any one of clauses 1-55, wherein Y^{A2} is heterocyclyl or heterocycloalkenyl of 3-16 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl ring is optionally substituted with 1-3 independently selected R^{b}, such as wherein Y^{A2} is heterocyclyl of 4-8 ring atoms, such as 4-6 ring atoms, wherein 1-2 ring atoms are heteroatoms, each independently selected from the group consisting of N, N(H), N(R^{d}), O, and S(O)₀₋₂, and wherein the heterocyclyl ring is optionally substituted with 1-2 independently selected R^{b}, such as wherein Y^{A2} is

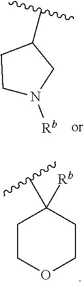

115. The compound of any one of clauses 1-55 or 114, wherein Y^{A2} is

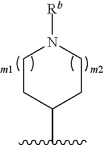

wherein m1 and m2 are independently 0, 1, or 2.

116. The compound of any one of clauses 1-55 or 100-115, wherein each occurrence of R^{b} substituent of Y^{A2} is independently selected from the group consisting of: C₁₋₁₀ alkyl optionally substituted with 1-6 independently selected R^{a}; C₁₋₄ haloalkyl; —F; —Cl; —Br; cyano; C₁₋₄ alkoxy; C₁₋₄ haloalkoxy; —C(═O)(C₁₋₁₀ alkyl); —C(═O)O(C₁₋₄ alkyl); —S(O)₁₋₂(C₁₋₄ alkyl); oxo; cyano; and -L¹-L²-R^{h}.

117. The compound of any one of clauses 1-55 or 100-115, wherein one occurrence of R^{b} substituent of Y^{A2} is C₁₋₁₀ alkyl which is optionally substituted with 1-6 independently selected R^{a}.

118. The compound of clause 117, wherein one occurrence of R^{b} substituent of Y^{A2} is unsubstituted C₁₋₁₀ alkyl (e.g., C₂, C₃, C₄, C₅, C₆, or C₇₋₁₀).

119. The compound of clause 118, wherein one occurrence of R^{b} substituent of Y^{A2} is ethyl, propyl (e.g., n-propyl), butyl (e.g., n-butyl; or sec-butyl; or tert-butyl; or iso-butyl), or octyl (e.g., n-octyl).

120. The compound of clause 117, wherein one occurrence of R^{b} substituent of Y^{A2} is C₁₋₁₀ alkyl which is substituted with 1-6 independently selected R^{a} (e.g., R^{b} is CF₃ or —CF₂CH₃).

121. The compound of clause 120, wherein each occurrence of R^{a} is independently selected from —F, —Cl, —Br, OH, C₁₋₄ alkoxy, and C₁₋₄ haloalkoxy.

122. The compound of any one of clauses 1-55 or 100-115, wherein one occurrence of R^{b} substituent of Y^{A2} is -L¹-L²-R^{h} (e.g., —R^{h} or —CH₂—R^{h} such as benzyl).

123. The compound of clause 122, wherein -L¹ is a bond.

124. The compound of clause 122, wherein -L¹ is CH₂.

125. The compound of any one of clauses 122-124, wherein L² is —O—.

126. The compound of any one of clauses 122-124, wherein L² is —N(H)— or —NH(C₁₋₃ alkyl)-, such as —N(H)—.

127. The compound of any one of clauses 122-124, wherein L² is a bond.

128. The compound of any one of clauses 1-55, 100-115, or 122, wherein one occurrence of R^{b} is R^{h}.

129. The compound of any one of clauses 1-55, 100-115, or 122, wherein one occurrence of R^{b} is —CH₂—R^{h}.

130. The compound of any one of clauses 1-55, 100-115, or 122, wherein one occurrence of R^{b} is —O—R^{h}, —N(H)—R^{h}, or —N(C₁₋₃ alkyl)-R^{h}.

131. The compound of any one of clauses 122-130, wherein R^{h} is C₆₋₁₀ aryl, which is optionally substituted with 1-4 substituents independently selected from the group consisting of halo, C₁₋₄ alkyl, and C₁₋₄ haloalkyl.

132. The compound of any one of clauses 122-131, wherein R^{h} is C₆ aryl, which is optionally substituted with 1-2 substituents independently selected from the group consisting of halo, C₁₋₄ alkyl, and C₁₋₄ haloalkyl (e.g.,

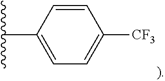 [SMILES: *c1ccc(C(F)(F)F)cc1]

133. The compound of any one of clauses 122-130, wherein R^{h} is heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy.

134. The compound of any one of clauses 122-130 or 133, wherein R^{h} is heteroaryl of 5-6 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy.

135. The compound of any one of clauses 122-130 or 133-134, wherein R^{h} is heteroaryl of 6 ring atoms, wherein 1-2 ring atoms are ring nitrogen atoms and wherein the heteroaryl ring is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy.

136. The compound of clause 135, wherein R^{h} is pyridyl optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy, such as pyridyl substituted with C₁₋₄ haloalkyl, such as, wherein R^{h} is

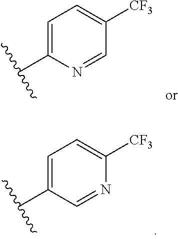 [SMILES: *c1ccc(C(F)(F)F)cn1 ; *c1ccc(C(F)(F)F)nc1]

137. The compound of any one of clauses 1-55, 100-115, or 122, wherein one occurrence of R^{b} is

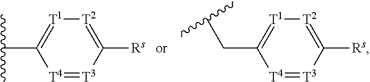

wherein T¹, T², T³, and T⁴ are each independently N, CH, or CR^{t}, provided that 1-4 (e.g., 2, 3, or 4) of T¹-T⁴ is CH, wherein each of R^{t} and R^{s} is independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy.

138. The compound of any one of clauses 1-55, 100-115, or 122, wherein one occurrence of R^{b} is

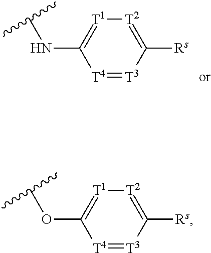

wherein T¹, T², T³, and T⁴ are each independently N, CH, or CR^{t}, provided that 1-4 (e.g., 2, 3, or 4) of T¹-T⁴ is CH, wherein each of R^{t} and R^{s} is independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy.

139. The compound of clauses 137 or 138, wherein each of T¹, T², T³, and T⁴ is independently CH or CR^{t}, such as each of T¹, T², T³, and T⁴ is CH.

140. The compound of clauses 137 or 138, wherein T¹ is N; and T², T³, and T⁴ are independently CH or CRt, such as wherein T¹ is N; and T², T³, and T⁴ are CH.

141. The compound of clauses 137 or 138, wherein T² is N; and T¹, T³, and T⁴ are independently CH or CRt, such as wherein T² is N; and T¹, T³, and T⁴ are CH.

142. The compound of any one of clauses 137-141, wherein R^{s} is C₁₋₄ alkyl, such as methyl.

143. The compound of any one of clauses 137-141, wherein R^{s} is C₁₋₄ haloalkyl, such as CF₃.

144. The compound of any one of clauses 1-55, 100-115, 122, or 137, wherein R^{b} is

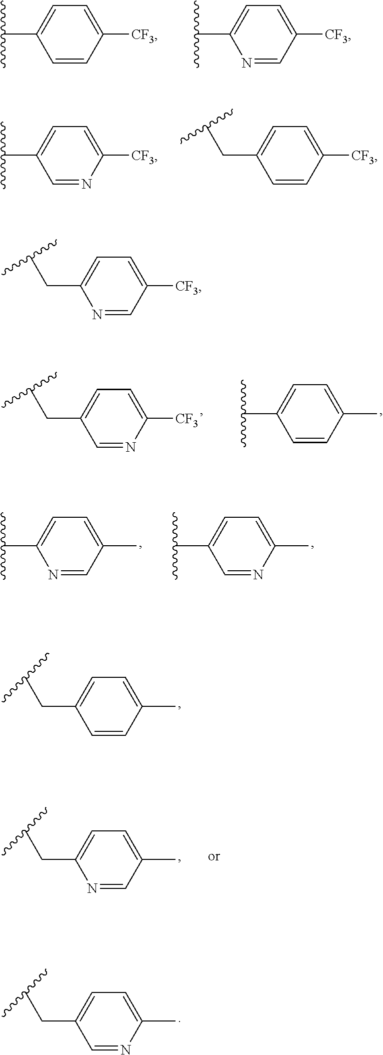

145. The compound of any one of clauses 1-55, 100-115, 122, or 137, wherein R^{b} is

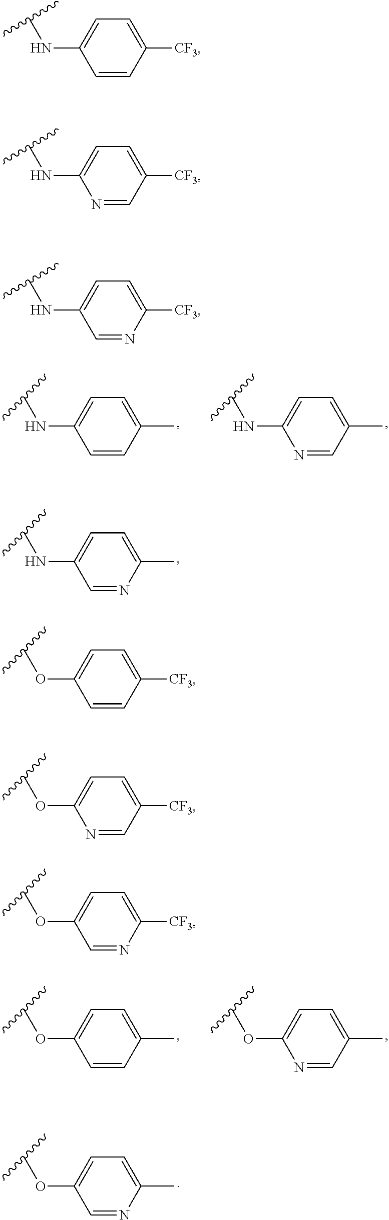

146. The compound of any one of clauses 1-55 or 100-115, wherein one occurrence of R^{b} substituent of Y^{A2} is C₁₋₄ alkoxy or C₁₋₄ haloalkoxy (e.g.,

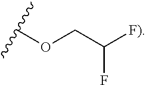

147. The compound of any one of clauses 1-55 or 100-115, wherein one occurrence of R^{b} is —F or —Cl (e.g., —F).

148. The compound of any one of clauses 117-147, wherein each remaining R^{b} substituent of Y^{A2} when present is —F, —Cl, or C₁₋₃ alkyl.

149. The compound of any one of clauses 1-55, wherein Y^{A2} is

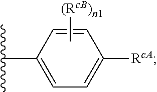

n1 is 0, 1, or 2; and each of R^{cA} and R^{cB} is an independently selected R^{c}, such as wherein Y^{A2} is

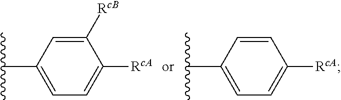

150. The compound of any one of clauses 1-55, wherein Y^{A2} is

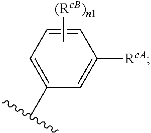

n1 is 0, 1, or 2; and each of R^{cA} and R^{cB} is an independently selected R^{c}, such as wherein Y^{A2} is

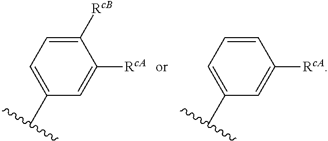

151. The compound of any one of clauses 1-55, wherein Y^{A2} is

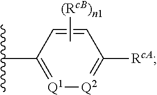

one of Q¹ and Q² is N; the other one of Q¹ and Q² is CH; n1 is 0, 1, or 2; and each of R^{cA} and R^{cB} is an independently selected R^{c}, such as wherein Y^{A2} is

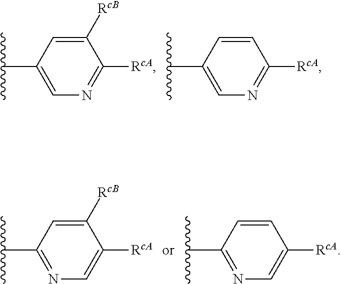

152. The compound of any one of clauses 1-55, wherein Y^{A2} is

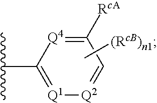

one of Q¹, Q², Q³, and Q⁴ is N; each of the remaining of Q¹, Q², Q³, and Q⁴ is CH; n1 is 0, 1, or 2; and each of R^{cA} and R^{cB} is an independently selected R^{c}, such as wherein Y^{A2} is

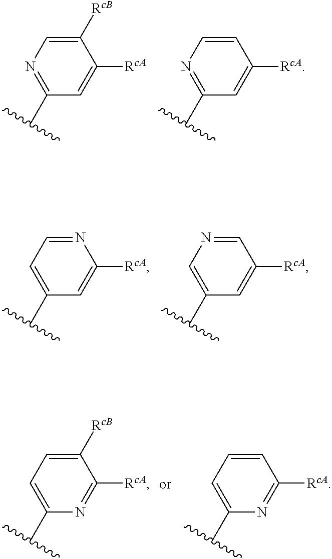

153. The compound of any one of clauses 149-152, wherein RCA is selected from the group consisting of: halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —NR^{I}R^{f}; —C₁₋₄ thioalkoxy; —C(═O)(C₁₋₁₀ alkyl); —C(═O)(OH); —C(═O)O(C₁₋₄ alkyl); and -L¹-L²-R^{h}.

154. The compound of any one of clauses 149-153, wherein R^{cA} is unsubstituted C₁₋₁₀ alkyl (e.g., C₂, C₃, C₄, C₅, C₆, or C₇₋₁₀), such as ethyl, propyl (e.g., n-propyl), butyl (e.g., n-butyl, iso-butyl, sec-butyl, tert-butyl), or octyl (e.g., n-octyl).

155. The compound of any one of clauses 149-153, wherein R^{cA} is C₁₋₁₀ alkyl which is substituted with 1-6 independently selected R^{a} (e.g., each occurrence of R^{a} is independently selected from —F, —Cl, —Br, OH, C₁₋₄ alkoxy, and C₁₋₄ haloalkoxy).

156. The compound of clause 155, wherein R^{cA} is C₁₋₁₀ alkyl which is substituted with 1-6 independently selected —F, —Cl, or -Br (e.g., R^{cA} is CF₃).

157. The compound of any one of clauses 149-153, wherein R^{cA} is -L¹-L²-R^{h}

158. The compound of clause 157, wherein -L¹ is a bond.

159. The compound of clauses 157 or 158, wherein -L² is a bond.

160. The compound of clauses 157 or 158, wherein -L² is —O—, —N(H)—, or —N(C₁₋₃ alkyl)-.

161. The compound of any one of clauses 157-160, wherein R^{h} is C₆₋₁₀ aryl, which is optionally substituted with 1-4 substituents independently selected from the group consisting of halo, C₁₋₄ alkyl, and C₁₋₄ haloalkyl,

such as C₆ aryl, which is optionally substituted with 1-2 substituents independently selected from the group consisting of halo, C₁₋₄ alkyl, and C₁₋₄ haloalkyl (e.g.,

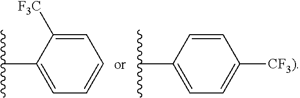

162. The compound of any one of clauses 157-160, wherein R^{h} is heteroaryl of 5-6 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy.

163. The compound of any one of clauses 157-160, wherein R^{h} is heterocyclyl or heterocycloalkenyl, wherein the heterocyclyl or heterocycloalkenyl has 3-10 (e.g., 5-6) ring atoms, wherein 1-3 (e.g., 1-2) ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo, C₁₋₄ alkyl, and C₁₋₄ haloalkyl, such as

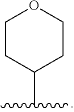 [SMILES: *C1CCOCC1]

164. The compound of any one of clauses 157-160, wherein R^{h} is C₃₋₈(e.g., C₃₋₆) cycloalkyl or C₃₋₈(e.g., C₃₋₆) cycloalkenyl, each optionally substituted with 1-4 substituents independently selected from the group consisting of halo, C₁₋₄ alkyl, and C₁₋₄ haloalkyl (e.g., R^{h} is cyclohexyl).

165. The compound of any one of clauses 149-153, wherein RCA is C₂₋₆ alkynyl (e.g.,

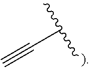 [SMILES: *C#C]

166. The compound of any one of clauses 149-165, wherein n1 is 0.

167. The compound of any one of clauses 149-165, wherein n1 is 1 or 2 (e.g., 1).

168. The compound of clause 167, wherein each occurrence of R^{cB} is independently halo or C₁₋₃ alkyl (e.g., halo).

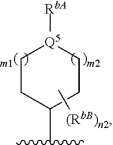

169. The compound of any one of clauses 1-55, wherein Y^{A2} is wherein Q⁵ is N or CH; m1 and m2 are independently 0, 1, or 2; n2 is 0, 1, or 2; and each of R^{bA} and R^{bB} is an independently selected R^{b}.

170. The compound of clause 169, wherein Q⁵ is CH.

171. The compound of any one of clauses 1-55 or 169-170, wherein Y^{A2} is

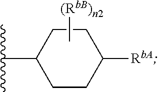

n2 is 0, 1, or 2; and each of R^{bA} and R^{bB} is an independently selected R^{b}.

172. The compound of any one of clauses 1-55 or 169-170, wherein Y^{A2} is

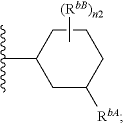

n2 is 0, 1, or 2; and each of R^{bA} and R^{bB} is an independently selected R^{b}.

173. The compound of any one of clauses 1-55 or 169-170, wherein Y^{A2} is

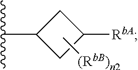

n2 is 0, 1, or 2; and each of R^{bA} and R^{bB} is an independently selected R^{b}.

174. The compound of any one of clauses 1-55 or 169-170, wherein Y^{A2} is

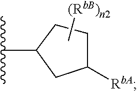

n2 is 0, 1, or 2; and each of R^{bA} and R^{bB} is an independently selected R^{b}.

175. The compound of clause 169, wherein Q⁵ is N.

176. The compound of any one of clauses 1-55, 169, or 175, wherein Y^{A2} is

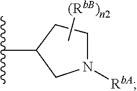

n2 is 0, 1, or 2; and each of R^{bA} and R^{bB} is an independently selected R^{b}.

177. The compound of any one of clauses 169-176, wherein R^{bA} is C₁₋₁₀ alkyl which is optionally substituted with 1-6 independently selected R^{a}

178. The compound of clause 177, wherein R^{bA} is unsubstituted C₁₋₁₀ alkyl (e.g., C₂, C₃, C₄, C₅, C₆, or C₇₋₁₀), such as ethyl, propyl (e.g., n-propyl), butyl (e.g., n-butyl; or sec-butyl; or tert-butyl; or iso-butyl), or octyl (e.g., n-octyl).

179. The compound of clause 177, wherein R^{bA} is C₁₋₁₀ alkyl which is substituted with 1-6 independently selected R^{a} (e.g., each R^{a} is selected from the group consisting of —F, —Cl, —Br, OH, C₁₋₄ alkoxy, and C₁₋₄ haloalkoxy) (e.g., R^{bA} is CF₃ or —CF₂CH₃).

180. The compound of any one of clauses 169-176, wherein R^{bA} is —F or —Cl.

181. The compound of any one of clauses 169-176, wherein R^{bA} is -L¹-L²-R^{h} (e.g., —R^{h} or —CH₂—R^{h} such as benzyl).

182. The compound of clause any one of clauses 169-176 or 181, wherein R^{bA} is R^{h} or —CH₂—R^{h}.

183. The compound of any one of clauses 169-176 or 181, wherein R^{bA} is —O—R^{h} or —N(H)—R^{h}.

184. The compound of any one of clauses 181-183, wherein R^{h} is selected from the group consisting of:

heteroaryl of 6 ring atoms, wherein 1-2 ring atoms are ring nitrogen atoms and wherein the heteroaryl ring is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy; and

C₆ aryl, which is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy.

185. The compound of any one of clauses 169-176, wherein R^{bA} is

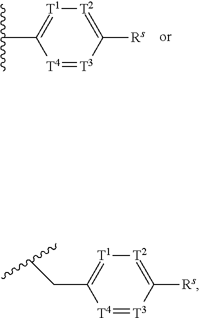

wherein T¹, T², T³, and T⁴ are each independently N, CH, or CR^{t}, provided that 1-4 (e.g., 2, 3, or 4) of T¹-T⁴ is CH, wherein each of R^{t} and R^{s} is independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy.

186. The compound of any one of clauses 169-174, wherein R^{bA} is

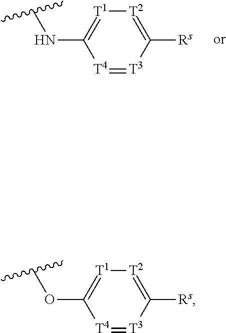

wherein T¹, T², T³, and T⁴ are each independently N, CH, or CR^{t}, provided that 1-4 (e.g., 2, 3, or 4) of T¹-T⁴ is CH, wherein each of R^{t} and R^{s} is independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy.

187. The compound of any one of clauses 169-174, wherein R^{bA} is C₁₋₄ alkoxy or C₁₋₄ haloalkoxy (e.g.,

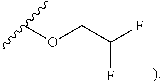 [SMILES: *OCC(F)F]

188. The compound of any one of clauses 169-187, wherein n2 is 0.

189. The compound of any one of clauses 169-188, wherein n2 is 1 or 2; optionally wherein each occurrence R^{bB} is selected from the group consisting of —F, —Cl, and C₁₋₃ alkyl.

190. The compound of any one of clauses 1-34, wherein Q-A is as defined according to (B).

191. The compound of clause 190, wherein E a ring of 5-8 ring atoms, wherein aside from the nitrogen atom present, 0-3 additional ring atoms are heteroatoms, each independently selected from the group consisting of N, N(H), N(R^{d}), O, and S(O)₀₋₂, and wherein the ring is optionally substituted with 1-4 independently selected R^{b}, such as:

wherein E is piperidinyl or piperazinyl, each of which is optionally substituted with 1-2 independently selected R^{b}.

192. The compound of clause 191, wherein E is

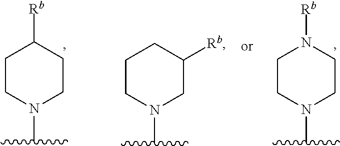

optionally wherein R^{b} is C1.6 alkyl or wherein R^{b} is R^{h}, —O—R^{h}, —CH₂R^{h}, or —N(H)R^{h}.

193. The compound of clause 1, wherein the compound has the following formula:

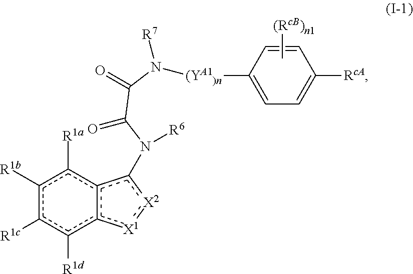

wherein n1 is 0, 1, or 2; each of R^{cA} and R^{cB} is an independently selected R^{c}; and R⁷ is H or C₁₋₄ alkyl,

optionally wherein the

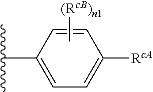

moiety is

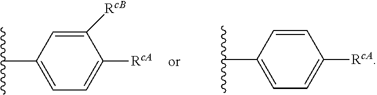

194. The compound of clause 1, wherein the compound has the following formula:

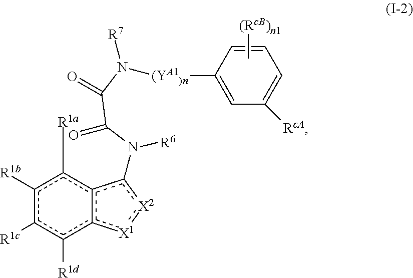

wherein n1 is 0, 1, or 2; each of R^{cA} and R^{cB} is an independently selected R^{c}; and R⁷ is H or C₁₋₄ alkyl, optionally wherein the

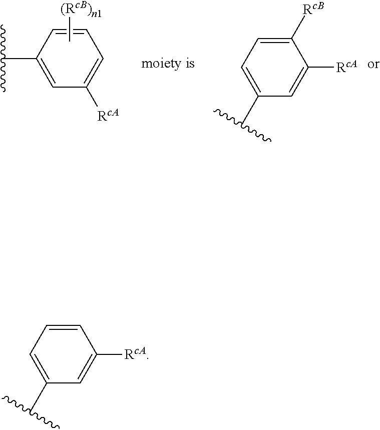

195. The compound of clause 1, wherein the compound has the following formula:

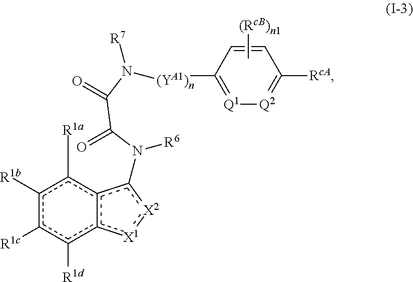

wherein one of Q¹ and Q² is N; the other one of Q¹ and Q² is CH; n1 is 0, 1, or 2; each of R^{cA} and R^{cB} is an independently selected R^{c}; and R⁷ is H or C₁₋₄ alkyl,

optionally wherein the

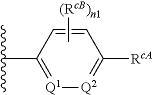

moiety is

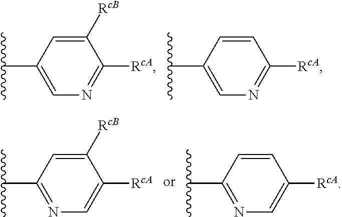

196. The compound of clause 1, wherein the compound has the following formula:

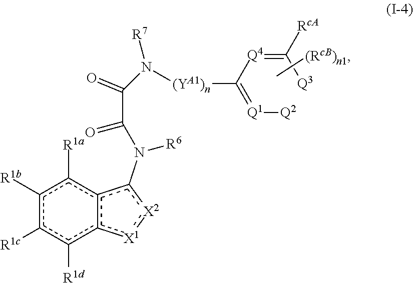

wherein one of Q¹, Q², Q³, and Q⁴ is N; each of the remaining of Q¹, Q², Q³, Q⁴ is CH; n1 is 0, 1, or 2; and each of R^{cA} and R^{cB} is an independently selected R^{c}; and R⁷ is H or C₁₋₄ alkyl,

optionally wherein the

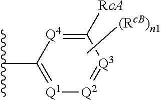

moiety is

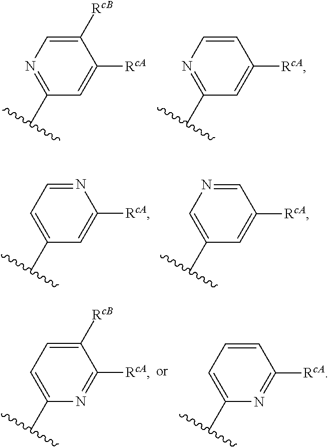

197. The compound of clause 1, wherein the compound has the following formula:

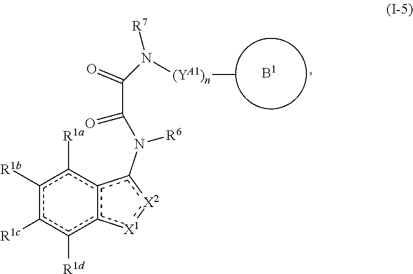

wherein B¹ is selected from the group consisting of:

(a) bicyclic or tricyclic heteroaryl of 7-14 (e.g., 9-12 (e.g., 9, 10, 11, or 12)) ring atoms, wherein 1-3 ring atoms are heteroatoms, each independently selected from the group consisting of N, N(H), N(R^{d}), O, and S(O)₀₋₂, and wherein the heteroaryl ring is optionally substituted with 1-4 independently selected R^{c}; and

(b) C₇₋₁₀ bicyclic aryl, which is optionally substituted with 1-3 R^{c};

and R⁷ is H or C₁₋₄ alkyl.

198. The compound of clause 197, wherein B1 is bicyclic or tricyclic heteroaryl of 9-10 (e.g., 10) ring atoms, wherein 1-3 ring atoms are heteroatoms, each independently selected from the group consisting of N, N(H), N(R^{d}), O, and S(O)₀₋₂, and wherein the heteroaryl ring is optionally substituted with 1-4 independently selected R^{c}.

199. The compound of clause 198, wherein B1 is selected from the group consisting of:

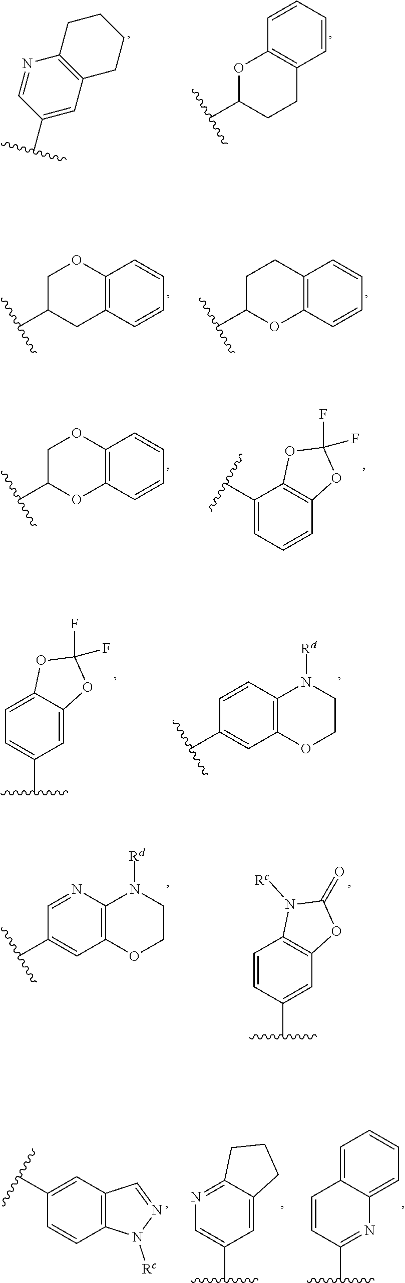

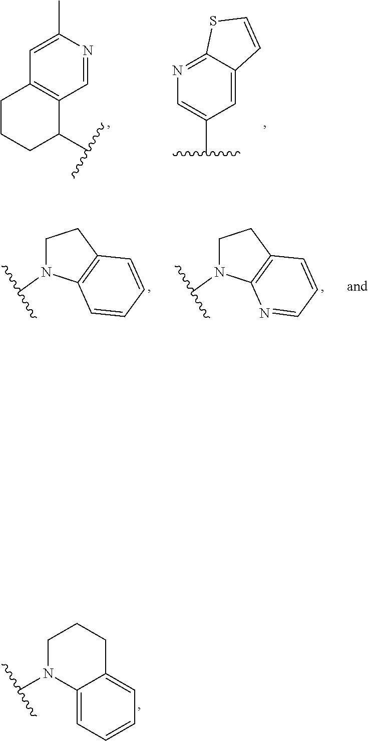 [SMILES: *C1CCCc2cc(C)ncc21 ; *c1cnc2sccc2c1 ; *N1CCc2ccccc21 ; *N1CCc2cccnc21 ; *N1CCCc2ccccc21]

each of which is further optionally substituted with 1-2 independently selected R^{c}.

200. The compound of any one of clauses 193-196, wherein R^{cA} is selected from the group consisting of: halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —NR^{I}R^{f}; —C₁₋₄ thioalkoxy; —C(═O)(C₁₋₁₀ alkyl); —C(═O)(OH); —C(═O)O(C₁₋₄ alkyl); and -L¹-L²-R^{h}.

201. The compound of any one of clauses 193-196 or 200, wherein R^{cA} is unsubstituted C₁₋₁₀ alkyl (e.g., C₂, C₃, C₄, C₅, C₆, or C₇₋₁₀), such as ethyl, propyl (e.g., n-propyl), butyl (e.g., n-butyl, iso-butyl, sec-butyl, tert-butyl), or octyl (e.g., n-octyl).

202. The compound of any one of clauses 193-196 or 200, wherein R^{cA} is C₁₋₁₀ alkyl which is substituted with 1-6 independently selected R^{a} (e.g., each occurrence of R^{a} is independently selected from —F, —Br, —Cl, OH, C₁₋₄ alkoxy, and C₁₋₄ haloalkoxy).

203. The compound of clause 202, wherein R^{cA} is C₁₋₁₀ alkyl which is substituted with 1-6 independently selected —F or —Cl (e.g., R^{cA} is CF₃).

204. The compound of any one of clauses 193-196 or 200, wherein R^{cA} is -L1-L²-R^{h}.

205. The compound of clause 204, wherein -L¹ is a bond.

206. The compound of any one of clauses 204-205, wherein -L² is a bond.

207. The compound of any one of clauses 204-206, wherein R^{h} is C₆₋₁₀ aryl, which is optionally substituted with 1-4 substituents independently selected from the group consisting of halo, C₁₋₄ alkyl, and C₁₋₄ haloalkyl,

such as C₆ aryl, which is optionally substituted with 1-2 substituents independently selected from the group consisting of halo, C₁₋₄ alkyl, and C₁₋₄ haloalkyl (e.g.,

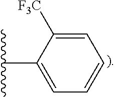 [SMILES: *c1ccccc1C(F)(F)F]

208. The compound of any one of clauses 204-206, wherein R^{h} is heterocyclyl or heterocycloalkenyl, wherein the heterocyclyl or heterocycloalkenyl has 3-10 (e.g., 5-6) ring atoms, wherein 1-3 (e.g., 1-2) ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo, C₁₋₄ alkyl, and C₁₋₄ haloalkyl, such as

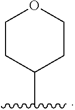 [SMILES: *C1CCOCC1]

209. The compound of any one of clauses 204-206, wherein R^{h} is C₃₋₈(e.g., C₃₋₆) cycloalkyl or C₃₋₈(e.g., C₃₋₆) cycloalkenyl, each optionally substituted with 1-4 substituents independently selected from the group consisting of halo, C₁₋₄ alkyl, and C₁₋₄ haloalkyl (e.g., R^{h} is cyclohexyl).

210. The compound of any one of clauses 193-196, wherein RCA is C₂₋₆ alkynyl (e.g.,

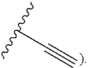 [SMILES: *C#C]

211. The compound of any one of clauses 193-210, wherein n1 is 0.

212. The compound of any one of clauses 193-210, wherein n1 is 1 or 2 (e.g., 1).

213. The compound of clause 212, wherein each occurrence of R^{cB} is independently halo or C₁₋₃ alkyl (e.g., halo).

214. The compound of clause 1, wherein the compound has the following formula:

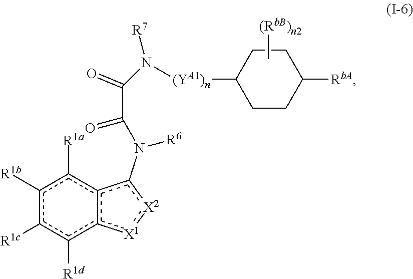

wherein n2 is 0, 1, or 2; each of R^{bA} and R^{bB} is an independently selected R^{b}; and R⁷ is H or C1.4 alkyl.

215. The compound of clause 1, wherein the compound has the following formula:

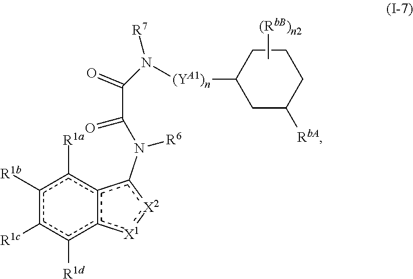

wherein n2 is 0, 1, or 2; each of R^{bA} and R^{bB} is an independently selected R^{b}; and R⁷ is H or C₁₋₄ alkyl.

216. The compound of clause 1, wherein the compound has the following formula:

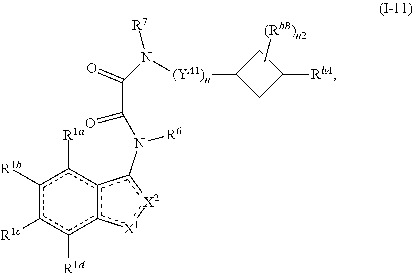

wherein n2 is 0, 1, or 2; each of R^{bA} and R^{bB} is an independently selected R^{b}; and R⁷ is H or C₁₋₄ alkyl.

217. The compound of clause 1, wherein the compound has the following formula:

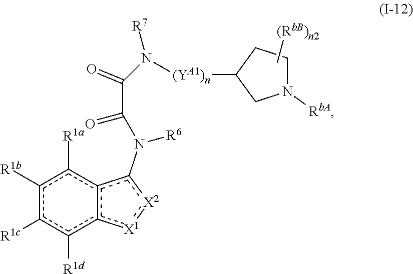

wherein n2 is 0, 1, or 2; each of R^{bA} and R^{bB} is an independently selected R^{b}; and R⁷ is H or C₁₋₄ alkyl.

218. The compound of clause 1, wherein the compound has the following formula:

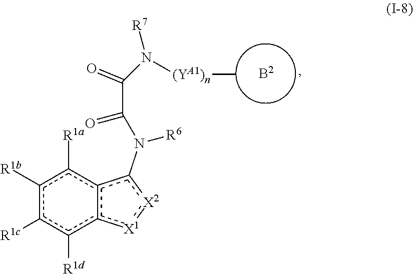

wherein B² is selected from the group consisting of:

bicyclic, tricyclic, or polycyclic C₇₋₂₀ (e.g., C₇₋₁₂) cycloalkyl or C₇₋₂₀ (e.g., C₇₋₁₂) cycloalkenyl, each optionally substituted with 1-2 R^{b}; and

bicyclic, tricyclic, or polycyclic heterocyclyl of 8-16 ring atoms, wherein 1-3 ring atoms are heteroatoms, each independently selected from the group consisting of N, N(H), N(R^{d}), O, and S(O)₀₋₂, and wherein the heterocyclyl ring is optionally substituted with 1-4 independently selected R^{b}; and

R⁷ is H or C₁₋₄ alkyl.

219. The compound of clause 218, wherein B² is selected from the group consisting of:

spirocyclic bicyclic C₇₋₁₂ cycloalkyl optionally substituted with 1-2 R^{b}, such as wherein Y^{A2} is: spiro[5.5]undecanyl (e.g.,

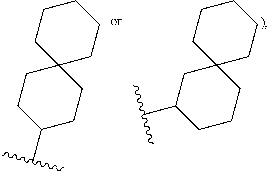 [SMILES: *C1CCC2(CCCCC2)CC1 ; *C1CCCC2(CCCCC2)C1]

spiro[4.4]nonanyl (e.g.,

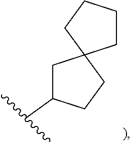 [SMILES: *C1CCC2(CCCC2)C1]

spiro[4.5]decanyl (e.g.,

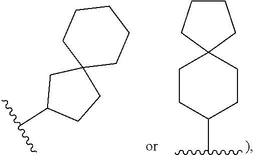 [SMILES: *C1CCC2(CCCCC2)C1 ; *C1CCC2(CCCC2)CC1]

or spiro[2.5]octanyl (e.g.,

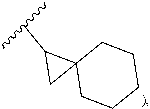 [SMILES: *C1CC12CCCCC2]

each of which is optionally substituted with R^{b}.

bridged bicyclic C₇₋₁₂ cycloalkyl optionally substituted with 1-2 R^{b}, such as wherein Y^{A2} is adamantly (e.g.,

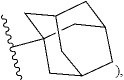 [SMILES: *C12CC3CC(CC(C3)C1)C2]

bicyclo[2.2.1]heptany (e.g.,

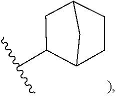 [SMILES: *C1CC2CCC1C2]

bicyclo[2.2.1]hept-2-enyl (e.g.,

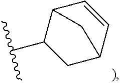 [SMILES: *C1CC2C=CC1C2]

or bicyclo[3.2.1]octanyl (e.g.,

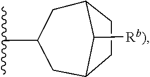

each of which is optionally substituted with R^{b}.

220. The compound of clause 218, wherein B² is bicyclic, tricyclic, or polycyclic heterocyclyl of 8-16 ring atoms, wherein 1-3 ring atoms are heteroatoms, each independently selected from the group consisting of N, N(H), N(R^{d}), O, and S(O)₀₋₂, and wherein the heterocyclyl ring is optionally substituted with 1-4 independently selected R^{b}, such as wherein B² is

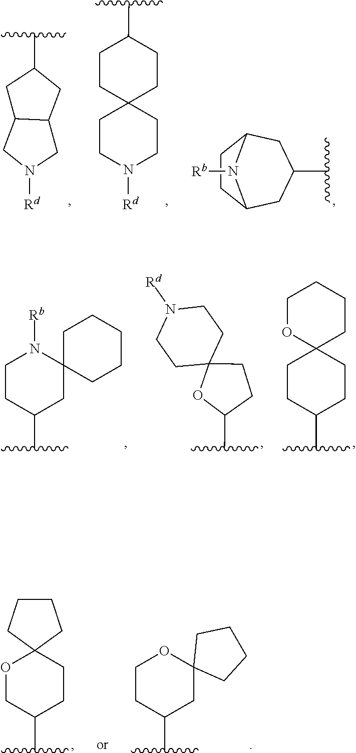

221. The compound of any one of clauses 214-217, wherein R^{bA} is C₁₋₁₀ alkyl which is optionally substituted with 1-6 independently selected R^{a}.

222. The compound of clause 221, wherein R^{bA} is unsubstituted C₁₋₁₀ alkyl (e.g., C₂, C₃, C₄, C₅, C₆, or C₇₋₁₀), such as ethyl, propyl (e.g., n-propyl), butyl (e.g., n-butyl; or sec-butyl; or tert-butyl; or iso-butyl), or octyl (e.g., n-octyl).

223. The compound of clause 221, wherein R^{bA} is C₁₋₁₀ alkyl which is substituted with 1-6 independently selected R^{a} (e.g., each R^{a} is selected from the group consisting of —F, —Cl, —Br, OH, C₁₋₄ alkoxy, and C₁₋₄ haloalkoxy) (e.g., R^{bA} is CF₃).

224. The compound of any one of clauses 214-217, wherein R^{bA} is —F or —Cl.

225. The compound of any one of clauses 214-217, wherein R^{bA} is -L¹-L²-R^{h} (e.g., —R^{h} or —CH₂—R^{h} such as benzyl).

226. The compound of any one of clauses 214-217 or 225, wherein R^{bA} is R^{h} or —CH₂—R^{h}.

227. The compound of any one of clauses 214-217 or 225, wherein R^{bA} is —O—R^{h} or —N(H)—R^{h}

228. The compound of any one of clauses 225-227, wherein R^{h} is selected from the group consisting of:

R^{h} is heteroaryl of 6 ring atoms, wherein 1-2 ring atoms are ring nitrogen atoms and wherein the heteroaryl ring is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy; and

C₆ aryl, which is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy.

229. The compound of any one of clauses 214-217, wherein R^{bA} is

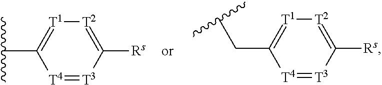

wherein T¹, T², T³, and T⁴ are each independently N, CH, or CR^{t}, provided that 1-4 (e.g., 2, 3, or 4) of T¹-T⁴ is CH, wherein each of R^{t} and R^{s} is independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy.

230. The compound of any one of clauses 214-217, wherein R^{bA} is

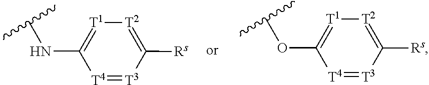

wherein T¹, T², T³, and T⁴ are each independently N, CH, or CR^{t}, provided that 1-4 (e.g., 2, 3, or 4) of T¹-T⁴ is CH, wherein each of R^{t} and R^{s} is independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy.

231. The compound of clauses 229 or 230, wherein each of T¹, T², T³, and T⁴ is independently CH or CR^{t}, such as each of Ti, T², T³, and T⁴ is CH.

232. The compound of clauses 229 or 230, wherein T¹ is N; and T², T³, and T⁴ are independently CH or CRt, such as wherein T¹ is N; and T², T³, and T⁴ are CH.

233. The compound of clauses 229 or 230, wherein T² is N; and T¹, T³, and T⁴ are independently CH or CR^{t}, such as wherein T² is N; and T¹, T³, and T⁴ are CH.

234. The compound of any one of clauses 229-233, wherein R⁵ is C₁₋₄ alkyl, such as methyl.

235. The compound of any one of clauses 229-233, wherein R⁵ is C₁₋₄ haloalkyl, such as CF₃.

236. The compound of any one of clauses 214-235, wherein n2 is 0.

237. The compound of any one of clauses 214-235, wherein n2 is 1 or 2.

238. The compound of clause 237, wherein each occurrence R^{bB} is selected from the group consisting of —F, —Cl, and C₁₋₃ alkyl.

239. The compound of clause 1, wherein the compound has the following formula:

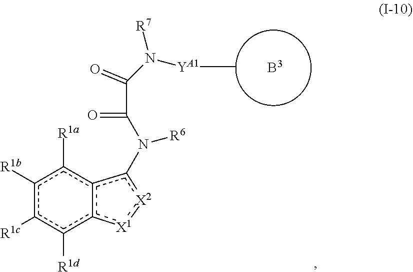

wherein B³ is:

(a) C₃₋₁₀ cycloalkyl or C₃₋₁₀ cycloalkenyl, each of which is optionally substituted with 1-2 R^{b},

(b) phenyl, which is optionally substituted with 1-2 R^{c};

(c) heteroaryl of 5-6 ring atoms, wherein 1-3 ring atoms are heteroatoms, each independently selected from the group consisting of N, N(H), N(R^{d}), O, and S(O)₀₋₂, and wherein the heteroaryl ring is optionally substituted with 1-2 independently selected R^{c}; and

R⁷ is H or C₁₋₄ alkyl.

240. The compound of clause 239, wherein B³ is C₃₋₁₀ cycloalkyl or C₃₋₁₀ cycloalkenyl, each of which is optionally substituted with 1-2 R^{b}.

241. The compound of clause 240, wherein B³ is C₅₋₇ cycloalkyl which is unsubstituted, such as unsubstituted cyclohexyl.

242. The compound of clause 239, wherein B³ is phenyl, which is optionally substituted with 1-2 R^{c}.

243. The compound of clause 242, wherein B³ is unsubstituted phenyl.

244. The compound of any one of clauses 193-243, wherein n is 0.

245. The compound of any one of clauses 193-243, wherein n is 1.

246. The compound of any one of clauses 193-243 or 245, wherein Y^{A1} is C1-6 alkylene, which is optionally substituted with 1-4 R^{a}.

247. The compound of clause 246, wherein Y^{A1} is —CH₂—, —CH₂CH₂—, —CH₂CH₂CH₂—, —CH(CF₃)—, —CH₂CH(OH)—,

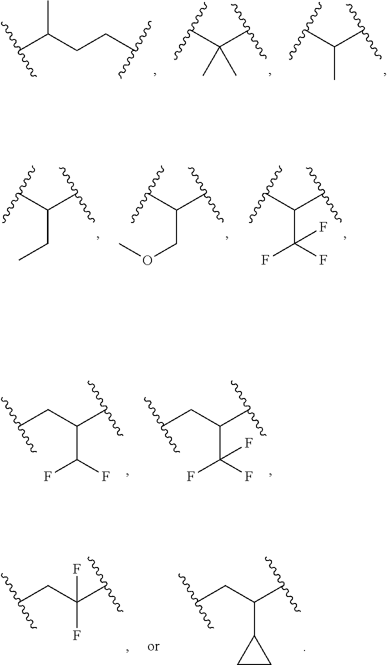

248. The compound of any one of clauses 193-243 or 245, wherein Y^{A1} is C₂₋₃ alkylene substituted with oxo, such as

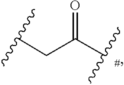 [SMILES: *CC(*)=O]

wherein # represents point of attachment to Y^{A2}.

249. The compound of any one of clauses 193-243 or 245, wherein Y^{A1} is Y^{A3}—Y^{A4}—Y^{A5}.

250. The compound of clause 249, wherein Y^{A3} is C₂₋₃ alkylene.

251. The compound of clauses 249 or 250, wherein Y^{A4} is —O—; or wherein Y^{A4} is —NH— or —N(C₁₋₃ alkyl), such as —O— or —N(C₁₋₃ alkyl).

252. The compound of any one of clauses 249-251, wherein Y^{A5} is a bond.

253. The compound of any one of clauses 249-252, wherein Y^{A1} is

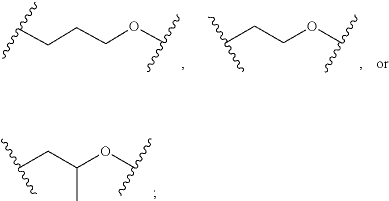

or wherein Y^{A1} is

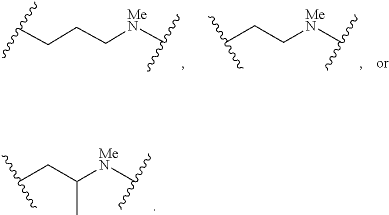

254. The compound of clause 1, wherein the compound has the following formula:

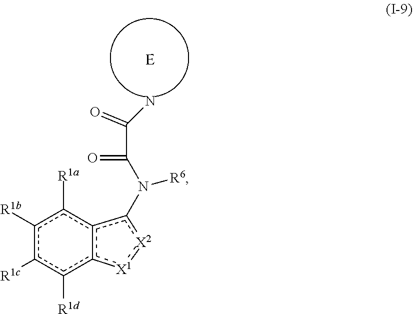

wherein:

E is a ring of 3-16 ring atoms, wherein aside from the nitrogen atom present, 0-3 additional ring atoms are heteroatoms, each independently selected from the group consisting of N, N(H), N(R^{d}), O, and S(O)₀₋₂, and wherein the ring is optionally substituted with 1-4 independently selected R^{b}.

255. The compound of clause 254, wherein E is a ring of 5-8 ring atoms, wherein aside from the nitrogen atom present, 0-3 additional ring atoms are heteroatoms, each independently selected from the group consisting of N, N(H), N(R^{d}), O, and S(O)₀₋₂, and wherein the ring is optionally substituted with 1-4 independently selected R^{b} (e.g., E is piperidinyl which is optionally substituted with 1-2 independently selected R^{b} (e.g., E is

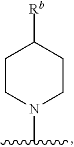

wherein R^{b} is C1.6 alkyl)).

256. The compound of any one of clauses 193-255, wherein the

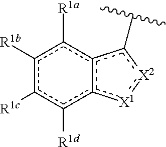

moiety is

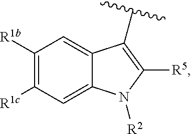

optionally wherein each of R^{1b} and R^{1c} is an independently selected substituent that is other than H, such as wherein each of R^{1b} and R^{1c} is an independently selected halo, such as —F or —Cl.

257. The compound of any one of clauses 193-255, wherein the

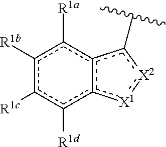

moiety is

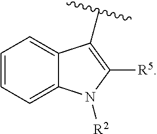

258. The compound of any one of clauses 193-255, wherein the

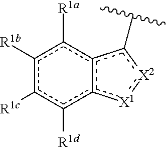

moiety is

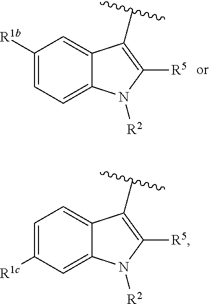

optionally wherein each of R^{1b} and R^{1c} is an independently selected substituent that is other than H, such as wherein each of R^{1b} and R^{1c} is an independently selected halo, such as —F or —Cl.

259. The compound of any one of clauses 193-258, wherein R² is H.

260. The compound of any one of clauses 193-259, wherein R⁵ is H.

261. The compound of any one of clauses 193-253 and 256-260, wherein R⁷ is H.

262. The compound of any one of clauses 193-261, wherein each of R¹, R^{1b}, R^{1c}, and R^{1d} is independently selected from the group consisting of: H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; -L³-L⁴-Rⁱ; —S(O)₁₋₂(C₁₋₄ alkyl); —S(O)(═NH)(C₁₋₄ alkyl); SF₅; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″).

263. The compound of clause 262, wherein each of R^{1a}, R^{1b}, R^{1c}, and R^{1d} is H.

264. The compound of clause 262, wherein 1-2 of R^{1a}, R^{1b}, R^{1c}, and R^{1d} is other than H.

265. The compound of clauses 262 or 264, wherein 1-2 of R^{1a}, R^{1b}, R^{1c}, and R^{1d} is halo (e.g., F or Cl), such as wherein each of R^{1b} and R^{1c} is independently F or Cl, such as F; or wherein R^{1c} is H; and R^{1b} is halo, such as —F or —Cl.

266. The compound of clauses 262 or 264, wherein one of R^{1a}, R^{1b}, R^{1c}, and R^{1d} is -L³-L⁴-Rⁱ (e.g., R^{1b} is -L³-L⁴-Rⁱ); and each remaining R^{1a}, R^{1b}, R^{1c}, and Rid is H.

267. The compound of clause 266, wherein one of R^{1a}, R^{1b}, R^{1c}, and R^{1d} (such as R^{1b}) is selected from the group consisting of:
heteroaryl of 5-6 ring atoms (such as pyrazolyl), wherein 1-4 ring atoms are heteroatoms, each independently selected from the group consisting of N, N(H), N(R^{d}), O, and S(O)₀₋₂ and wherein the heteroaryl ring is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy (e.g.,

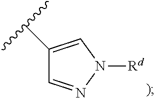

and
phenyl, which is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy (e.g.,).

268. The compound of any one of clauses 1-267, wherein R⁶ is H.

269. The compound of clause 1, wherein the compound is a compound of Formula (I-13):

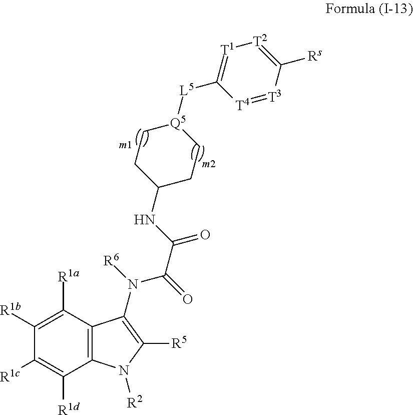

wherein:

m1 and m2 are independently 0, 1, or 2;

Q⁵ is N or CH;

L⁵ is a bond, CH₂, —O—, —N(H)—, or —N(C₁₋₃ alkyl), provided that when Q⁵ is N, then L⁵ is a bond or CH₂;

T¹, T², T³, and T⁴ are each independently N, CH, or CR^{t}, provided that 1-4, such as 2, 3, or 4, of T1-T⁴ is CH; and

each of R^{t} and R^{s} is independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy.

270. The compound of clause 269, wherein R² is H; and R⁵ is H.

271. The compound of clauses 269 or 270, wherein R⁶ is H.

272. The compound of any one of clauses 269-271, wherein Q⁵ is CH.

273. The compound of clause 272, wherein L⁵ is —O—.

274. The compound of clause 272, wherein L⁵ is —N(H)— or -N(C₁₋₃ alkyl), such as —N(H)—.

275. The compound of clause 272, wherein L⁵ is CH₂ or a bond.

276. The compound of any one of clauses 269-271, wherein Q⁵ is N.

277. The compound of clause 276, wherein L⁵ is CH₂.

278. The compound of clause 276, wherein L⁵ is a bond.

279. The compound of any one of clauses 269-278, wherein ml is 1; and m2 is 1.

280. The compound of any one of clauses 269-278, wherein ml is 1; and m2 is 0.

281. The compound of any one of clauses 269-278, wherein ml is 2; and m2 is 1.

282. The compound of any one of clauses 269-278, wherein ml is 0; and m2 is 0.

283. The compound of any one of clauses 269-271, wherein ml is 1; m2 is 1; Q⁵ is CH; and L⁵ is —O—.

284. The compound of any one of clauses 269-271, wherein ml is 0; m2 is 0; Q⁵ is CH; and L⁵ is —O—.

285. The compound of any one of clauses 269-271, wherein ml is 1; m2 is 0; Q⁵ is N; and L⁵ is a bond or CH₂.

286. The compound of any one of clauses 269-285, wherein each of T¹, T², T³, and T⁴ is independently CH or CR^{t}, such as wherein each of T¹, T², T³, and T⁴ is CH.

287. The compound of any one of clauses 269-285, wherein T¹ is N; and T², T³, and T⁴ are independently CH or CR^{t}, such as wherein T¹ is N; and T², T³, and T⁴ are CH.

288. The compound of any one of clauses 269-285, wherein T² is N; and T¹, T³, and T⁴ are independently CH or CR^{t}, such as wherein T² is N; and Ti, T³, and T⁴ are CH.

289. The compound of any one of clauses 269-288, wherein R⁵ is C₁₋₄ alkyl, such as methyl.

290. The compound of any one of clauses 269-288, wherein R⁵ is C₁₋₄ haloalkyl, such as CF₃.

291. The compound of any one of clauses 269-290, wherein R^{1a} is H; and R^{1d} is H or halo, such as: wherein R^{1a} is H, and R^{1d} is H; or wherein R^{1a} is H, and R^{1d} is halo such as —F or —Cl.

292. The compound of any one of clauses 269-291, wherein R^{1b} is halo; and R^{1c} is H, such as: wherein R^{1b} is —Cl, and R^{1c} is H; or wherein R^{1b} is —F, and R^{1c} is H; or

wherein R^{1b} is H; and R^{1c} is halo, such as: wherein R^{1b} is H, and R^{1c} is —F; or wherein R^{1b} is H, and R^{1c} is —Cl.

293. The compound of any one of clauses 269-291, wherein each of R^{1b} and R^{1c} is an independently selected halo, such as wherein R^{1b} is —Cl; and R^{1c} is —F; or wherein R^{1b} is —F; and R^{1c} is —F.

294. The compound of any one of clauses 269-291, wherein R^{1b} is R; and R^{1c} is H or halo, such as H; such as: wherein R^{1b} is selected from the group consisting of:
heteroaryl of 5-6 ring atoms (such as pyrazolyl), wherein 1-4 ring atoms are heteroatoms, each independently selected from the group consisting of N, N(H), N(R^{d}), O, and S(O)₀₋₂ and wherein the heteroaryl ring is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy (e.g.,

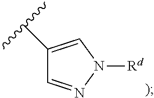

and
phenyl, which is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy (e.g.,

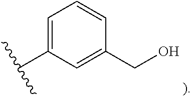 [SMILES: *c1cccc(CO)c1]

295. The compound of any one of clauses 269-291, wherein one of R^{1b} and R^{1c} is selected from the group consisting of: cyano, C₁₋₃ alkyl optionally substituted with R^{a}, and C₁₋₃ haloalkyl; and the other of R^{1b} and R^{1c} is H or halo, such as —H, —F, or —Cl.

296. The compound of clause 1, wherein the compound is selected from the group consisting of the compounds delineated in Table C1 or a pharmaceutically acceptable salt thereof.

297. A pharmaceutical composition comprising a compound of clauses 1-296 or a pharmaceutically acceptable salt thereof, and one or more pharmaceutically acceptable excipients.

298. A method for inhibiting STING activity, the method comprising contacting STING with a compound as defined in any one of clauses 1-296.

299. The method of clause 298, wherein the inhibiting comprises antagonizing STING.

300. The method of any one of clauses 298-299, which is carried out in vitro.

301. The method of clause 300, wherein the method comprises contacting a sample comprising one or more cells comprising STING with the compound.

302. The method of clause 300 or 301, wherein the one or more cells are one or more cancer cells.

303. The method of clause 301 or 302, wherein the sample further comprises one or more cancer cells (e.g., wherein the cancer is selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma).

304. The method of clause 302 or 303, which is carried out in vivo.

305. The method of clause 304, wherein the method comprises administering the compound to a subject having a disease in which increased (e.g., excessive) STING signaling contributes to the pathology and/or symptoms and/or progression of the disease.

306. The method of clause 305, wherein the subject is a human.

307. The method of clause 305, wherein the disease is cancer.

308. The method of clause 307, wherein the cancer is selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma.

309. The method of clause 307 or 308, wherein the cancer is a refractory cancer.

310. The method of clause 305, wherein the compound is administered in combination with one or more additional cancer therapies.

311. The method of clause 310, wherein the one or more additional cancer therapies comprises surgery, radiotherapy, chemotherapy, toxin therapy, immunotherapy, cryotherapy or gene therapy, or a combination thereof.

312. The method of clause 311, wherein chemotherapy comprises administering one or more additional chemotherapeutic agents.

313. The method of clause 312, wherein the one or more additional chemotherapeutic agents is selected from an alkylating agent (e.g., cisplatin, carboplatin, mechlorethamine, cyclophosphamide, chlorambucil, ifosfamide and/or oxaliplatin); an anti-metabolite (e.g., azathioprine and/or mercaptopurine); a terpenoid (e.g., a vinca alkaloid and/or a taxane; e.g., Vincristine, Vinblastine, Vinorelbine and/or Vindesine Taxol, Pacllitaxel and/or Docetaxel); a topoisomerase (e.g., a type I topoisomerase and/or a type 2 topoisomerase; e.g., camptothecins, such as irinotecan and/or topotecan; amsacrine, etoposide, etoposide phosphate and/or teniposide); a cytotoxic antibiotic (e.g., actinomycin, anthracyclines, doxorubicin, daunorubicin, valrubicin, idarubicin, epirubicin, bleomycin, plicamycin and/or mitomycin); a hormone (e.g., a lutenizing hormone releasing hormone agonist; e.g., leuprolidine, goserelin, triptorelin, histrelin, bicalutamide, flutamide and/or nilutamide); an antibody (e.g., Abciximab, Adalimumab, Alemtuzumab, Atlizumab, Basiliximab, Belimumab, Bevacizumab, Bretuximab vedotin, Canakinumab, Cetuximab, Ceertolizumab pegol, Daclizumab, Denosumab, Eculizumab, Efalizumab, Gemtuzumab, Golimumab, Golimumab, Ibritumomab tiuxetan, Infliximab, Ipilimumab, Muromonab-CD3, Natalizumab, Ofatumumab, Omalizumab, Palivizumab, Panitumuab, Ranibizumab, Rituximab, Tocilizumab, Tositumomab and/or Trastuzumab); an anti-angiogenic agent; a cytokine; a thrombotic agent; a growth inhibitory agent; an anti-helminthic agent; and an immune checkpoint inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155 (e.g., CTLA-4 or PD1 or PD-L1).

314. The method of any one of clauses 305-313, wherein the compound is administered intratumorally.

315. A method of treating cancer, comprising administering to a subject in need of such treatment an effective amount of a compound as defined in any one of clauses 1-296, or a pharmaceutical composition as defined in clause 297.

316. The method of clause 315, wherein the cancer is selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma.

317. The method of clause 315 or 316, wherein the cancer is a refractory cancer.

318. The method of clause 315, wherein the compound is administered in combination with one or more additional cancer therapies.

319. The method of clause 318, wherein the one or more additional cancer therapies comprises surgery, radiotherapy, chemotherapy, toxin therapy, immunotherapy, cryotherapy or gene therapy, or a combination thereof.

320. The method of clause 319, wherein chemotherapy comprises administering one or more additional chemotherapeutic agents.

321. The method of clause 320, wherein the one or more additional chemotherapeutic agents is selected from an alkylating agent (e.g., cisplatin, carboplatin, mechlorethamine, cyclophosphamide, chlorambucil, ifosfamide and/or oxaliplatin); an anti-metabolite (e.g., azathioprine and/or mercaptopurine); a terpenoid (e.g., a vinca alkaloid and/or a taxane; e.g., Vincristine, Vinblastine, Vinorelbine and/or Vindesine Taxol, Pacllitaxel and/or Docetaxel); a topoisomerase (e.g., a type I topoisomerase and/or a type 2 topoisomerase; e.g., camptothecins, such as irinotecan and/or topotecan; amsacrine, etoposide, etoposide phosphate and/or teniposide); a cytotoxic antibiotic (e.g., actinomycin, anthracyclines, doxorubicin, daunorubicin, valrubicin, idarubicin, epirubicin, bleomycin, plicamycin and/or mitomycin); a hormone (e.g., a lutenizing hormone releasing hormone agonist; e.g., leuprolidine, goserelin, triptorelin, histrelin, bicalutamide, flutamide and/or nilutamide); an antibody (e.g., Abciximab, Adalimumab, Alemtuzumab, Atlizumab, Basiliximab, Belimumab, Bevacizumab, Bretuximab vedotin, Canakinumab, Cetuximab, Ceertolizumab pegol, Daclizumab, Denosumab, Eculizumab, Efalizumab, Gemtuzumab, Golimumab, Golimumab, Ibritumomab tiuxetan, Infliximab, Ipilimumab, Muromonab-CD3, Natalizumab, Ofatumumab, Omalizumab, Palivizumab, Panitumuab, Ranibizumab, Rituximab, Tocilizumab, Tositumomab and/or Trastuzumab); an anti-angiogenic agent; a cytokine; a thrombotic agent; a growth inhibitory agent; an anti-helminthic agent; and an immune checkpoint inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II -LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155 (e.g., CTLA-4 or PD1 or PD-L1).

322. The method of any one of clauses 315-321, wherein the compound is administered intratumorally.

323. A method of inducing an immune response in a subject in need thereof, the method comprising administering to the subject an effective amount of a compound as defined in any one of clauses 1-296, or a pharmaceutical composition as defined in clause 297.

324. The method of clause 323, wherein the subject has cancer.

325. The method of clause 324, wherein the subject has undergone and/or is undergoing and/or will undergo one or more cancer therapies.

326. The method of clause 324, wherein the cancer selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma.

327. The method of clause 326, wherein the cancer is a refractory cancer.

328. The method of clause 323, wherein the immune response is an innate immune response.

329. The method of clause 328, wherein the at least one or more cancer therapies comprises surgery, radiotherapy, chemotherapy, toxin therapy, immunotherapy, cryotherapy or gene therapy, or a combination thereof.

330. The method of clause 329, wherein chemotherapy comprises administering one or more additional chemotherapeutic agents.

331. The method of clause 330, wherein the one or more additional chemotherapeutic agents is selected from alkylating agent (e.g., cisplatin, carboplatin, mechlorethamine, cyclophosphamide, chlorambucil, ifosfamide and/or oxaliplatin); an anti-metabolite (e.g., azathioprine and/or mercaptopurine); a terpenoid (e.g., a vinca alkaloid and/or a taxane; e.g., Vincristine, Vinblastine, Vinorelbine and/or Vindesine Taxol, Pacllitaxel and/or Docetaxel); a topoisomerase (e.g., a type I topoisomerase and/or a type 2 topoisomerase; e.g., camptothecins, such as irinotecan and/or topotecan; amsacrine, etoposide, etoposide phosphate and/or teniposide); a cytotoxic antibiotic (e.g., actinomycin, anthracyclines, doxorubicin, daunorubicin, valrubicin, idarubicin, epirubicin, bleomycin, plicamycin and/or mitomycin); a hormone (e.g., a lutenizing hormone releasing hormone agonist; e.g., leuprolidine, goserelin, triptorelin, histrelin, bicalutamide, flutamide and/or nilutamide); an antibody (e.g., Abciximab, Adalimumab, Alemtuzumab, Atlizumab, Basiliximab, Belimumab, Bevacizumab, Bretuximab vedotin, Canakinumab, Cetuximab, Ceertolizumab pegol, Daclizumab, Denosumab, Eculizumab, Efalizumab, Gemtuzumab, Golimumab, Golimumab, Ibritumomab tiuxetan, Infliximab, Ipilimumab, Muromonab-CD3, Natalizumab, Ofatumumab, Omalizumab, Palivizumab, Panitumuab, Ranibizumab, Rituximab, Tocilizumab, Tositumomab and/or Trastuzumab); an anti-angiogenic agent; a cytokine; a thrombotic agent; a growth inhibitory agent; an anti-helminthic agent; and an immune checkpoint inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155 (e.g., CTLA-4 or PD1 or PD-L1).

332. A method of treatment of a disease in which increased (e.g., excessive) STING signaling contributes to the pathology and/or symptoms and/or progression of the disease, comprising administering to a subject in need of such treatment an effective amount of a compound as defined in any one of clauses 1-296, or a pharmaceutical composition as defined in clause 297.

333. A method of treatment comprising administering to a subject having a disease in which increased (e.g., excessive) STING signaling contributes to the pathology and/or symptoms and/or progression of the disease an effective amount of a compound as defined in any one of clauses 1-296, or a pharmaceutical composition as defined in clause 297.

334. A method of treatment comprising administering to a subject a compound as defined in any one of clauses 1-296, or a pharmaceutical composition as defined in clause 297, wherein the compound or composition is administered in an amount effective to treat a disease in which increased (e.g., excessive) STING signaling contributes to the pathology and/or symptoms and/or progression of the disease, thereby treating the disease.

335. The method of any one of clauses 332-334, wherein the disease is cancer.

336. The method of clause 335, wherein the cancer is selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma.

337. The method of clause 335 or 336, wherein the cancer is a refractory cancer.

338. The method of any one of clauses 335-337, wherein the compound is administered in combination with one or more additional cancer therapies.

339. The method of clause 338, wherein the one or more additional cancer therapies comprises surgery, radiotherapy, chemotherapy, toxin therapy, immunotherapy, cryotherapy or gene therapy, or a combination thereof.

340. The method of clause 339, wherein chemotherapy comprises administering one or more additional chemotherapeutic agents.

341. The method of clause 340, wherein the one or more additional chemotherapeutic agents is selected from an alkylating agent (e.g., cisplatin, carboplatin, mechlorethamine, cyclophosphamide, chlorambucil, ifosfamide and/or oxaliplatin); an anti-metabolite (e.g., azathioprine and/or mercaptopurine); a terpenoid (e.g., a vinca alkaloid and/or a taxane; e.g., Vincristine, Vinblastine, Vinorelbine and/or Vindesine Taxol, Pacllitaxel and/or Docetaxel); a topoisomerase (e.g., a type I topoisomerase and/or a type 2 topoisomerase; e.g., camptothecins, such as irinotecan and/or topotecan; amsacrine, etoposide, etoposide phosphate and/or teniposide); a cytotoxic antibiotic (e.g., actinomycin, anthracyclines, doxorubicin, daunorubicin, valrubicin, idarubicin, epirubicin, bleomycin, plicamycin and/or mitomycin); a hormone (e.g., a lutenizing hormone releasing hormone agonist; e.g., leuprolidine, goserelin, triptorelin, histrelin, bicalutamide, flutamide and/or nilutamide); an antibody (e.g., Abciximab, Adalimumab, Alemtuzumab, Atlizumab, Basiliximab, Belimumab, Bevacizumab, Bretuximab vedotin, Canakinumab, Cetuximab, Ceertolizumab pegol, Daclizumab, Denosumab, Eculizumab, Efalizumab, Gemtuzumab, Golimumab, Golimumab, Ibritumomab tiuxetan, Infliximab, Ipilimumab, Muromonab-CD3, Natalizumab, Ofatumumab, Omalizumab, Palivizumab, Panitumuab, Ranibizumab, Rituximab, Tocilizumab, Tositumomab and/or Trastuzumab); an anti-angiogenic agent; a cytokine; a thrombotic agent; a growth inhibitory agent; an anti-helminthic agent; and an immune checkpoint inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155 (e.g., CTLA-4 or PD1 or PD-L1).

342. The method of any one of clauses 332-341, wherein the compound is administered intratumorally.

343. A method of treatment of a disease, disorder, or condition associated with STING, comprising administering to a subject in need of such treatment an effective amount of a compound as defined in any one of clauses 1-296, or a pharmaceutical composition as defined in clause 297.

344. The method of clause 343, wherein the disease, disorder, or condition is selected from type I interferonopathies, Aicardi-Goutieres Syndrome (AGS), genetic forms of lupus, inflammation-associated disorders, and rheumatoid arthritis.

345. The method of clause 344, wherein the disease, disorder, or condition is a type I interferonopathy (e.g., STING-associated vasculopathy with onset in infancy (SAVI)).

346. The method of clause 345, wherein the type I interferonopathy is STING-associated vasculopathy with onset in infancy (SAVI)).

347. The method of clause 344, wherein the disease, disorder, or condition is Aicardi-Goutieres Syndrome (AGS).

348. The method of clause 344, wherein the disease, disorder, or condition is a genetic form of lupus.

349. The method of clause 344, wherein the disease, disorder, or condition is inflammation-associated disorder.

350. The method of clause 349, wherein the inflammation-associated disorder is systemic lupus erythematosus.

351. The method of any one of clauses 298-350, wherein the method further comprises identifying the subject.

352. A combination comprising a compounds defined in any one of clauses I to 296 or a pharmaceutically acceptable salt or tautomer thereof, and one or more therapeutically active agents.

353. A compound defined in any one of clauses 1 to 296 or a pharmaceutically acceptable salt or tautomer thereof, or a pharmaceutical composition defined in clause 297, for use as a medicament.

354. A compound defined in any one of clauses 1 to 296 or a pharmaceutically acceptable salt or tautomer thereof, or a pharmaceutical composition defined in clause 297, for use in the treatment of a disease, condition or disorder modulated by STING inhibition.

355. A compound defined in any one of clauses 1 to 296 or a pharmaceutically acceptable salt or tautomer thereof, or the pharmaceutical composition defined in clause 297, for use in the treatment of a disease mentioned in any one of clauses 298 to 350.

356. Use of a compound defined in any one of clauses 1 to 296 or a pharmaceutically acceptable salt or tautomer thereof, or a pharmaceutical composition defined in clause 297, in the manufacture of a medicament for the treatment of a disease mentioned in in any one of clauses 298 to 350.

What is claimed is:

1. A compound of Formula I: 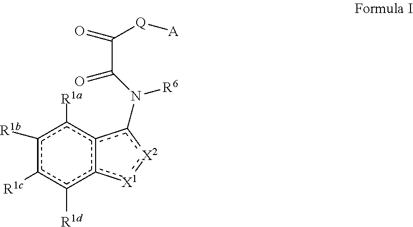
or a pharmaceutically acceptable salt thereof or a tautomer thereof, wherein:
X¹ is selected from the group consisting of O, S, N, NR², and CR⁵;
X² is selected from the group consisting of O, S, N, NR⁴, and CR⁵;
each [symbol] is independently a single bond or a double bond, provided that the five-membered ring comprising X¹ and X² is heteroaryl; and
the 6-membered ring 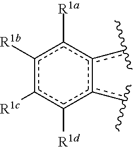
is aromatic;
Q-A is defined according to (A) or (B) below:
(A)
Q is selected from the group consisting of: NH and N(C₁₋₆ alkyl) wherein the C₁₋₆ alkyl is optionally substituted with 1-2 independently selected R^{a}; and
A is:
(i) —(Y^{A1})ₙ—Y^{A2}, wherein:
n is 0 or 1;
Y^{A1} is C1-6 alkylene, which is optionally substituted with 1-6 substituents each independently selected from the group consisting of:
oxo;
R^{a};
C₆₋₁₀ aryl optionally substituted with 1-4 independently selected C₁₋₄ alkyl; and
heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl ring is optionally substituted with 1-4 independently selected C₁₋₄ alkyl; or
Y^{A1} is —Y^{A3}—Y^{A4}—Y^{A5} which is connected to Q via Y^{A3} wherein:
Y^{A3} is a C₁₋₃ alkylene optionally substituted with 1-2 substituents each independently selected from the group consisting of oxo and R^{a};
Y^{A4} is —O—, —NH—, —N(C₁₋₆ alkyl)-, or —S—; and
Y^{A5} is a bond or C₁₋₃ alkylene which is optionally substituted with 1-2 independently selected R^{a}; and
Y^{A2} is:
(a) C₃₋₂₀ cycloalkyl or C₃₋₂₀ cycloalkenyl, each of which is optionally substituted with 1-4 R^{b},
(b) C₆₋₂₀ aryl which is optionally substituted with 1-4 R^{c};
(c) heteroaryl of 5-20 ring atoms, wherein 1-3 ring atoms are heteroatoms, each independently selected from the group consisting of N, N(H), N(R^{d}), 0, and S(O)₀₋₂, and wherein the heteroaryl ring is optionally substituted with 1-4 independently selected R^{c}; or
(d) heterocyclyl or heterocycloalkenyl of 3-16 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl ring is optionally substituted with 1-4 independently selected R^{b},
or
(ii) —Z¹—Z²—Z³, wherein:
Z¹ is C₁₋₃ alkylene, which is optionally substituted with 1-4 R^{a};
Z² is —N(H)—, —N(R^{d})—, —O—, or —S—; and
Z³ is C₂₋₇ alkyl, which is optionally substituted with 1-4 R^{a};
or
(iii) C₁₋₂₀ alkyl, which is optionally substituted with 1-6 independently selected R^{a}, or
(B)
Q and A, taken together, form: 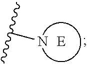 [SMILES: *N]
and
E is a ring of 3-16 ring atoms, wherein 0-3 ring atoms are heteroatoms (in addition to the nitrogen atom this is already present), each independently selected from the group consisting of N, N(H), N(R^{d}), O, and S(O)₀₋₂, and wherein the ring is optionally substituted with 1-4 independently selected R^{b},
each of R^{1a}, R^{1b}, R^{1c}, and R^{1d} is independently selected from the group consisting of: H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; -L³-L⁴-Rⁱ; —S(O)₁₋₂(C₁₋₄ alkyl); —S(O)(═NH)(C₁₋₄ alkyl); SF₅; —NR^{e}R^{f}; —OH; oxo; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″); or
R^{1a} and R^{1b}, R^{1b} and R^{1c}, or R^{1c} and R^{1d}, taken together with the atoms connecting them, form a ring of 3-10 ring atoms, wherein 0-2 ring atoms are heteroatoms each independently selected from the group consisting of N, N(H), N(R^{d}), 0, and S(O)₀₋₂; and wherein the ring is optionally substituted with 1-4 substituents each independently selected from the group consisting of C₁₋₆ alkyl, halo, C₁₋₆ haloalkyl, —OH, NR^{e}R^{f}, C1-6 alkoxy, and C₁₋₆ haloalkoxy,
each occurrence of R² is independently selected from the group consisting of:
(i) C₁₋₆ alkyl, which is optionally substituted with 1-2 independently selected R^{a};
(ii) C₃₋₆ cycloalkyl or C₃₋₆ cycloalkenyl;
(iii) heterocyclyl or heterocycloalkenyl of 3-10 ring atoms, wherein 1-3 ring atoms are heteroatoms, each independently selected from the group consisting of N, N(H), N(R^{d}), 0, and S(O)₀₋₂;
(iv) C₆₋₁₀ aryl;
(v) heteroaryl of 5-10 ring atoms, wherein 1-3 ring atoms are heteroatoms, each independently selected from the group consisting of N, N(H), N(R^{d}), O, and S(O)₀₋₂;
(vi) —C(O)(C₁₋₄ alkyl);
(vii) —C(O)O(C1.4 alkyl);
(viii) —CON(R′)(R″);
(ix) —S(O)i-2(NR′R″);
(x) —S(O)₁₋₂(C₁₋₄ alkyl);
(xi) —OH;
(xii) C₁₋₄ alkoxy; and
(xiii) H;
R⁴ is selected from the group consisting of H and C₁₋₆ alkyl optionally substituted with 1-3 independently selected R^{a};
R⁵ is selected from the group consisting of H; halo; —OH; —C₁₋₄ alkyl; —C₁₋₄ haloalkyl;
C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and C₃₋₆ cycloalkyl or C₃₋₆ cycloalkenyl, each optionally substituted with 1-4 independently selected C₁₋₄ alkyl;
R⁶ is selected from the group consisting of H; C₁₋₆ alkyl optionally substituted with 1-3 independently selected R^{a}; —OH; C₁₋₄ alkoxy; C(═O)H; C(═O)(C₁₋₄ alkyl); C₆₋₁₀ aryl optionally substituted with 1-4 independently selected C₁₋₄ alkyl; and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 independently selected C₁₋₄ alkyl;
each occurrence of R^{a} is independently selected from the group consisting of: —OH; —F; —Cl; —Br; —NR^{I}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano, and C₃₋₆ cycloalkyl or C₃₋₆ cycloalkenyl, each optionally substituted with 1-4 independently selected C₁₋₄ alkyl;
each occurrence of R^{b} is independently selected from the group consisting of: C₁₋₁₀ alkyl optionally substituted with 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₁₀ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};
each occurrence of R^{c} is independently selected from the group consisting of: halo;
cyano; C₁₋₁₀ alkyl which is optionally substituted with 1-6 independently selected R^{a}; C₂₋₆ alkenyl; C₂₋₆ alkynyl; oxo; C₁₋₄ alkoxy optionally substituted with 1-2 independently selected R^{a}; C₁₋₄ haloalkoxy; —S(O)₁₋₂(C₁₋₄ alkyl) or —S(O)₁₋₂(C₁₋₄ haloalkyl); —NR^{e}R^{f}; —OH; —S(O)₁₋₂(NR′R″); —C₁₋₄ thioalkoxy or —C₁₋₄ thiohaloalkoxy; —NO₂; —SF₅; —C(═O)(C₁₋₁₀ alkyl); —C(═O)O(C1-4 alkyl); —C(═O)OH; —C(═O)N(R′)(R″); and -L¹-L²-R^{h};
R^{d} is selected from the group consisting of: C₁₋₆ alkyl optionally substituted with 1-3 substituents each independently selected from the group consisting of halo and OH; C₃₋₆ cycloalkyl or C₃₋₆ cycloalkenyl, each optionally substituted with 1-3 substituents each independently selected from the group consisting of halo and OH; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;
each occurrence of R^{e} and R^{f} is independently selected from the group consisting of: H; C₁₋₆ alkyl; C₁₋₆ haloalkyl; C₃₋₆ cycloalkyl or C₃₋₆ cycloalkenyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or R^{e} and R^{f} together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R^{e} and Rr), which are each independently selected from the group consisting of N(R^{d}), NH, 0, and S;
-L¹ is a bond or C₁₋₃ alkylene;
-L² is —O—, —N(H)—, —N(C₁₋₃ alkyl)-, —S(O)₀₋₂-, or a bond;
R^{h} is selected from the group consisting of:
C₃₋₈ cycloalkyl or C₃₋₈ cycloalkenyl, each optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy; and
C₆₋₁₀ aryl, which is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy;
-L³ is a bond or C₁₋₃ alkylene;
-L⁴ is —O—, —N(H)—, —N(C₁₋₃ alkyl)-, —S(O)₀₋₂-, or a bond;
Rⁱ is selected from the group consisting of:
C₃₋₈ cycloalkyl or C₃₋₈ cycloalkenyl, each optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy; and
each occurrence of R′ and R″ is independently selected from the group consisting of: H, C₁₋₄ alkyl, C₆₋₁₀ aryl optionally substituted with 1-2 substituents selected from the group consisting of halo, C₁₋₄ alkyl, and C₁₋₄ haloalkyl, and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo, —OH, NH₂, NH(C₁₋₄ alkyl), N(C₁₋₄ alkyl)₂, C₁₋₄ alkyl, and C₁₋₄ haloalkyl; or R′ and R″ together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R′ and R″), which are each independently selected from the group consisting of N(H), N(C₁₋₆ alkyl), O, and S.

2. The compound of claim 1, wherein X¹ is NR², optionally wherein R² is H.

3. The compound of claim 1 or 2, wherein X² is CR⁵, optionally wherein R⁵ is H.

4. The compound of any one of claims 1-3, wherein the 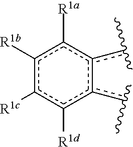
moiety is 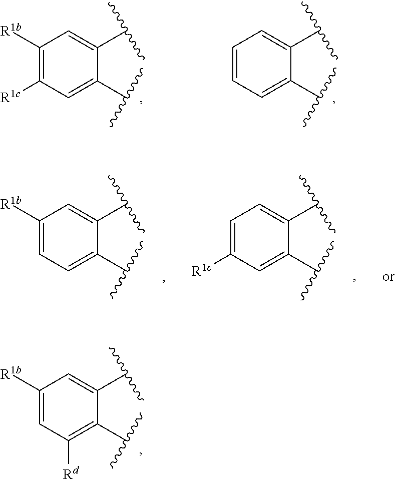
wherein each of R^{1b}, R^{1c}, and R^{1d} in the above formulae is an independently selected substituent that is other than H, optionally wherein each of R^{1b}, R^{1c}, and R^{1d} is an independently selected halo, such as —F or —Cl.

5. The compound of any one of claims 1-4, wherein Q-A is defined according to (A).

6. The compound of any one of claims 1-5, wherein A is —(Y^{A1})ₙ—Y^{A2}.

7. The compound of any one of claims 1-6, wherein Y^{A2} is C₆₋₁₀ aryl, which is optionally substituted with 1-3 R^{c}; or
wherein Y^{A2} is heteroaryl of 5-14 ring atoms, wherein 1-3 ring atoms are heteroatoms, each independently selected from the group consisting of N, N(H), N(R^{d}), O, and S(O)₀₋₂, and
wherein the heteroaryl ring is optionally substituted with 1-4 independently selected R^{c}; or
wherein Y^{A2} is monocyclic C₃₋₁₀ cycloalkyl or C3-10 cycloalkenyl, each of which is optionally substituted with 1-4 R^{b}; or
wherein Y^{A2} is heterocyclyl or heterocycloalkenyl of 3-16 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl ring is optionally substituted with 1-3 independently selected R^{b}.

8. The compound of any one of claims 1-7, wherein Y^{A2} is C₃₋₆ cycloalkyl or C₃₋₆ cycloalkenyl, each of which is substituted with 1-4, such as 1-2, R^{b}, such as:
wherein Y^{A2} is cyclopropyl, cyclobutyl, cyclopentyl, or cyclohexyl, each of which is optionally substituted with 1-2 R^{b}, such as:
wherein Y^{A2} is

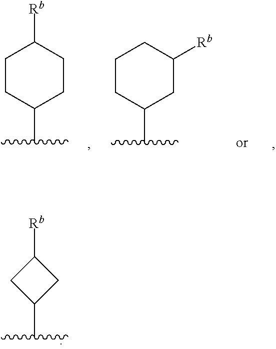

9. The compound of any one of claims 1-7, wherein Y^{A2} is heterocyclyl or heterocycloalkenyl of 3-16 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl ring is optionally substituted with 1-3 independently selected R^{b}, such as:
wherein Y^{A2} is heterocyclyl of 4-8 ring atoms, such as 4-6 ring atoms, wherein 1-2 ring atoms are heteroatoms, each independently selected from the group consisting of N, N(H), N(R^{d}), O, and S(O)₀₋₂, and wherein the heterocyclyl ring is optionally substituted with 1-2 independently selected R^{b}, such as:
wherein Y^{A2} is 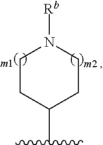
wherein m1 and m2 are independently 0, 1, or 2, such as: wherein Y^{A2} is

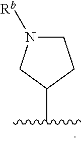

10. The compound of any one of claims 1-9, wherein each occurrence of R^{b} is independently selected from the group consisting of: C₁₋₁₀ alkyl optionally substituted with 1-6 independently selected R^{a}; C₁₋₄ haloalkyl; —F; —Cl; —Br; cyano; C₁₋₄ alkoxy; C₁₋₄ haloalkoxy; —C(═O)(C₁₋₁₀ alkyl); —C(═O)O(C₁₋₄ alkyl); —S(O)₁₋₂(C₁₋₄ alkyl); oxo; cyano; and -L¹-L²-R^{h},
optionally wherein one occurrence of R^{b} is -L¹-L²-R^{h},
optionally wherein L¹ is a bond, and L² is —O— or a bond; and
optionally wherein R^{h} is C₆₋₁₀ aryl, which is optionally substituted with 1-4 substituents independently selected from the group consisting of halo, C₁₋₄ alkyl, and C₁₋₄ haloalkyl; or
wherein R^{h} is heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy.

11. The compound of any one of claims 1-4, wherein Q-A is as defined according to (B).

12. The compound of claim 1, wherein the compound is a compound of Formula (I-1), (I-2), (I-3), (I-4), or (I-5), or a pharmaceutically acceptable salt thereof: 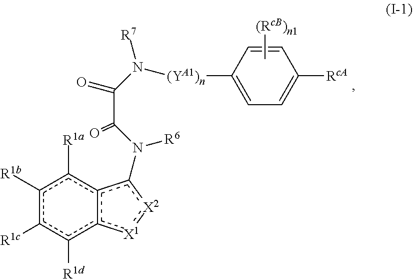
wherein n1 is 0, 1, or 2; each of R^{cA} and R^{cB} is an independently selected R^{c}; and R⁷ is H or C₁₋₄ alkyl; 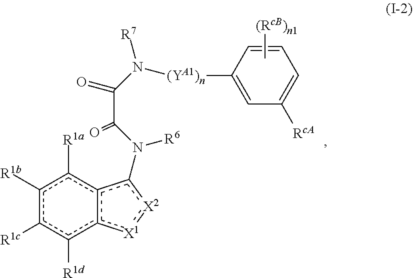
wherein n1 is 0, 1, or 2; each of R^{cA} and R^{cB} is an independently selected R^{c}; and R⁷ is H or C₁₋₄ alkyl; 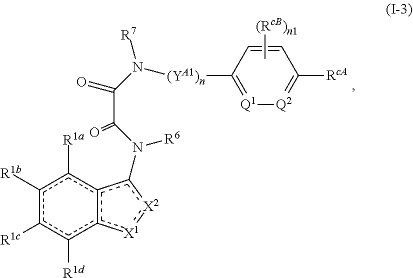
wherein one of Q¹ and Q² is N; the other one of Q¹ and Q² is CH; n1 is 0, 1, or 2; each of R^{cA} and R^{cB} is an independently selected R^{c}; and R⁷ is H or C1.4 alkyl; 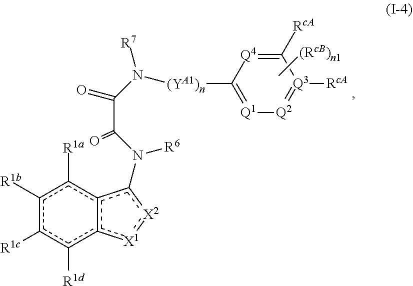
wherein one of Q¹, Q², Q³, and Q⁴ is N; each of the remaining of Q¹, Q², Q³, Q⁴ is CH; n1 is 0, 1, or 2; and each of R^{cA} and R^{cB} is an independently selected R^{c}; and R⁷ is H or C₁₋₄ alkyl; or 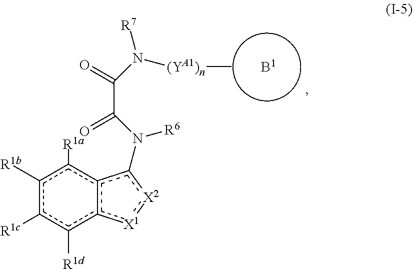
wherein B¹ is selected from the group consisting of:
(a) bicyclic or tricyclic heteroaryl of 7-14 ring atoms, wherein 1-3 ring atoms are heteroatoms, each independently selected from the group consisting of N, N(H), N(R^{d}), O, and S(O)₀₋₂, and wherein the heteroaryl ring is optionally substituted with 1-4 independently selected R^{c}; and
(b) C₇₋₁₀ bicyclic aryl, which is optionally substituted with 1-3 R^{c}, and
R⁷ is H or C₁₋₄ alkyl;
optionally wherein X¹ is NH; and X² is CH in Formula (I-1), (I-2), (I-3), (I-4), or (I-5); and
optionally wherein R^{cA} in Formula (I-1), (1-2), (1-3), or (I-4) is selected from the group consisting of: C₁₋₁₀ alkyl which is optionally substituted with 1-6 independently selected R^{a}; C₂₋₆ alkynyl; C₁₋₄ alkoxy; C₁₋₄ haloalkoxy; and -L¹-L²-R^{h}.

13. The compound of claim 1, wherein the compound is a compound of Formula (I-6), (I-7), (I-11), (I-12), or (I-8), or a pharmaceutically acceptable salt thereof: 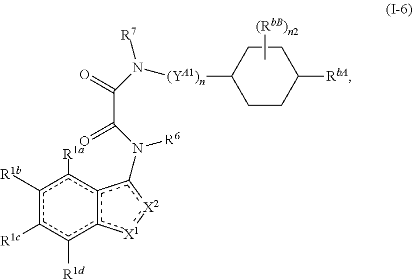
wherein n2 is 0, 1, or 2; each of R^{bA} and R^{bB} is an independently selected R^{b}; and R⁷ is H or C₁₋₄ alkyl; 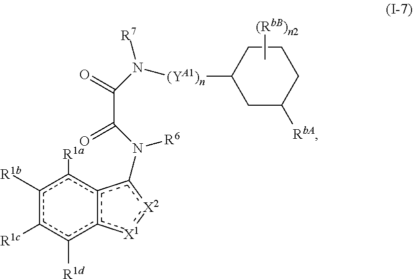
wherein n2 is 0, 1, or 2; each of R^{bA} and R^{bB} is an independently selected R^{b}; and R⁷ is H or C₁₋₄ alkyl; 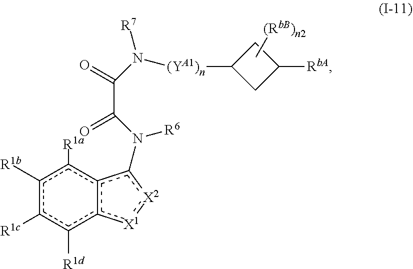
wherein n2 is 0, 1, or 2; each of R^{bA} and R^{bB} is an independently selected R^{b}; and R⁷ is H or C₁₋₄ alkyl; 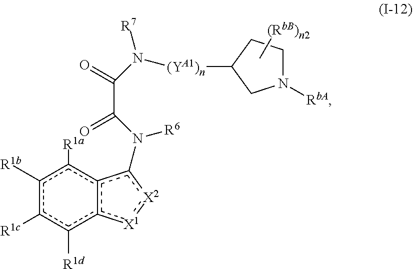
wherein n2 is 0, 1, or 2; each of R^{bA} and R^{bB} is an independently selected R^{b}; and R⁷ is H or C₁₋₄ alkyl; 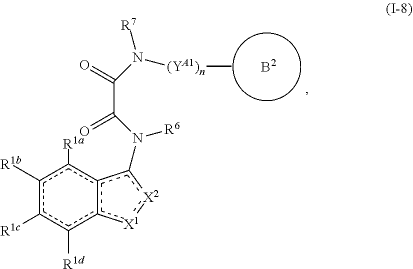
wherein B² is selected from the group consisting of:
bicyclic, tricyclic, or polycyclic C₇₋₂₀ cycloalkyl or C₇₋₂₀ cycloalkenyl, each optionally substituted with 1-2 independently selected R^{b}; and
bicyclic, tricyclic, or polycyclic heterocyclyl of 8-16 ring atoms, wherein 1-3 ring atoms are heteroatoms, each independently selected from the group consisting of N, N(H), N(R^{d}), O, and S(O)₀₋₂, and wherein the heterocyclyl ring is optionally substituted with 1-4 independently selected R^{b}, and
R⁷ is H or C₁₋₄ alkyl,
optionally wherein X¹ is NH; and X² is CH in Formula (I-6), (I-7), (I-11), (I-12), or (I-8); and
optionally wherein R^{bA} is —R^{h} in Formula (I-6), (I-7), (I-11), or (I-12), such as wherein R^{bA} is: -L¹-L²-R^{h}, such as —R^{h} or —O—R^{h}, and
optionally wherein R^{h} is selected from the group consisting of:
heteroaryl of 6 ring atoms, wherein 1-2 ring atoms are ring nitrogen atoms and wherein the heteroaryl ring is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy; and
C₆ aryl, which is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy.

14. The compound of any one of claims 1-13, wherein n is 0.

15. The compound of claim 1, wherein the compound is a compound of Formula (I-13): 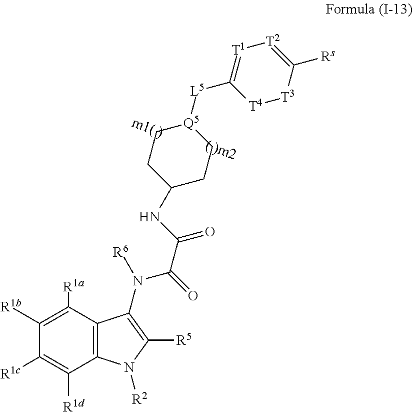
wherein:
m1 and m2 are independently 0, 1, or 2;
Q⁵ is N or CH;
L⁵ is a bond, CH₂, —O—, —N(H)—, or —N(C₁₋₃ alkyl), provided that when Q⁵ is N, then L⁵ is a bond or CH₂;
T¹, T², T³, and T⁴ are each independently N, CH, or CR^{t}, provided that 1-4, such as 2, 3, or 4, of T¹-T⁴ is CH; and
each of R^{t} and R^{s} is independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy,
optionally wherein R² is H, and R⁵ is H; and
optionally wherein R^{1b} is halo, such as —F or —Cl; R^{1c} is H or halo, such as —H or —F; and R^{1a} and R^{1d} are H.

16. The compound of claim 1, wherein the compound is selected from the group consisting of the compounds delineated in Table C1, or a pharmaceutically acceptable salt thereof.

17. A pharmaceutical composition comprising a compound of claims 1-16 or a pharmaceutically acceptable salt thereof, and one or more pharmaceutically acceptable excipients.

18. A method for inhibiting STING activity, the method comprising contacting STING with a compound as claimed in any one of claims 1-16, or a pharmaceutically acceptable salt thereof; or a pharmaceutical composition as claimed in claim 17.

19. A method of inducing an immune response in a subject in need thereof, the method comprising administering to the subject an effective amount of a compound as claimed in any one of claims 1-16, or a pharmaceutically acceptable salt thereof; or a pharmaceutical composition as claimed in claim 17.

20. A method of treatment of disease, disorder, or condition associated with STING, such as a disease, disorder, or condition, in which increased STING signaling, such as excessive STING signaling, contributes to the pathology and/or symptoms and/or progression of the disease, such as cancer, comprising administering to a subject in need of such treatment an effective amount of a compound as claimed in any one of claims 1-16, or a pharmaceutically acceptable salt thereof, or a pharmaceutical composition as claimed in claim 17.
